(19) United States
(12) Patent Application Publication
(10) Pub. No.: US 2025/0295761 A1
(43) Pub. Date: Sep. 25, 2025

(54) METHODS FOR PREVENTING DENGUE AND HEPATITIS A

(71) Applicant: Takeda Vaccines, Inc., Cambridge, MA (US)

(72) Inventor: Derek Wallace, Brookline, MA (US)

(73) Assignee: Takeda Vaccines, Inc., Cambridge, MA (US)

(21) Appl. No.: 18/980,441

(22) Filed: Dec. 13, 2024

Related U.S. Application Data
(63) Continuation of application No. 17/869,776, filed on Jul. 20, 2022, now Pat. No. 12,201,683.
Application No. 17/869,776 is a continuation of application No. 16/809,268, filed on Mar. 4, 2020, now Pat. No. 11,426,461.
Application No. 16/809,268 is a continuation-in-part of application No. 16/561,953, filed on Sep. 5, 2019, now Pat. No. 11,590,221.

(30) Foreign Application Priority Data
Sep. 5, 2018 (EP) 18192701.3
Sep. 5, 2018 (EP) 18192711.2
Sep. 5, 2018 (EP) 18192717.9
Sep. 5, 2018 (EP) 18192776.5
Sep. 5, 2018 (EP) 18192787.2
Sep. 5, 2018 (EP) 18192793.0
Sep. 5, 2018 (EP) 18192800.3
Sep. 5, 2018 (EP) 18192814.4
Jan. 29, 2019 (EP) 19154334.7
Mar. 7, 2019 (EP) 19161184.7

(51) Int. Cl. A61K 39/295; A61K 9/00; A61K 39/29; A61K 39/39; A61P 31/14

(52) U.S. Cl. CPC A61K 39/295; A61K 9/0019; A61K 39/29; A61K 39/39; A61P 31/14

(57) ABSTRACT

The invention relates to a method for preventing dengue disease and hepatitis A in a subject or subject population by simultaneously administering a unit dose of a dengue vaccine composition and a hepatitis A vaccine on the same day. The unit dose of a dengue vaccine composition includes constructs of each dengue serotype, such as TDV-1, TDV-2, TDV-3 and TDV-4, at various concentrations in order to improve protection from dengue infection.

### CROSS REFERENCE TO RELATED APPLICATIONS

This Application is a Continuation of U.S. application Ser. No. 17/869,776 filed on Jul. 20, 2022, which is a Continuation of U.S. application Ser. No. 16/809,268 filed on Mar. 4, 2020, which is a Continuation-in-part of U.S. application Ser. No. 16/561,953 filed on Sep. 5, 2019, which claims the benefit of priority from European Application 19161184.7 filed on Mar. 7, 2019, European Application 19154334.7 filed on Jan. 29, 2019, European Application 18192701.3 filed on Sep. 5, 2018, European Application 18192776.5 filed on Sep. 5, 2018, European Application 18192787.2 filed on Sep. 5, 2018, European Application 18192793.0 filed on Sep. 5, 2018, European Application Ser. No. 18/192,800.3 filed on Sep. 5, 2018, European 18192711.2 filed on Sep. 5, 2018, European Application 18192717.9 filed on Sep. 5, 2018, and European Application 18192814.4 filed on Sep. 5, 2018. The entire contents of these applications are incorporated herein by reference in their entirety.

### SEQUENCE LISTING

This application incorporated by reference in its entirety the Sequence Listing entitled “36429US3.xml” which was created on Jul. 21, 2022, and having 127 KB of data and electronically filed herewith.

### FIELD OF THE INVENTION

The present invention relates to a method for administering a unit dose of a dengue vaccine composition to a subject or a subject population simultaneously on the same day with a hepatitis A vaccine. The unit dose according to this invention provides immune responses against all serotypes of dengue virus, i.e. DENV-1, DENV-2, DENV-3 and DENV-4 and against hepatitis A virus.

### BACKGROUND OF THE INVENTION

Vaccines for protection against viral infections have been effectively used to reduce the incidence of human disease. One of the most successful technologies for viral vaccines is to immunize animals or humans with a weakened or attenuated virus strain (a “live attenuated virus”). Due to limited replication after immunization, the attenuated virus strain does not cause disease. However, the limited viral replication is sufficient to express the full repertoire of viral antigens and can generate potent and long-lasting immune responses to the virus. Thus, upon subsequent exposure to a pathogenic virus strain, the immunized individual is protected from the disease. These live attenuated viral vaccines are among the most successful vaccines used in public health.

Dengue disease is a mosquito-borne disease caused by infection with a dengue virus. Dengue virus infections can lead to debilitating and painful symptoms, including a sudden high fever, headaches, joint and muscle pain, nausea, vomiting and skin rashes. To date, four serotypes of dengue virus have been identified: dengue-l (DENV-I), dengue-2 (DENV-2), dengue-3 (DENV-3) and dengue-4 (DENV-4). Dengue virus serotypes 1˜4 can also cause dengue hemorrhagic fever (DHF) and dengue shock syndrome (DSS). In the most severe cases, DHF and DSS can be life threatening. Dengue viruses cause 50-100 million cases of debilitating dengue fever, 500,000 cases of DHF/DSS, and more than 20,000 deaths each year, a large portion of which are children. All four dengue virus serotypes are endemic throughout the tropical regions of the world and constitute the most significant mosquito-borne viral threat to humans there. Dengue viruses are transmitted to humans primarily by Aedes aegypti mosquitoes, but also by Aedes albopictus mosquitoes. Infection with one dengue virus serotype results in life-long protection from re-infection by that serotype, but does not prevent secondary infection by one of the other three dengue virus serotypes. In fact, previous infection with one dengue virus serotype may lead to an increased risk of severe disease (DHF/DSS) upon secondary infection with a different serotype.

To date, only one vaccine, a tetravalent dengue vaccine based on a yellow fever backbone, CYD-TDV (Dengvaxia®, Sanofi Pasteur, Lyon, France), has been licensed in several countries based on the clinical demonstration of an overall vaccine efficacy (VE) against virologically-confirmed dengue (VCD) of 56-61% in children in Asia and Latin America (Capeding M R et al. Clinical efficacy and safety of a novel tetravalent dengue vaccine in healthy children in Asia: a phase 3, randomised, observer-masked, placebo-controlled trial. Lancet 2014, 384:1358-65; Villar L A et al. Safety and immunogenicity of a recombinant tetravalent dengue vaccine in 9-16 year olds: a randomized, controlled, phase II trial in Latin America. Pediatr Infect Dis J 2013, 32:1102-9). However, clinical trials have shown that Dengvaxia® can enhance, rather than reduce, the risk of severe disease due to dengue infection in individuals who had not been previously infected by a dengue virus (seronegative populations). Therefore, Dengvaxia® is only recommended for use in individuals who had been previously infected with at least one dengue virus serotype (seropositive populations). More specifically, according to the European Medicine Agencys European Public Assessment report (EPAR) for the product, Dengvaxia® is only for use in people from 9 to 45 years of age who have been infected with dengue virus before and who live in areas where this infection is endemic. Endemic areas are areas where the disease occurs regularly throughout the year. See also Sridhar S et al. Effect of Dengue Serostatus on Dengue Vaccine Safety and Efficacy. N Engl J Med 2018, 379:327-40; and World Health Organization. Dengue vaccine: WHO position paper-September 2018. Wkly. Epidemiol. Rec. 2018, 93:457-476. S. R. Hadinegoro et al. report in the New England Journal of Medicine, Vol. 373, page 1195, in “Efficacy and Long-Term Safety of a Dengue Vaccine in Regions of Endemic Disease” a pooled risk of hospitalization for virologically-confirmed dengue disease among those under the age of 9 years of 1.58 indicating an increased risk for the vaccinated group with respect to severe dengue. This leaves a substantial unmet need for an effective vaccine with a good safety profile in both dengue-naïve and seropositive individuals, including those dengue-naïve populations living in endemic areas, younger individuals who may not have developed any seropositive response to dengue or been exposed to dengue, and travelers and individuals from non-endemic regions. There is also a need for outbreak control or travel vaccination, offering a reduction in the risk of dengue after only one dose.

One further disadvantage of the only currently approved dengue vaccine, Dengvaxia®, is that it must only be given to people who have had a positive test result showing a previous infection with dengue virus (EPAR), i.e. individuals with known serostatus for dengue. Thus, individuals with unknown serostatus for dengue cannot be vaccinated with Dengvaxia®.

There is hence a need for a dengue vaccine and corresponding method of inoculation that stimulates an immune response to all dengue serotypes, preferably a balanced immune response to all serotypes, and protects against dengue disease of any severity (including DSS, DHF), both in seronegative and seropositive populations, which is safe for a larger group of ages, in particular also for subjects of 9 years and younger. The development of a safe and effective vaccine capable of protecting all populations, including both seronegative and seropositive populations, and in particular children and young adults and elderly subjects in endemic settings and for the purpose of traveling, represents an important approach to the prevention and control of this global disease.

There is thus a medical need for a dengue vaccine and corresponding method of inoculation which, as well as being safe and efficacious irrespective of serostatus and in a broad age group. There is a need for a dengue vaccine and corresponding method of inoculation that avoids costly and time consuming serostatus tests or seroprevalence considerations. There is a need for a dengue vaccine and corresponding method of inoculation that can be used in an outbreak situation. Furthermore there is a medical need for a dengue vaccine which as well as being safe and effective can also be administered to individuals with unknown dengue serostatus, children under 9 years and seronegative individuals.

There is also a need for a vaccine that is administered in fewer doses than the current Dengvaxia® dosing schedule of 3 doses, 6 months apart, such as a vaccine that can be administered in only two doses or one dose to be efficacious.

The above objects are commensurate with the research priorities provided by the WHO in the Dengue Vaccine: WHO position paper-September 2018 (Wkly. Epidemiol. Rec. 2018, 93:457-476).

Hepatitis A is a liver disease caused by the hepatitis A virus (HAV). The virus is primarily spread when an uninfected (and unvaccinated) person ingests food or water that is contaminated with the feces of an infected person. The disease is closely associated with unsafe water or food, inadequate sanitation and poor personal hygiene. The virus can also be transmitted through close physical contact with an infectious person. Unlike hepatitis B and C, hepatitis A infection does not cause chronic liver disease and is rarely fatal, but it can cause debilitating symptoms and fulminant hepatitis (acute liver failure), which is often fatal. Hepatitis A occurs sporadically and in epidemics worldwide, with a tendency for cyclic recurrences.

The hepatitis A virus is one of the most frequent causes of foodborne infection. Epidemics related to contaminated food or water can erupt explosively, such as the epidemic in Shanghai in 1988 that affected about 300,000 people. Hepatitis A viruses persist in the environment and can withstand food-production processes routinely used to inactivate and/or control bacterial pathogens. The disease can lead to significant economic and social consequences in communities. It can take weeks or months for people recovering from the illness to return to work, school, or daily life. The impact on food establishments identified with the virus, and local productivity in general, can be substantial. In developing countries with poor sanitary conditions and hygienic practices, most children (90%) have been infected with the hepatitis

A virus before the age of 10 years.

The number of people traveling internationally has grown substantially in recent decades. According to the United Nations World Tourism Organization (UNWTO), over 1.1 billion tourists travelled abroad in 2014. The risk of becoming ill during international travel depends on many factors, such as the region of the world visited, the length of the trip, and the diversity of planned activities. Vaccine recommendations are a prominent part of health preparations before international travel. Vaccination against hepatitis A virus is commonly recommended for travelers to at-risk areas around the world including Asia, Africa, and Latin America.

For routine hepatitis A vaccination, a two-dose schedule is recommended, particularly in travelers at substantial risk of contracting hepatitis A and in immunocompromised individuals. However, in healthy individuals, comparable effectiveness has been achieved with a single dose. The vaccination schedule for children/adolescents (12 months through 18 years of age) as well as for adults (>19 years of age) consists of a primary dose administered intramuscularly, and a further booster dose administered intramuscularly 6 to 18 months later.

Available hepatitis A vaccines include HAVRIX® and VAQTAR.

Hence, there is a need for a safe and effective method of simultaneously preventing dengue disease and hepatitis A. In particular, there is a need for hepatitis A and dengue vaccines which provide non-inferiority when administered simultaneously to a subject or subject population and a suitable administration schedule for achieving synergy.

Furthermore, there is a need of effectively and safely preventing dengue disease and hepatitis A in subjects being unaware of their hepatitis A and/or dengue serostatus, in particular in subjects from non-endemic countries which travel into dengue and hepatitis A endemic countries.

### Objects and Summary

It is an object of the present invention to provide a safe and effective protection against dengue disease and hepatitis A.

It is an object of the present invention to provide a method of administration for preventing hepatitis A and dengue disease which is useful in typical vaccination settings wherein the subjects are unaware of their serostatus for dengue and/or hepatitis A and a corresponding serotest is unavailable, unpractical or unreliable.

It is an object of the present invention to provide a safe and effective protection against dengue disease and hepatitis A for travelers from hepatitis A and dengue non-endemic countries, in particular for travelers being vaccinated in travel clinics. In this context it is beneficial if multiple during the same medical appointments are avoided and vaccination can be conducted simultaneously for more than one disease.

It is an object of the present invention to provide a safe and effective vaccine for preventing hepatitis A and dengue disease in a subject or subject population and a corresponding method of preventing hepatitis A and dengue disease in a subject or a subject population from a dengue-endemic and dengue non-endemic region and for a broad range of ages, in particular for subjects between 2 to 60 years of age, preferably for subjects between 18 and 60 years of age, and independent of previous exposure to any dengue virus serotype and/or to hepatitis A virus and independent of corresponding seropositivity or seronegativity with respect to dengue and/or hepatitis A before vaccination.

It is an object of the invention to provide vaccines and a corresponding method of preventing hepatitis A and dengue disease which avoids testing for individual dengue and/or hepatitis A serostatus before individual administration of a hepatitis A and a dengue vaccine to a subject or subject population, or analysis of seroprevalence rates of dengue and/or hepatitis A in subjects or subject populations to be vaccinated.

It is an object of the present invention to provide a dengue vaccine and a hepatitis A vaccine which can be safely co-administered with TDV as travel vaccines before an international travel of a subject to HAV and dengue endemic countries and a method of safely administering these vaccines.

Therefore, the present invention is directed to a method of preventing dengue disease as well as hepatitis A.

The present invention is further directed to a method of preventing hepatitis A and dengue disease in a subject or subject population, the method comprising simultaneously on the same day administering a hepatitis A vaccine and a unit dose of a dengue vaccine composition, wherein said unit dose comprises a tetravalent dengue virus composition including four live, attenuated dengue virus strains.

### Definitions

In describing the present invention, the following terms are to be used as indicated below. As used herein, the singular forms “a,” “an,” and “the” include plural references unless the context clearly indicates otherwise.

As used herein, the terms “unit dose of a dengue vaccine composition”, “unit dose” and “unit dose of the invention as described herein” refer to the amount of a dengue vaccine which is administered to a subject in a single dose. In one embodiment, one unit dose is present in a vial and this unit dose is administered to a subject, e.g. optionally after reconstitution. In one embodiment, more than one unit dose of the dengue vaccine composition may be present in a vial so that with the content of one vial more than one subject can be vaccinated.

A “lyophilized unit dose” or “unit dose in lyophilized form” refers to the unit dose that is obtained by subjecting a given volume of the liquid dengue vaccine composition, such as 0.5 mL, to lyophilization. Thus, the aqueous formulations of the dengue vaccine composition being produced by combining the pharmaceutically acceptable excipients and the dengue virus composition comprising the four dengue virus strains, preferably TDV-1 to TDV-4, is subjected to lyophilization to obtain the lyophilized unit dose.

A “reconstituted unit dose” or “unit dose in reconstituted form” is obtained from the lyophilized dose by reconstitution with a pharmaceutically acceptable diluent. The diluent does not contain dengue virus. The reconstituted unit dose is a liquid which can be administered to a subject, for example by injection, such as subcutaneous injection.

As used herein, the term “upon reconstitution with 0.5 mL” is not limiting the reconstitution to be performed using 0.5 mL of the diluent, but refers to the concentration of the dengue viruses that will be present in the reconstituted unit dose when 0.5 mL diluent are used for reconstitution. While using a different volume for reconstitution (e.g. 0.8 mL) will result in a different concentration of dengue viruses in the reconstituted unit dose, the administration of the total volume of the unit dose (e.g. 0.8 mL) will result in the same total amount of dengue virus being administered.

As used herein, a “concentration of at least X log 10 pfu/0.5 mL” refers to the concentration of a dengue serotype in 0.5 mL, but is not limiting the unit dose to be 0.5 mL. If the unit dose has a volume different than 0.5 mL, or is lyophilized from a volume different than 0.5 mL, or is reconstituted with a volume different than 0.5 mL, said concentration will differ from the “concentration of at least X log 10 pfu/0.5 mL”. However, if the unit dose has a volume of 0.5 mL, or is lyophilized from a volume of 0.5 mL, or is reconstituted with a volume of 0.5 mL, said concentration will be the “concentration of at least X log 10 pfu/0.5 mL”. Thus, while the concentration may differ, the total amount of virus in the unit dose remains the same.

As used herein, the term “dengue serotype” refers to a species of dengue virus which is defined by its cell surface antigens and therefore can be distinguished by serological methods known in the art. At present, four serotypes of dengue virus are known, i.e. dengue serotype 1 (DENV-1), dengue serotype 2 (DENV-2), dengue serotype 3 (DENV-3) and dengue serotype 4 (DENV-4).

As used herein, the term “tetravalent dengue virus composition” refers to a dengue virus composition comprising four different immunogenic components from the four different dengue serotypes DENV-1, DENV-2, DENV-3 and DENV-4, preferably comprising four different live, attenuated dengue viruses, each representing one dengue serotype, and which aims to stimulate immune responses to all four dengue serotypes.

As used herein, the term “live attenuated dengue virus” refers to a viable dengue virus which is mutated to provide reduced virulence. The live attenuated dengue virus can be a dengue virus in which all components are derived from the same dengue serotype or it can be a chimeric dengue virus having parts from two or more dengue serotypes or a mixed chimeric dengue virus having parts from other flaviviruses.

A “virus strain” and in particular a “dengue virus strain” is a genetic subtype of a virus, in particular of a dengue virus, which is characterized by a specific nucleic acid sequence. A dengue serotype may comprise different strains with different nucleic acid sequences which have the same cell surface antigens. A dengue virus strain can be a dengue virus in which all components are derived from the same dengue serotype or it can be a chimeric dengue virus having parts from two or more dengue serotypes.

As used herein, “TDV-2” refers to a molecularly characterized and cloned dengue serotype 2 strain derived from the live attenuated DEN-2 PDK-53 virus strain. The PDK-53 strain is described for example in Bhamarapravati et al. (1987) Bulletin of the World Health Organization 65 (2): 189-195. In one embodiment, the TDV-2 strain served as a backbone for the chimeric TDV-1, TDV-3 and TDV-4 strains into which parts from the TDV-1, TDV-3 and TDV-4 strains were introduced.

A “non-chimeric dengue virus” or “non-chimeric dengue serotype strain” or “non-chimeric dengue strain” comprises only parts from one dengue serotype. In particular, a non-chimeric dengue virus does not include parts from a different flavivirus such as yellow fever virus, Zika virus, West Nile virus, Japanese encephalitis virus, St. Louis encephalitis virus, tick-borne encephalitis virus. TDV-2 is an example of a non-chimeric dengue virus.

A “chimeric dengue virus” or “chimeric dengue serotype strain” or “chimeric dengue strain” comprises parts from at least two different dengue serotypes. As used herein, the chimeric dengue virus does not include parts from a different flavivirus such as yellow fever virus, Zika virus, West Nile virus, Japanese encephalitis virus, St. Louis encephalitis virus, tick-borne encephalitis virus. In particular, the chimeric dengue virus described herein does not include parts from the yellow fever virus. As used herein, a “chimeric dengue serotype 2/1 strain” or “DENV-2/1 chimera” or “TDV-1” refers to a dengue virus chimeric construct which comprises parts from both DENV-2 and DENV-1. In particular, in the chimeric dengue serotype 2/1 strain the prM and E proteins from DENV-1 replace the prM and E proteins from DENV-2 as detailed below. As used herein, a “chimeric dengue serotype ⅔ strain” or “DENV-⅔ chimera” or “TDV-3” refers to a dengue virus chimeric construct which comprises parts from both DENV-2 and DENV-3. In particular, in the chimeric dengue serotype ⅔ strain the prM and E proteins from DENV-3 replace the prM and E proteins from DENV-2 as detailed below. As used herein, a “chimeric dengue serotype 2/4 strain” or “DENV- 2/4 chimera” or “TDV-4” refers to a dengue virus chimeric construct which comprises parts from both DENV-2 and DENV-4. In particular, in the chimeric dengue serotype 2/4 strain the prM and E proteins from DENV-4 replace the prM and E proteins from DENV-2 as detailed below. A mixed chimeric dengue virus has parts from other flaviviruses.

As used herein, “TDV” refers to a tetravalent live attenuated dengue vaccine that comprises a mixture of the four live attenuated dengue virus strains TDV-1, TDV-2, TDV-3 and TDV-4 expressing surface antigens from the four dengue serotypes DENV-1, DENV-2, DENV-3 and DENV-4, respectively. In one embodiment (e.g. also in the examples), TDV-1 has the nucleotide sequence according to SEQ ID No. 1 and/or the amino acid sequence according to SEQ ID No. 2. In one embodiment, TDV-2 has the nucleotide sequence according to SEQ ID No. 3 and/or the amino acid sequence according to SEQ ID No. 4. In one embodiment, TDV-3 has the nucleotide sequence according to SEQ ID No. 5 and/or the amino acid sequence according to SEQ ID No. 6. In one embodiment, TDV-4 has the nucleotide sequence according to SEQ ID No. 7 and/or the amino acid sequence according to SEQ ID No. 8.

As used herein, the term “dengue disease” refers to the disease which is caused by infection with dengue virus. Symptoms of dengue disease include sudden high fever, headaches, joint and muscle pain, nausea, vomiting and skin rashes. The term dengue disease also includes the more severe forms of dengue hemorrhagic fever (DHF) and dengue shock syndrome (DSS). Symptoms of DHF include increased vascular permeability, hypovolemia and abnormal blood clotting mechanisms. Subjects with DHF may present with severe manifestations of plasma leakage and hemorrhage. When a subject with DHF experiences shock he or she will be categorized as having DSS. Symptoms of DSS include bleeding that may appear as tiny spots of blood on the skin and larger patches of blood under the skin. Prolonged shock is the main factor associated with complications including massive gastrointestinal hemorrhage that can lead to death. As used herein, DHF cases are defined as VCD cases meeting WHO 1997 DHF criteria. In the context of preventing dengue disease in elderly subjects, the term “preventing dengue disease” preferably includes preventing DHF and/or DSS. In the context of preventing dengue disease in elderly subjects, the term “preventing dengue disease” preferably includes preventing severe end-organ manifestations of dengue such as hepatomegaly and acute renal failure.

As used herein, “preventing dengue disease” refers to preventing a subject from developing one or more symptoms of dengue disease because of an infection with a dengue virus. In particular, preventing dengue disease is achieved by vaccinating or inoculating a subject with a dengue vaccine composition, such as the reconstituted unit dose described herein. As used herein, the term “prophylactically treating dengue disease” is equivalent to “preventing dengue disease”. In a particular embodiment, preventing dengue disease includes preventing DHS and/or DSS.

As used herein, the terms “virologically-confirmed dengue disease”, “VCD case”, or “VCD fever” refer to febrile illness or illness clinically suspected to be dengue disease with a positive serotype-specific reverse transcriptase polymerase chain reaction (RT-PCR). The term “virologically confirmable dengue” disease refers to a subject having febrile illness or illness clinically suspected to be dengue disease, wherein testing the subject, e.g. using RT-PCR, would confirm the presence of at least one dengue serotype. Severe forms of VCD fever will be identified as follows: Dengue Hemorrhagic Fever (DHF) was defined according to the WHO 1997 criteria. Severe dengue was defined through an assessment of an independent Dengue Case Adjudication Committee which will assess all hospitalized VCD cases (severe/non-severe) based on criteria redefined in a charter. All non-hospitalized cases are considered non-severe.

As used herein, the term “febrile illness” is defined as temperature≥38° C. on any 2 of 3 consecutive days.

As used herein, the terms “virologically-confirmed dengue disease with hospitalization”, is considered to be a surrogate for severe dengue and the “incidence of virologically-confirmed dengue disease with hospitalization” is used as a safety parameter. As used herein, the “relative risk with respect to virologically-confirmed dengue disease with hospitalization” means the number of events of virologically confirmed dengue disease with hospitalization divided by the number of subjects treated with the unit dose as disclosed herein over the number of events of virologically confirmed dengue disease with hospitalization divided by the number of subjects treated with placebo. If the “relative risk with respect to virologically-confirmed dengue disease with hospitalization” is 1 or lower the vaccine provides for the same or less risk for virologically-confirmed dengue disease with hospitalization as placebo and is considered “safe”. In this context the risk of virologically-confirmed dengue disease with hospitalization may be also 0.9 or less, 0.8 or less, 0.7 or less, 0.6 or less, 0.5 or less, 0.4 or less, 0.3 or less, 0.2 or less, or 0.1 or less, in particular when determined from 30 days after a second administration until 12 months after a second administration, in particular when determined in age groups selected from the age group of 4 to 16 year old subjects, the age group of 4 to under 9 year old subjects, the age group of 2 to under 9 year old subjects, the age group of 4 to 5 year old subjects, the age group of 6 to 11 year old subjects, and the age group of 12 to 16 year old subjects.

As used herein, alternatively a vaccine is considered “safe” when the vaccine efficacy (VE) with respect to virologically-confirmed dengue disease with hospitalization is 0% or higher. This means that the vaccine provides for the same likelihood or less for virologically-confirmed dengue disease with hospitalization as placebo. In particular considered “safe” is the combined vaccine efficacy against virologically-confirmed dengue with hospitalization against all four serotypes with a 2-sided 95% confidence interval, wherein the lower bound is more than 25%, in particular when measured against placebo in a subject population of at least 1,500 or at least 2,000 healthy subjects (in particular when measured in age groups selected in particular from the age group of 4 to 16 year old subjects, the age group of 4 to under 9 year old subjects, the age group of 2 to under 9 year old subjects, the age group of 4 to 5 year old subjects, the age group of 6 to 11 year old subjects, and the age group of 12 to 16 year old subjects) being seronegative against all serotypes at baseline or being seropositive against at least one serotype at baseline, in particular when said unit dose or said placebo is administered at least twice within less than 6 months, such as within 3 months, about from first administration or from 30 days after the second or last administration of the administration schedule until at least 12 months, until 12 to 18 months, until 12 months, or until 18 months after the second or last administration of the administration schedule. In particular, the lower bound may be more than 30%, more than 40%, more than 50%, more than 60%, more than 65%, more than 66%, more than 67%, more than 68% more than 70%, or more than 75%. In particular, the 2-sided 95% confidence interval of the combined vaccine efficacy against virologically-confirmed dengue with hospitalization against all four serotypes when comparing seropositive and seronegative subjects provides for lower bounds of the 2-sided confidence interval which are within 10% points or within 15% points or within 20% points. In a particular embodiment “safe” means providing a combined vaccine efficacy against virologically-confirmed dengue with hospitalization against all four serotypes with a 2-sided 95% confidence interval, wherein the lower bound is more than 65%, when measured against placebo in a subject population of at least 5,000 healthy 4 to 16 year old subjects irrespective of serostatus at baseline from first administration of the administration schedule until 12 to 18 months after the last administration of the administration schedule.

If one of the criteria as defined above for the term “safe” is fulfilled, the vaccine is considered safe within the meaning of this invention. In this context, safe in particular refers to a vaccine that is safe for all subjects irrespective of their serostatus at baseline. This means that the vaccine can be administered without the need to determine the occurrence of a previous dengue infection in the subject before administration. Preferably, the vaccine is safe as defined above with respect to all age groups starting from 4 years of age and preferably irrespective of the serostatus, in particular from 4 years of age to 60 years of age, or 4 years of age to 16 years of age. Relevant subgroups in this context are under 9 years of age, from 2 years of age to under 9 years of age, from 4 years of age to under 9 years of age, 4 to 5 years of age, 6 to 11 years of age and 12 to 16 years of age or any age group within 4 to 16 years of age. For further definitions of VE against virologically-confirmed dengue disease with hospitalization reference is made to the disclosure below with respect to certain methods of treatment.

As used herein, “vaccine efficacy” or “VE” measure the proportionate reduction in cases among vaccinated persons. Vaccine efficacy (VE) is measured by calculating the risk of disease among vaccinated and unvaccinated persons and determining the percentage reduction in risk of disease among vaccinated persons relative to unvaccinated persons. The greater the percentage reduction of illness in the vaccinated group, the greater the vaccine efficacy. For example, a VE of 90% indicates a 90% reduction in disease occurrence among the vaccinated group, or a 90% reduction from the number of cases you would expect if they have not been vaccinated. The vaccine efficiency is calculated by the formula:

100*(1−HR),

wherein HR is the Hazard Ratio which is defined as the Hazard rate of vaccine (λv) divided by the Hazard rate of placebo (λc), i.e. HR=λv/λc. Av denote the hazard rate for the subjects vaccinated with a tetravalent dengue vaccine composition as disclosed herein and λc denote the hazard rate for unvaccinated subjects, i.e. subjects receiving placebo. The hazard rate ratio HR is estimated from a Cox proportional hazard model with study vaccine as a factor, adjusted for age, and stratified by region. As used herein the term “combined vaccine efficacy against all four serotypes” is defined as the vaccine efficacy in relation to the risk of dengue disease irrespective of the serotype being responsible for the virologically-confirmed dengue disease and the subject baseline serostatus. A vaccine is considered “effective” in case the combined vaccine efficacy is above 30%. In this context the combined vaccine efficacy may be also 40% or more, 50% or more, 60% or more, 70% or more, 72% or more, or 80% or more, in particular when determined from 30 days after a second administration until 12 months after a second administration or 18 months after a second vaccination, in particular when determined in age groups selected from the age group of 4 to 16 year old subjects, the age group of 4 to under 9 year old subjects, the age group of 2 to under 9 year old subjects, the age group of 4 to 5 year old subjects, the age group of 6 to 11 year old subjects, and the age group of 12 to 16 year old subjects. In this context, effective in particular refers to a vaccine that is effective for all subjects irrespective of their serostatus at baseline. Preferably, the vaccine is effective with respect to all age groups starting from 4 years of age and preferably irrespective of the serostatus, in particular from 4 years of age to 60 years of age or from 4 years of age to 16 years of age and irrespective of the serostatus. Relevant subgroups in this context are under 9 years of age, from 2 years of age to under 9 years of age, from 4 years of age to under 9 years of age, 4 to 5 years of age, 6 to 11 years of age and 12 to 16 years of age or any age group within 4 to 16 years of age. In certain embodiments “effective” means providing a combined vaccine efficacy against all four serotypes, in preventing virologically confirmable dengue disease with a 2-sided 95% confidence interval, wherein the lower bound is more than 60%, when measured against placebo in a subject population of at least 5,000 healthy subjects irrespective of serostatus at baseline and 4 to 16 years of age, from the first administration of the administration schedule until 18 months after the last administration of the administration schedule. Further specific efficacies can be defined. As used herein, “combined vaccine efficacy against all four serotypes in seronegative subjects” refers to the efficacy measured in subjects which are seronegative at baseline. As used herein, “vaccine efficacy against a specific serotype, e.g. serotype 1” refers to the efficacy in relation to a specific serotype being responsible for the virologically-confirmed dengue disease. As used herein, “combined vaccine efficacy against all four serotypes against virologically-confirmed dengue with hospitalization” refers to the efficacy wherein only virologically-confirmed dengue cases with hospitalization are considered. Such vaccine efficacies can be determined with respect to subjects being seronegative or seropositive at baseline and for different age groups.

As used herein, the “relative risk” means the number of events of virologically confirmed dengue disease divided by the number of subjects treated with the unit dose as disclosed herein over the number of events of virologically confirmed dengue disease divided by the number of subjects treated with placebo. As used herein the term “combined relative risk against all four serotypes” is defined as the relative risk in relation to the risk of dengue disease irrespective of the serotype being responsible for the virologically-confirmed dengue disease and the subject baseline serostatus.

As used herein, “vaccinating” or “inoculating” refers to the administration of a vaccine to a subject, with the aim to prevent the subject, from developing one or more symptoms of a disease. As used herein, “vaccinating against dengue disease” or “inoculating against dengue disease” refers to the administration of a dengue vaccine composition to a subject, with the aim to prevent the subject, from developing one or more symptoms of dengue disease. In principle the method comprises a primary vaccination and optionally one or more booster vaccinations. The primary vaccination is defined as the primary administration schedule for administering the composition or unit dose as disclosed herein to establish a protective immune response and e.g. consists of two administrations e.g. within three months. Whenever an administration is mentioned within this disclosure such administration refers to the primary vaccination unless it is specified as booster vaccination. The booster vaccination refers to an administration or administration schedule which takes place after the primary vaccination e.g. at least 1 year, or 4 to 4.5 years, or even 5 or 10 years after the last administration, e.g. the second administration, of the primary vaccination schedule. The booster administration attempts at enhancing or reestablishing the immune response of the primary vaccination.

As used herein, the terms “subject” or “subjects” are limited to human subjects (e.g. infants, children or adults). The terms “elderly subject” or “elderly subjects” refer to subjects with an age of more than 60 years, such as 61 years to 100 years, 61 years to 90 years, 61 years to 80 years, 61 years to 75 years, or 61 years to 70 years.

As used herein, “subject population” refers to a group of subjects. The subject population may refer to least 40 subjects, at least 50 subjects, at least 60 subjects, at least 100 subjects or at least 1000 subjects and is defined by certain parameters. The parameters that may be used to define a subject population include, but are not limited to, the age of the subjects, whether the subjects are from a dengue endemic region or from a dengue non-endemic region and the serostatus of the subjects.

As used herein, “endemic region” refers to a region where a disease or infectious agent is constantly present and/or usually prevalent in a population within this region. As used herein, “non-endemic region” refers to a region from which the disease is absent or in which it is usually not prevalent. Accordingly, a “dengue endemic region” refers to geographic areas in which an infection with dengue virus is constantly maintained at a baseline level. A “dengue non-endemic region” is a geographic area in which an infection with dengue virus is not constantly maintained at a baseline level. Accordingly, subject populations or subjects “from a dengue endemic region” or “from a dengue non-endemic region” refer to subject populations or subjects living in geographic areas as defined above. Whether a geographic area or a subject population is dengue-endemic or not can be determined by different calculatory methods such as the ones described in Bhatt et al. (2013) Nature 496 (7446): 504-507 and supplementary material and in Stanaway et al. (2016) Lancet Infect Dis. 16 (6): 712-723 and supplementary material. Overviews of dengue endemic regions and dengue epidemiology are regularly published, for example, by the WHO or CDC. Typical dengue-endemic regions are in Latin America, Southeast Asia and the Pacific islands and dengue endemic countries include, but are not limited to, Australia, Brazil, Bangladesh, Colombia, China, Dominican Republic, Indonesia, India, Mexico, Malaysia, Nicaragua, Nigeria, Pakistan, Panama, Philippines, Puerto Rico, Singapore, Sri Lanka, Thailand and Vietnam. The area's force of infection is measured by seroprevalence surveys provided as seroprevalence rate. Areas with very high force of infection are considered to have a seroprevalence rate of more than 80%. As used herein the term “region” when it concerns seroprevalence rates refers to a geographic area where the seroprevalence rate could be determined or is known, e.g. a village, a town, a city, a region, a county, a state, a province or parts of the foregoing or a whole country.

As used herein, “serostatus” refers to the amount of antibodies a subject has with respect to a certain infectious agent, in particular dengue virus. As used herein, “seronegative” or “seronaïve” means that the subject does not have neutralizing antibodies against any one of dengue serotypes DENV-1, DENV-2, DENV-3 and DENV-4 in the serum. A seronegative or seronaïve subject or subject population is defined by a neutralizing antibody titer of less than 10 for each one of the four dengue serotypes. A subject or subject population having a neutralizing antibody titer of equal to or more than 10 for at least one dengue serotype is defined as being “seropositive” with respect to said dengue serotype. Serostatus at baseline refers to the serostatus before the administration of a dengue vaccine composition as described herein.

As used herein, a “neutralizing antibody titer” refers to the amount of antibodies in the serum of a subject that neutralize the respective dengue serotype. The neutralizing antibody titer against DENV-1, DENV-2, DENV-3 and DENV-4 is determined in a serum sample of the subject using known methods such as the plaque reduction neutralization test (PRNT) as described in the WHO Guidelines (World Health Organization Department of Immunization Vaccines Biologicals (2007) Guidelines for plaque reduction neutralization testing of human antibodies to dengue viruses, WHO/IVB/07.07) or a microneutralization (MNT50) assay as described herein. As used herein, the “ratio of not more than 20 for the neutralizing antibody titer of dengue serotype 2 to the neutralizing antibody titer of dengue serotype 4” means that the neutralizing antibody titer of dengue serotype 2 is divided by the neutralizing antibody titer of dengue serotype 4 and that the ratio obtained hereby is no more than 20. In other words, the neutralizing antibody titer of dengue serotype 2 is not more than 20-times higher than the neutralizing antibody titer of dengue serotype 4 in the subject.

As used herein, the terms “geometric mean neutralizing antibody titer” and “GMT” refer to the geometric mean value of the titer of neutralizing antibodies against the corresponding dengue serotype in the serum of subjects in a subject population. The geometric mean value is calculated by a well-known formula. As used herein, the “ratio of not more than 20 for the GMT of dengue serotype 2 to the GMT of dengue serotype 4” means that the geometric mean neutralizing antibody titer of dengue serotype 2 (GMT DENV-2) is divided by the geometric mean neutralizing antibody titer of dengue serotype 4 (GMT DENV-4) and that the ratio obtained hereby is no more than 20. In other words, the geometric mean neutralizing antibody titer of dengue serotype 2 is not more than 20-times higher than the geometric mean neutralizing antibody titer of dengue serotype 4 in the subject population.

As used herein, an “immune response” refers to a subject's response to the administration of the dengue vaccine. In particular, the immune response includes the formation of neutralizing antibodies to one or more dengue serotypes. It may also include the stimulation of a cell-mediated response or the formation of antibodies to non-structural proteins such as NS1. An immune response is stimulated by the administration of a unit dose of the invention as described herein, if the titer of neutralizing antibodies against at least one dengue virus serotype and preferably against all four dengue virus serotypes is increased after said administration of said unit dose. An immune response is stimulated by the administration of a unit dose of the invention as described herein, if the secretion of interferon gamma by peripheral blood mononuclear cells stimulated with peptides from dengue virus proteins is increased after said administration of said unit dose. An immune response is stimulated by the administration of a unit dose of the invention as described herein, if the titer of antibodies to non-structural proteins such as NS1 is increased after said administration of said unit dose. In a particular embodiment, the administration of a reconstituted unit dose of the present invention as described herein stimulates the formation of neutralizing antibodies to one or more dengue serotypes, a cell-mediated response and the formation of antibodies to non-structural proteins such as NS1.

As used herein, a “balanced immune response” means that the immune response to the four dengue serotypes is sufficient to provide protection against infection by all four dengue serotypes and preferably the immune response to the four dengue serotypes has a similar strength. In particular, the neutralizing antibody titer against the four dengue serotypes at day 180 or day 365 after administration of a first reconstituted unit dose of the invention as described herein is similar, i.e. it differs by less than factor 30, by less than factor 25 or by less than factor 20.

The “total concentration in pfu/0.5 ml” which serves as a base value for the calculation of the percentage concentration for each individual component of a tetravalent dengue vaccine is shown for one exemplary tetravalent vaccine composition comprising dengue serotype 1 in a concentration of 3.60 log 10 pfu/0.5 ml, a dengue serotype 2 concentration of 4.00 log 10 pfu/0.5 ml, a dengue serotype 3 concentration of 4.60 log 10 pfu/0.5 ml and a dengue serotype 4 concentration of 5.11 log 10 pfu/0.5 ml. Primarily, the logarithmic values of the concentrations are converted into numerical values. The results of this conversion are 4×10³ pfu/0.5 ml for serotype 1, 1×10⁴ pfu/0.5 ml for serotype 2, 4×10⁴ pfu/0.5 ml for serotype 3 and 1.3×10⁵ pfu/0.5 ml for serotype 4. The total concentration in pfu/0.5 ml is the sum of the preceding numerical values resulting in 1.84×10⁵ pfu/0.5 ml.

The “percentage concentration” for each of the serotypes 1, 2, 3 and 4 is obtained by dividing the numerical concentration value (expressed as pfu/0.5 ml) of an individual serotype by the total concentration (expressed in pfu/0.5 ml) and multiplying the result by 100 i.e.: Percentage concentration of serotype 1=(4×10³ pfu/0.5 ml=1.84×10⁵ pfu/0.5 ml)×100=2% Percentage concentration of serotype 2=(1×10⁴ pfu/0.5 ml=1.84×10⁵ pfu/0.5 ml)×100=5% Percentage concentration of serotype 3=(4×10⁴ pfu/0.5 ml=1.84×10⁵ pfu/0.5 ml)×100=22% Percentage concentration of serotype 4=(1.3×10⁵ pfu/0.5 ml=1.84×10⁵ pfu/0.5 ml)×100=71%. The percentage concentrations are rounded to whole numbers.

As used herein “simultaneous” administration means an administration of at least two different vaccines such as a dengue vaccine and a hepatitis A vaccine on the same day. “On the same day” has the ordinary meaning of within 24 hours, such as e.g. within one calendar day. The simultaneous administration may be administered by the same medical practitioner, such as during the same medical appointment.

As used herein “sequential” administration means an administration of at least two different vaccines, such as a dengue vaccine and a yellow fever vaccine, or a dengue vaccine and a hepatitis A vaccine on different or subsequent days, such as within 90 days, but in a combined administration schedule.

As used herein, the term “chronic disease or condition” includes those diseases and conditions which persist in an elderly subject for three months or more. In particular, it includes diabetes, hypertension, allergies, previous strokes, ischemic heart disease, chronic renal impairment and chronic obstructive pulmonary disease.

As used herein, the term “impaired immune system” means that at least one function of at least one component of the immune system is weaker than in younger subjects, i.e. in subjects with an age of less than 60 years. These functions include a lower antioxidant response of monocytes against oxidative stress induced by dengue virus and lower T cell responses and cytokine production in response to dengue virus infection.

As used herein, “solicited systemic adverse events” in children under 6 years are defined as fever, irritability/fussiness, drowsiness and loss of appetite that occurred within 14 days after each vaccination, and in children of 6 years or more are defined as fever, headache, asthenia, malaise and myalgia that occurred within 14 days after each vaccination.

As used herein, “solicited local adverse events” are injection site pain, injection site erythema and injection site swelling that occurred within 7 days after each vaccination.

As used herein, “unsolicited adverse events” are any adverse events (AEs) that are not solicited local or systemic AEs, as defined above.

As used herein, a “serious adverse event” or “SAE” is any untoward medical occurrence or effect that at any dose results in death, is life-threatening, requires inpatient hospitalization or prolongation of existing hospitalization, results in persistent or significant disability/incapacity, is a congenital anomaly/birth defect or is medically important due to other reasons than the above mentioned criteria.

The relationship of each AE, including solicited systemic AEs (solicited local AEs are considered as related) to trial vaccine(s) will be assessed using the following categories: As used herein, “IP-Related AE” or “vaccine related AE” means that there is suspicion that there is a relationship between the vaccine and the AE (without determining the extent of probability); there is a reasonable possibility that the vaccine contributed to the AE. As used herein, “Non-IP Related” or “non-vaccine related” means that there is no suspicion that there is a relationship between the vaccine and the AE; there are other more likely causes and administration of the vaccine is not suspected to have contributed to the AE.

As used herein, a subject or subject population being “2 to 60 years of age” “or 18 to 60 years of age” refers to a subject or subject population being 2 to 60 years of age or 18 to 60 years of age on the first day of the administration of the dengue vaccine composition as described herein.

As used herein “%-points” refers to the difference of two %-values in a %-value. For example two values in % which are within 5%-points refers to e.g. one value at 1% and a second value at 6%.

As used herein, the term “determination of the previous dengue infection in the subject before administration” means that a previous dengue infection has to be assessed before vaccination in that there is a laboratory confirmed history of dengue or through an appropriately validated serological test e.g. by the method as disclosed herein such as the MNT50 test described in Example 2 or any serotesting with adequate performance in terms of specificity and cross reactivity based on the locale disease epidemiology.

As used herein % w/v refers to % mg/ml wherein e.g. 150 mg/ml are 15% w/v.

As used herein, the term “hepatitis A virus” may be abbreviated as “HAV”.

As used herein, the term “placebo” may be abbreviated as “Pbo”.

As used herein, “hepatitis A seronegative at baseline” or “hepatitis A naïve (at baseline)” each mean that a subject does not have a predefined amount of anti-hepatitis A antibodies in the serum. Quantitatively, the hepatitis A seronegativity of a subject is defined as an anti-hepatitis A antibody level of<10 mIU/ml. When anti-hepatitis A antibody levels are determined by ELISA, the lower level of quantification is 12.5 mIU/ml which is effectively the lower anti-HAV antibody level for determining seronegativity. Subjects having anti-hepatitis A antibody levels of ≥12.5 mIU/ml are defined as hepatitis A seropositive. An ELISA for determining the anti-hepatitis A antibodies is for example disclosed in Beck et al. J Travel Med 2004; 11:201-207.

As used herein, “at baseline” refers to the time point of the last measurement of a subject's serostatus prior to the first vaccination.

As used herein, the unit “mIU/ml” refers to milli-international unit per milliliter. This concentration unit refers to a quantity of anti-hepatitis A antibodies in a subject's serum (e.g. when measured prior or after vaccination). As used herein, the “viral antigen activity of hepatitis A vaccines” of the present invention is expressed in terms of a standard recommendation of the WHO using an enzyme-linked immunosorbent assay (ELISA). According to this recommendation of the WHO (see WHO Information Sheet “Observed Rate of Vaccine Reactions-Hepatitis A Vaccine”, published June 2012), the viral antigen activity of a hepatitis A vaccine is expressed in terms of ELISA Units (EL.U.). The viral antigen activity of a hepatitis A vaccine can for example be determined by an ELISA according to Andre F E., Hepburn A. D′Hondt E., “Inactivated candidate vaccines for hepatitis”, A. Prog Med Virol 1990; 37:72-95.

As used herein, the term “CCID” refers to the quantity of virus (e.g. vaccinal virus) infecting 50% of the cell culture. The CCID50 assay is a limit dilution assay with statistical titer calculation (Morrison D et al, J Infect Dis. 2010; 201 (3): 370-7)).

“Non-inferiority”, as used herein, with respect to a simultaneous on the same day administration of a hepatitis A vaccine and a tetravalent dengue vaccine is in particular concluded, if the seroprotection rate (SPR) difference between the SPR of a subject group receiving HAV and placebo (simultaneously on the same day, i.e. control subject population) and the SPR of a subject group receiving HAV and TDV (simultaneously on the same day) has an upper bound of a two-sided 95% confidence interval which is lower than the non-inferiority margin set at 10%, wherein seroprotection rates are based on measurements on day 30 after the simultaneous administration on day 1, calculated using the Newcombe score method. A non-inferiority clinical study is a study designed to provide a comparison between at least two methods of treatments, in the present case between a simultaneous administration of a dengue vaccine and a hepatitis A vaccine and a mono-administration of either a dengue vaccine or a hepatitis A vaccine.

As used herein, the term “seroprotection rate”, abbreviated “SPR”, is defined by the proportion/percentage of HAV or DEN-naive subjects at baseline who are seroprotected against HAV or DENV, respectively, at day 30 (month 1) after the first vaccination.

As used herein, the term “control subject population” refers to a group of subjects which does not receive a simultaneous administration of a hepatitis A vaccine and a unit dose of a dengue vaccine composition, but a single verum (such as a hepatitis A vaccine or a unit dose of a dengue vaccine composition) and a placebo on the same day in a clinical study setting as e.g. in a non-inferiority clinical study.

As used herein, the term “synergism” or “synergy” is defined as an effect of simultaneously on the same day administering the hepatitis A vaccine and the unit dose of the dengue vaccine composition to a subject or subject population, wherein said administering provides a higher anti-hepatitis A antibody concentration and/or a higher mean titer of neutralizing antibodies against each of the dengue virus serotypes than the corresponding simultaneous administration of a hepatitis A vaccine and a placebo on the same day and/or the simultaneous administration of a unit dose of the dengue vaccine composition and a placebo on the same day (mono-administrations). Such higher antibody concentrations after simultaneous administration in comparison to the mono-administrations are signs in favor of the simultaneous administration.

### BRIEF DESCRIPTION OF THE DRAWINGS

FIG. 1: Genetic structure of the four dengue strains contained in TDV. The solid red triangles indicate the three attenuating mutations present in the 5′NCR, NS1 and NS3 proteins. The TDV-1, TDV-3 and TDV-4 strains are chimeric viruses where the prM and E genes from dengue serotype 1, 3 and 4, respectively, are inserted into the TDV-2 backbone.

FIG. 2: Schematic drawing illustrating the microneutralization test (MNT) used to determine the titer of neutralizing antibodies.

FIG. 3: Flow diagram of the clinical trial of Example 3.

FIG. 4: Cumulative incidence of A) virologically-confirmed dengue cases and B) hospitalized virologically-confirmed dengue cases over time during Part 1 study period by baseline serostatus (safety set data; data presented truncated at Month 18). Tables show numbers of participants under follow-up at various time points to end of Part 1 study period.

FIG. 5: Study design of phase III study described in example 3.

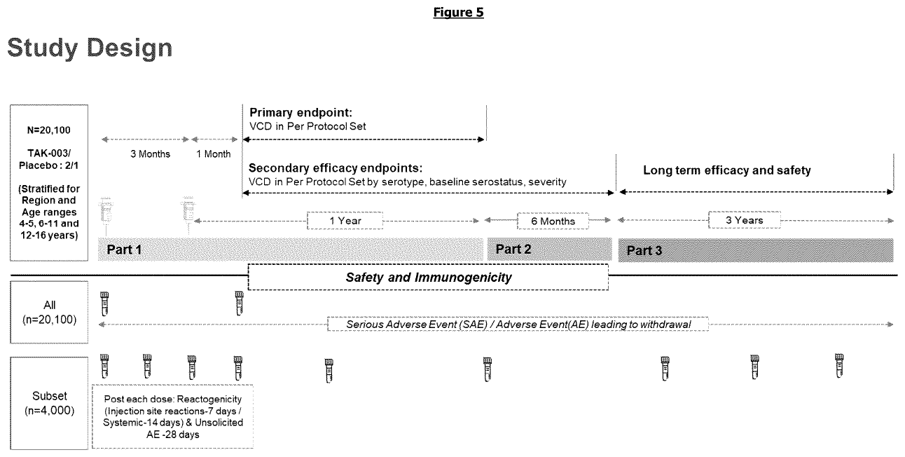

FIG. 6: Scheme of the trial design of the simultaneous HAV and TDV administration study described in Example 4.

### DETAILED DESCRIPTION

### Dengue Virus Strains

The dengue virus is a single stranded, positive sense RNA virus of the family flaviviridae. The taxonomy is outlined in Table 1. The family flaviviridae includes three genera, flavivirus, hepacivirus and pestivirus. The genus flavivirus contains highly pathogenic and potentially hemorrhagic fever viruses, such as yellow fever virus and dengue virus, encephalitic viruses, such as Japanese encephalitis virus, Murray Valley encephalitis virus and West Nile virus, and a number of less pathogenic viruses.

TABLE 1
Dengue Virus Taxonomy of the GMO Parental Strain

| | |
|---|---|
| Family | Flaviviridae |
| Genus | Flavivirus |
| Species | Dengue virus |
| Strains | Dengue Serotype 2 (Strain 16681), Strain DEN-2 PDK-53 |
| GMO parent | TDV-2 |

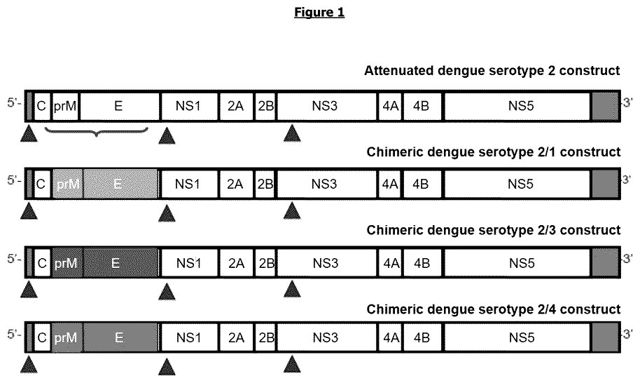

The flavivirus genome comprises in 5′ to 3′ direction (see FIG. 1):
a 5′-noncoding region (5′-NCR),
a capsid protein (C) encoding region,
a pre-membrane protein (prM) encoding region,
an envelope protein (E) encoding region,
a region encoding nonstructural proteins (NSI, NS2A, NS2B, NS3, NS4A, NS4B, NS5) and
a 3′ noncoding region (3′-NCR).

The viral structural proteins are C, prM and E, and the nonstructural proteins are NSI to NS5. The structural and nonstructural proteins are translated as a single polyprotein and processed by cellular and viral proteases.

The unit dose of the invention as described herein comprises a dengue virus composition that comprises four live attenuated dengue virus strains (tetravalent dengue virus composition) representing dengue serotype 1, dengue serotype 2, dengue serotype 3 and dengue serotype 4. Preferably the composition comprises chimeric dengue viruses and optionally at least one non-chimeric dengue virus, in particular a molecularly characterized and cloned dengue serotype 2 strain derived from the live attenuated DEN-2 PDK-53 virus strain (TDV-2), and three chimeric dengue strains derived from the TDV-2 strain by replacing the structural proteins prM and E from TDV-2 with the corresponding structural proteins from the other dengue serotypes, resulting in the following chimeric dengue strains:
a DENV-2/1 chimera (TDV-1),
a DENV-⅔ chimera (TDV-3) and
a DENV- 2/4 chimera (TDV-4).

The genetically modified tetravalent dengue vaccine TDV is based on a molecularly characterized and cloned dengue-2 virus strain (TDV-2). This attenuated TDV-2 strain was generated by cDNA cloning of the attenuated laboratory-derived DEN-2 PDK-53 virus strain that was originally isolated at Mahidol University, Bangkok, Thailand (Kinney et al. (1997) Virology 230 (2): 300-308). DEN-2 PDK-53 was generated by 53 serial passages in primary dog kidney (PDK) cells at 32° C. (Bhamarapravati et al. (1987) Bull. World Health Organ. 65 (2): 189-195).

The attenuated DEN-2 PDK-53 strain (the precursor of TDV-2) was derived from the wild type virus strain DEN-2 16681 (SEQ ID NO 11) and differs in nine nucleotides from the wild type as follows (Kinney et al. (1997) Virology 230 (2): 300-308):
(i) 5′-noncoding region (NCR)-57 (nt-57 C-to-T): major attenuation locus
(ii) prM-29 Asp-to-Val (nt-524 A-to-T)
(iii) nt-2055 C-to-T (E gene) silent mutation
(iv) NS1-53 Gly-to-Asp (nt-2579 G-to-A): major attenuation locus
(v) NS2A-181 Leu-to-Phe (nt-4018 C-to-T)
(vi) NS3-250 Glu-to-Val (nt-5270 A-to-T): major attenuation locus
(vii) nt-5547 (NS3 gene) T-to-C silent mutation
(viii) NS4A-75 Gly-to-Ala (nt-6599 G-to-C)
nt-8571 C-to-T (NS5 gene) silent mutation

The three nucleotide changes located in the 5′ noncoding region (NCR) (nucleotide 57) (mutation (i)), the NS-1 (amino acid 828 of SEQ ID NO. 4) (mutation (iv)) and NS-3 genes (amino acid 1725 of SEQ ID NO. 4) (mutation (vi)) form the basis for the attenuation phenotype of the DEN-2 PDK-53 strain (Butrapet et al. (2000) J. Virol. 74 (7): 3111-3119) (Table 2). These three mutations are referred to herein as the “attenuating mutations” and are comprised in TDV-1, TDV-2, TDV-3 and TDV-4.

TABLE 2
Attenuating mutations in the common
genetic backbone of all TDV strains

| | Nucleotide | Amino Acid |
|---|---|---|
| Location of Mutation | Change in TDV-2 | Change in TDV-2 |
| 5′ Noncoding Region (5′NCR) | 57 C to T | Not applicable (silent) |
| Nonstructural Protein 1 (NS1) | 2579 G to A | 828 Gly to Asp |
| Nonstructural Protein 3 (NS3) | 5270 A to T | 1725 Glu to Val |

In one embodiment, TDV-2 comprises in addition to the three attenuating mutations one or more mutations selected from:
a) a mutation in the prM gene at nucleotide 524 from adenine to thymine resulting in an amino acid change at position 143 from aspartic acid to valine, and/or
b) a silent mutation in the E gene at nucleotide 2055 from cytosine to thymine, and/or
c) a mutation in the NS2A gene at nucleotide 4018 from cytosine to thymine resulting in an amino acid change at position 1308 from leucine to phenylalanine, and/or
d) a silent mutation in the NS3 gene at nucleotide 5547 from thymine to cytosine, and/or e) a mutation in the NS4A gene at nucleotide 6599 from guanine to cytosine resulting in an amino acid change at position 2168 from glycine to alanine, and/or
f) a silent mutation in the prM gene at nucleotide 900 from thymine to cytosine.

The silent mutation in the NS5 gene at nucleotide 8571 from cytosine to thymine of DEN-2 PDK-53 is not present in the TDV-2 strain.

In another embodiment, TDV-2 comprises in addition to the three attenuating mutations one or more mutations selected from:
g) a mutation in the prM gene at nucleotide 592 from adenine to guanine resulting in an amino acid change at position 166 from lysine to glutamic acid, and/or
h) a mutation in the NS5 gene at nucleotide 8803 from adenine to guanine resulting in an amino acid change at position 2903 from isoleucine to valine.

In another embodiment, TDV-2 comprises in addition to the three attenuating mutations the mutations a) and g), preferably the mutations a), g), c), e) and h), more preferably the mutations a), g), c), e), h) and b), even more preferably the mutations a), g), c), e), h), b) and d), and most preferably the mutations a) to h). The nucleotide positions and amino acids positions of TDV-2 refer to the nucleotide sequence as shown in SEQ ID NO. 3 and amino acid sequence as shown in SEQ ID NO. 4.

The dengue virus structural envelope (E) protein and pre-membrane (prM) protein have been identified as the primary antigens that elicit a neutralizing protective antibody response (Plotkin 2001). For creation of the tetravalent dengue vaccine (TDV), TDV-2 was modified by replacing the nucleic acid sequence encoding the DENV-2 prM and E glycoproteins with the nucleic acid sequence encoding the corresponding wild type prM and E glycoproteins from the DENV-1, DENV-3, and DENV-4 wild type strains DENV-1 16007, DENV-3 16562 or DENV-4 1036 virus, respectively, (see Table 3) using standard molecular genetic engineering methods (Huang et al. (2003) J. Virol. 77 (21): 11436-11447).

TABLE 3
Viral origin of prM/E gene regions of the TDV virus strains

| | | | | | Nucleotide | Amino acid |
|---|---|---|---|---|---|---|
| Virus | Strain | Origin | Source | Reference | sequence | sequence |
| DENV-1 | 16007 | Thailand, 196 [symbol] | DHF/DSS | Halstead and | SEQ ID NO. 9 | SEQ ID NO. 10 |
| | | | patient | Simasthien, 197 [symbol] | | |
| DENV-2 | 16681 | Thailand, 196 [symbol] | DHF/DSS | Halstead and | SEQ ID NO. 11 | SEQ ID NO. 12 |
| | | | patient | Simasthien, 197 [symbol] | | |
| DENV-3 | 16562 | Philippines, 1 [symbol] | DHF patient | Halstead and | SEQ ID NO. 13 | SEQ ID NO. 14 |
| | | | | Simasthien, 197 [symbol] | | |
| DENV-4 | 1036 | Indonesia, 19 [symbol] | DF patient | Gubler et al., 19 [symbol] | SEQ ID NO. 15 | SEQ ID NO. 16 |

[symbol]

indicates data missing or illegible when filed

A diagram of the four TDV strains comprised in the dengue vaccine composition is shown in FIG. 1.

The chimeric dengue strains TDV-1, TDV-3 and TDV-4 express the surface antigens prM and E of the DENV-1, DENV-3 or DENV-4 viruses, as depicted in Table 3 respectively, and retain the genetic alterations responsible for the attenuation of TDV-2. Thus, each of the TDV-1, TDV-3 and TDV-4 strains comprises the attenuating mutations described in Table 2.

In one embodiment, TDV-1 comprises in addition to the three attenuating mutations one or more mutations selected from:
c) a mutation in the NS2A gene at nucleotide 4018 from cytosine to thymine resulting in an amino acid change at position 1308 from leucine to phenylalanine, and/or
d) a silent mutation in the NS3 gene at nucleotide 5547 from thymine to cytosine, and/or
e) a mutation in the NS4A gene at nucleotide 6599 from guanine to cytosine resulting in an amino acid change at position 2168 from glycine to alanine, and/or
i) a silent mutation in the E gene at nucleotide 1575 from thymine to cytosine, and/or
j) a silent mutation in the junction site between the prM-E gene and the DEN-2 PDK-53 backbone at nucleotide 453 from adenine to guanine, and/or
k) a mutation in the junction site between the prM-E gene and the DEN-2 PDK-53 backbone at nucleotides 2381/2382 from thymine-guanine to cytosine-cytosine resulting in an amino acid change at position 762 from valine to alanine.

In another embodiment, TDV-1 comprises in addition to the three attenuating mutations one or more mutations selected from:
l) a mutation in the NS2A gene at nucleotide 3823 from adenine to cytosine resulting in an amino acid change at position 1243 from isoleucine to leucine, and/or
m) a mutation in the NS2B gene at nucleotide 4407 from adenine to thymine resulting in an amino acid change at position 1437 from glutamic acid to aspartic acid, and/or
n) a silent mutation in the NS4B gene at nucleotide 7311 from adenine to guanine.

In another embodiment, the TDV-1 strain comprises in addition to the three attenuating mutations the mutations I) and m), preferably the mutations I), m), c) and e), even more preferably the mutations I), m), c), e), d) and n), and most preferably the mutations I), m), c), e), d), n), i), j) and k). The nucleotide positions and amino acids positions of TDV-1 refer to the nucleotide sequence as shown in SEQ ID NO. 1 and amino acid sequence as shown in SEQ ID NO. 2.

In one embodiment, TDV-3 comprises in addition to the three attenuating mutations one or more mutations selected from:
c) a mutation in the NS2A gene at nucleotide 4012 from cytosine to thymine resulting in an amino acid change at position 1306 from leucine to phenylalanine, and/or
d) a silent mutation in the NS3 gene at nucleotide 5541 from thymine to cytosine, and/or
e) a mutation in the NS4A gene at nucleotide 6593 from guanine to cytosine resulting in an amino acid change at position 2166 from glycine to alanine, and/or
j) a silent mutation in the junction site between the prM-E gene and the DEN-2 PDK-53 backbone at nucleotide 453 from adenine to guanine, and/or
k) a mutation in the junction site between the prM-E gene and the DEN-2 PDK-53 backbone at nucleotides 2375/2376 from thymine-guanine to cytosine-cytosine resulting in an amino acid change at position 760 from valine to alanine, and/or
o) a silent mutation in the prM gene at nucleotide 552 from cytosine to thymine, and/or
p) a mutation in the E gene at nucleotide 1970 from adenine to thymine resulting in an amino acid change at position 625 from histidine to leucine.

In another embodiment, TDV-3 comprises in addition to the three attenuating mutations one or more mutations selected from:
q) a mutation in the E gene at nucleotide 1603 from adenine to thymine resulting in an amino acid change at position 503 from threonine to serine, and/or
r) a silent mutation in the NS5 gene at nucleotide 7620 from adenine to guanine.

In another embodiment, TDV-3 comprises in addition to the three attenuating mutations the mutations p) and q), preferably the mutations p), q), c) and e), even more preferably the mutations p), q), c), e), d) and r), and most preferably the mutations p), q), c), e), d), r), j), k) and o). The nucleotide positions and amino acids positions of TDV-3 refer to the nucleotide sequence as shown in SEQ ID NO. 5 and amino acid sequence as shown in SEQ ID NO. 6.

In one embodiment, TDV-4 comprises in addition to the three attenuating mutations one or more mutations selected from:
c) a mutation in the NS2A gene at nucleotide 4018 from cytosine to thymine resulting in an amino acid change at position 1308 from leucine to phenylalanine, and/or
d) a silent mutation in the NS3 gene at nucleotide 5547 from thymine to cytosine, and/or
e) a mutation in the NS4A gene at nucleotide 6599 from guanine to cytosine resulting in an amino acid change at position 2168 from glycine to alanine, and/or
j) a silent mutation in the junction site between the prM-E gene and the DEN-2 PDK-53 backbone at nucleotide 453 from adenine to guanine, and/or
k) a mutation in the junction site between the prM-E gene and the DEN-2 PDK-53 backbone at nucleotides 2381/2382 from thymine-guanine to cytosine-cytosine resulting in an amino acid change at position 762 from valine to alanine, and/or
s) a mutation in the C gene at nucleotide 396 from adenine to cytosine resulting in an amino acid change at position 100 from arginine to serine, and/or
t) a silent mutation in the E gene at nucleotide 1401 from adenine to guanine, and/or
u) a mutation in the E gene at nucleotide 2027 from cytosine to thymine resulting in an amino acid change at position 644 from alanine to valine, and/or
v) a mutation in the E gene at nucleotide 2275 from adenine to cytosine resulting in an amino acid change at position 727 from methionine to leucine.

In another embodiment, TDV-4 comprises in addition to the three attenuating mutations one or more mutations selected from:
w) a silent mutation in the C gene at nucleotide 225 from adenine to thymine, and/or
x) a mutation in the NS2A gene at nucleotide 3674 from adenine to guanine resulting in an amino acid change at position 1193 from aspartic acid to glycine, and/or
y) a mutation in the NS2A gene at nucleotide 3773 from adenine to an adenine/guanine mix resulting in an amino acid change at position 1226 from lysine to a lysine/arginine_mix, and/or
z) a silent mutation in the NS3 gene at nucleotide 5391 from cytosine to thymine, and/or
aa) a mutation in the NS4A gene at nucleotide 6437 from cytosine to thymine resulting in an amino acid change at position 2114 from alanine to valine, and/or
bb) a silent mutation in the NS4B gene at nucleotide 7026 from thymine to a thymine/cytosine mix, and/or
cc) a silent mutation in the NS5 gene at nucleotide 9750 from adenine to cytosine.

In another embodiments, TDV-4 comprises in addition to the three attenuating mutations the mutation s), u) and v), preferably the mutations s), u), v), c), e), x), y) and aa), even more preferably the mutations s), u), v), c), e), x), y), aa) and w), even more preferably the mutations s), u), v), c), e), x), y), aa), w), d), z), bb) and cc), and most preferably the mutations s), u), v), c), e), x), y), aa), w), d), z), bb), cc), j), k) and t). The nucleotide positions and amino acids positions of TDV-4 refer to the nucleotide sequence as shown in SEQ ID NO. 7 and amino acid sequence as shown in SEQ ID NO. 8.

In a preferred embodiment, TDV-1 has the nucleotide sequence of SEQ ID NO. 1, TDV-2 has the nucleotide sequence of SEQ ID NO. 3, TDV-3 has the nucleotide sequence of SEQ ID NO. 5, and/or TDV-4 has the nucleotide sequence of SEQ ID NO. 7. In a further preferred embodiment, TDV-1 has the amino acid sequence of SEQ ID NO. 2, TDV-2 has the amino acid sequence of SEQ ID NO. 4, TDV-3 has the amino acid sequence of SEQ ID NO. 6, and TDV-4 has the amino acid sequence of SEQ ID NO. 8. In a further preferred embodiment, TDV-1 has a nucleotide sequence encoding the amino acid sequence of SEQ ID NO. 2, TDV-2 has a nucleotide sequence encoding the amino acid sequence of SEQ ID NO. 4, TDV-3 has a nucleotide sequence encoding the amino acid sequence of SEQ ID NO. 6, and TDV-4 has a nucleotide sequence encoding the amino acid sequence of SEQ ID NO. 8.

TABLE 4
Sequences of the TDV virus strains

| SEQ ID NO. | dengue virus strain | sequence type |
|---|---|---|
| SEQ ID NO. 1 | TDV-1 | nucleotide sequence |
| SEQ ID NO. 2 | TDV-1 | amino acid sequence |
| SEQ ID NO. 3 | TDV-2 | nucleotide sequence |
| SEQ ID NO. 4 | TDV-2 | amino acid sequence |
| SEQ ID NO. 5 | TDV-3 | nucleotide sequence |
| SEQ ID NO. 6 | TDV-3 | amino acid sequence |
| SEQ ID NO. 7 | TDV-4 | nucleotide sequence |
| SEQ ID NO. 8 | TDV-4 | amino acid sequence |

Thus, in a particularly preferred embodiment, the unit dose of the invention as described herein comprises the live attenuated dengue virus strains TDV-1, TDV-2, TDV-3 and TDV-4, wherein TDV-1, TDV-3 and TDV-4 are based on TDV-2 and comprise the prM and E regions of DENV-1,-3 and -4, respectively. In another particularly preferred embodiment, TDV-1 is characterized by the nucleotide sequence according to SEQ ID No. 1 and the amino acid sequence according to SEQ ID No. 2, TDV-2 is characterized by the nucleotide sequence according to SEQ ID No. 3 and the amino acid sequence according to SEQ ID No. 4, TDV-3 is characterized by the nucleotide sequence according to SEQ ID No. 5 and the amino acid sequence according to SEQ ID No. 6 and TDV-4 is characterized by the nucleotide sequence according to SEQ ID No. 7 and the amino acid sequence according to SEQ ID No. 8.

The E protein of DENV-3 has two fewer amino acids than the E protein of DENV-2. Therefore, the nucleotides and encoded amino acid backbone of TDV-2 starting after the E region of DENV-3 at nucleotide 2374 of SEQ ID NO. 5 and amino acid 760 of SEQ ID NO. 6 are 6 nucleotides less and 2 amino acids less than the original TDV-2 nucleotide and amino acid positions, respectively.

### Dengue Vaccine Composition

The present invention is in part directed to a unit dose of a dengue vaccine composition as described. The dengue vaccine composition comprises a tetravalent dengue virus composition, also referred to as dengue virus composition, and pharmaceutically acceptable excipients.

### Dengue Virus Composition, Virus Concentrations and %-Concentrations

The present invention is in part directed to a unit dose of a dengue vaccine composition, wherein the dengue vaccine composition comprises a tetravalent dengue virus composition including four live attenuated dengue virus strains:
(i) a dengue serotype 1 preferably in a concentration of at least 3.3 log 10 pfu/0.5 mL,
(ii) a dengue serotype 2 preferably in a concentration of at least 2.7 log 10 pfu/0.5 mL,
(iii) a dengue serotype 3 preferably in a concentration of at least 4.0 log 10 pfu/0.5 mL, and
(iv) a dengue serotype 4 preferably strain in a concentration of at least 4.5 log 10 pfu/0.5 mL.

In one embodiment, the dengue vaccine composition comprises a tetravalent dengue virus composition including four live attenuated dengue virus strains:
(i) a dengue serotype 1 preferably in a concentration of at least 3.3 log 10 pfu/0.5 ml to 3.8 log 10 pfu/0.5 mL,
(ii) a dengue serotype 2 preferably in a concentration of at least 2.7 log 10 pfu/0.5 mL,
(iii) a dengue serotype 3 preferably in a concentration of at least 4.0 log 10 pfu/0.5 mL, and
(iv) a dengue serotype 4 preferably strain in a concentration of at least 4.5 log 10 pfu/0.5 ml or 4.6 log 10 pfu/0.5 mL, optionally to 6.2 log 10 pfu/0.5 ml.

The present invention is further in part directed to a unit dose of a dengue vaccine composition, wherein the dengue vaccine composition comprises a tetravalent dengue virus composition including four live attenuated dengue virus strains:
(i) a chimeric dengue serotype 2/1 strain in a concentration of at least 3.3 log 10 pfu/0.5 mL,
(ii) a dengue serotype 2 strain in a concentration of at least 2.7 log 10 pfu/0.5 mL,
(iii) a chimeric dengue serotype ⅔ strain in a concentration of at least 4.0 log 10 pfu/0.5 mL, and
(iv) a chimeric dengue serotype 2/4 strain in a concentration of at least 4.5 log 10 pfu/0.5 mL.

In one embodiment, the dengue vaccine composition comprises a tetravalent dengue virus composition including four live attenuated dengue virus strains:
(i) a chimeric dengue serotype 2/1 strain in a concentration of at least 3.3 log 10 pfu/0.5 ml to 3.8 log 10 pfu/0.5 ml,
(ii) a dengue serotype 2 strain in a concentration of at least 2.7 log 10 pfu/0.5 mL,
(iii) a chimeric dengue serotype ⅔ strain in a concentration of at least 4.0 log 10 pfu/0.5 mL, and
(iv) a chimeric dengue serotype 2/4 strain in a concentration of at least 4.5 log 10 pfu/0.5 mL or at least 4.6 log 10 pfu/0.5 mL to optionally 6.2 log 10 pfu/0.5 ml.

Preferably, the chimeric dengue serotype 2/1 strain is TDV-1, the dengue serotype 2 strain is TDV-2, the chimeric dengue serotype ⅔ strain is TDV-3 and the chimeric dengue serotype 2/4 strain is TDV-4.

In one embodiment, the dengue vaccine composition comprises a tetravalent dengue virus composition including four live attenuated dengue virus strains wherein:
(i) the dengue serotype 1 (e.g. chimeric dengue serotype 2/1 strain) has a concentration of 3.3 log 10 pfu/0.5 mL to 5.3 log 10 pfu/0.5 mL,
(ii) the dengue serotype 2 (e.g. dengue serotype 2 strain) has a concentration of 2.7 log 10 pfu/0.5 mL to 5.0 log 10 pfu/0.5 mL,
(iii) the dengue serotype 3 (e.g. chimeric dengue serotype ⅔ strain) has a concentration of 4.0 log 10 pfu/0.5 mL to 6.0 log 10 pfu/0.5 mL, and
(iv) the dengue serotype 4 (e.g. chimeric dengue serotype 2/4 strain) has a concentration of 4.5 log 10 pfu/0.5 mL to 6.5 log 10 pfu/0.5 mL.

In one such embodiment, the dengue vaccine composition comprises a tetravalent dengue virus composition including four live attenuated dengue virus strains wherein:
(i) the dengue serotype 1 (e.g. chimeric dengue serotype 2/1 strain) has a concentration of 3.3 log 10 pfu/0.5 mL to 5.0 log 10 pfu/0.5 mL,
(ii) the dengue serotype 2 (e.g. dengue serotype 2 strain) has a concentration of 2.7 log 10 pfu/0.5 mL to 4.9 log 10 pfu/0.5 mL,
(iii) the dengue serotype 3 (e.g. chimeric dengue serotype ⅔ strain) has a concentration of 4.0 log 10 pfu/0.5 mL to 5.7 log 10 pfu/0.5 mL, and
(iv) the dengue serotype 4 (e.g. chimeric dengue serotype 2/4 strain) has a concentration of 4.5 log 10 pfu/0.5 mL to 6.2 log 10 pfu/0.5 mL.

In a further such embodiment, the dengue vaccine composition comprises a tetravalent dengue virus composition including four live attenuated dengue virus strains wherein:
(i) a dengue serotype 1 (e.g. chimeric dengue serotype 2/1 strain) has a concentration of 3.3 log 10 pfu/dose to 5.0 log 10 pfu/dose,
(ii) a dengue serotype 2 (e.g. dengue serotype 2 strain) has a concentration of 2.7 log 10 pfu/dose to 4.9 log 10 pfu/dose,
(iii) a dengue serotype 3 (e.g. chimeric dengue serotype ⅔ strain) has a concentration of 4.0 log 10 pfu/dose to 5.7 log 10 pfu/dose, and
(iv) a dengue serotype 4 (e.g. chimeric dengue serotype 2/4 strain) has a concentration of 4.5 log 10 pfu/dose to 5.5 log 10 pfu/dose.

In a further such embodiment, the dengue vaccine composition comprises a tetravalent dengue virus composition including four live attenuated dengue virus strains wherein:
(i) a dengue serotype 1 (e.g. chimeric dengue serotype 2/1 strain) has a concentration of 3.3 log 10 pfu/dose to 4.1 log 10 pfu/dose,
(ii) a dengue serotype 2 (e.g. dengue serotype 2 strain) has a concentration of 2.7 log 10 pfu/dose to 3.6 log 10 pfu/dose,
(iii) a dengue serotype 3 (e.g. chimeric dengue serotype ⅔ strain) has a concentration of 4.0 log 10 pfu/dose to 4.7 log 10 pfu/dose, and
(iv) a dengue serotype 4 (e.g. chimeric dengue serotype 2/4 strain) has a concentration of 4.5 log 10 pfu/dose to 5.3 log 10 pfu/dose.

In a further such embodiment, the dengue vaccine composition comprises a tetravalent dengue virus composition including four live attenuated dengue virus strains wherein:
(i) a dengue serotype 1 (e.g. chimeric dengue serotype 2/1 strain) has a concentration of 3.3 log 10 pfu/0.5 mL to 3.6 log 10 pfu/0.5 mL,
(ii) a dengue serotype 2 (e.g. dengue serotype 2 strain) has a concentration of 2.7 log 10 pfu/0.5 mL to 4.0 log 10 pfu/0.5 mL,
(iii) a dengue serotype 3 (e.g. chimeric dengue serotype ⅔ strain) has a concentration of 4.0 log 10 pfu/0.5 mL to 4.6 log 10 pfu/0.5 mL, and
(iv) a dengue serotype 4 (e.g. chimeric dengue serotype 2/4 strain) has a concentration of 4.5 log 10 pfu/0.5 ml or 4.6 log 10 pfu/0.5 mL to 5.1 log 10 pfu/0.5 mL.

In another embodiment, the dengue vaccine composition comprises a tetravalent dengue virus composition including four live attenuated dengue virus strains wherein:
(i) the dengue serotype 1 (e.g. chimeric dengue serotype 2/1 strain) has a concentration of 4.3 log 10 pfu/0.5 mL to 4.4 log 10 pfu/0.5 mL,
(ii) the dengue serotype 2 (e.g. dengue serotype 2 strain) has a concentration of 3.7 log 10 pfu/0.5 mL to 3.8 log 10 pfu/0.5 mL,
(iii) the dengue serotype 3 (e.g. chimeric dengue serotype ⅔ strain) has a concentration of 4.5 log 10 pfu/0.5 mL to 5.0 log 10 pfu/0.5 mL, and
(iv) the dengue serotype 4 (e.g. chimeric dengue serotype 2/4 strain) has a concentration of 5.5 log 10 pfu/0.5 mL to 5.6 log 10 pfu/0.5 mL.

In a particularly preferred embodiment, the dengue vaccine composition comprises a tetravalent dengue virus composition including four live attenuated dengue virus strains wherein:
(i) the dengue serotype 1 (e.g. chimeric dengue serotype 2/1 strain) has a concentration of 4.4 log 10 pfu/0.5 mL,
(ii) the dengue serotype 2 (e.g. dengue serotype 2 strain) has a concentration of 3.8 log 10 pfu/0.5 mL,
(iii) the dengue serotype 3 (e.g. chimeric dengue serotype ⅔ strain) has a concentration of 4.5 log 10 pfu/0.5 mL, and
(iv) the dengue serotype 4 (e.g. chimeric dengue serotype 2/4 strain) has a concentration of 5.6 log 10 pfu/0.5 mL.

In another particularly preferred embodiment, the dengue vaccine composition comprises a tetravalent dengue virus composition including four live attenuated dengue virus strains wherein:
(i) the dengue serotype 1 (e.g. chimeric dengue serotype 2/1 strain) has a concentration of 3.6 log 10 pfu/0.5 mL,
(ii) the dengue serotype 2 (e.g. dengue serotype 2 strain) has a concentration of 4.0 log 10 pfu/0.5 mL,
(iii) the dengue serotype 3 (e.g. chimeric dengue serotype ⅔ strain) has a concentration of 4.6 log 10 pfu/0.5 mL, and
(iv) the dengue serotype 4 (e.g. chimeric dengue serotype 2/4 strain) has a concentration of 5.1 log 10 pfu/0.5 mL.

In another preferred embodiment, the dengue vaccine composition comprises a tetravalent dengue virus composition including four live attenuated dengue virus strains wherein the arithmetic sum of all four serotypes is less than 6.7 log 10 pfu/0.5 mL, preferably less than 5.5 log 10 pfu/0.5 mL. In certain such embodiments, the arithmetic sum of all four serotypes is at least 4.6 log 10 pfu/0.5 mL. In a preferred embodiment, the dengue vaccine composition comprises a tetravalent dengue virus composition including four live attenuated dengue virus strains wherein the arithmetic sum of all four serotypes is in the range of 4.6 log 10 pfu/0.5 mL to 6.7 log 10 pfu/0.5 mL, preferably in the range of 4.6 log 10 pfu/0.5 ml to 5.5 log 10 pfu/0.5 mL.

Preferably, in said embodiments the chimeric dengue serotype 2/1 strain is TDV-1, the dengue serotype 2 strain is TDV-2, the chimeric dengue serotype ⅔ strain is TDV-3 and the chimeric dengue serotype 2/4 strain is TDV-4. More preferably, TDV-1 is characterized by the nucleotide sequence according to SEQ ID No. 1 and the amino acid sequence according to SEQ ID No. 2, TDV-2 is characterized by the nucleotide sequence according to SEQ ID No. 3 and the amino acid sequence according to SEQ ID No. 4, TDV-3 is characterized by the nucleotide sequence according to SEQ ID No. 5 and the amino acid sequence according to SEQ ID No. 6 and TDV-4 is characterized by the nucleotide sequence according to SEQ ID No. 7 and the amino acid sequence according to SEQ ID No. 8.

The present invention is in part directed to a unit dose of a dengue vaccine composition, wherein the dengue vaccine composition comprises a tetravalent dengue virus composition including four live attenuated dengue virus strains:
(i) a dengue serotype 1 (e.g. chimeric dengue serotype 2/1 strain) in a concentration of at least 3.3 log 10 pfu/dose,
(ii) a dengue serotype 2 (e.g. dengue serotype 2 strain) in a concentration of at least 2.7 log 10 pfu/dose,
(iii) a dengue serotype 3 (e.g. chimeric dengue serotype ⅔ strain) in a concentration of at least 4.0 log 10 pfu/dose, and
(iv) a dengue serotype 4 (e.g. chimeric dengue serotype 2/4 strain) in a concentration of at least 4.5 log 10 pfu/dose.

In one embodiment, the dengue vaccine composition comprises a tetravalent dengue virus composition including four live attenuated dengue virus strains wherein:
(i) the dengue serotype 1 (e.g. chimeric dengue serotype 2/1 strain) has a concentration of 3.3 log 10 pfu/dose to 5.3 log 10 pfu/dose,
(ii) the dengue serotype 2 (e.g. dengue serotype 2 strain) has a concentration of 2.7 log 10 pfu/dose to 5.0 log 10 pfu/dose,
(iii) the dengue serotype 3 (e.g. chimeric dengue serotype ⅔ strain) has a concentration of 4.0 log 10 pfu/dose to 6.0 log 10 pfu/dose, and
(iv) the dengue serotype 4 (e.g. chimeric dengue serotype 2/4 strain) has a concentration of 4.5 log 10 pfu/dose to 6.5 log 10 pfu/dose.

In one such embodiment, the dengue vaccine composition comprises a tetravalent dengue virus composition including four live attenuated dengue virus strains wherein:
(i) the dengue serotype 1 (e.g. chimeric dengue serotype 2/1 strain) has a concentration of 3.3 log 10 pfu/dose to 5.0 log 10 pfu/dose,
(ii) the dengue serotype 2 (e.g. dengue serotype 2 strain) has a concentration of 2.7 log 10 pfu/dose to 4.9 log 10 pfu/dose,
(iii) the dengue serotype 3 (e.g. chimeric dengue serotype ⅔ strain) has a concentration of 4.0 log 10 pfu/dose to 5.7 log 10 pfu/dose, and
(iv) the dengue serotype 4 (e.g. chimeric dengue serotype 2/4 strain) has a concentration of 4.5 log 10 pfu/dose to 6.2 log 10 pfu/dose.

In a further such embodiment, the dengue vaccine composition comprises a tetravalent dengue virus composition including four live attenuated dengue virus strains wherein:
(i) a dengue serotype 1 (e.g. chimeric dengue serotype 2/1 strain) has a concentration of 3.3 log 10 pfu/dose to 5.0 log 10 pfu/dose,
(ii) a dengue serotype 2 (e.g. dengue serotype 2 strain) has a concentration of 2.7 log 10 pfu/dose to 4.9 log 10 pfu/dose,
(iii) a dengue serotype 3 (e.g. chimeric dengue serotype ⅔ strain) has a concentration of 4.0 log 10 pfu/dose to 5.7 log 10 pfu/dose, and
(iv) a dengue serotype 4 (e.g. chimeric dengue serotype 2/4 strain) has a concentration of 4.5 log 10 pfu/dose to 5.5 log 10 pfu/dose.

In a further such embodiment, the dengue vaccine composition comprises a tetravalent dengue virus composition including four live attenuated dengue virus strains wherein:
(i) a dengue serotype 1 (e.g. chimeric dengue serotype 2/1 strain) has a concentration of 3.3 log 10 pfu/dose to 4.1 log 10 pfu/dose,
(ii) a dengue serotype 2 (e.g. dengue serotype 2 strain) has a concentration of 2.7 log 10 pfu/dose to 3.6 log 10 pfu/dose,
(iii) a dengue serotype 3 (e.g. chimeric dengue serotype ⅔ strain) has a concentration of 4.0 log 10 pfu/dose to 4.7 log 10 pfu/dose, and
(iv) a dengue serotype 4 (e.g. chimeric dengue serotype 2/4 strain) has a concentration of 4.5 log 10 pfu/dose to 5.3 log 10 pfu/dose.

In a further such embodiment, the dengue vaccine composition comprises a tetravalent dengue virus composition including four live attenuated dengue virus strains wherein:
(i) a dengue serotype 1 (e.g. chimeric dengue serotype 2/1 strain) has a concentration of 3.3 log 10 pfu/dose to 3.6 log 10 pfu/dose,
(ii) a dengue serotype 2 (e.g. dengue serotype 2 strain) has a concentration of 2.7 log 10 pfu/dose to 4.0 log 10 pfu/dose,
(iii) a dengue serotype 3 (e.g. chimeric dengue serotype ⅔ strain) has a concentration of 4.0 log 10 pfu/dose to 4.6 log 10 pfu/dose, and
(iv) a dengue serotype 4 (e.g. chimeric dengue serotype 2/4 strain) has a concentration of 4.5 log 10 pfu/dose 4.6 log 10 pfu/dose to 5.1 log 10 pfu/dose.

In another embodiment, the dengue vaccine composition comprises a tetravalent dengue virus composition including four live attenuated dengue virus strains wherein:
(i) the dengue serotype 1 (e.g. chimeric dengue serotype 2/1 strain) has a concentration of 4.3 log 10 pfu/dose to 4.4 log 10 pfu/dose,
(ii) the dengue serotype 2 (e.g. dengue serotype 2 strain) has a concentration of 3.7 log 10 pfu/dose to 3.8 log 10 pfu/dose,
(iii) the dengue serotype 3 (e.g. chimeric dengue serotype ⅔ strain) has a concentration of 4.5 log 10 pfu/dose to 5.0 log 10 pfu/dose, and
(iv) the dengue serotype 4 (e.g. chimeric dengue serotype 2/4 strain) has a concentration of 5.5 log 10 pfu/dose to 5.6 log 10 pfu/dose.

In a particularly preferred embodiment, the dengue vaccine composition comprises a tetravalent dengue virus composition including four live attenuated dengue virus strains wherein:
(i) the dengue serotype 1 (e.g. chimeric dengue serotype 2/1 strain) has a concentration of 4.4 log 10 pfu/dose,
(ii) the dengue serotype 2 (e.g. dengue serotype 2 strain) has a concentration of 3.8 log 10 pfu/dose,
(iii) the dengue serotype 3 (e.g. chimeric dengue serotype ⅔ strain) has a concentration of 4.5 log 10 pfu/dose, and
(iv) the dengue serotype 4 (e.g. chimeric dengue serotype 2/4 strain) has a concentration of 5.6 log 10 pfu/dose.

In another particularly preferred embodiment, the dengue vaccine composition comprises a tetravalent dengue virus composition including four live attenuated dengue virus strains wherein:
(i) the dengue serotype 1 (e.g. chimeric dengue serotype 2/1 strain) has a concentration of 3.6 log 10 pfu/dose,
(ii) the dengue serotype 2 (e.g. dengue serotype 2 strain) has a concentration of 4.0 log 10 pfu/dose,
(iii) the dengue serotype 3 (e.g. chimeric dengue serotype ⅔ strain) has a concentration of 4.6 log 10 pfu/dose, and
(iv) the dengue serotype 4 (e.g. chimeric dengue serotype 2/4 strain) has a concentration of 5.1 log 10 pfu/dose.

In another preferred embodiment, the dengue vaccine composition comprises a tetravalent dengue virus composition including four live attenuated dengue virus strains wherein the arithmetic sum of all four serotypes is less than 6.7 log 10 pfu/dose, preferably less than 5.5 log 10 pfu/dose. In certain such embodiments, the arithmetic sum of all four serotypes is at least 4.6 log 10 pfu/dose. in a preferred embodiment, the dengue vaccine composition comprises a tetravalent dengue virus composition including four live attenuated dengue virus strains wherein the arithmetic sum of all four serotypes is in the range of 4.6 log 10 pfu/dose to 6.7 log 10 pfu/dose, preferably in the range of 4.6 log 10 pfu/dose to 5.5 log 10 pfu/dose.

In one embodiment in the composition (i), (ii), (iii), and (iv) provide a total concentration of pfu/0.5 mL and based on said concentration, the concentration of (iii) at least 10% of the total concentration in pfu/0.5 mL.

In one embodiment in the composition (i), (ii), (iii), and (iv) provide a total concentration of pfu/0.5 mL and based on said total concentration the concentration of (ii) in pfu/0.5 mL is less than 10%, and the concentration of (iv) in pfu/0.5 mL is at least 50%, and the concentration of (i) in pfu/0.5 mL is at least 1%, and the concentration of (iii) in pfu/0.5 mL is at least 6%, or at least 8%, or at least 10%, or at least 12%, or at least 14%, or at least 16%, or at least 18%.

It is preferred that the concentration in the reconstituted unit dose of (iii) in pfu/0.5 mL is at least 10%.

In one embodiment in the composition (i), (ii), (iii), and (iv) provide a total concentration of pfu/0.5 mL and based on said total concentration the concentration of (ii) in pfu/0.5 mL is less than 2%, the concentration of (iv) in pfu/0.5 mL is at least 50%, the concentration of (i) in pfu/0.5 mL is at least 1%, and the concentration of (iii) in pfu/0.5 ml is at least 6%.

Preferably, in said embodiments the chimeric dengue serotype 2/1 strain is TDV-1, the dengue serotype 2 strain is TDV-2, the chimeric dengue serotype ⅔ strain is TDV-3 and the chimeric dengue serotype 2/4 strain is TDV-4. More preferably, TDV-1 is characterized by the nucleotide sequence according to SEQ ID No. 1 and the amino acid sequence according to SEQ ID No. 2, TDV-2 is characterized by the nucleotide sequence according to SEQ ID No. 3 and the amino acid sequence according to SEQ ID No. 4, TDV-3 is characterized by the nucleotide sequence according to SEQ ID No. 5 and the amino acid sequence according to SEQ ID No. 6 and TDV-4 is characterized by the nucleotide sequence according to SEQ ID No. 7 and the amino acid sequence according to SEQ ID No. 8.

The concentration of the different dengue viruses is preferably determined by an immuno-focus assay known in the art. For example, the concentration may be determined by an immuno-focus assay wherein serial dilutions of dengue virus are applied to monolayers of adherent cells, such as Vero cells. After a period of time which allows infectious viruses to bind to the cells and to be taken up by the cells, an overlay containing thickening agents, such as agarose or carboxymethylcellulose, is added to prevent diffusion of viruses so that progeny viruses can only infect cells adjacent to the original infected cells. After a period of incubation to allow viral replication, cells are fixed and stained using serotype-specific anti-dengue monoclonal antibodies and a secondary antibody such as an antibody labeled with alkaline phosphatase. The foci are stained by adding a suitable substrate for the enzyme attached to the secondary antibody, such as 5-bromo-4-chloro-3-indolyl-phosphate/nitro blue tetrazolium phosphatase substrate. The number of plaques on the plate corresponds to the plaque forming units of the virus in the solutions applied to the cells. For example, a concentration of 1,000 pfu/μl indicates that 1 μl of the solution applied to the cells contains enough viruses to produce 1,000 plaques in a cell monolayer.

The dengue vaccine composition comprises a tetravalent dengue virus composition including four live attenuated dengue virus strains, wherein a chimeric dengue serotype 2/1 strain, a dengue serotype 2 strain, a chimeric dengue serotype ⅔ strain, and a chimeric dengue serotype 2/4 strain provide a total concentration in pfu/0.5 mL. The term “total concentration in pfu/0.5 mL” or “total concentration in pfu/dose” is the sum of the concentrations of the dengue serotype 1 (e.g. chimeric dengue serotype 2/1 strain), dengue serotype 2 (e.g. the dengue serotype 2 strain), the dengue serotype 3 (e.g. chimeric dengue serotype ⅔ strain) and the dengue serotype 4 (e.g. chimeric dengue serotype 2/4 strain), preferably the sum of the concentrations of TDV-1, TDV-2, TDV-3 and TDV-4, and is defined as 100% of the dengue virus concentration as determined by pfu (plaque forming units) in 0.5 mL or in a dose.

In one embodiment, the dengue vaccine composition comprises a tetravalent dengue virus composition including four live attenuated dengue virus strains, wherein a dengue serotype 1 (e.g. chimeric dengue serotype 2/1 strain), a dengue serotype 2 (e.g. dengue serotype 2 strain), a dengue serotype 3 (e.g. chimeric dengue serotype ⅔ strain), and a dengue serotype 4 (e.g. chimeric dengue serotype 2/4 strain) provide a total concentration in pfu/0.5 mL, wherein based on said total concentration the concentration of a dengue serotype 2 (e.g. dengue serotype 2 strain) measured in pfu/0.5 mL is less than 10% of the total concentration, or less than 8%, or less than 6% of the total concentration, and wherein the concentration of a dengue serotype 4 (e.g. chimeric dengue serotype 2/4 strain) measured in pfu/0.5 mL is at least 50% or at least 60% or at least 65% of the total concentration. In one embodiment, based on said total concentration the concentration of a dengue serotype 2 (e.g. dengue serotype 2 strain) measured in pfu/0.5 mL is 0.3 to 10% or 0.5 to 8% of the total concentration and the concentration of a dengue serotype 4 (e.g. chimeric dengue serotype 2/4 strain) measured in pfu/0.5 mL is 50% to 90% or 60% to 88% of the total concentration. This means that the concentration of the dengue serotype 2 (e.g. dengue serotype 2 strain) is lower than the concentration of the dengue serotype 4 (e.g. chimeric dengue serotype 2/4 strain).

In one such embodiment, the concentration of a dengue serotype 1 (e.g. chimeric dengue serotype 2/1 strain) measured in pfu/0.5 ml is at least 1% of the total concentration, and/or the concentration of a dengue serotype 3 (e.g. chimeric dengue serotype ⅔ strain) measured in pfu/0.5 mL is at least 6% of the total concentration, or at least 7% or 8%, 10%, 12%, 14%, 16% or 18% of the total concentration. In one such embodiment, the concentration of a dengue serotype 2 (e.g. chimeric dengue serotype 2/1 strain) measured in pfu/0.5 mL is 1% to 7% or 2% to 6% or 2.0% to 5.0% of the total concentration, and/or the concentration of a dengue serotype 3 (e.g. chimeric dengue serotype ⅔ strain) measured in pfu/0.5 mL is 6% to 25% or 7% to 25% or 10% to 25% or 18% to 25% of the total concentration. This means that the concentration of the dengue serotype 1 (e.g. chimeric dengue serotype 2/1 strain) is lower than the concentration of the dengue serotype 3 (e.g. chimeric dengue serotype ⅔ strain).

In a preferred embodiment, the concentration of a dengue serotype 2 strain, such as TDV-2, measured in pfu/0.5 mL is less than 10% of the total concentration, preferably less than 6% or less than 2%, the concentration of a dengue serotype 4 (e.g. chimeric dengue serotype 2/4 strain), such as TDV-4, measured in pfu/0.5 mL is at least 50% of the total concentration, preferably at least 65%, the concentration of a dengue serotype 1 (e.g. chimeric dengue serotype 2/1 strain), such as TDV-1, measured in pfu/0.5 mL is at least 1% of the total concentration, preferably between 1% and 7% or 2.0% to 5.0%, and the concentration of a dengue serotype 3 (e.g. chimeric dengue serotype ⅔ strain), such as TDV-3, measured in pfu/0.5 mL is at least 6% of the total concentration, preferably between 6% and 25% or 10% to 25% or 18% to 25%.

In a further preferred embodiment, a dengue virus composition comprising a dengue serotype 1 (e.g. chimeric dengue serotype 2/1 strain), a dengue serotype 2 (e.g. dengue serotype 2 strain), a dengue serotype 1 (e.g. chimeric dengue serotype ⅔ strain), and a dengue serotype 4 (e.g. chimeric dengue serotype 2/4 strain), such as TDV-1, TDV-2, TDV-3 and TDV-4, is provided, wherein the concentration of the dengue serotype 1 (e.g. chimeric dengue serotype 2/1 strain) measured in pfu/0.5 mL is at least 1% of the total concentration, preferably between 1% and 7% or 2.0% and 5.0%, the concentration of the dengue serotype 2 (e.g. dengue serotype 2 strain) measured in pfu/0.5 mL is less than 10% of the total concentration, preferably less than 6% or less than 2% and the concentration of the dengue serotype 3 (e.g. chimeric dengue serotype ⅔ strain) measured in pfu/0.5 mL is at least 6% of the total concentration, preferably between 6% and 25% or 10% to 25% or 18% to 25%. It is particularly preferred that the dengue serotype 4 (e.g. chimeric dengue serotype 2/4 strain) has the highest concentration of all four dengue serotypes.

In a further preferred embodiment, the dengue vaccine composition comprises a tetravalent dengue virus composition including four live attenuated dengue virus strains, wherein the concentration of the dengue serotype 1 (e.g. chimeric dengue serotype 2/1 strain) measured in pfu/0.5 mL is 1% to 7% of the total concentration, the concentration of the dengue serotype 2 (e.g. dengue serotype 2 strain) measured in pfu/0.5 mL is less than 8% of the total concentration, such as in the range of 1% to 8% of the total concentration, the concentration of the dengue serotype 3 (e.g. chimeric dengue serotype ⅔ strain) measured in pfu/0.5 mL is at least 10% of the total concentration, and the concentration of the dengue serotype 4 (e.g. chimeric dengue serotype 2/4 strain) measured in pfu/0.5 mL is at least 65% of the total concentration, such as in the range of 65% to 80%. In certain such embodiments, the arithmetic sum of all four serotypes is in the range of 4.6 log 10 pfu/0.5 mL to 6.7 log 10 pfu/0.5 mL, preferably in the range of 4.6 log 10 pfu/0.5 mL to 5.5 log 10 pfu/0.5 mL.

In a further preferred embodiment the dengue serotype 1 (e.g. chimeric dengue serotype 2/1 strain) such as TDV-1 and the dengue serotype 2 (e.g. dengue serotype 2 strain) such as TDV-2 are present each in a concentration based on the total concentration in pfu/0.5 mL which is within 5%-points of each other and/or are together less than about 10% of the total concentration in pfu/0.5 mL. In certain such embodiments the dengue serotype 3 (e.g. chimeric dengue serotype ⅔ strain) such as TDV-3 is preferably at least about 10% of the total concentration in pfu/0.5 mL and more preferably the dengue serotype 4 (e.g. chimeric dengue serotype 2/4 strain) such as TDV-4 is at least about 70% of the total concentration in pfu/0.5 mL. In certain such embodiments the dengue serotype 4 (e.g. chimeric dengue serotype 2/4 strain) such as TDV-4 represents the highest concentration in the composition of all four serotypes, preferably with at least about 70% of the total concentration in pfu/0.5 mL, dengue serotype 3 (e.g. chimeric dengue serotype ⅔ strain) such as TDV-3 represents the second highest concentration in the composition of all four serotypes, preferably with at least about 10% of the total concentration in pfu/0.5 mL, and dengue serotype 1 (e.g. chimeric dengue serotype 2/1 strain) such as TDV-1 and dengue serotype 2 (e.g. dengue serotype 2 strain) such as TDV-2 each represent lower concentrations than the concentration of serotype 3 (e.g. chimeric dengue serotype ⅔ strain) such as TDV-3, and optionally together represent less than about 10% of the total concentration in pfu/0.5 mL.

Preferably, in said embodiments the chimeric dengue serotype 2/1 strain is TDV-1, the dengue serotype 2 strain is TDV-2, the chimeric dengue serotype ⅔ strain is TDV-3 and the chimeric dengue serotype 2/4 strain is TDV-4. More preferably, TDV-1 is characterized by the nucleotide sequence according to SEQ ID No. 1 and the amino acid sequence according to SEQ ID No. 2, TDV-2 is characterized by the nucleotide sequence according to SEQ ID No. 3 and the amino acid sequence according to SEQ ID No. 4, TDV-3 is characterized by the nucleotide sequence according to SEQ ID No. 5 and the amino acid sequence according to SEQ ID No. 6 and TDV-4 is characterized by the nucleotide sequence according to SEQ ID No. 7 and the amino acid sequence according to SEQ ID No. 8.

According to a further embodiment, the chimeric dengue serotype 2/4 strain, preferably TDV-4, has the highest concentration in the dengue vaccine composition, followed by the chimeric dengue serotype ⅔ strain, preferably TDV-3, followed by the chimeric dengue serotype 2/1 strain, preferably TDV-1, followed by the dengue serotype 2 strain, preferably TDV-2. It is particularly preferred that the dengue serotype 2 strain has the lowest concentration of the four strains present in the dengue vaccine composition.

Whenever reference is made to a concentration/0.5 ml, this does not limit the volume of the unit dose described herein to 0.5 ml. 0.5 ml is the reference volume for the determination of the concentrations of the virus strains in the composition in pfu/ml. The volume and/or amount per unit dose is described in the respective chapter.

### Pharmaceutically Acceptable Excipients

The present invention is in part directed to a unit dose of a dengue vaccine composition, wherein the dengue vaccine composition comprises one or more pharmaceutically acceptable excipients. In one embodiment, the dengue vaccine composition comprises a non-reducing sugar, a surfactant, a protein and an inorganic salt. Preferably, the non-reducing sugar is trehalose, the surfactant is poloxamer 407, the protein is human serum albumin and the inorganic salt is sodium chloride.

In one embodiment, the unit dose of a dengue vaccine composition comprises the following pharmaceutically acceptable excipients:
from about 10% w/v to about 20% w/v α,α-trehalose dihydrate or an equimolar amount of other
forms of α,α-trehalose,
from about 0.5% w/v to about 1.5% w/v poloxamer 407,
from about 0.05% w/v to about 2% w/v human serum albumin, and
from about 70 mM to 140 mM sodium chloride.

In one embodiment, the unit dose of a dengue vaccine composition comprises the following pharmaceutically acceptable excipients when measured in 0.5 ml:
from about 10% w/v to about 20% w/v α,α-trehalose or an equimolar amount of other forms of α,α-trehalose,
from about 0.5% w/v to about 1.5% w/v poloxamer 407,
from about 0.05% w/v to about 2% w/v human serum albumin, and
from about 70 mM to 140 mM sodium chloride, and preferably
has a pH of 7 to 8.5.

In one embodiment, the unit dose of a dengue vaccine composition comprises the following pharmaceutically acceptable excipients when measured in 0.5 ml:
from about 143 mg/ml to about 185 mg/ml α,α-trehalose dihydrate or an equimolar amount of other forms of α,α-trehalose,
from about 9.1 mg/ml to about 12.4 mg/ml poloxamer 407,
from about 0.88% mg/ml to about 1.32 mg/ml human serum albumin, and
from about 70 mM to 140 mM sodium chloride, and preferably has a pH of 7 to 8.5.

In a preferred embodiment, the lyophilized unit dose of the invention as described herein comprises the following pharmaceutically acceptable excipients:
about 15% w/v α,α-trehalose dihydrate,
about 1% w/v poloxamer 407,
about 0.1% w/v human serum albumin, and
about 100 mM sodium chloride.

In a preferred embodiment, the lyophilized unit dose of the invention as described herein comprises the following pharmaceutically acceptable excipients when measured in 0.5 ml:
about 15% w/v α,α-trehalose,
about 1% w/v poloxamer 407,
about 0.1% w/v human serum albumin, and
about 100 mM sodium chloride.

In a preferred embodiment, the lyophilized unit dose of the invention as described herein comprises the following pharmaceutically acceptable excipients:
about 82.9 mg a, α-trehalose dihydrate,
about 5 mg poloxamer 407,
about 0.5 mg human serum albumin, and
about 50 μmoles sodium chloride.

In a preferred embodiment, the reconstituted unit dose of the invention as described herein comprises the following pharmaceutically acceptable excipients:
about 15% w/v α,α-trehalose dihydrate,
about 1% w/v poloxamer 407,
about 0.1% w/v human serum albumin, and
about 137 mM sodium chloride, and preferably has a pH of 7 to 8.5

In a preferred embodiment, the reconstituted unit dose of the invention as described herein comprises the following pharmaceutically acceptable excipients when measured in 0.5 ml:
about 15% w/v α,α-trehalose,
about 1% w/v poloxamer 407,
about 0.1% w/v human serum albumin, and preferably
about 137 mM sodium chloride and preferably has a pH of 7 to 8.5.

In a preferred embodiment, the reconstituted unit dose of the invention as described herein comprises the following pharmaceutically acceptable excipients:
about 82.9 mg α,α-trehalose dihydrate,
about 5 mg poloxamer 407,
about 0.5 mg human serum albumin, and preferably
about 68.5 μmoles sodium chloride, and preferably
has a pH of 7 to 8.5.

The human serum albumin may be a native or recombinant human serum albumin (rHSA). The poloxamer 407 may be e.g. Pluronic F127.

In one embodiment, the unit dose further comprises a buffer. The buffer may be phosphate buffered saline (PBS). The buffer may include at least one of sodium chloride (NaCl), monosodium dihydrogen phosphate (NaH₂PO₄), disodium hydrogen phosphate (Na₂HPO₄), potassium chloride (KCl), and potassium dihydrogen phosphate (KH₂PO₄). In a preferred embodiment, the buffer may include disodium hydrogen phosphate (Na₂HPO₄), potassium chloride (KCl), and potassium dihydrogen phosphate (KH₂PO₄). The buffer may have a pH in the range of 7.0 to 8.5 at 25° C.

### Unit Dose

The present invention is directed in part to a unit dose of a dengue vaccine composition comprising a tetravalent dengue virus composition as described herein and pharmaceutically acceptable excipients as described herein.

The present invention is directed in part to a unit dose of a dengue vaccine composition as described above e.g. of
(i) a dengue serotype 1 (e.g. chimeric dengue serotype 2/1 strain) with a concentration of at least 3.3 log 10 pfu/0.5 mL,
(ii) a dengue serotype 2 (e.g. dengue serotype 2 strain) with a concentration of at least 2.7 log 10 pfu/0.5 mL,
(iii) a dengue serotype 3 (e.g. chimeric dengue serotype ⅔ strain) with a concentration of at least 4.0 log 10 pfu/0.5 mL, and
(iv) a dengue serotype 4 (e.g. chimeric dengue serotype 2/4 strain) with a concentration of at least 4.5 log 10 pfu/0.5 mL.

Preferably, the chimeric dengue serotype 2/1 strain is TDV-1, the dengue serotype 2 strain is TDV-2, the chimeric dengue serotype ⅔ strain is TDV-3, and the chimeric dengue serotype 2/4 strain is TDV-4. More preferably, TDV-1 is characterized by the nucleotide sequence according to SEQ ID No. 1 and the amino acid sequence according to SEQ ID No. 2, TDV-2 is characterized by the nucleotide sequence according to SEQ ID No. 3 and the amino acid sequence according to SEQ ID No. 4, TDV-3 is characterized by the nucleotide sequence according to SEQ ID No. 5 and the amino acid sequence according to SEQ ID No. 6 and TDV-4 is characterized by the nucleotide sequence according to SEQ ID No. 7 and the amino acid sequence according to SEQ ID No. 8.

In one embodiment, the unit dose is lyophilized. In one such embodiment, the lyophilized unit dose is obtained by subjecting a volume of 0.5 mL of the aqueous dengue vaccine composition produced by combining pharmaceutically acceptable excipients as described herein and the dengue vaccine composition as described herein comprising the four dengue virus strains, in particular TDV-1 to TDV-4, to lyophilization. In a preferred embodiment the residual moisture content as determined by Karl Fischer Determination is equal to or less than 5.0%, preferably equal to or less than 3%.

In another embodiment, the unit dose is reconstituted. The reconstituted unit dose is obtained by subjecting the lyophilized unit dose to reconstitution with a pharmaceutically acceptable diluent, preferably before administration of the dengue vaccine. In one such embodiment, reconstitution will be accomplished by adding a pharmaceutically acceptable diluent, such as water for injection, phosphate buffered saline or an aqueous sodium chloride solution, to the lyophilized unit dose. In one embodiment, an aqueous sodium chloride solution, such as a 37 mM aqueous sodium chloride solution, is added to the lyophilized unit dose for reconstitution. In one such embodiment, the lyophilized unit dose will be reconstituted with 0.3 to 0.8 mL, or 0.4 to 0.7 mL, or 0.5 ml of diluent. In a preferred embodiment, the lyophilized unit dose is reconstituted with 0.3 to 0.8 mL, 0.4 to 0.7 mL or 0.5 mL of 37 mM aqueous sodium chloride solution. In a more preferred embodiment, the lyophilized unit dose is reconstituted with 0.5 ml of 37 mM aqueous sodium chloride solution. The reconstituted unit dose can subsequently be administered subcutaneously.

It is preferred that the unit dose in lyophilized form is the final product after manufacture of the unit dose and the storage form of the unit dose, wherein the unit dose in reconstituted form is prepared before administration of the unit dose to a subject.

The present invention is, moreover, directed in part to a unit dose of a dengue vaccine composition comprising:

a tetravalent virus composition including four live attenuated dengue virus strains, wherein the unit dose is lyophilized and upon reconstitution with 0.5 ml of a pharmaceutically acceptable diluent comprises:
(i) a dengue serotype 1, such as a chimeric dengue serotype 2/1 strain, in a concentration of at least 3.3 log 10 pfu/0.5 ml,
(ii) a dengue serotype 2, such as a dengue serotype 2 strain, in a concentration of at least 2.7 log 10 pfu/0.5 ml,
(iii) a dengue serotype 3, such as a chimeric dengue serotype ⅔ strain, in a concentration of at least 4.0 log 10 pfu/0.5 ml, and
(iv) a dengue serotype 4, such as a chimeric dengue serotype 2/4 strain, in a concentration of at least 4.5 log 10 pfu/0.5 ml.

In one embodiment, the reconstituted unit dose has a volume of e.g. 0.5 mL, wherein upon reconstitution with a pharmaceutically acceptable diluent (i), (ii), (iii), and (iv) provide a total concentration of pfu/0.5 mL and based on said concentration, the concentration of (iii) at least 10% of the total concentration in pfu/0.5 mL.

In another embodiment the reconstituted unit dose has a volume of e.g. 0.5 mL, wherein upon reconstitution with a pharmaceutically acceptable diluent (i), (ii), (iii), and (iv) provide a total concentration of pfu/0.5 mL and based on said total concentration the concentration of (ii) in pfu/0.5 mL is less than 10%, and the concentration of (iv) in pfu/0.5 mL is at least 50%, and the concentration of (i) in pfu/0.5 mL is at least 1%, and the concentration of (iii) in pfu/0.5 mL is at least 6%, or at least 8%, or at least 10%, or at least 12%, or at least 14%, or at least 16%, or at least 18%.

It is preferred that the concentration in the reconstituted unit dose of (iii) in pfu/0.5 mL is at least 10%.

In one embodiment the reconstituted unit dose has a volume of e.g. 0.5 mL, wherein upon reconstitution with a pharmaceutically acceptable diluent (i), (ii), (iii), and (iv) provide a total concentration of pfu/0.5 mL and based on said total concentration the concentration of (ii) in pfu/0.5 mL is less than 2%, the concentration of (iv) in pfu/0.5 mL is at least 50%, the concentration of (i) in pfu/0.5 mL is at least 1%, and the concentration of (iii) in pfu/0.5 mL is at least 6%.

In one embodiment, the present invention is directed to a lyophilized unit dose of a dengue vaccine composition comprising upon reconstitution with 0.5 ml of a pharmaceutically acceptable diluent a dengue serotype 1 (e.g. chimeric dengue serotype 2/1 strain) with a concentration of at least 3.3 log 10 pfu/0.5 mL, a dengue serotype 2 (e.g. dengue serotype 2 strain) with a concentration of at least 2.7 log 10 pfu/0.5 mL, a dengue serotype 3 (e.g. chimeric dengue serotype ⅔ strain) with a concentration of at least 4.0 log 10 pfu/0.5 mL, and a dengue serotype 4 (e.g. chimeric dengue serotype 2/4 strain) with a concentration of at least 4.5 log 10 pfu/0.5 mL and pharmaceutically acceptable excipients as described herein, wherein the unit dose is preferably formulated in 0.5 mL before lyophilization. Preferably, the chimeric dengue serotype 2/1 strain is TDV-1, the dengue serotype 2 strain is TDV-2, the chimeric dengue serotype ⅔ strain is TDV-3 and the chimeric dengue serotype 2/4 strain is TDV-4. More preferably, TDV-1 is characterized by the nucleotide sequence according to SEQ ID No. 1 and the amino acid sequence according to SEQ ID No. 2, TDV-2 is characterized by the nucleotide sequence according to SEQ ID No. 3 and the amino acid sequence according to SEQ ID No. 4, TDV-3 is characterized by the nucleotide sequence according to SEQ ID No. 5 and the amino acid sequence according to SEQ ID No. 6 and TDV-4 is characterized by the nucleotide sequence according to SEQ ID No. 7 and the amino acid sequence according to SEQ ID No. 8.

In one such embodiment, the lyophilized unit dose is obtained by lyophilizing 0.5 ml of a dengue vaccine composition comprising a dengue serotype 1 (e.g. chimeric dengue serotype 2/1 strain) in a concentration of 3.3 log 10 pfu/dose to 5.0 log 10 pfu/0.5 mL, a dengue serotype 2 (e.g. dengue serotype 2 strain) in a concentration of 2.7 log 10 pfu/dose to 4.9 log 10 pfu/0.5 mL, a dengue serotype 3 (e.g. chimeric dengue serotype ⅔ strain) in a concentration of 4.0 log 10 pfu/dose to 5.7 log 10 pfu/0.5 mL, and a dengue serotype 4 (e.g. chimeric dengue serotype 2/4 strain) in a concentration of 4.5 log 10 pfu/dose to 5.5 log 10 pfu/0.5 mL and pharmaceutically acceptable excipients as described herein. Preferably, the chimeric dengue serotype 2/1 strain is TDV-1, the dengue serotype 2 strain is TDV-2, the chimeric dengue serotype ⅔ strain is TDV-3 and the chimeric dengue serotype 2/4 strain is TDV-4.

In one such embodiment, the lyophilized unit dose is obtained by lyophilizing 0.5 ml of a dengue vaccine composition comprising a dengue serotype 1 (e.g. chimeric dengue serotype 2/1 strain) in a concentration of 3.3 log 10 pfu/0.5 mL to 3.6 log 10 pfu/0.5 mL, a dengue serotype 2 (e.g. dengue serotype 2 strain) in a concentration of 2.7 log 10 pfu/0.5 mL to 4.0 log 10 pfu/0.5 mL, a dengue serotype 3 (e.g. chimeric dengue serotype ⅔ strain) in a concentration of 4.0 log 10 pfu/0.5 mL to 4.6 log 10 pfu/0.5 mL, and a dengue serotype 4 (e.g. chimeric dengue serotype 2/4 strain) in a concentration of 4.5 log 10 pfu/0.5 mL or 4.6 log 10 pfu/0.5 mL to 5.1 log 10 pfu/0.5 mL and pharmaceutically acceptable excipients as described herein. Preferably, the chimeric dengue serotype 2/1 strain is TDV-1, the dengue serotype 2 strain is TDV-2, the chimeric dengue serotype ⅔ strain is TDV-3 and the chimeric dengue serotype 2/4 strain is TDV-4.

In certain embodiments, the lyophilized unit dose refers to 0.5 mL before lyophilization, wherein TDV-2 and TDV-4 are present in certain relative amounts, based on the total concentration of TDV-1, TDV-2, TDV-3 and TDV-4 in pfu/0.5 mL, and the concentration of TDV-2 measured in pfu/0.5 ml is less than 10% or less than 8% or less than 6%, and the concentration of TDV-4 measured in pfu/0.5 mL is at least 50% or at least 65%. In some of these embodiments, the concentration of TDV-1 measured in pfu/0.5 ml is at least 1% and/or the concentration of TDV-3 measured in pfu/0.5 mL is at least 6%, 7%, 8%, 10%, 12%, 14%, 16% or at least 18%.

In certain embodiments, the reconstituted unit dose has a volume of 0.5 mL and TDV-2 and TDV-4 are present in certain relative amounts, based on the total concentration of TDV-1, TDV-2, TDV-3 and TDV-4 in pfu/0.5 mL, and the concentration of TDV-2 measured in pfu/0.5 mL is less than 10% or less than 8% or less than 6%, and the concentration of TDV-4 measured in pfu/0.5 ml is at least 50% or at least 65%. In some of these embodiments, the concentration of TDV-1 measured in pfu/0.5 mL is at least 1% and/or the concentration of TDV-3 measured in pfu/0.5 ml is at least 6%, 7%, 8%, 10%, 12%, 14%, 16% or at least 18%.

In a further preferred embodiment, the reconstituted unit dose has a volume of 0.5 ml and comprises a tetravalent dengue virus composition including four live attenuated dengue virus strains, wherein the concentration of the dengue serotype 1 (e.g. dengue serotype 2/1 strain) measured in pfu/0.5 mL is 1% to 7% of the total concentration, the concentration of the dengue serotype 2 (e.g. dengue serotype 2 strain) measured in pfu/0.5 mL is less than 8% of the total concentration, such as in the range of 1% to 8% of the total concentration, the concentration of the dengue serotype 3 (e.g. dengue serotype ⅔ strain) measured in pfu/0.5 mL is at least 10% of the total concentration, and the concentration of the dengue serotype 4 (e.g. dengue serotype 2/4 strain) measured in pfu/0.5 mL is at least 65% of the total concentration, such as in the range of 65% to 80%. In certain such embodiments, the arithmetic sum of all four serotypes is in the range of 4.6 log 10 pfu/0.5 mL to 6.7 log 10 pfu/0.5 mL, preferably in the range of 4.6 log 10 pfu/0.5 mL to 5.5 log 10 pfu/0.5 mL.

In a further preferred embodiment, the reconstituted unit dose has a volume of 0.5 ml and comprises a tetravalent dengue virus composition including four live attenuated dengue virus strains, wherein the dengue serotype 1 (e.g. chimeric dengue serotype 2/1 strain) such as TDV-1 and the dengue serotype 2 (e.g. dengue serotype 2 strain) such as TDV-2 are present each in a concentration based on the total concentration in pfu/0.5 mL which is within 5%-points of each other and/or are together less than about 10% of the total concentration in pfu/0.5 mL. In certain such embodiments the dengue serotype 3 (e.g. chimeric dengue serotype ⅔ strain) such as TDV-3 is preferably at least about 10% of the total concentration in pfu/0.5 mL and more preferably the dengue serotype 4 (e.g. chimeric dengue serotype 2/4 strain) such as TDV-4 is at least about 70% of the total concentration in pfu/0.5 mL. In certain such embodiments the dengue serotype 4 (e.g. chimeric dengue serotype 2/4 strain) such as TDV-4 represents the highest concentration in the composition of all four serotypes, preferably with at least about 70% of the total concentration in pfu/0.5 mL, dengue serotype 3 (e.g. chimeric dengue serotype ⅔ strain) such as TDV-3 represents the second highest concentration in the composition of all four serotypes, preferably with at least about 10% of the total concentration in pfu/0.5 mL, and dengue serotype 1 (e.g. chimeric dengue serotype 2/1 strain) such as TDV-1 and dengue serotype 2 (e.g. dengue serotype 2 strain) such as TDV-2 each represent lower concentrations than the concentration of serotype 3 (e.g. chimeric dengue serotype ⅔ strain) such as TDV-3, and optionally together represent less than about 10% of the total concentration in pfu/0.5 mL.

The lyophilized unit dose reconstituted in 0.5 mL will provide the above concentrations for the four dengue serotypes. While the unit dose of a dengue vaccine composition as described herein refers to the concentrations of the dengue serotypes in 0.5 mL, the lyophilized unit dose can be reconstituted with other volumes of a pharmaceutically acceptable diluent, such as an aqueous sodium chloride solution, without changing the absolute virus amount administered or the ratios of the viruses to one another.

In certain embodiments, the lyophilized unit dose of the invention is prepared from a solution comprising a non-reducing sugar, a surfactant, a protein and an inorganic salt.

In certain embodiments, the lyophilized unit dose of the invention is prepared from a solution comprising trehalose, poloxamer 407, human serum albumin and sodium chloride.

In certain embodiments, the lyophilized unit dose of the invention is prepared from a solution comprising about 10% w/v to about 20% w/v α,α-trehalose dihydrate or an equimolar amount of other forms of α,α-trehalose, from about 0.5% w/v to about 1.5% w/v poloxamer 407, from about 0.05% w/v to about 2% w/v human serum albumin, and about 70 mM to about 120 mM sodium chloride.

In preferred embodiments, the lyophilized unit dose of the invention as described herein is prepared from a solution comprising about 15% w/v α,α-trehalose dihydrate, about 1% w/v poloxamer 407, about 0.1% w/v human serum albumin and about 100 mM sodium chloride.

In one embodiment, the solution from which the lyophilized unit dose is prepared further comprises a buffer. The buffer may be phosphate buffered saline (PBS). The buffer may include at least one of sodium chloride (NaCl), monosodium dihydrogen phosphate (NaH₂PO₄), disodium hydrogen phosphate (Na₂HPO₄), potassium chloride (KCl), and potassium dihydrogen phosphate (KH₂PO₄). In a preferred embodiment, the buffer may include disodium hydrogen phosphate (Na₂HPO₄), potassium chloride (KCl), and potassium dihydrogen phosphate (KH₂PO₄). The buffer may have a pH in the range of about 7.0 to about 8.5 at 25° C. or a pH of about 6.8 to about 7.6 at 25° C., preferably a pH of about 7.2 at 25° C.

In preferred embodiments, the reconstituted unit dose of the invention as described herein comprising about 15% w/v α,α-trehalose dihydrate, about 1% w/v poloxamer 407, about 0.1% w/v human serum albumin and about 137 mM sodium chloride. The reconstituted unit dose may have a pH of about 7.0 to about 8.5 at 25° C., preferably a pH of about 7.2 at 25° C.

The unit dose of the invention as described herein activates multiple arms of the immune system neutralizing antibodies, cellular immunity and anti-NS1 antibodies-in both seronegative and seropositive subject populations or in both seronegative and seropositive subjects. Thus, the unit dose of the invention as described herein protects both dengue seronegative and dengue seropositive subject populations or subjects against dengue disease.

In one embodiment, one unit dose is present in a container, preferably a vial, and said unit dose is administered to a subject after reconstitution. In one embodiment, more than one unit dose of the dengue vaccine composition may be present in a container, preferably a vial, so that with the content of one container, preferably a vial, more than one subject can be vaccinated. In one embodiment, the container comprising more than one unit doses of the invention as described herein is used for providing the reconstituted unit dose to be used in the methods of the invention as described herein.

The certain embodiments, the container comprising the unit dose of the invention is part of a kit. Thus, the invention is directed in part to a kit for preparing a reconstituted unit dose comprising a lyophilized unit dose of the present invention as described herein, and a pharmaceutically acceptable diluent for reconstitution.

In certain embodiments, the diluent for reconstitution provided in a container, preferably a vial, or a pre-filled syringe. In some embodiments, the diluent for reconstitution is selected from water for injection, phosphate buffered saline or an aqueous sodium chloride solution. In a preferred embodiment, the diluent for reconstitution is 30 to 40 mM sodium chloride, such as 37 mM sodium chloride.

In certain embodiments, the kit may further comprise a hepatitis A vaccine, such as HAVRIX® or VAQTAR. In some embodiments, the hepatitis A vaccine may be in a separate container, such as a vial. In another embodiment, the hepatitis A vaccine and the unit dose of the invention may be in the same container. Thus, the invention is directed in part to a combined dengue/hepatitis A vaccine, wherein the unit dose of the invention as described herein is combined with a hepatitis A vaccine. Such a combined dengue/hepatitis A vaccine comprises the unit dose of the invention as described herein and a hepatitis A vaccine, such as HAVRIX® or VAQTAR, in the same formulation. In certain embodiments, the invention is directed to a kit comprising such a combined dengue/hepatitis A vaccine and a unit dose of the invention as described herein.

### Hepatitis a Vaccine

In certain embodiments, the hepatitis A vaccine is an inactivated hepatitis A vaccine.

In certain embodiments, the hepatitis A vaccine comprises a hepatitis A virus derived from a hepatitis A virus strain HM-175.

In certain embodiments, wherein the hepatitis A vaccine comprises an inactivated hepatitis A virus and the inactivated hepatitis A virus is derived from a wild-type hepatitis A virus strain HM-175.

In certain embodiments, the inactivated hepatitis A virus is adsorbed on a carrier aluminum. In some of these embodiments, the aluminum is aluminum hydroxide or aluminum hydroxyphosphate sulfate.

In certain embodiments, wherein the hepatitis A vaccine comprises a phosphate-buffered saline solution and excipients dissolved therein in the form of an amino acid and in the form of polysorbate. In such embodiments, the amino acid is present at a concentration of 0.2 to 0.8% w/v and/or the polysorbate is present at a concentration of 0.01 to 0.09 mg/ml.

In certain embodiments, the hepatitis A vaccine includes a hepatitis A virus expressing a viral antigen in a concentration ranging from 500 ELISA Units (EL.U.) to 2000 ELISA Units (EL.U.), preferably from 700 EL.U. to 1600 EL.U., most preferably from 1300 to 1550 EL.U., Alternatively, the concentration ranges from 500 EL.U. to 900 EL.U., In a further embodiment, the concentration ranges from 200 to 400 EL.U.

In certain embodiments, the hepatitis A vaccine is included in a liquid 1 ml dose or in a 0.5 ml dose.

An example of such an hepatitis A vaccine is HAVRIX®, from GlaxoSmithKline, which is a sterile suspension of inactivated virus for intramuscular administration. HAVRIX® makes use of the hepatitis A virus strain HM-175 which is derived from a wild-type hepatitis A virus (HAV) HM-175 of which the complete nucleotide sequence is disclosed in Cohen et al., Journal of Virology, Vol. 61, No. 1, published January 1987, p. 50 to 59 (in particular, the entire sequence of the wild-type hepatitis A virus HM-175 is provided in FIG. 1 of said publication).

The virus (strain HM175) is propagated in MRC-5 human diploid cells. After removal of the cell culture medium, the cells are lysed to form a suspension. This suspension is purified through ultrafiltration and gel permeation chromatography procedures. Treatment of this lysate with formalin ensures viral inactivation. Viral antigen activity is referenced to a standard using an enzyme linked immunosorbent assay (ELISA), and is therefore expressed in terms of ELISA Units (EL.U.). Each 1-mL dose for adults (≥18 years of age) of vaccine contains 1440 EL.U. of viral antigen, adsorbed on 0.5 mg of aluminum as aluminum hydroxide. Each 0.5-ml dose for children and adolescents (12 months through 18 years of age) of vaccine contains 720 EL.U. of viral antigen, adsorbed onto 0.25 mg of aluminum as aluminum hydroxide. HAVRIX® contains the following excipients: Amino acid supplement (0.3% w/v) in a phosphate-buffered saline solution and polysorbate 20 (0.05 mg/mL). From the manufacturing process, HAVRIX® also contains residual MRC-5 cellular proteins (not more than 5 μg/mL), formalin (not more than 0.1 mg/ml), and neomycin sulfate (not more than 40 ng/ml), an aminoglycoside antibiotic included in the cell growth media. HAVRIX® is formulated without preservatives.

Another useful hepatitis A vaccine is VAQTAR from Merck Sharp & Dohme Corp., which is an inactivated whole virus vaccine derived from hepatitis A virus grown in cell culture in human MRC-5 diploid fibroblasts. It contains inactivated virus of a strain, which was originally derived by further serial passage of a proven attenuated strain. The virus is grown, harvested, purified by a combination of physical and high performance liquid chromatographic techniques developed at the Merck Research Laboratories, formalin inactivated, and then adsorbed onto amorphous aluminum hydroxyphosphate sulfate. VAQTA® is a sterile suspension for intramuscular injection. One milliliter of the vaccine contains approximately 50 U of hepatitis A virus antigen, which is purified and formulated without a preservative. Within the limits of current assay variability, the 50 U dose of VAQTAR contains less than 0.1 μg of non-viral protein, less than 4×10⁻⁶ μg of DNA, less than 10⁻⁴ μg of bovine albumin, and less than 0.8 μg of formaldehyde. Other process chemical residuals are less than 10 parts per billion (ppb), including neomycin. Each 0.5-mL pediatric dose contains 25 U of hepatitis A virus antigen and adsorbed onto approximately 0.225 mg of aluminum provided as amorphous aluminum hydroxyphosphate sulfate, and 35 μg of sodium borate as a pH stabilizer, in 0.9% sodium chloride. Each 1-mL adult dose contains 50 U of hepatitis A virus antigen and adsorbed onto approximately 0.45 mg of aluminum provided as amorphous aluminum hydroxyphosphate sulfate, and 70 μg of sodium borate as a pH stabilizer, in 0.9% sodium chloride.

### Yellow Fever Vaccine

YF-VAX®, a yellow fever vaccine from Sanofi, for subcutaneous use, is prepared by culturing the YF-17D strain of yellow fever virus in living avian leukosis virus-free (ALV-free) chicken embryos. The vaccine contains sorbitol and gelatin as a stabilizer and is lyophilized. No preservative is added. YF-VAX is formulated to contain not less than 4.74 log₁₀ pfu per 0.5 ml dose throughout the life of the product.

### Combined Vaccine Composition

The present invention is also directed in part to a combined vaccine composition comprising a hepatitis A antigen as in HAVRIX® or VAQTAR, and a dengue antigen such as the tetravalent dengue vaccine, TDV, as disclosed herein or any other suitable tetravalent live attenuated dengue virus vaccine.

In certain embodiments, the invention is directed to the combined vaccine composition, wherein the dengue vaccine composition comprises a tetravalent dengue virus composition including four live attenuated dengue virus strains:
(i) a dengue serotype 1 preferably in a concentration of at least 3.3 log 10 pfu/0.5 mL,
(ii) a dengue serotype 2 preferably in a concentration of at least 2.7 log 10 pfu/0.5 mL,
(iii) a dengue serotype 3 preferably in a concentration of at least 4.0 log 10 pfu/0.5 mL, and
(iv) a dengue serotype 4 preferably strain in a concentration of at least 4.5 log 10 pfu/0.5 mL.

In certain embodiments, the invention is directed to the combined vaccine composition, wherein the dengue vaccine composition comprises a tetravalent dengue virus composition including four live attenuated dengue virus strains:
(i) a chimeric dengue serotype 2/1 strain in a concentration of at least 3.3 log 10 pfu/0.5 mL to 3.8 log 10 pfu/0.5 ml,
(ii) a dengue serotype 2 strain in a concentration of at least 2.7 log 10 pfu/0.5 mL,
(iii) a chimeric dengue serotype ⅔ strain in a concentration of at least 4.0 log 10 pfu/0.5 mL, and
(iv) a chimeric dengue serotype 2/4 strain in a concentration of at least 4.5 log 10 pfu/0.5 mL or at least 4.6 log 10 pfu/0.5 mL to optionally 6.2 log 10 pfu/0.5 ml.

Preferably, in said embodiments the chimeric dengue serotype 2/1 strain is TDV-1, the dengue serotype 2 strain is TDV-2, the chimeric dengue serotype ⅔ strain is TDV-3 and the chimeric dengue serotype 2/4 strain is TDV-4. More preferably, TDV-1 is characterized by the nucleotide sequence according to SEQ ID No. 1 and the amino acid sequence according to SEQ ID No. 2, TDV-2 is characterized by the nucleotide sequence according to SEQ ID No. 3 and the amino acid sequence according to SEQ ID No. 4, TDV-3 is characterized by the nucleotide sequence according to SEQ ID No. 5 and the amino acid sequence according to SEQ ID No. 6 and TDV-4 is characterized by the nucleotide sequence according to SEQ ID No. 7 and the amino acid sequence according to SEQ ID No. 8.

In certain embodiments, the invention is directed to the combined vaccine composition, wherein upon reconstitution of the dengue vaccine composition with a pharmaceutically acceptable diluent (i), (ii), (iii), and (iv) provide a total concentration of pfu/0.5 mL and based on said total concentration of pfu/0.5 ml the concentration of (ii) in pfu/0.5 mL is less than 10%, and the concentration of (iv) in pfu/0.5 mL is at least 50%, and the concentration of (i) in pfu/0.5 ml is at least 1%, and the concentration of (iii) in pfu/0.5 mL is at least 6%, at least 8%, or at least 10%, or at least 12%, or at least 14%, or at least 16%, or at least 18%.

In certain embodiments, the invention is directed to the combined vaccine composition, wherein the dengue vaccine composition comprises one or more pharmaceutically acceptable excipients. In one embodiment, the dengue vaccine composition comprises a non-reducing sugar, a surfactant, a protein and an inorganic salt. Preferably, the non-reducing sugar is trehalose, the surfactant is poloxamer 407, the protein is human serum albumin and the inorganic salt is sodium chloride.

Furthermore, any vaccine excipients or combinations thereof known to the person skilled in the art, e.g. disclosed in WO 2018/027075 A1, can be used for the combined vaccine composition.

In one embodiment, the unit dose of a dengue vaccine composition comprises the following pharmaceutically acceptable excipients:
from about 10% w/v to about 20% w/v α,α-trehalose dihydrate or an equimolar amount of other forms of a, α-trehalose,
from about 0.5% w/v to about 1.5% w/v poloxamer 407,
from about 0.05% w/v to about 2% w/v human serum albumin, and
from about 70 mM to 140 mM sodium chloride.

In certain embodiments, the invention is directed to the combined vaccine composition, wherein the dengue vaccine composition comprises other dengue vaccines such as Dengvaxia®. Dengvaxia® is a tetravalent dengue vaccine with mixed chimeric dengue viruses based on a yellow fever backbone, CYD-TDV (Dengvaxia®, Sanofi Pasteur, Lyon, France), and has been licensed in several countries based on the clinical demonstration of an overall vaccine efficacy (VE) against virologically-confirmed dengue (VCD) of 56-61% in children in Asia and Latin America (Capeding M R et al. Clinical efficacy and safety of a novel tetravalent dengue vaccine in healthy children in Asia: a phase 3, randomised, observer-masked, placebo-controlled trial. Lancet 2014, 384:1358-65; Villar L A et al. Safety and immunogenicity of a recombinant tetravalent dengue vaccine in 9-16 year olds: a randomized, controlled, phase II trial in Latin America. Pediatr Infect Dis J 2013, 32:1102-9). The preparation of these particular strains CYD1, CYD2, CYD3 and CYD4 has been described in detail in international patent applications WO 98/37911, WO 03/101397, WO07/021672, WO 08/007021, WO 08/047023 and WO 08/065315, to which reference may be made for a precise description of the processes for their preparation. The corresponding nucleotide sequences of the prM-E regions of CYD1, CYD2, CYD3 and CYD4 are provided in WO2016034629 and SEQID NOs are set out in Table 16 of this reference.

In certain embodiments, the invention is directed to the combined vaccine composition, wherein the quantity of a chimeric dengue virus within CYD-TDV comprised in a vaccine composition of the present invention lies within a range of about 10⁵ CCID50 to about 106 CCID50. The quantity of a live attenuated chimeric dengue virus of each of serotypes 1 to 4 comprised in the CYD dosage form, e.g. Dengvaxia®, is preferably equal.

In such embodiments, the CYD-TDV is dissolved/dissolvable in a solution containing 0.4% NaCl.

In certain embodiments, the invention is directed to the combined vaccine composition, wherein the dengue vaccine composition comprises other dengue vaccines such as TV003 or TV005. TV003, developed by the U.S. National Institute of Allergy and Infectious Diseases, comprises vaccine components rDEN1A30, rDEN 2/4A30, rDEN3A30/31 and rDEN4430, wherein each of these components is present at a concentration of 3 log₁₀ PFU. TV005 is similar to TV003 with the difference that the concentration of rDEN 2/4430 in TV005 is 4 log₁₀ PFU. The vaccines TV003 and TV005 and their vaccine components as well as their production are described in more detail in WO 2008/022196 A2 and S. S. Whitehead, Expert Rev Vaccines, 2016, 15 (4): 509 to 517. Using recombinant DNA technology, two attenuation strategies were utilized for the vaccine components of TV003 or TV005: deletions in the 3′ untranslated region and structural gene chimerization. For example, the component rDEN4A30 contains all the structural and non-structural proteins of a wild type DENV-4, but is attenuated by a 30-nucleotide deletion in the 3′ untranslated region (denoted “A30”). The other vaccine components are also attenuated due to the 30-nucleotide deletion in the 3′ untranslated region. In addition, rDEN3A30/31 includes a 31 nucleotide deletion in the 3′ untranslated region (shown in detail in FIG. 1c and FIG. 13 of WO 2008/022196 A2). The rDEN 2/4A30 component was created by substituting the prM and E genes of DENV-2 into the rDEN4A30 genome. The complete genomic sequences of dengue strains which can be used to produce TV003 or TV005 are available under the Genbank accession numbers in Table A of WO 2008/022196 A1.

In certain embodiments, the invention is directed to the combined vaccine composition, wherein the hepatitis A vaccine is an inactivated hepatitis A vaccine.

In certain embodiments, wherein the hepatitis A vaccine comprises a hepatitis A virus derived from a hepatitis A virus strain HM-175.

In certain embodiments, the invention is directed to the combined vaccine composition, wherein the hepatitis A vaccine comprises an inactivated hepatitis A virus and the inactivated hepatitis A virus is derived from a wild-type hepatitis A virus strain HM-175.

In certain embodiments, the invention is directed to the combined vaccine composition, the inactivated hepatitis A virus is adsorbed on a carrier aluminum. In some of these embodiments, the aluminum is aluminum hydroxide or aluminum hydroxyphosphate sulfate.

In certain embodiments, the invention is directed to the combined vaccine composition, wherein the hepatitis A vaccine comprises a phosphate-buffered saline solution and excipients dissolved therein in the form of an amino acid and in and in the form of polysorbate. In such embodiments, the amino acid is present at a concentration of 0.2 to 0.8% w/v and/or the polysorbate is present at a concentration of 0.01 to 0.09 mg/ml.

In certain embodiments, the invention is directed to the combined vaccine composition, wherein the hepatitis A vaccine includes a hepatitis A virus expressing a viral antigen in a concentration ranging from 500 ELISA Units (EL.U.) to 2000 ELISA Units (EL.U.), preferably from 700 EL.U. to 1600 EL.U., most preferably from 1300 to 1550 EL.U., Alternatively, the concentration ranges from 500 EL.U. to 900 EL.U., In a further embodiment, the concentration ranges from 200 to 400 EL.U . . .

In certain embodiments, the invention is directed to the combined vaccine composition, wherein the combined vaccine is included in a dose comprising a liquid, wherein the liquid has a volume of 0.5 ml, 1 ml, or 1.5 ml.

In certain embodiments, the combined vaccine composition is provided in one single vial in a liquid form or in a dehydrated form, such as a lyophilized form.

In certain embodiments, the combined vaccine composition is obtained from mixing a unit dose of a dengue vaccine composition and a dose of a hepatitis A vaccine in a syringe.

The invention is also directed in part to a method of administering any of the above combined vaccine compositions to a subject or subject population.

In certain embodiments, the invention is directed to said methods, wherein the combined vaccine composition is administered subcutaneously or intramuscularly.

### Method of Preventing Dengue Disease and Hepatitis a, Corresponding Uses, and Corresponding Kit

The present invention is directed to a method of preventing hepatitis A and dengue disease.

The present invention is directed in part to a method of preventing hepatitis A and dengue disease in a subject or subject population, the method comprising simultaneously on the same day administering a hepatitis A vaccine, such as HAVRIX® or VAQTAR, and a unit dose of a dengue vaccine composition, wherein said unit dose comprises a tetravalent dengue virus composition including four live, attenuated dengue virus strains.

In certain embodiments, the invention is directed to said method, wherein the hepatitis A vaccine, such as HAVRIX®, comprises an inactivated virus. Preferably, the hepatitis A vaccine comprises an inactivated hepatitis A virus and the inactivated hepatitis A virus is derived from a hepatitis A virus strain HM-175.

In certain embodiments, the hepatitis A vaccine, such as HAVRIX®, is derived from a hepatitis A virus strain HM-175.

In certain embodiments, the invention is directed to said methods, wherein the hepatitis A vaccine, such as HAVRIX®, which is preferably a virus derived from a hepatitis A virus strain HM-175, is adsorbed on aluminum. According to some of these embodiments, the aluminum is aluminum hydroxide or aluminum hydroxyphosphate sulfate.

In certain embodiments, the invention is directed to said method, wherein the hepatitis A vaccine, such as HAVRIX®, which is preferably derived from a hepatitis A virus strain HM-175, comprises a phosphate-buffered saline solution and excipients dissolved therein in the form of an amino acid and in and in the form of polysorbate.

In certain embodiments, the invention is directed to said method, wherein the hepatitis A vaccine, such as HAVRIX®, includes a hepatitis A virus expressing a viral antigen in a concentration ranging from 500 ELISA Units (EL.U.) to 2000 ELISA Units (EL.U.), preferably from 700 EL.U. to 1600 EL.U., most preferably from 1300 to 1550 EL.U., Alternatively, the concentration ranges from 500 EL.U. to 900 EL.U., In a further embodiment, the concentration ranges from 200 to 400 EL.U . . .

For example, viral antigen activity of a hepatitis A vaccine can be measured according to a method disclosed in Andre F E., Hepburn A., D′Hondt E., “Inactivated candidate vaccines for hepatitis”, A. Prog Med Virol 1990; 37:72-95.

In certain embodiments, the invention is directed to said method, wherein the dengue vaccine composition upon reconstitution with 0.5 ml of a pharmaceutically acceptable diluent comprises
(i) a chimeric dengue serotype 2/1 strain in a concentration of at least 3.3 log 10 pfu/0.5 mL,
(ii) a dengue serotype 2 strain in a concentration of at least 2.7 log 10 pfu/0.5 mL,
(iii) a chimeric dengue serotype ⅔ strain in a concentration of at least 4.0 log 10 pfu/0.5 mL, and
(iv) a chimeric dengue serotype 2/4 strain in a concentration of at least 4.5 log 10 pfu/0.5 mL.

According to some of these embodiments, upon reconstitution of the dengue vaccine composition with a pharmaceutically acceptable diluent, (i), (ii), (iii), and (iv) provide a total concentration of pfu/0.5 mL and based on said total concentration of pfu/0.5 ml the concentration of (ii) in pfu/0.5 ml is less than 10%, and the concentration of (iv) in pfu/0.5 mL is at least 50%, and the concentration of (i) in pfu/0.5 mL is at least 1%, and the concentration of (iii) in pfu/0.5 ml is at least 6%, at least 8%, or at least 10%, or at least 12%, or at least 14%, or at least 16%, or at least 18%.

In certain embodiments, the invention is directed to said methods, wherein the subject population or subject is seronegative with respect to all dengue serotypes. According to some of these embodiments, the subject population or subject is seronegative with respect to hepatitis A at baseline.

In certain embodiments the invention is directed to said methods, wherein the unit dose of the invention as described herein and the hepatitis A vaccine, such as HAVRIX® or VAQTA®, are administered on day 0/1.

In certain embodiments, the invention is directed to said methods, wherein the unit dose of the invention as described herein is administered by subcutaneous injection and wherein the hepatitis A vaccine, such as HAVRIX® or VAQTAR, is administered by intramuscular injection. According to some embodiments, the injections are administered to the arm, preferably to the deltoid region of the arm. According to some of these embodiments, the subcutaneous injection of the unit dose of the invention as described herein and the intramuscular injection of the hepatitis A vaccine, such as HAVRIX® or VAQTA®, are administered to different anatomical sites, such as to opposite arms.

In certain embodiments, the invention is directed to said methods, wherein two unit doses of the dengue vaccine composition of the invention as described herein are administered. In some embodiments, the two unit doses of the invention as described herein are administered within 12 month or more, or within 6 month, or within three months, such as at day 0/1 and day 90. According to some of these embodiments, a further third unit dose of the invention as described herein is administered after the second administration. Such a third administration may be administered between 6 to 12 months after the first administration, such as 12 months after the first administration, or later than 12 month after the first administration, such as 12 months (1 year) after the second administration or even 5 years or longer after the first or second administration and may act as a booster.

In certain embodiments, the invention is directed to said methods, wherein two unit doses of the invention as described herein and one dose of a hepatitis A vaccine, such as HAVRIX® or VAQTA®, are administered, in particular according to the following schedule
a first simultaneous administration of the first unit dose and said hepatitis A vaccine on day 0/1, and
a second administration of the second unit dose after said first simultaneous administration, such as about 3 months later such as on day 90.

In certain embodiments, the invention is directed to said method, wherein the unit dose of the invention as described herein is administered subcutaneously to a subject or subject population and the hepatitis A vaccine, such as HAVRIX® or VAQTAR, is administered intramuscularly to a subject or subject population, and wherein the subject or the subject population is seronegative with respect to all dengue serotypes. In other embodiments, the subject or subject population is seropositive with respect to at least one dengue serotype.

In certain embodiments, the invention is directed to said method, wherein the unit dose of the invention as described herein and the hepatitis A vaccine, such as HAVRIX® or VAQTA®, are administered to a subject or subject population from a dengue endemic region. In certain embodiments, the unit dose of the invention as described herein is administered subcutaneously and the hepatitis A vaccine, such as HAVRIX® or VAQTA®, is administered intramuscularly to a subject or subject population from a dengue endemic region.

In certain embodiments, the invention is directed to said method, wherein the subject or subject population is from a dengue non-endemic region, preferably from a dengue non-endemic and a hepatitis A non-endemic region.

According to some embodiments, a second dose of a hepatitis A vaccine, such as HAVRIX® or VAQTAR, is administered. The second dose of the hepatitis A vaccine may be administered after the first administration of the hepatitis A vaccine. Such a second administration may act as a booster and may be administered 6 to 12 months or 6 to 18 months, such as 9 months after the first administration of the hepatitis A vaccine, such as on day 270.

In certain embodiments, the invention is directed to said method, wherein the unit dose of the invention as described herein is administered subcutaneously and wherein the hepatitis A vaccine, such as HAVRIX® or VAQTAR, is administered intramuscularly to a subject or subject population of more than 17 years, or more than 18 years, or 18 to 60 years of age. In further embodiments, the subjects or subject population are adults of more than 21 years, or 21 to 60 years, or 21 to 45 years of age. In some embodiments, the subject or subject population is from a dengue endemic region. In another embodiment, the subject or subject population is from a dengue non-endemic region, preferably from a dengue non-endemic and a hepatitis A non-endemic region. According to certain embodiments, the subject or subject population is seronegative for all four dengue serotypes.

In certain embodiments, the invention is directed to said method, wherein the method does not include a step of determination whether there was a previous dengue infection and/or a previous hepatitis A infection in the subject population or in the subject before the administration of the hepatitis A vaccine and before the administration of the unit dose of the dengue vaccine composition or wherein the hepatitis A serostatus and/or the dengue serostatus of the subject population or of the subject is unknown before the administration of the hepatitis A vaccine and before the administration of the unit dose of the dengue vaccine composition. According to certain embodiments, the method does not include a step of determination whether there was a previous dengue infection and/or a previous hepatitis A infection in the subject population or in the subject at any time before, during and after the steps of administration of the hepatitis A vaccine and of the unit dose of the dengue vaccine composition or wherein the hepatitis A serostatus and/or the dengue serostatus of the subject population or of the subject is unknown at any time before, during or after the steps of administration of the hepatitis A vaccine and of the unit dose of the dengue vaccine composition.

In certain embodiments, the invention is directed to said method, wherein the method comprises a primary vaccination consisting of the steps of:
(A) selecting a subject for administration of the unit doses of the tetravalent dengue virus composition and the hepatitis A vaccine in need for protection against dengue infection and hepatitis A infection without determination whether there was a previous dengue infection and/or a previous hepatitis A infection, and
(B) administering simultaneously on the same day a first unit dose of the tetravalent dengue virus composition and a hepatitis A vaccine to the subject, and optionally
(C) administering at least one further unit dose of the tetravalent dengue virus composition to the subject within 3 to 12 months of administration of the first unit dose and optionally
(D) administering at least one further dose of the hepatitis A vaccine to the subject within 6 to 18 months of administration of the first unit dose.

In certain embodiments, the invention is directed to said method, the method comprises a primary vaccination consisting of the steps of:
(A) selecting a subject for administration of the unit doses of the tetravalent dengue virus composition and the hepatitis A vaccine in need for protection against dengue infection and hepatitis A infection, and
(B) administering simultaneously on the same day a first unit dose of the tetravalent dengue virus composition and a hepatitis A vaccine to the subject, and
(C) administering two further unit doses of the tetravalent dengue virus composition to the subject at about 6 and about 12 months of administration of the first unit dose and administering a further hepatitis A vaccine to the subject at either about 6 or about 12 months of administration of the first unit dose. In some of these embodiments, step (A), the selecting of the subject, is carried out without determination whether there was a previous hepatitis A infection.

In certain embodiments, the invention is directed to said method, wherein upon reconstitution of the unit dose with a pharmaceutically acceptable diluent (i), (ii), (iii), and (iv) provide a total concentration of pfu/0.5 mL and based on said total concentration of pfu/0.5 ml the concentration of (ii) in pfu/0.5 mL is less than 10%, and the concentration of (iv) in pfu/0.5 ml is at least 50%, and the concentration of (i) in pfu/0.5 mL is at least 1%, and the concentration of (iii) in pfu/0.5 mL is at least 6%, at least 8%, or at least 10%, or at least 12%, or at least 14%, or at least 16%, or at least 18%.

In certain embodiments, the method provides compatibility between the dengue vaccine composition and the hepatitis A vaccine. Compatibility means in particular that the immune response after simultaneous administration is not inferior in comparison with a mono-administration of these vaccines.

In certain embodiments, the method provides synergy between the dengue vaccine composition and the hepatitis A vaccine. Synergy means in particular that the immune response after simultaneous administration is better for one or both vaccines in comparison with a mono administration of these vaccines.

In certain embodiments, the invention is directed to said method, wherein the method provides non-inferiority in a non-inferiority clinical study including at least 60 or at least 120 healthy subjects divided into one subject population and into one control subject population, wherein the subject population receives simultaneously on the same day the hepatitis A vaccine and the unit dose of the dengue vaccine composition and the control subject population receives simultaneously on the same day a hepatitis A vaccine and a placebo administration

In certain embodiments, the invention is directed to said methods, wherein the hepatitis A vaccine provides a hepatitis A seroprotection rate of at least 95% or of at least 98% on day 30 after an administration (on day 0/1) to a subject population of at least 30 or at least 50 healthy subjects receiving simultaneously on the same day the hepatitis A vaccine and the unit dose of the dengue vaccine composition and being seronegative with respect to hepatitis A at baseline and being seronegative with respect to all dengue virus serotypes at baseline.

In certain embodiments, the invention is directed to said method, wherein the method provides a hepatitis A seroprotection rate difference with respect to a hepatitis A mono-administration, the difference being determined in a non-inferiority clinical study including at least 60 or at least 120 healthy subjects being seronegative with respect to hepatitis A at baseline and seronegative with respect to all dengue virus serotypes at baseline, the healthy subjects being divided into
a) a subject population of at least 30 or at least 50 healthy subjects receiving simultaneously on the same day an administration (on day 0/1) of the hepatitis A vaccine and the unit dose of the dengue vaccine composition, and
b) a control subject population of at least 30 or at least 50 healthy subjects receiving simultaneously on the same day an administration (on day 0/1) of a hepatitis A vaccine and a placebo,
wherein the difference is determined between the hepatitis A seroprotection rate of the control subject population on day 30 after the administration (on day 0/1) and the hepatitis A seroprotection rate of the subject population on day 30 after the administration (on day 0/1), and
wherein the difference has an upper bound within a two-sided 95% confidence interval which is lower than 10%.

In certain embodiments, the invention is directed to said method, wherein the hepatitis A vaccine provides a hepatitis A seroprotection rate of at least 95% or of at least 98% or of at least 99% on day 30 after an administration (on day 0/1) to a subject population of at least 30 or at least 50 healthy subjects receiving simultaneously on the same day the hepatitis A vaccine and the unit dose of the dengue vaccine composition and being seronegative with respect to hepatitis A at baseline, wherein the healthy subjects include healthy subject(s) which are seropositive with respect to at least one dengue virus serotype at baseline and healthy subject(s) which are seronegative with respect to all dengue virus serotypes at baseline.

In certain embodiments, the invention is directed to said method, wherein the method provides a hepatitis A seroprotection rate difference with respect to a hepatitis A mono-administration, the difference being determined in a non-inferiority clinical study including at least 60 or at least 120 healthy subjects being seronegative with respect to hepatitis A at baseline, wherein the healthy subjects include healthy subject(s) which are seropositive with respect to at least one dengue virus serotype at baseline and healthy subject(s) which are seronegative with respect to all dengue virus serotypes at baseline, the healthy subjects being divided into
a) a subject population of at least 30 or at least 50 healthy subjects receiving simultaneously on the same day an administration (on day 0/1) of the hepatitis A vaccine and the unit dose of the dengue vaccine composition, wherein the subject population includes healthy subject(s) which are seropositive with respect to at least one dengue virus serotype at baseline and healthy subject(s) which are seronegative with respect to all dengue virus serotypes at baseline, and
b) a control subject population of at least 30 or at least 50 healthy subjects receiving simultaneously on the same day an administration (on day 0/1) of a hepatitis A vaccine and a placebo, wherein the control subject population includes healthy subject(s) which are seropositive with respect to at least one dengue virus serotype at baseline and healthy subject(s) which are seronegative with respect to all dengue virus serotypes at baseline,
wherein the difference is determined between the hepatitis A seroprotection rate of the control subject population on day 30 after the administration (on day 0/1) and the hepatitis A seroprotection rate of the subject population on day 30 after the administration (on day 0/1), and
wherein the difference has an upper bound within a two-sided 95% confidence interval which is lower than 10%.

In certain embodiments, the invention is directed to said method, wherein the subject or subject population is exposed to a hepatitis A virus outbreak and/or a dengue virus outbreak.

In certain embodiments, the invention is directed to said method, wherein the method provides an anti-hepatitis A virus antibody Geometric Mean Concentration (GMC) of at least 70 mIU/ml or at least 80 mIU/ml or at least 90 mIU/ml on day 30 after an administration (on day 0/1) to a subject population of at least 30 or at least 50 healthy subjects receiving simultaneously on the same day the hepatitis A vaccine and the unit dose of the dengue vaccine composition and being seronegative with respect to hepatitis A at baseline and being seronegative with respect to all dengue virus serotypes at baseline.

An ELISA for determining the anti-hepatitis A antibodies is for example disclosed in Beck et al. J Travel Med 2004; 11:201-207.

In certain embodiments, the invention is directed to said method, wherein the simultaneous administration of the hepatitis A vaccine and the unit dose of the dengue vaccine composition to the subject or the subject population does not provide serious adverse events related to the simultaneous administration. Additionally, there are no deaths related to the simultaneous administration.

In certain embodiments, the invention is directed to said methods, wherein the method provides the Geometric Mean Titer (GMT) of neutralizing antibodies measured by MNT50 of
at least 110 or at least 140 or at least 150 for dengue serotype 1,
at least 3000 or at least 3500 or at least 3900 for dengue serotype 2,
at least 100 or at least 120 or at least 140 for dengue serotype 3, and/or
at least 80 or at least 110 or at least 140 for dengue serotype 4, on day 30 after an administration (on day 0/1) to a subject population of at least 30 or at least 50 healthy subjects receiving simultaneously on the same day the hepatitis A vaccine and the unit dose of the dengue vaccine composition and being seronegative with respect to hepatitis A at baseline and being seronegative with respect to all dengue virus serotypes at baseline.

In some embodiments, the geometric mean neutralizing antibody titers (GMTs) of a subject population or the neutralizing antibody titers of a subject are determined in accordance with a microneutralization test, for example according to the method described in Example 2.

The present invention is directed in part to a method of preventing hepatitis A and dengue disease in a subject or subject population, the method comprising simultaneously on the same day administering a hepatitis A vaccine, and a unit dose of a dengue vaccine composition, wherein said unit dose comprises a tetravalent dengue virus composition including four live, attenuated dengue virus strains, wherein the four live, attenuated dengue virus strains are different from the ones used in the unit dose as defined above.

In one embodiment of the invention, the method is directed to a simultaneous on the same day administration of a hepatitis A vaccine with other dengue vaccines such as Dengvaxia®. Dengvaxia® is a tetravalent dengue vaccine based on a yellow fever backbone, CYD-TDV (Dengvaxia®, Sanofi Pasteur, Lyon, France), and has been licensed in several countries based on the clinical demonstration of an overall vaccine efficacy (VE) against virologically-confirmed dengue (VCD) of 56-61% in children in Asia and Latin America (Capeding M R et al. Clinical efficacy and safety of a novel tetravalent dengue vaccine in healthy children in Asia: a phase 3, randomised, observer-masked, placebo-controlled trial. Lancet 2014, 384:1358-65; Villar L A et al. Safety and immunogenicity of a recombinant tetravalent dengue vaccine in 9-16 year olds: a randomized, controlled, phase II trial in Latin America. Pediatr Infect Dis J 2013, 32:1102-9). The preparation of these particular strains CYD1, CYD2, CYD3 and CYD4 has been described in detail in international patent applications WO 98/37911, WO 03/101397, WO07/021672, WO 08/007021, WO 08/047023 and WO 08/065315, to which reference may be made for a precise description of the processes for their preparation. The corresponding nucleotide sequences of the prM-E regions of CYD1, CYD2, CYD3 and CYD4 are provided in WO2016034629 and SEQID NOs are set out in Table 16 of this reference.

In one such embodiment, the method comprises a vaccination consisting of the steps of:
(A) selecting a subject for administration of the equal doses of the CYD-TDV composition, such as Dengvaxia®, and the hepatitis A vaccine, such as HAVRIX® or VAQTAR, in need for protection against dengue infection and hepatitis A infection, and
(B) administering a first dose of the CYD-TDV composition, such as Dengvaxia®, and the hepatitis A vaccine, such as HAVRIX® or VAQTAR to the subject at month 0,
(C) administering a further dose of the CYD-TDV composition, such as Dengvaxia®, and optionally the hepatitis A vaccine, such as HAVRIX® or VAQTAR to the subject within 3 to 11 months, in particular at about month 6 of the administration of the first CYD-TDV dose, and
(D) administering a final dose of the CYD-TDV, such as Dengvaxia®, and optionally the hepatitis A vaccine, such as HAVRIX® or VAQTAR to the subject at about month 12.

In certain embodiments, the subject is from 2 to 60 years of age.

In particular embodiments, the subject is 2 to 18 years of age, or 4 to 16 years of age, or 18 to 60 years of age.

Preferably, the exact quantity of each component of the CYD-TDV to be administered may vary according to the age and the weight of the subject being vaccinated, the frequency of administration as well as the other ingredients in the composition. The quantity of a chimeric dengue virus within CYD-TDV comprised in a dose of a vaccine composition lies within a range of about 10⁵ CCID50 to about 106 CCID50. The quantity of a live attenuated chimeric dengue virus of each of serotypes 1 to 4 comprised in the CYD dosage form, e.g. Dengvaxia®, is preferably equal. Advantageously, a vaccine composition, as described in this section, comprises an effective amount of a dengue antigen as defined herein.

In certain embodiments, the invention is directed to said method, wherein the dengue vaccine composition comprises other dengue vaccines such as TV003 or TV005. TV003, developed by the U.S. National Institute of Allergy and Infectious Diseases, comprises vaccine components rDEN1A30, rDEN2/4A30, rDEN3A30/31 and rDEN4A30, wherein each of these components is present at a concentration of 3 log₁₀ PFU. TV005 is similar to TV003 with the difference that the concentration of rDEN2/4A30 in TV005 is 4 log₁₀ PFU. The vaccines TV003 and TV005 and their vaccine components as well as their production are described in more detail in WO 2008/022196 A2 and S. S. Whitehead, Expert Rev Vaccines, 2016, 15 (4): 509 to 517. Using recombinant DNA technology, two attenuation strategies were utilized for the vaccine components of TV003 or TV005: deletions in the 3′ untranslated region and structural gene chimerization. For example, the component rDEN4430 contains all the structural and non-structural proteins of a wild type DENV-4, but is attenuated by a 30-nucleotide deletion in the 3′ untranslated region (denoted “A30”). The other vaccine components are also attenuated due to the 30-nucleotide deletion in the 3′ untranslated region. In addition, rDEN3A30/31 includes a 31 nucleotide deletion in the 3′ untranslated region (shown in detail in FIG. 1c and FIG. 13 of WO 2008/022196 A2). The rDEN2/4430 component was created by substituting the prM and E genes of DENV-2 into the rDEN4A30 genome. The complete genomic sequences of dengue strains which can be used to produce TV003 or TV005 are available under the Genbank accession numbers in Table A of WO 2008/022196 A1.

In certain embodiments, the invention is directed to said methods, wherein the unit dose disclosed herein, which in particular comprises a chimeric dengue serotype 2/1 strain, a live attenuated dengue serotype 2 strain, a chimeric dengue serotype 2/3 strain and a chimeric dengue serotype 2/4 strain, and Dengvaxia® disclosed herein and the hepatitis A vaccine disclosed herein are simultaneously on the same day administered to the subject or to the subject population.

In certain embodiments, the invention is directed to said methods, wherein the unit dose disclosed herein, which in particular comprises a chimeric dengue serotype 2/1 strain, a live attenuated dengue serotype 2 strain, a chimeric dengue serotype 2/3 strain and a chimeric dengue serotype 2/4 strain, and the hepatitis A vaccine disclosed herein are simultaneously on the same day administered to the subject or to the subject population on day 0/1 as a first administration and Dengvaxia® disclosed herein is subsequently administered to the subject or to the subject population within three months from the first administration, such as on day 90 from the first administration, as a second administration. Alternatively, Dengvaxia® disclosed herein and the hepatitis A vaccine disclosed herein are simultaneously on the same day administered to the subject or to subject population on day 0/1 as a first administration and the unit dose disclosed herein, which in particular comprises a chimeric dengue serotype 2/1 strain, a live attenuated dengue serotype 2 strain, a chimeric dengue serotype 2/3 strain and a chimeric dengue serotype 2/4 strain, is administered subsequently to the subject or to the subject population within three months from the first administration, such as on day 90 from the first administration, as a second administration.

In certain embodiments, the invention is directed to said methods, wherein the unit dose disclosed herein, which in particular comprises a chimeric dengue serotype 2/1 strain, a live attenuated dengue serotype 2 strain, a chimeric dengue serotype 2/3 strain and a chimeric dengue serotype 2/4 strain, and TV003 or TV005 disclosed herein and the hepatitis A vaccine disclosed herein are simultaneously on the same day administered to the subject or to the subject population.

In certain embodiments, the invention is directed to said methods, wherein the unit dose disclosed herein, which in particular comprises a chimeric dengue serotype 2/1 strain, a live attenuated dengue serotype 2 strain, a chimeric dengue serotype 2/3 strain and a chimeric dengue serotype 2/4 strain, and the hepatitis A vaccine disclosed herein are simultaneously on the same day administered to the subject or the subject population on day 0/1 as a first administration and wherein TV003 or TV005 disclosed herein is subsequently administered to the subject or to the subject population within three months from the first administration, such as on day 90 from the first administration, as a second administration. Alternatively, TV003 or TV005 disclosed herein and the hepatitis A vaccine disclosed herein are simultaneously on the same day administered to the subject or to the subject population on day 0/1 as a first administration and the unit dose disclosed herein, which in particular comprises a chimeric dengue serotype 2/1 strain, a live attenuated dengue serotype 2 strain, a chimeric dengue serotype 2/3 strain and a chimeric dengue serotype 2/4 strain, is administered subsequently to the subject or to the subject population within three months from the first administration, such as on day 90 from the first administration, as the second administration.

The above method is also to be considered in the context of a use of the unit dose of dengue vaccine as disclosed herein for such methods of preventing dengue disease and hepatitis A or in the context of the use of the unit dose of dengue vaccine for the manufacture of a medicament for such methods of preventing dengue disease and hepatitis A.

Furthermore, the present invention is directed to a kit against hepatitis A and dengue disease comprising

a box containing at least
(a) a first container holding a hepatitis A vaccine, as defined above such as HAVRIX®, and
(b) a second container holding a unit dose of a dengue vaccine composition as defined above, wherein said unit dose comprises a tetravalent dengue virus composition including four live, attenuated dengue virus strains.

### Method of Preventing Dengue Disease and Yellow Fever and Uses

The present invention is directed in part to a method of preventing dengue disease as well as yellow fever in a subject. Thus, in certain embodiments the invention is directed to a method of preventing dengue disease in a subject, comprising administering to the subject a reconstituted unit dose of the invention as described herein, wherein the method further comprises preventing yellow fever in the subject by concomitant administration of a yellow fever vaccine, in particular YF-17D, to the subject.

The present invention is directed in part to a method of preventing dengue disease as well as yellow fever in a subject population. Thus, in certain embodiments the invention is directed to a method of preventing dengue disease in a subject population, comprising administering to the subject population a reconstituted unit dose of the invention as described herein, wherein the method further comprises preventing yellow fever in the subject population by concomitant administration of a yellow fever vaccine, in particular YF-17D, to the subject population.

In certain embodiments the invention is directed to said methods, wherein the unit dose of the invention as described herein and the yellow fever vaccine, in particular YF-17D, are administered simultaneously. In some of these embodiments the simultaneous administration is on day 0 or day 90, preferably on day 0. In other embodiments the administration of the unit dose of the invention as described herein and the yellow fever vaccine, in particular YF-17D, are done sequentially such as wherein the yellow fever vaccine is administered before or after the unit dose of dengue vaccine as described herein, such as within about 6 weeks, or such as within about 4 weeks, or such as within about 2 weeks, or such as about within 1 week.

In certain embodiments the invention is directed to said methods, wherein the reconstituted unit dose of the invention as described herein is administered and the yellow fever vaccine, in particular YF-17D, are administered by subcutaneous injection. According to some embodiments, the subcutaneous injections are administered to the arm, preferably to the deltoid region of the arm. According to some of these embodiments the subcutaneous injections of the unit dose of the invention as described herein and yellow fever vaccine, in particular YF-17D, are administered to different anatomical sites, such as to opposite arms, in particular when the vaccines are administered simultaneously.

In certain embodiments the invention is directed to said methods, wherein two unit doses of the invention as described herein are administered. In some embodiments the two unit doses of the invention as described herein are administered within 12 month or more, or within 6 month, or within three months, such as at day 0/1 and day 90. According to some of these embodiments a further third unit dose of the invention as described herein is administered after the second. Such a third administration may act as a booster and may be administered between 6 to 12 months after the first administration, such as 12 months after the first administration, or later than 12 month after the first administration, such as 12 months (1 year) after the second administration or even 5 years or longer after the first or second administration.

In certain embodiments the invention is directed to said methods, wherein two reconstituted unit doses of the invention as described herein and one dose of a yellow fever vaccine, in particular YF-17D, are administered, in particular according to the following schedule
an administration of said yellow fever vaccine on day 0,
a first administration of the first reconstituted unit dose after said yellow fever vaccine administration, such as 3 months later and preferably on day 90, and
a second administration of the second reconstituted unit dose after said first administration of the reconstituted unit dose, such as 3 months later and preferably on day 180.

In certain embodiments the invention is directed to said methods, wherein two reconstituted unit doses of the invention as described herein and one dose of a yellow fever vaccine, in particular YF-17D, are administered, in particular according to the following schedule
a first administration of the first reconstituted unit dose on day 0,
a second administration of the second reconstituted unit dose after said first administration of the reconstituted unit dose, such as 3 months later and preferably on day 90, and
an administration of said yellow fever vaccine after said second administration of the reconstituted unit dose, such as 3 months later and preferably on day 180.

In certain embodiments the invention is directed to said methods, wherein two reconstituted unit doses of the invention as described herein and one dose of a yellow fever vaccine, in particular YF-17D, are administered, in particular according to the following schedule
a simultaneous administration of the first reconstituted unit dose and said yellow fever vaccine on day 0, and
a second administration of the second reconstituted unit dose after said simultaneous administration, such as 3 months later and preferably on day 90.

In a preferred embodiment, the yellow fever vaccine and unit dose of the invention as described herein are administered simultaneously on day 0 or simultaneously on day 90.

In certain embodiments, the invention is directed to said methods, wherein the subject or subject population is seronegative to all dengue serotypes. In certain embodiments, the invention is directed to said methods, wherein the reconstituted unit dose of the invention as described herein is administered subcutaneously to a subject or subject population and the yellow fever vaccine, in particular YF-17D vaccine, is administered subcutaneously to a subject or subject population, and wherein the subject or the subject population is seronegative with respect to all dengue serotypes. In other embodiments, the subject or subject population is seropositive with respect to at least one dengue serotype.

In certain embodiments, the invention is directed to said methods, wherein the unit dose of the invention as described herein and the yellow fever vaccine, in particular YF-17D, are administered to a subject or subject population from a dengue endemic region. In certain embodiments, the reconstituted unit dose of the invention as described herein and the yellow fever vaccine, in particular YF-17D, are administered subcutaneously to a subject or subject population from a dengue endemic region. In other embodiments, the subject or subject population is from a dengue non-endemic region. Such a subject population or such a subject may be vaccinated according to the present invention in the context of traveling to a dengue endemic region and yellow fever endemic region.

In certain embodiments the invention is directed to said methods, wherein the reconstituted unit dose of the invention as described herein and of the yellow fever vaccine, in particular YF-17D, are administered subcutaneously to a subject or subject population of more than 17 years, or more than 18 years, or 18 to 60 years of age. In further embodiments, the subjects or subject population are adults of more than 21 years, or 21 to 60 years, or 21 to 45 years of age. In some embodiments, the subject or subject population is from a dengue endemic region. In another embodiment, the subject or subject population is from a dengue non-endemic region, preferably from a dengue non-endemic and yellow fever non-endemic region. According to some of these embodiments, the subject or subject population are seronegative for all four dengue serotypes.

The above method is also to be considered in the context of a use of the unit dose of dengue vaccine as disclosed herein for such methods or in the context of the use of the unit dose of dengue vaccine for the manufacture of a medicament for such methods.

### Method of Preventing and Uses, Method of Inoculating Against Dengue Disease and Uses

The present invention is directed in part to a method of preventing dengue disease (in particular virologically confirmable dengue, VCD) in a subject. Thus, in certain embodiments the invention is directed to a method of preventing dengue disease in a subject, comprising administering to the subject, a unit dose/tetravalent dengue virus composition, in particular a reconstituted unit dose of the invention as described herein.

The present invention is directed in part to a method of preventing dengue hemorrhagic fever (DHF) and dengue shock syndrome (DSS). Thus, in certain embodiments the invention is directed to a method of preventing dengue hemorrhagic fever (DHF) and dengue shock syndrome (DSS), comprising administering to the subject a reconstituted unit dose/tetravalent dengue virus composition of the invention as described herein.

The present invention is therefore directed to a method of inoculating a subject against virologically confirmable dengue disease with a tetravalent dengue virus composition including four live attenuated dengue virus strains representing serotype 1, serotype 2, serotype 3 and serotype 4, wherein in particular the tetravalent dengue virus composition includes a chimeric dengue serotype 2/1 strain and a dengue serotype 2 strain and a chimeric dengue serotype 2/3 strain and a chimeric dengue serotype 2/4 strain, wherein in particular the dengue serotype 2 strain is derived from the wild type virus strain DEN-2 16681 (SEQ ID NO 11) and differs in at least three nucleotides from the wild type as follows:
a) 5′-noncoding region (NCR)-57 (nt-57 C-to-T): major attenuation locus
b) NS1-53 Gly-to-Asp (nt-2579 G-to-A): major attenuation locus
c) NS3-250 Glu-to-Val (nt-5270 A-to-T): major attenuation locus; and

wherein the three chimeric dengue strains are derived from the serotype 2 strain by replacing the structural proteins prM and E from serotype 2 strain with the corresponding structural proteins from the other dengue serotypes, resulting in the following chimeric dengue strains:
a DENV-2/1 chimera,
a DENV-2/3 chimera and
a DENV-2/4 chimera.

Further information regarding the serotypes of the tetravalent composition can be derived from section “Dengue virus strains” above.

The tetravalent dengue virus composition for such a method may be in the form of a unit dose comprising:
(i) a dengue serotype 1 in a concentration of at least 3.3 log 10 pfu/0.5 ml,
(ii) a dengue serotype 2, in a concentration of at least 2.7 log 10 pfu/0.5 ml,
(iii) a dengue serotype 3, in a concentration of at least 4.0 log 10 pfu/0.5 ml, and
(iv) a dengue serotype 4, in a concentration of at least 4.5 log 10 pfu/0.5 ml.

The present invention is in particular directed to such a method wherein the unit dose is lyophilized and upon reconstitution with 0.5 mL of a pharmaceutically acceptable diluent comprises:
(i) a dengue serotype 1 in a concentration of at least 3.3 log 10 pfu/0.5 ml,
(ii) a dengue serotype 2, in a concentration of at least 2.7 log 10 pfu/0.5 ml,
(iii) a dengue serotype 3, in a concentration of at least 4.0 log 10 pfu/0.5 ml, and
(iv) a dengue serotype 4, in a concentration of at least 4.5 log 10 pfu/0.5 ml.

Further information regarding the tetravalent composition or the unit dose can be derived from section “Dengue vaccine composition” and “Unit dose” above.

The present invention is therefore directed to a method and corresponding use, the method comprising a primary vaccination with only two administrations of the unit dose comprising the steps of:
(A) administering a first unit dose of the tetravalent dengue virus composition to the subject, and
(B) administering a second unit does of the tetravalent dengue virus composition to the subject within 3 months of administration of the first unit dose.

According to this embodiment the administration of only two doses within 3 months is sufficient to provide effective protection against a subsequent dengue infection.

Such method preferably provides a combined vaccine efficacy against all four serotypes in preventing virologically confirmable dengue disease with a 2-sided 95% confidence interval, wherein the lower bound is more than 60%, when measured against placebo in a subject population of at least 5,000 healthy subjects irrespective of serostatus at baseline and 14 to 16 years of age, from the first administration of the administration schedule until 18 months after the second administration of the administration schedule.

Such method also preferably provides a combined vaccine efficacy against all four serotypes, in preventing virologically confirmable dengue disease with a 2-sided 95% confidence interval, wherein the lower bound is more than 45%, when measured against placebo in a subject population of at least 1,500 or at least 2,000 healthy subjects seronegative against all serotypes at baseline and 14 to 16 years of age, from 30 days after the second administration of the administration schedule until 18 months after the second administration of the administration schedule.

According to certain embodiments the method of inoculation against the virologically confirmable dengue disease is due to a dengue serotype 2, and/or due to a dengue serotype 1. The method has very high efficacy against dengue serotype 2 and dengue serotype 1 and the highest efficacy against dengue serotype 2.

In certain embodiments, the invention is directed to said methods having a vaccine efficacy against serotype 1, in preventing virologically confirmable dengue disease with a 2-sided 95% confidence interval, wherein the lower bound is more than 25%, when measured against placebo in a subject population of at least 1,500, or at least 2,000, or at least 5,000 healthy subjects (or at least 10,000, or at least 15,000 healthy subjects) irrespective of serostatus at baseline and 4 to 16 years of age from 30 days post second administration until 12 to 18 months (e.g. at 12 or at 18 months) after the second administration. In certain such embodiments, the lower bound is more than 30%, is more than 35% is more than 40%, is more than 45%, is more than 50%, or is more than 54%. In certain such embodiments the subject population of at least 1,500 is seronegative against all serotypes at base line and the lower bound is more than 35%. In certain such embodiments the seronegative and seropositive population each provide a vaccine efficacy against serotype 1 with a 2-sided 95% confidence interval, wherein the lower bounds are within 10%-points.

In certain embodiments, the invention is directed to said methods having a vaccine efficacy against serotype 1, in preventing virologically confirmable dengue disease, when measured against placebo in a subject population of at least 1,500, or at least 2,000, or at least 5,000 healthy subjects (or at least 10,000, or at least 15,000 healthy subjects) irrespective of serostatus at baseline and 4 to 16 years of age from 30 days post second administration until 12 to 18 months (e.g. at 12 or at 18 months) after the second administration. In certain such embodiments, the vaccine efficacy is more than 40%, is more than 50%, is more than 60%, or is more than 65%. In certain such embodiments the subject population of at least 1,500 is seronegative against all serotypes at base line. In certain such embodiments the seronegative and seropositive population each provide a vaccine efficacy against serotype 1 which are within 5%-points.

In certain embodiments, the invention is directed to said methods having a vaccine efficacy against serotype 2, in preventing virologically confirmable dengue disease with a 2-sided 95% confidence interval, wherein the lower bound is more than 25%, when measured against placebo in a subject population of at least 1,500, or at least 2,000, or at least 5,000 healthy subjects (or at least 10,000, or at least 15,000 healthy subjects) irrespective of serostatus at baseline and 4 to 16 years of age from 30 days post second administration until 12 to 18 months (e.g. at 12 or at 18 months) after the second administration. In certain such embodiments, the lower bound is more than 50%, is more than 60%, is more than 70%, is more than 80%, or is more than 85%. In certain such embodiments the subject population of at least 1,500, is seronegative against all serotypes. In certain such embodiments the seronegative and seropositive population each provide a vaccine efficacy against serotype 2 with a 2-sided 95% confidence interval, wherein the lower bounds are within 5%-points.

In certain embodiments, the invention is directed to said methods having a vaccine efficacy against serotype 2, in preventing virologically confirmable dengue disease, when measured against placebo in a subject population of at least 1,500, or at least 2,000, or at least 5,000 healthy subjects (or at least 10,000, or at least 15,000 healthy subjects) irrespective of serostatus at baseline and 4 to 16 years of age from 30 days post second administration until 12 to 18 months (e.g. at 12 or at 18 months) after the second administration. In certain such embodiments, the vaccine efficacy is more than 60%, is more than 70%, is more than 80%, or is more than 90%. In certain such embodiments the subject population of at least 1,500 is seronegative against all serotypes at base line. In certain such embodiments the seronegative and seropositive population each provide a vaccine efficacy against serotype 2 which are within 5%-points.

The efficacy of the method is further described in more detail below in this the section.

In certain embodiments the unit dose is reconstituted and administered by subcutaneous injection. According to some of these embodiments, the subcutaneous injection is administered to the arm, preferably to the deltoid region of the arm.

According to one embodiment such a method does not include a step of determination whether there was a previous dengue infection in the subject before administration of the unit dose or wherein the serostatus of the subject is unknown before administration of the unit dose.

According to one embodiment such a method does not include a step of determination of a previous dengue infection in the subjects preferably at any time before, during or after the steps of administration or wherein the serostatus of the subject is unknown preferably at any time before, during or after the steps of administration.

The method according to the invention does not require the testing of the serostatus before vaccination and thus allows immediate treatment and outbreak control. According to certain embodiments the use is for a method wherein the subject is exposed to a dengue outbreak. In certain such embodiments the outbreak is due to a dengue serotype 2, and/or due to a serotype 1.

According to one embodiment such a method the subject is from a region wherein the seroprevalence rate is unknown and/or wherein the seroprevalence rate is below 80%, or below 70%, or below 60%.

According to one embodiment of such a method the subject is seronegative at baseline and is from a region or travels to a region wherein the seroprevalence rate is high with respect to serotype 1 and/or serotype 2 i.e. 80%, or 90% or above.

According this embodiment the vaccine and corresponding method is safe for seronegative and seropositive subjects and thus does not require an analysis of the serostatus or a determination of a previous dengue infection or a high seroprevalence rate in the region. Such a method preferably provides a combined vaccine efficacy against virologically-confirmed dengue with hospitalization against all four serotypes with a 2-sided 95% confidence interval, wherein the lower bound is more than 65%, when measured against placebo in a subject population of at least 5,000 healthy 4 to 16 year old subjects irrespective of serostatus at baseline, preferably in at least 1,500 healthy 4 to 16 year old subjects seronegative at baseline, from first administration of the administration schedule until 12 to 18 months after the second administration of the administration schedule. Preferably, the 2-sided 95% confidence interval of the combined vaccine efficacy against virologically-confirmed dengue with hospitalization against all four serotypes when comparing seropositive and seronegative subjects provides for lower bounds of the 2-sided confidence interval which are within 10% points or within 15% points or within 20% points. The method is preferably safe with respect to serotype 1 and serotype 2 which may therefore be used in outbreak situations due to serotype 1 and/or serotype 2 or even for seronegative subjects (e.g. travelers) or subjects with unknown serostatus in regions with very high seroprevalence rates (>80%) due to serotype 1 and/or serotype 2.

The safety of the method is further described in more detail in the section “method of preventing, method of inoculating”.

According to one embodiment such a method does not include the active surveillance with respect to febrile illness of the subject after the administration of the first- and second-unit dose. During active surveillance any subject with febrile illness (defined as fever≥38° C. on any 2 of 3 consecutive days) will be asked to return to the site for dengue fever evaluation by the Investigator. Subjects/guardians will be contacted at least weekly to ensure robust identification of febrile illness by reminding subjects/guardians of their obligation to return to the site in case of febrile illness. This contact will be implemented through appropriate methods that may differ in each trial site (eg, phone calls, text messaging, home visits, school-based surveillance).

According to one embodiment such a method does not include vaccine immunogenicity analysis including GMTs for dengue neutralizing antibodies.

According to one embodiment such a method does not include a reactogenicity analysis. Such a reactogenicity analysis relates to solicited local AEs (injection site pain, injection site erythema, and injection site swelling) and solicited systemic AEs (child<6 years: fever, irritability/fussiness, drowsiness and loss of appetite; child>6 years: asthenia, fever, headache, malaise and myalgia) which will e.g. be assessed for 7 days and 14 days, respectively, following each vaccination (vaccination day included) via collection of diary cards.

According to one embodiment the method does not include an active surveillance, an immunogenicity analysis and a reactogenicity analysis.

According to such embodiments the vaccine and the corresponding method of inoculation are safe and therefore do not require further steps of surveillance or analysis.

In view of the above the method according to one embodiment comprises a primary vaccination consisting of the steps of:
(A) selecting a subject for administration of the unit doses of the tetravalent dengue virus composition in need for protection against dengue infection without determination of a previous dengue infection, and
(B) administering a first unit dose of the tetravalent dengue virus composition to the subject, and
(C) administering a second unit dose of the tetravalent dengue virus composition to the subject within 3 months of administration of the first unit dose.

Therefore the method of inoculating is finalized without determination of a previous dengue infection. The method further optionally comprises at least 1 years after the administration of the second unit dose a booster dose of the unit dose.

Selecting the subject may include all types of considerations but preferably not the determination of a previous dengue infection. The selection may include consideration of the age, health conditions, and threat of infection. The threat of infection includes consideration of the seroprevalence rate in the region in which the subject normally lives or intends to travel, the serotype specific seroprevalence rate and an outbreak situation or serotype specific outbreak situations. The subject may be selected due to its exposure to serotype 1 and/or serotype 2 or due to the fact it requires protection against a specific dengue serotype, i.e. serotype 1 and/or serotype 2.

According to the invention the method is applicable to subjects of all kinds of ages. According to one embodiment the subject is under 9 years of age, or 4 to 5 years of age, or 6 to 11 years of age or 12 to 16 years, or 6 to 16 years of age or 4 to 16 years of age, or 2 to 17 years of age, or 9 years of age, or over 9 years of age, or 9 to 17 years of age, or 18 to 45 years of age, or 46 to 60 years of age, or over 60 years of age.

In particular the present invention is directed to such a method wherein the method which is safe.

In particular the present invention is directed to such a method providing a combined vaccine efficacy against virologically-confirmed dengue with hospitalization against all four serotypes with a 2-sided 95% confidence interval, wherein the lower bound is more than 65%, when measured against placebo in a subject population of at least 5,000 healthy 4 to 16 year old subjects irrespective of serostatus at baseline from first administration of the administration schedule until 12 to 18 months after the last administration of the administration schedule.

In particular the present invention is directed to such a method wherein the method which is effective.

In particular the present invention is directed to such a method providing a combined vaccine efficacy against all four serotypes, in preventing virologically confirmable dengue disease with a 2-sided 95% confidence interval, wherein the lower bound is more than 60%, when measured against placebo in a subject population of at least 5,000 healthy subjects irrespective of serostatus at baseline and 14 to 16 years of age, from the first administration of the administration schedule until 18 months after the last administration of the administration schedule.

In certain embodiments, the invention is directed to said methods, wherein the subject is seronegative to all dengue serotypes.

The present invention is directed in part to a method of preventing dengue disease (in particular virologically confirmable dengue, VCD) in a subject population. Thus, in certain embodiments the invention is directed to a method of preventing dengue disease in a subject population, comprising administering to the subject population a unit dose, in particular a reconstituted unit dose of the invention as described herein.

The present invention is in part directed to said method for preventing dengue disease (in particular virologically confirmable dengue, VCD) in a subject population comprising administering to the subject population at least a first reconstituted unit dose of the invention as described herein, wherein certain ratios of geometric mean neutralizing antibody titers (GMTs) at day 180 or 365 after administration of said first unit dose to the subject population are achieved. According to some embodiments, the geometric mean neutralizing antibody titer for dengue serotype 2 (GMT DENV-2) and the geometric mean neutralizing antibody titer for dengue serotype 4 (GMT DENV-4) when tested in at least 40, or at least 50, or at least 60 subjects at day 180 or day 365 after at least a first administration of said reconstituted unit dose of the invention as described herein, and optionally a second administration of a reconstituted unit dose of the invention as described herein 90 days after said first administration, provide a ratio of GMT DENV-2: GMT DENV-4 of not more than 50, or not more than 40, or not more than 30, or not more than 20. In some of these embodiments, the ratio of GMT DENV-2: GMT DENV-1 is not more than 20, or not more than 18, or not more than 15 at day 180 or 365 after administration of said first reconstituted unit dose, and/or the ratio of GMT DENV-2: GMT DENV-3 is not more than 20, or not more than 18, or not more than 15 at day 180 or 365 after administration of said first reconstituted unit dose.

The present invention is in part directed to said method for preventing dengue disease (in particular virologically confirmable dengue, VCD) in a subject comprising administering to the subject at least a first reconstituted unit dose of the invention as described herein, wherein certain ratios of neutralizing antibody titers at day 180 or 365 after administration of said first unit dose to the subject are achieved. According to some embodiments, the neutralizing antibody titer for dengue serotype 2 and the neutralizing antibody titer for dengue serotype 4 at day 180 or day 365 after at least a first administration of the reconstituted unit dose of the invention as described herein, and optionally a second administration of a reconstituted unit dose of the invention as described herein 90 days after said first administration, provide a ratio of neutralizing antibody titer for DENV-2: neutralizing antibody titer for GMT DENV-4 of not more than 50, or not more than 40, or not more than 30, or not more than 20. In some of these embodiments, the ratio of the neutralizing antibody titers of DENV-2: DENV-1 is not more than 20, or not more than 18, or not more than 15 at day 180 or 365 after administration of said first reconstituted unit dose, and/or the ratio of the neutralizing antibody titers of DENV-2: DENV-3 is not more than 20, or not more than 18, or not more than 15 at day 180 or 365 after administration of said first reconstituted unit dose.

The geometric mean neutralizing antibody titers (GMTs) of a subject population or the neutralizing antibody titers of a subject are determined in accordance with the microneutralization test disclosed herein, for example according to the method described in Example 2. Without wishing to be bound to any theory, it is presently understood that a method inducing a more balanced immune response due to the administration of the reconstituted unit dose of the invention as described herein, in terms of less differences between the geometric mean neutralizing antibody titers (GMTs) against the four dengue serotypes or the neutralizing antibody titers against the four dengue serotypes, is beneficial to the subject or subject population to be vaccinated. In particular, it is understood that a much greater response to any one of the four serotypes, such as to DENV-2 in comparison to the other serotypes, is less beneficial.

The present invention is in part directed to said method for preventing dengue disease (in particular virologically confirmable dengue, VCD) in a subject or subject population wherein the method provides a seropositivity rate in a subject population of at least 50 subjects including the administration of two unit doses subcutaneously at day 1 and at day 90, wherein the subjects of the subject population are seronegative to all dengue serotypes at baseline. In certain such embodiments, at least 80% of the subject population are seropositive for all four dengue serotypes at least one month after administration of the first unit dose, such as at day 30, and/or at least 80% of the subject population are seropositive for all four dengue serotypes before or at the time of the administration of the second unit dose, such as at day 90, and/or at least 80%, or at least 85%, or at least 90%, or at least 95% of the subject population are seropositive for all four dengue serotypes after the administration of the second unit dose, such as at day 120, and/or at least 80%, or at least 85%, or at least 90% of the subject population are seropositive for all four dengue serotypes after the administration of the second unit dose, such as at day 270.

The present invention is in part directed to said method for preventing dengue disease (in particular virologically confirmable dengue, VCD) in a subject or subject population wherein the method provides a seropositivity rate in a subject population of at least 100 subjects including administration of two unit doses subcutaneously at day 1 and at day 90, wherein the subjects of the subject population comprises from 20% to 40% subjects who are seronegative to all dengue serotypes and from 60% to 80% subjects who are seropositive to at least one dengue serotype at base line, wherein at day 120 and/or day 270 the seropositivity rate for all four dengue serotypes in the seronegative part of the subject population and the seropositivity rate for all four dengue serotypes in the seropositive part of the subject population do not deviate more than 10%-points and/or wherein at day 120 the seropositivity rate for all four dengue serotypes in the seronegative part of the subject population and the seropositivity rate for all four dengue serotypes in the seropositive part of the subject population do not deviate more than 5%-points.

The present invention is in part directed to a method of preventing virologically confirmable dengue disease in a subject or subject population comprising administering to the subject or subject population a reconstituted unit dose of a tetravalent dengue virus composition including four live, attenuated dengue serotypes, in particular the virus strains as described herein.

The present invention is in part directed to a method of preventing virologically confirmable dengue disease with hospitalization in a subject or subject population comprising administering to the subject or subject population a reconstituted unit dose of a tetravalent dengue virus composition including four live, attenuated dengue serotypes, in particular the virus strains as described herein.

In certain embodiments, the method includes a reconstituted unit dose/tetravalent dengue virus composition of a dengue vaccine composition administered for preventing dengue disease in a subject or a subject population, the reconstituted unit dose comprising: a tetravalent virus composition including four live attenuated dengue virus strains, wherein a unit dose is lyophilized and upon reconstitution with 0.5 ml of a pharmaceutically acceptable diluent the reconstituted unit dose is obtained which comprises:
(i) a dengue serotype 1, such as a chimeric dengue serotype 2/1 strain, in a concentration of at least 3.3 log 10 pfu/0.5 ml,
(ii) a dengue serotype 2, such as a dengue serotype 2 strain, in a concentration of at least 2.7 log 10 pfu/0.5 ml,
(iii) a dengue serotype 3, such as a chimeric dengue serotype 2/3 strain, in a concentration of at least 4.0 log 10 pfu/0.5 ml, and
(vi) a dengue serotype 4, such as a chimeric dengue serotype 2/4 strain, in a concentration of at least 4.5 log 10 pfu/0.5 ml.

It is preferred that the reconstituted unit dose/tetravalent dengue virus composition is used in the method of preventing dengue disease of the present invention, wherein upon reconstitution of the unit dose with a pharmaceutically acceptable diluent (i), (ii), (iii), and (iv) provide a total concentration of pfu/0.5 mL and based on said total concentration the concentration of (ii) in pfu/0.5 mL is less than 2%, the concentration of (iv) in pfu/0.5 mL is at least 50%, the concentration of (i) in pfu/0.5 ml is at least 1%, and the concentration of (iii) in pfu/0.5 mL is at least 6% and wherein the subject or subject population is of 18 to 60 years of age.

In another preferred embodiment, the reconstituted unit dose/tetravalent dengue virus composition is used in the method of preventing dengue disease of the present invention, wherein upon reconstitution with a pharmaceutically acceptable diluent (i), (ii), (iii), and (iv) provide a total concentration of pfu/0.5 mL and based on said total concentration the concentration of (ii) in pfu/0.5 ml is less than 10%, and the concentration of (iv) in pfu/0.5 mL is at least 50%, and the concentration of (i) in pfu/0.5 mL is at least 1%, and the concentration of (iii) in pfu/0.5 mL is at least 8% and wherein the subject or subject population is of 2 to 17 years of age.

In certain embodiments, the invention is directed to said methods, wherein said unit dose comprises a tetravalent dengue virus composition including four live attenuated dengue serotypes, in particular the virus strains described herein wherein the serotypes have certain concentrations as described herein with respect to the virus composition and unit dose such as:
(i) a dengue serotype 1 (e.g. chimeric dengue serotype 2/1 strain) has a concentration of 3.3 log 10 pfu/dose to 5.0 log 10 pfu/dose, or 3.3 log 10 pfu/0.5 mL to 5.0 log 10 pfu/0.5 mL
(ii) a dengue serotype 2 (e.g. dengue serotype 2 strain) has a concentration of 2.7 log 10 pfu/dose to 4.9 log 10 pfu/0.5 dose, or 2.7 log 10 pfu/0.5 ml to 4.9 log 10 pfu/0.5 ml
(iii) a dengue serotype 3 (e.g. chimeric dengue serotype 2/3 strain) has a concentration of 4.0 log 10 pfu/dose to 5.7 log 10 pfu/0.5 dose, or 4.0 log 10 pfu/0.5 mL to 5.7 log 10 pfu/0.5 mL and
(iv) a dengue serotype 4 (e.g. chimeric dengue serotype 2/4 strain) has a concentration of 4.5 log 10 pfu/dose to 5.5 log 10 pfu/0.5 dose, or 4.5 log 10 pfu/0.5 mL to 5.5 log 10 pfu/0.5 mL.

In preferred such embodiments, the subject or subject population is of 2 to 17 years of age, such as 4 to 16 years of age, and preferably less than 9 years of age. In other preferred embodiments, the subject or subject population is 4-5 years of age, 6-11 years of age or 12-16 years of age.

In certain embodiments, the invention is directed to said methods, wherein said unit dose upon reconstitution with 0.5 mL of a pharmaceutically acceptable diluent has a concentration of 3.3 log 10 pfu/0.5 mL to 3.6 log 10 pfu/0.5 mL for dengue serotype 1 (e.g. chimeric dengue serotype 2/1 strain), has a concentration of 2.7 log 10 pfu/0.5 mL to 4.0 log 10 pfu/0.5 mL for dengue serotype 2 (e.g. dengue serotype 2 strain), has a concentration of 4.0 log 10 pfu/0.5 mL to 4.6 log 10 pfu/0.5 mL for dengue serotype 3 (e.g. chimeric dengue serotype 2/3 strain) and has a concentration of 4.5 log 10 pfu/0.5 mL or 4.6 log 10 pfu/0.5 mL to 5.1 log 10 pfu/0.5 mL for dengue serotype 4 (e.g. chimeric dengue serotype 2/4 strain). In preferred such embodiments, the subject or subject population is of 2 to 17 years of age, such as 4 to 16 years of age, and preferably less than 9 years of age. In other preferred embodiments, the subject or subject population is 4-5 years of age, 6-11 years of age or 12-16 years of age.

In certain embodiments, the invention is directed to said methods, wherein the concentration of the dengue serotype 1 (e.g. chimeric dengue serotype 2/1 strain) measured in pfu/0.5 mL is 1% to 7% of the total concentration, the concentration of the dengue serotype 2 (e.g. dengue serotype 2 strain) measured in pfu/0.5 mL is less than 8% of the total concentration, such as in the range of 1% to 8% of the total concentration, the concentration of the dengue serotype 3 (e.g. chimeric dengue serotype 2/3 strain) measured in pfu/0.5 mL is at least 10% of the total concentration, and the concentration of the dengue serotype 4 (e.g. chimeric dengue serotype 2/4 strain) measured in pfu/0.5 mL is at least 65% of the total concentration, such as in the range of 65% to 80%. In certain such embodiments, the arithmetic sum of all four serotypes is in the range of 4.6 log 10 pfu/0.5 mL to 6.7 log 10 pfu/0.5 mL, preferably in the range of 4.6 log 10 pfu/0.5 mL to 5.5 log 10 pfu/0.5 mL Preferably, in said embodiments the subject or subject population is of 2 to 17 years of age, such as 4 to 16 years of age, and even more preferably less than 9 years of age. In other preferred embodiments, the subject or subject population is 4-5 years of age, 6-11 years of age or 12-16 years of age.

In a further preferred embodiment, the invention is directed to said methods, wherein the dengue serotype 1 (e.g. chimeric dengue serotype 2/1 strain) such as TDV-1 and the dengue serotype 2 (e.g. dengue serotype 2 strain) such as TDV-2 are present each in a concentration based on the total concentration in pfu/0.5 mL which is within 5%-points of each other and/or are together less than about 10% of the total concentration in pfu/0.5 mL. In certain such embodiments the dengue serotype 3 (e.g. chimeric dengue serotype 2/3 strain) such as TDV-3 is preferably at least about 10% of the total concentration in pfu/0.5 mL and more preferably the dengue serotype 4 (e.g. chimeric dengue serotype 2/4 strain) such as TDV-4 is at least about 70% of the total concentration in pfu/0.5 mL. In certain such embodiments the dengue serotype 4 (e.g. chimeric dengue serotype 2/4 strain) such as TDV-4 represents the highest concentration in the composition of all four serotypes, preferably with at least about 70% of the total concentration in pfu/0.5 mL, dengue serotype 3 (e.g. chimeric dengue serotype 2/3 strain) such as TDV-3 represents the second highest concentration in the composition of all four serotypes, preferably with at least about 10% of the total concentration in pfu/0.5 mL, and dengue serotype 1 (e.g. chimeric dengue serotype 2/1 strain) such as TDV-1 and dengue serotype 2 (e.g. dengue serotype 2 strain) such as TDV-2 each represent lower concentrations than the concentration of serotype 3 (e.g. chimeric dengue serotype 2/3 strain) such as TDV-3, and optionally together represent less than about 10% of the total concentration in pfu/0.5 mL.

Preferably, the chimeric dengue serotype 2/1 strain is TDV-1, the dengue serotype 2 strain is TDV-2, the chimeric dengue serotype 2/3 strain is TDV-3 and the chimeric dengue serotype 2/4 strain is TDV-4. More preferably, TDV-1 is characterized by the nucleotide sequence according to SEQ ID No. 1 and the amino acid sequence according to SEQ ID No. 2, TDV-2 is characterized by the nucleotide sequence according to SEQ ID No. 3 and the amino acid sequence according to SEQ ID No. 4, TDV-3 is characterized by the nucleotide sequence according to SEQ ID No. 5 and the amino acid sequence according to SEQ ID No. 6 and TDV-4 is characterized by the nucleotide sequence according to SEQ ID No. 7 and the amino acid sequence according to SEQ ID No. 8.

In certain embodiments, the invention is directed to said methods, wherein the reconstituted unit dose of the invention as described herein is administered by subcutaneous injection. According to some of these embodiments, the subcutaneous injection is administered to the arm, preferably to the deltoid region of the arm.

In certain embodiments, the invention is directed to said methods, wherein the reconstituted unit dose is administered to a subject of unknown serostatus and/or wherein no test has been carried out to determine whether the subject is seropositive or seronegative (before) the unit dose as described herein is administered. In certain embodiments, the invention is directed to said methods which do not include a step of determination of a previous dengue infection in the subject or subjects. In certain embodiments, the invention is directed to said methods which do not include the analysis of the seroprevalence rate in the region or is conducted in a region with a seroprevalence of below 80%, below 70% or below 60%. In certain embodiments the invention is directed to a method wherein the serostatus of the subject is unknown. In such embodiments the serostatus is not determined at any time before and after administration in relation to this method. In certain embodiments of the invention the method is used in an outbreak situation. In certain embodiments, the invention is directed to said methods being conducted outside a clinical trial

In certain embodiments, the invention is directed to said methods, wherein the subject, or subject population is seronegative to all dengue serotypes.

In certain embodiments, the invention is directed to said methods, wherein two unit doses of the invention as described herein are administered. In some embodiments the two unit doses are administered within 12 months or more, or within six months, or within three months, and optionally at least 4 weeks apart such as at day 0 and day 90 or at day 1 and day 90. According to some of these embodiments, a further third unit dose of the invention as described herein is administered after the second administration. Such a third administration may act as a booster and may be administered between 6 to 12 months after the first administration, such as 12 months after the first administration, or later than 12 month after the first administration, such as 12 months (1 year) after the second administration or even 5 years or longer after the first or second administration.

In certain embodiments, the method of the invention comprises or consists of a single unit dose of the invention being administered.

In certain embodiments, the invention is directed to said methods, wherein the reconstituted unit dose of the invention as described herein is administered subcutaneously to a subject or subject population that is seronegative with respect to all dengue serotypes. In other embodiments, the subject or subject population is seropositive with respect to at least one dengue serotype.

In certain embodiments, the invention is directed to said methods, wherein the unit dose of the invention as described herein is administered to a subject or subject population from a dengue endemic region. In some of these embodiments, the subject or subject population is from Singapore, Dominican Republic, Panama, Philippines, Colombia, Puerto Rico or Thailand, in particular from Singapore, Dominican Republic, Panama, or Philippines. In a preferred embodiment, the subject or subject population is from Asia Pacific or from Latin America. In some other of these embodiments, the subject or subject population is from Thailand, Sri Lanka, Philippines, Panama, Nicaragua, Dominican Republic, Colombia or Brazil. In other embodiments, the subject, or subject population is from a dengue non-endemic region. Such a subject population or such a subject may be vaccinated according to the present invention in the context of traveling to a dengue endemic region. In certain embodiments, the reconstituted unit dose of the invention as described herein is administered subcutaneously to a subject, or subject population that is from a dengue endemic region or a dengue non-endemic region.

In certain embodiments, the invention is directed to said methods, wherein the reconstituted unit dose of the invention as described herein is administered subcutaneously to a subject or subject population of 2 to 60 years of age. In some embodiments, the subjects or subject population are adults of more than 17 years, or more than 18 years, or 18 to 60 years. In further specific embodiments, the subjects or subject population are adults of more than 21 years, or 21 to 60 years, or 21 to 45 years of age.

In certain embodiments, the invention is directed to said methods, wherein the reconstituted unit dose of the invention as described herein is administered subcutaneously to children and adolescents of 2 to 17 years of age. In some embodiments, the subjects or subject population are less than 9 years of age, or less than 4 years of age. In some embodiments, the subjects or subject population are from 2 to 9 years of age, or from 2 to 5 years of age, or from 4 to 9 years of age or from 6 to 9 years of age. In other embodiment, the subject or subject population is 4 to 16 years of age. In some such embodiments, the subject or subject population is 4-5 years of age, 6-11 years of age or 12-16 years of age. Optionally, the subject or subject population is seronegative with respect to all dengue serotypes.

In certain embodiments, the invention is directed to said methods, wherein the unit dose of the invention as described herein is administered to a pediatric subject or pediatric subject population of less than 2 years of age, preferably of 2 months to 2 years or 2 months to 1.5 years or 2 months to 1 year. According to some of these embodiments, the pediatric subject or pediatric subject population is seronegative and from a dengue endemic region.

In certain embodiments, the invention is directed to said methods, wherein the reconstituted unit dose of the invention as described herein is administered to a pediatric subject or pediatric subject population of less than 2 years of age, preferably of 2 months to 2 years or 2 months to 1.5 years or 2 months to 1 year, preferably by subcutaneous injection. According to some of these embodiments, the pediatric subject or pediatric subject population is seronegative and from a dengue endemic region.

In a certain embodiments, the invention is directed to said methods, wherein the subject or subject population is 4-5 years of age and from Asia Pacific, 6-11 years of age and from Asia Pacific, or 12-16 years of age and from Asia Pacific. In other embodiments, the subject or subject population is 4-5 years of age and from Latin America, 6-11 years of age and from Latin America, or 12-16 years of age and from Latin America.

In a certain embodiments, the invention is directed to said methods, wherein the subject or subject population is 4-5 years of age and seropositive for at least 1 dengue serotype, 6-11 years of age and seropositive for at least 1 dengue serotype, or 12-16 years of age and seropositive for at least 1 dengue serotype. In other embodiments, the subject or subject population is 4-5 years of age and seronegative for all dengue serotypes, 6-11 years of age and seronegative for all dengue serotypes, or 12-16 years of age and seronegative for all dengue serotypes.

In a certain embodiments, the invention is directed to said methods, wherein the subject or subject population is from Asia Pacific or Latin America and seropositive for at least one dengue serotype at baseline. In other embodiments, the subject or subject population is from Asia Pacific or Latin America and seronegative for at all dengue serotype at baseline.

In certain embodiments, the invention is directed to said methods, wherein the subject or subject population is from Asia Pacific, seropositive for at least one dengue serotype at baseline and 4-5 years of age, 6-11 years of age, or 12-16 years of age. In other embodiments, the subject or subject population is from Asia Pacific, seronegative for all dengue serotypes at baseline and 4-5 years of age, 6-11 years of age, or 12-16 years of age. In yet other embodiments, the subject or subject population is from Latin America, seropositive for at least one dengue serotype at baseline and 4-5 years of age, 6-11 years of age, or 12-16 years of age. In other embodiments, the subject or subject population is from America, seronegative for all dengue serotypes at baseline and 4-5 years of age, 6-11 years of age, or 12-16 years of age.

In certain embodiments, the invention is directed to said methods, wherein the subject or subject population had prior vaccination against Yellow Fever. In other embodiments, the subject or subject population had prior vaccination against Japanese Encephalitis. In yet other embodiments, the subject or subject population had no prior vaccination against Yellow Fever. In other embodiments, the subject or subject population had no prior vaccination against Japanese Encephalitis. Prior vaccination indicates a vaccination prior to 30 days after a second administration, such as within 4 months after the first administration, with the reconstituted unit dose as described herein. For example for vaccine efficacy (VE) as determined in Example 6 from 30 days post-second vaccination, a prior vaccination of Yellow Fever is defined as a Yellow Fever vaccination occurring before 30 days post-second vaccination. In certain embodiments, the subject or subject population received Dengvaxia® within the administration regimen as described herein or within 4.5 years after administration of the first dose.

Particularly unbalanced titers of neutralizing antibodies against the four dengue serotypes are observed in seronegative populations or subjects after administration of the commercially available dengue vaccine. The present invention shows that in particular seronegative subjects show a more balanced immune response to the four dengue serotypes after administration of the reconstituted unit dose of the invention as described herein. It is therefore contemplated that the unit dose of the invention as described herein and methods of the present invention as described herein may provide a more robust immune response in a subject population including both seropositive and seronegative subjects. This balanced response and balanced efficacy and safety is required to allow inoculation without prior serostatus analysis which is a major advantage in vaccination programs and in particular in outbreak situations.

The present invention is directed in part to a method of preventing virologically confirmable dengue disease in a subject comprising administering to the subject a tetravalent dengue virus composition including four dengue virus strains representing serotype 1, serotype 2, serotype 3 and serotype 4, wherein the virus strains are optionally live, attenuated dengue virus strains.

The present invention is directed in part to a method of preventing virologically confirmable dengue disease in a subject consisting of administering to the subject a tetravalent dengue virus composition including four dengue virus strains representing serotype 1, serotype 2, serotype 3 and serotype 4, wherein the virus strains are optionally live, attenuated dengue virus strains.

In certain embodiments, the invention is directed to said methods, wherein there is no step of determining the serostatus of the subject at baseline, in other words, said methods do not comprise a determination of a previous dengue infection of the subject at baseline before the administration of the tetravalent dengue virus composition. In particular, such methods are safe and effective. Thus, in certain such embodiments, the subject has not been tested for the presence a previous dengue infection.

In certain embodiments, the invention is directed to said methods, wherein the vaccine administration is safe irrespective of whether there is a determination that the subject had a previous dengue infection before the administration of the tetravalent dengue virus composition. In particular, such methods are also effective.

In certain embodiments, the invention is directed to said methods, wherein the method is safe and/or effective.

In certain embodiments, the invention is directed to said methods, wherein the composition includes at least one chimeric dengue virus. In certain such embodiments, the invention is directed to said methods, wherein the composition includes at least one non-chimeric dengue virus and at least one chimeric dengue virus, in particular a chimeric dengue serotype 2/1 strain and a dengue serotype 2 strain and a chimeric dengue serotype 2/3 strain and a chimeric dengue serotype 2/4 strain. The details of the composition are described above.

Therefore, in certain embodiments, the invention is directed to said methods having a vaccine efficacy, preferably a combined vaccine efficacy against all four serotypes, in preventing virologically confirmable dengue disease with a 2-sided 95% confidence interval, wherein the lower bound is more than 25%, when measured against placebo in a subject population of at least 5,000 healthy subjects (or at least 10,000, or at least 15,000 healthy subjects) irrespective of serostatus at baseline and e.g. 14 to 16 years of age, wherein a reconstituted unit dose/tetravalent dengue virus composition as described herein or placebo is administered e.g. at least twice within less than 6 months, such as within 3 months, after first administration or 30 days after the second/last administration until at least 12 to 18 months (e.g. at 12 or at 18 months) after the second/last administration. In embodiments, the invention is directed to said methods having a vaccine efficacy, preferably a combined vaccine efficacy against all four serotypes, in preventing virologically confirmable dengue disease with a 2-sided 95% confidence interval, wherein the lower bound is more than 25%, when measured against placebo in a subject population of at least 5,000 healthy subjects (or at least 10,000, or at least 15,000 healthy subjects) irrespective of serostatus at baseline, wherein a reconstituted unit dose or tetravalent dengue virus composition as described herein or placebo is administered at least once, until 15 to 21 months (e.g. 15 or 21 months) after the first administration of the administration schedule. In certain such embodiments, the lower bound is more than 30%, more than 40%, more than 50%, more than 55%, more than 60%, more than 65%, more than 70% or more than 72%. Preferably said reconstituted unit dose or placebo is administered subcutaneously within about 3 months, such as on days 0 and 90.

Therefore, in certain embodiments, the invention is directed to said methods having a combined vaccine efficacy against all four serotypes, in preventing virologically confirmable dengue disease with a 2-sided 95% confidence interval, wherein the lower bound is more than 60%, when measured against placebo in a subject population of at least 5,000 healthy subjects (or at least 10,000, or at least 15,000 healthy subjects) irrespective of serostatus at baseline and 4 to 16 years of age, wherein a reconstituted unit dose/tetravalent dengue virus composition as described herein or placebo is administered e.g. at least twice within less than 6 months, such as within 3 months, after the first administration until 18 months after the last administration. In these embodiments, the lower bound is e.g. more than 62%, more than 64%, more than 66%, more than 68%, or more than 69%.

In certain embodiments, the invention is directed to said methods having a vaccine efficacy, preferably a combined vaccine efficacy against all four serotypes, in preventing virologically confirmable dengue disease of more than 30%, when measured against placebo in a subject population of at least 5,000 healthy subjects (or at least 10,000, or at least 15,000 healthy subjects) irrespective of serostatus at baseline and e.g. 14 to 16 years of age, wherein a reconstituted unit dose or tetravalent dengue virus composition as described herein or placebo is administered at least twice within less than 6 months, such as within 3 months, after first administration or 30 days after the second administration/last administration until at least 12 months or until 12 to 18 months (e.g. at 12 or at 18 months) after the second administration/last administration. In certain embodiments, the invention is directed to said methods having a vaccine efficacy, preferably a combined vaccine efficacy against all four serotypes, in preventing virologically confirmable dengue disease of more than 30%, when measured against placebo in a subject population of at least 5,000 healthy subjects (or at least 10,000, or at least 15,000 healthy subjects) irrespective of serostatus at baseline, wherein a reconstituted unit dose or tetravalent dengue virus composition as described herein or placebo is administered at least once, until 15 months after the first administration of the administration schedule. In certain such embodiments, the vaccine efficacy is more than 40%, more than 50%, more than 55%, more than 60%, more than 65%, more than 70%, more than 75%, more than 78%, more than 79% or about 80%. Preferably said reconstituted unit dose or placebo is administered subcutaneously within about 3 month, such as on days 0 and 90.

Therefore, in certain embodiments, the invention is directed to said methods having a combined vaccine efficacy against all four serotypes, in preventing virologically confirmable dengue disease of more than 66%, when measured against placebo in a subject population of at least 5,000 healthy subjects (or at least 10,000, or at least 15,000 healthy subjects) irrespective of serostatus at baseline and 14 to 16 years of age, wherein a reconstituted unit dose/tetravalent dengue virus composition as described herein or placebo is administered e.g. at least twice within less than 6 months, such as within 3 months, after the first administration until 18 months after the last administration. In these embodiments, the vaccine efficacy is e.g. more than 68%, more than 70%, more than 72%, or more than 74%.

In certain embodiments, the invention is directed to said methods having a vaccine efficacy, preferably a combined vaccine efficacy against all four serotypes, in preventing virologically confirmable dengue disease with hospitalization with a 2-sided 95% confidence interval, wherein the lower bound is more than 0%, when measured against placebo in a subject population of at least 5,000 healthy subjects (or at least 10,000, or at least 15,000 healthy subjects) irrespective of serostatus at baseline, wherein a reconstituted unit dose or tetravalent dengue virus composition as described herein or placebo is administered at least twice within less than 6 months, such as within 3 months, 30 days after the second administration until at least 18 months after the second administration. In certain such embodiments, the lower bound is more than 10%, is more than 20%, is more than 30%, is more than 40%, is more than 50%, is more than 55%, is more than 60%, is more than 65%, is more than 70% or is more than 80%, or more than 90%.

In certain embodiments, the invention is directed to said methods having a combined vaccine efficacy against all four dengue serotypes in seronegative subjects with a 2-sided 95% confidence interval, wherein the lower bound is more than 25%, when measured against placebo in a subject population of at least 1,500 or at least 2,000 healthy subjects being seronegative against all serotypes at baseline, wherein said unit dose/tetravalent dengue virus composition or said placebo is administered at least twice within less than 6 months, such as within 3 months, about 30 days after the second administration of the administration schedule until at least 12 months or until 12 to 18 months (e.g. at 12 or at 18 months) after the second administration of the administration schedule. In certain such embodiments, the lower bound is more than 30%, is more than 40%, is more than 50%, or is more than 55%.

In certain embodiments, the invention is directed to said methods having a combined vaccine efficacy against all four serotypes, in preventing virologically confirmable dengue disease with a 2-sided 95% confidence interval, wherein the lower bound is more than 25%, when measured against placebo in a subject population of at least 1,500 or at least 2,000 or at least 5,000 healthy subjects (or at least 10,000, or at least 15,000 healthy subjects) being seronegative against all serotypes at baseline and 4 to 16 years of age, wherein a reconstituted unit dose/tetravalent dengue virus composition as described herein or placebo is e.g. administered at least twice within less than 6 months, such as within 3 months, from 30 days post last administration until 12 to 18 months (e.g. at 12 months or at 18 months) after the last administration. In certain such embodiments, the lower bound is more than 30%, is more than 35%, is more than 40%, or is more than 45%.

In certain embodiments, the invention is directed to said methods having a combined vaccine efficacy against all four serotypes, in preventing virologically confirmable dengue disease with a 2-sided 95% confidence interval, wherein the lower bound is more than 25%, when measured against placebo in a subject population of at least 5,000 healthy subjects (or at least 10,000, or at least 15,000 healthy subjects) being seropositive at baseline and 4 to 16 years of age, wherein a reconstituted unit dose/tetravalent dengue virus composition as described herein or placebo is e.g. administered at least twice within less than 6 months, such as within 3 months, from 30 days post last administration until 12 to 18 months (e.g. at 12 months or at 18 months) after the last administration. In certain such embodiments, the lower bound is more than 40%, is more than 45%, is more than 50%, is more than 60%, or is more than 65%.

In certain embodiments, the invention is directed to said methods having a combined vaccine efficacy against all four serotypes, in preventing virologically confirmable dengue disease with a 2-sided 95% confidence interval, wherein the lower bound is more than 25%, when measured against placebo in a subject population of at least 5,000 healthy subjects (or at least 10,000, or at least 15,000 healthy subjects) being seropositive at baseline being or seronegative against all serotypes at baseline and 4 to 16 years of age, wherein a reconstituted unit dose/tetravalent dengue virus composition as described herein or placebo is e.g. administered at least twice within less than 6 months, such as within 3 months, from 30 days post last administration until 12 to 18 months (e.g. at 12 months or at 18 months) after the last administration. In certain such embodiments, the difference between the lower bound provided by the seropositive subjects at baseline and the subjects seronegative against all serotypes at baseline is no more than 15%-points.

In certain embodiments, the invention is directed to said methods having a combined vaccine efficacy against all four dengue serotypes in seronegative subjects of more than 30%, when measured against placebo in a subject population of at least 1,500 or at least 2,000 healthy subjects being seronegative against all serotypes at baseline, wherein said unit dose/tetravalent dengue virus composition or said placebo is administered at least twice within less than 6 months, such as within 3 months, 30 days after the second administration until at least 12 months or until 12 to 18 months (e.g. at 12 or at 18 months) after the second administration. In certain such embodiments, the combined vaccine efficacy against all four dengue serotypes in seronegative subjects is more than 40%, is more than 50%, is more than 60%, is more than 65%, or is more than 70%.

In certain embodiments, the invention is directed to said methods having a combined vaccine efficacy against all four serotypes, in preventing virologically confirmable dengue disease, when measured against placebo in a subject population of at least 1,500 or at least 2,000 or at least 5,000 healthy subjects (or at least 10,000, or at least 15,000 healthy subjects) being seronegative against all serotypes at baseline and 4 to 16 years of age, wherein a reconstituted unit dose/tetravalent dengue virus composition as described herein or placebo is e.g. administered at least twice within less than 6 months, such as within 3 months, from 30 days post last administration until 12 to 18 months (e.g. at 12 months or at 18 months) after the last administration. In certain such embodiments the said vaccine efficacy is more than 30%, is more than 40%, is more than 50%, is more than 55%, is more than 60%, or is more than 65%.

In certain embodiments, the invention is directed to said methods having a combined vaccine efficacy against all four serotypes, in preventing virologically confirmable dengue disease, when measured against placebo in a subject population of at least 5,000 healthy subjects (or at least 10,000, or at least 15,000 healthy subjects) being seropositive at baseline and 4 to 16 years of age, wherein a reconstituted unit dose/tetravalent dengue virus composition as described herein or placebo is e.g. administered at least twice within less than 6 months, such as within 3 months, from 30 days post last administration until 12 to 18 months (e.g. at 12 months or at 18 months) after the last administration. In certain such embodiments the said vaccine efficacy is more than 40%, is more than 50%, is more than 60%, is more than 65%, is more than 70%, or is more than 75%.

In certain embodiments, the invention is directed to said methods having a combined vaccine efficacy against all four serotypes, in preventing virologically confirmable dengue disease with a 2-sided 95% confidence interval, wherein the lower bound is more than 25%, when measured against placebo in a subject population of at least 5,000 healthy subjects (or at least 10,000, or at least 15,000 healthy subjects) being seropositive at baseline being or seronegative against all serotypes at baseline and 4 to 16 years of age, wherein a reconstituted unit dose/tetravalent dengue virus composition as described herein or placebo is e.g. administered at least twice within less than 6 months, such as within 3 months, from 30 days post last administration until 12 to 18 months (e.g. at 12 months or at 18 months) after the last administration. In certain such embodiments, the difference between the lower bound provided by the seropositive subjects at baseline and the subjects seronegative against all serotypes at baseline is no more than 15%-points, or is no more than 10%-points.

In certain embodiments, the invention is directed to said methods having a combined vaccine efficacy against all four dengue serotypes with a 2-sided 95% confidence interval, wherein the lower bound is more than 25%, when measured against placebo in a subject population of at least 1,000 healthy subjects 4 to 5 years of age at the time of randomization and irrespective of serostatus at baseline, wherein said unit dose/tetravalent dengue virus composition or said placebo is administered at least twice within less than 6 months, such as within 3 months, about 30 days after the second administration of the administration schedule until at least 12 months or until 12 to 18 months (e.g. at 12 or at 18 months) after the second administration of the administration schedule. In certain such embodiments, the lower bound is more than 30%, is more than 40%, is more than 45%.

In certain embodiments, the invention is directed to said methods having a combined vaccine efficacy against all four dengue serotypes of more than 30%, when measured against placebo in a subject population of at least 1,000 healthy subjects 4 to 5 years of age at the time of randomization and irrespective of serostatus at baseline, wherein said unit dose/tetravalent dengue virus composition or said placebo is administered at least twice within less than 6 months, such as within 3 months, 30 days after the second administration until at least 12 months or until 12 to 18 months (e.g. at 12 or at 18 months) after the second administration. In certain such embodiments, the combined vaccine efficacy against all four dengue serotypes is more than 40%, is more than 50%, is more than 60%, is more than 65%, or is more than 70%.

In certain embodiments, the invention is directed to said methods having a combined vaccine efficacy against all four dengue serotypes with a 2-sided 95% confidence interval, wherein the lower bound is more than 25%, when measured against placebo in a subject population of at least 1,000 healthy subjects 6 to 11 years of age at the time of randomization and irrespective of serostatus at baseline, wherein said unit dose/tetravalent dengue virus composition or said placebo is administered at least twice within less than 6 months, such as within 3 months, about 30 days after the second administration of the administration schedule until at least 12 months or until 12 to 18 months (e.g. at 12 or at 18 months) after the second administration of the administration schedule. In certain such embodiments, the lower bound is more than 30%, is more than 40%, is more than 50%, is more than 60%, or is more than 70%.

In certain embodiments, the invention is directed to said methods having a combined vaccine efficacy against all four dengue serotypes of more than 30%, when measured against placebo in a subject population of at least 1,000 healthy subjects 6 to 11 years of age at the time of randomization and irrespective of serostatus at baseline, wherein said unit dose/tetravalent dengue virus composition or said placebo is administered at least twice within less than 6 months, such as within 3 months, 30 days after the second administration until at least 12 months or until 12 to 18 months (e.g. at 12 or at 18 months) after the second administration. In certain such embodiments, the combined vaccine efficacy against all four dengue serotypes is more than 40%, is more than 50%, is more than 60%, is more than 70%, is more than 75%, or is more than 80%.

In certain embodiments, the invention is directed to said methods having a combined vaccine efficacy against all four dengue serotypes with a 2-sided 95% confidence interval, wherein the lower bound is more than 25%, when measured against placebo in a subject population of at least 1,000 healthy subjects 12 to 16 years of age at the time of randomization and irrespective of serostatus at baseline, wherein said unit dose/tetravalent dengue virus composition or said placebo is administered at least twice within less than 6 months, such as within 3 months, about 30 days after the second administration of the administration schedule until at least 12 months or until 12 to 18 months (e.g. at 12 or at 18 months) after the second administration of the administration schedule. In certain such embodiments, the lower bound is more than 30%, is more than 40%, is more than 50%, is more than 60%, is more than 65%, or is more than 68%.

In certain embodiments, the invention is directed to said methods having a combined vaccine efficacy against all four dengue serotypes of more than 30%, when measured against placebo in a subject population of at least 1,000 healthy subjects 12 to 16 years of age at the time of randomization and irrespective of serostatus at baseline, wherein said unit dose/tetravalent dengue virus composition or said placebo is administered at least twice within less than 6 months, such as within 3 months, 30 days after the second administration until at least 12 months or until 12 to 18 months (e.g. at 12 or at 18 months) after the second administration. In certain such embodiments, the combined vaccine efficacy against all four dengue serotypes is more than 40%, is more than 50%, is more than 60%, is more than 70%, is more than 75%, or is more than 80%.

In certain embodiments, the invention is directed to said methods having a vaccine efficacy against dengue serotype 1 with a 2-sided 95% confidence interval, wherein the lower bound is more than 25%, when measured against placebo in a subject population of at least 5,000 healthy subjects, or at least 10,000 healthy subjects, or at least 15,000 healthy subjects irrespective of serostatus at baseline, wherein said unit dose/tetravalent dengue virus composition or said placebo is administered at least twice within less than 6 months, such as within 3 months, about 30 days after the second administration of the administration schedule until at least 12 months or until 12 to 18 months (e.g. at 12 or at 18 months) after the second administration of the administration schedule. In certain such embodiments, the lower bound is more than 30%, is more than 40%, or is more than 50%.

In certain embodiments, the invention is directed to said methods having a vaccine efficacy against serotype 1, in preventing virologically confirmable dengue disease with a 2-sided 95% confidence interval, wherein the lower bound is more than 25%, when measured against placebo in a subject population of at least 1,500, or at least 2,000, or at least 5,000 healthy subjects (or at least 10,000, or at least 15,000 healthy subjects) irrespective of serostatus at baseline and 4 to 16 years of age, wherein a reconstituted unit dose/tetravalent dengue virus composition as described herein or placebo is e.g. administered at least twice within less than 6 months, such as within 3 months, from 30 days post last administration until 12 to 18 months (e.g. at 12 or at 18 months) after the last administration. In certain such embodiments, the lower bound is more than 30%, is more than 35% is more than 40%, is more than 45%, is more than 50%, or is more than 54%. In certain such embodiments the subject population of at least 1,500 is seronegative against all serotypes at base line and the lower bound is more than 35%. In certain such embodiments the seronegative and seropositive population each provide a vaccine efficacy against serotype 1 with a 2-sided 95% confidence interval, wherein the lower bounds are within 10%-points.

In certain embodiments, the invention is directed to said methods having a vaccine efficacy against dengue serotype 1 of more than 30%, when measured against placebo in a subject population of at least 5,000 healthy subjects, or at least 10,000 healthy subjects, or at least 15,000 healthy subjects irrespective of serostatus at baseline, wherein said unit dose/tetravalent dengue virus composition or said placebo is administered at least twice within less than 6 months, such as within 3 months, 30 days after the second administration until at least 12 months or until 12 to 18 months (e.g. at 12 or at 18 months) after the second administration. In certain such embodiments, the vaccine efficacy against dengue serotype 1 is more than 40%, is more than 50%, is more than 60%, is more than 65%, or is more than 70%.

In certain embodiments, the invention is directed to said methods having a vaccine efficacy against serotype 1, in preventing virologically confirmable dengue disease, when measured against placebo in a subject population of at least 1,500, or at least 2,000, or at least 5,000 healthy subjects (or at least 10,000, or at least 15,000 healthy subjects) irrespective of serostatus at baseline and 4 to 16 years of age, wherein a reconstituted unit dose/tetravalent dengue virus composition as described herein or placebo is e.g. administered at least twice within less than 6 months, such as within 3 months, from 30 days post last administration until 12 to 18 months (e.g. at 12 or at 18 months) after the last administration. In certain such embodiments, the vaccine efficacy is more than 40%, is more than 50%, is more than 60%, or is more than 65%. In certain such embodiments the subject population of at least 1,500 is seronegative against all serotypes at base line. In certain such embodiments the seronegative and seropositive population each provide a vaccine efficacy against serotype 1 which are within 5%-points.

In certain embodiments, the invention is directed to said methods having a vaccine efficacy against dengue serotype 2 with a 2-sided 95% confidence interval, wherein the lower bound is more than 25%, when measured against placebo in a subject population of at least 5,000 healthy subjects, or at least 10,000 healthy subjects, or at least 15,000 healthy subjects irrespective of serostatus at baseline, wherein said unit dose/tetravalent dengue virus composition or said placebo is administered at least twice within less than 6 months, such as within 3 months, about 30 days after the second administration of the administration schedule until at least 12 months or until 12 to 18 months (e.g. at 12 or at 18 months) after the second administration of the administration schedule. In certain such embodiments, the lower bound is more than 30%, is more than 40%, is more than 50, is more than 60, is more than 70, is more than 80, or is more than 90%.

In certain embodiments, the invention is directed to said methods having a vaccine efficacy against serotype 2, in preventing virologically confirmable dengue disease with a 2-sided 95% confidence interval, wherein the lower bound is more than 25%, when measured against placebo in a subject population of at least 1,500, or at least 2,000, or at least 5,000 healthy subjects (or at least 10,000, or at least 15,000 healthy subjects) irrespective of serostatus at baseline and 4 to 16 years of age, wherein a reconstituted unit dose/tetravalent dengue virus composition as described herein or placebo is e.g. administered at least twice within less than 6 months, such as within 3 months, from 30 days post last administration until 12 to 18 months (e.g. at 12 or at 18 months) after the last administration. In certain such embodiments, the lower bound is more than 50%, is more than 60%, is more than 70%, is more than 80%, or is more than 85%. In certain such embodiments the subject population of at least 1,500, is seronegative against all serotypes. In certain such embodiments the seronegative and seropositive population each provide a vaccine efficacy against serotype 2 with a 2-sided 95% confidence interval, wherein the lower bounds are within 5%-points.

In certain embodiments, the invention is directed to said methods having a vaccine efficacy against dengue serotype 2 of more than 30%, when measured against placebo in a subject population of at least 5,000 healthy subjects, or at least 10,000 healthy subjects, or at least 15,000 healthy subjects irrespective of serostatus at baseline, wherein said unit dose/tetravalent dengue virus composition or said placebo is administered at least twice within less than 6 months, such as within 3 months, 30 days after the second administration until at least 12 months or until 12 to 18 months (e.g. at 12 or at 18 months) after the second administration. In certain such embodiments, the vaccine efficacy against dengue serotype 2 is more than 40%, is more than 50%, is more than 60%, is more than 70%, is more than 80, or is more than 90%.

In certain embodiments, the invention is directed to said methods having a vaccine efficacy against serotype 2, in preventing virologically confirmable dengue disease, when measured against placebo in a subject population of at least 1,500, or at least 2,000, or at least 5,000 healthy subjects (or at least 10,000, or at least 15,000 healthy subjects) irrespective of serostatus at baseline and 4 to 16 years of age, wherein a reconstituted unit dose/tetravalent dengue virus composition as described herein or placebo is e.g. administered at least twice within less than 6 months, such as within 3 months, from 30 days post last administration until 12 to 18 months (e.g. at 12 or at 18 months) after the last administration. In certain such embodiments, the vaccine efficacy is more than 60%, is more than 70%, is more than 80%, or is more than 90%. In certain such embodiments the subject population of at least 1,500 is seronegative against all serotypes at base line. In certain such embodiments the seronegative and seropositive population each provide a vaccine efficacy against serotype 2 which are within 5%-points.

In certain embodiments, the invention is directed to said methods having a vaccine efficacy against dengue serotype 3 with a 2-sided 95% confidence interval, wherein the lower bound is more than 25%, when measured against placebo in a subject population of at least 5,000 healthy subjects, or at least 10,000 healthy subjects, or at least 15,000 healthy subjects irrespective of serostatus at baseline, wherein said unit dose/tetravalent dengue virus composition or said placebo is administered at least twice within less than 6 months, such as within 3 months, about 30 days after the second administration of the administration schedule until at least 12 months after the second administration of the administration schedule. In certain such embodiments, the lower bound is more than 30%, is more than 40%.

In certain embodiments, the invention is directed to said methods having a vaccine efficacy against dengue serotype 3 of more than 30%, when measured against placebo in a subject population of at least 5,000 healthy subjects, or at least 10,000 healthy subjects, or at least 15,000 healthy subjects irrespective of serostatus at baseline, wherein said unit dose/tetravalent dengue virus composition or said placebo is administered at least twice within less than 6 months, such as within 3 months, 30 days after the second administration until at least 12 months after the second administration. In certain such embodiments, the vaccine efficacy against dengue serotype 3 is more than 40%, is more than 50%, is more than 55%, or is more than 60%.

In certain embodiments, the invention is directed to said methods having a combined vaccine efficacy against all four serotypes, in preventing virologically confirmable dengue disease with hospitalization with a 2-sided 95% confidence interval, wherein the lower bound is more than 25%, when measured against placebo in a subject population of at least 5,000 healthy subjects (or at least 10,000, or at least 15,000 healthy subjects) irrespective of serostatus at baseline and 4 to 16 years of age, wherein a reconstituted unit dose/tetravalent dengue virus composition as described herein or placebo is e.g. administered at least twice within less than 6 months, such as within 3 months, from first administration until 12 to 18 months (e.g. at 12 months or at 18 months) after the last administration, or from 30 days post last administration until 12 to 18 months (e.g. at 12 or at 18 months) after the last administration. In certain such embodiments, the lower bound is more than 10%, is more than 20%, is more than 30%, is more than 40%, is more than 50%, is more than 55%, is more than 60%, is more than 65%, is more than 66%, is more than 67%, is more than 70%, is more than 75%, is more than 77%, or is more than 80%.

In certain embodiments, the invention is directed to said methods having a combined vaccine efficacy against all four serotypes, in preventing virologically confirmable dengue disease with hospitalization, when measured against placebo in a subject population of at least 5,000 healthy subjects (or at least 10,000, or at least 15,000 healthy subjects) irrespective of serostatus at baseline and 4 to 16 years of age, wherein a reconstituted unit dose/tetravalent dengue virus composition as described herein or placebo is e.g. administered at least twice within less than 6 months, such as within 3 months, from first administration until 12 to 18 months (e.g. at 12 months or at 18 months) after the last administration, or from 30 days post last administration until 12 to 18 months (e.g. at 12 or at 18 months) after the last administration. In certain such embodiments, the vaccine efficacy is more than is more than 70%, is more than 75%, is more than 80%, or is more than 82%, or is more than 85%, more than 88%.

In certain embodiments, the invention is directed to said methods having a combined vaccine efficacy against virologically-confirmed dengue with hospitalization against all four serotypes with a 2-sided 95% confidence interval, wherein the lower bound is more than 25%, when measured against placebo in a subject population of at least 1,500 or at least 2,000 healthy subjects being seronegative against all serotypes at baseline, wherein said unit dose/tetravalent dengue virus composition or said placebo is administered at least twice within less than 6 months, such as within 3 months, about 30 days after the second administration of the administration schedule until at least 12 months or until 12 to 18 months (e.g. at 12 or at 18 months) after the second administration of the administration schedule. In certain such embodiments, the lower bound is more than 30%, is more than 40%, is more than 50%, is more than 60%, is more than 70%, or is more than 75%.

In certain embodiments, the invention is directed to said methods having a combined vaccine efficacy against all four serotypes, in preventing virologically confirmable dengue disease with hospitalization with a 2-sided 95% confidence interval, wherein the lower bound is more than 25%, when measured against placebo in a subject population of at least 5,000 healthy subjects (or at least 10,000, or at least 15,000 healthy subjects) being seronegative against all serotypes at baseline and 4 to 16 years of age, wherein a reconstituted unit dose/tetravalent dengue virus composition as described herein or placebo is e.g. administered at least twice within less than 6 months, such as within 3 months, from 30 days post last administration until 12 to 18 months (e.g. at 12 months or at 18 months) after the last administration.

In certain such embodiments, the lower bound is more than 60%, is more than 65%, is more than 66%, is more than 67%, is more than 70%, is more than 75%, is more than 77% or is more than 80%.

In certain embodiments, the invention is directed to said methods having a combined vaccine efficacy against virologically-confirmed dengue with hospitalization against all four serotypes of more than 30%, when measured against placebo in a subject population of at least 1,500 or at least 2,000 healthy subjects, healthy subjects being seronegative against all serotypes at baseline, wherein said unit dose/tetravalent dengue virus composition or said placebo is administered at least twice within less than 6 months, such as within 3 months, 30 days after the second administration until at least 12 months or until 12 to 18 months (e.g. at 12 or at 18 months) after the second administration. In certain such embodiments, the combined vaccine efficacy against virologically-confirmed dengue with hospitalization against all four serotypes is more than 40%, is more than 50%, is more than 60%, is more than 70%, is more than 80%, or is more than 90%.

In certain embodiments, the invention is directed to said methods having a combined vaccine efficacy against all four serotypes, in preventing virologically confirmable dengue disease with hospitalization, when measured against placebo in a subject population of at least 5,000 healthy subjects (or at least 10,000, or at least 15,000 healthy subjects) being seronegative against all serotypes at baseline and 4 to 16 years of age, wherein a reconstituted unit dose/tetravalent dengue virus composition as described herein or placebo is e.g. administered at least twice within less than 6 months, such as within 3 months, from 30 days post last administration until 12 to 18 months (e.g. at 12 months or at 18 months) after the last administration. In certain such embodiments, the said vaccine efficacy is more than 60%, is more than 65%, is more than 66%, is more than 67%, is more than 70%, is more than 75%, is more than 77%, is more than 80, or is more than 85%.

In certain embodiments, the invention is directed to said methods having a combined vaccine efficacy against virologically-confirmed dengue with hospitalization against all four serotypes with a 2-sided 95% confidence interval, wherein the lower bound is more than 25%, when measured against placebo in a subject population of at least 1,500 or at least 2,000 healthy subjects being seropositive at baseline, wherein said unit dose/tetravalent dengue virus composition or said placebo is administered at least twice within less than 6 months, such as within 3 months, about 30 days after the second administration of the administration schedule until at least 12 months or until 12 to 18 months (e.g. at 12 or at 18 months) after the second administration of the administration schedule. In certain such embodiments, the lower bound is more than 30%, is more than 40%, is more than 50%, is more than 60%, is more than 70%, or is more than 80%.

In certain embodiments, the invention is directed to said methods having a combined vaccine efficacy against all four serotypes, in preventing virologically confirmable dengue disease with hospitalization with a 2-sided 95% confidence interval, wherein the lower bound is more than 25%, when measured against placebo in a subject population of at least 5,000 healthy subjects (or at least 10,000, or at least 15,000 healthy subjects) being seropositive at baseline and 4 to 16 years of age, wherein a reconstituted unit dose/tetravalent dengue virus composition as described herein or placebo is e.g. administered at least twice within less than 6 months, such as within 3 months, from 30 days post last administration until 12 to 18 months (e.g. at 12 months or at 18 months) after the last administration. In certain such embodiments, the lower bound is more than 60%, is more than 65%, is more than 70%, is more than 75%, or is more than 80%.

In certain embodiments, the invention is directed to said methods having a combined vaccine efficacy against virologically-confirmed dengue with hospitalization against all four serotypes of more than 30%, when measured against placebo in a subject population at least 1,500 or of at least 2,000 healthy subjects, healthy subjects being seropositive at baseline, wherein said unit dose/tetravalent dengue virus composition or said placebo is administered at least twice within less than 6 months, such as within 3 months, 30 days after the second administration until at least 12 months or until 12 to 18 months (e.g. at 12 or at 18 months) after the second administration. In certain such embodiments, the combined vaccine efficacy against virologically-confirmed dengue with hospitalization against all four serotypes is more than 40%, is more than 50%, is more than 60%, is more than 70%, is more than 80%, or is more than 90%.

In certain embodiments, the invention is directed to said methods having a combined vaccine efficacy against all four serotypes, in preventing virologically confirmable dengue disease with hospitalization, when measured against placebo in a subject population of at least 5,000 healthy subjects (or at least 10,000, or at least 15,000 healthy subjects) being seropositive at baseline and 4 to 16 years of age, wherein a reconstituted unit dose/tetravalent dengue virus composition as described herein or placebo is e.g. administered at least twice within less than 6 months, such as within 3 months, from 30 days post last administration until 12 to 18 months (e.g. at 12 months or at 18 months) after the last administration. In certain such embodiments, the vaccine efficacy is more than 75%, is more than 70%, is more than 80%, is more than 85%, or is more than 90%.

In certain embodiments, the invention is directed to said methods having a combined vaccine efficacy against all four serotypes, in preventing virologically confirmable dengue disease with hospitalization with a 2-sided 95% confidence interval, wherein the lower bound is more than 25%, when measured against placebo in a subject population of at least 5,000 healthy subjects (or at least 10,000, or at least 15,000 healthy subjects) being seropositive at baseline being or seronegative against all serotypes at baseline and 4 to 16 years of age, wherein a reconstituted unit dose/tetravalent dengue virus composition as described herein or placebo is e.g. administered at least twice within less than 6 months, such as within 3 months, from 30 days post last administration until 12 to 18 months (e.g. at 12 months or at 18 months) after the last administration. In certain such embodiments, the difference between the lower bound provided by the seropositive subjects at baseline and the subjects seronegative against all serotypes at baseline is no more than 15%-points.

In certain embodiments, the invention is directed to said methods having a combined vaccine efficacy against all four serotypes, in preventing virologically confirmable dengue disease with hospitalization, when measured against placebo in a subject population of at least 5,000 healthy subjects (or at least 10,000, or at least 15,000 healthy subjects) being seropositive at baseline being or seronegative against all serotypes at baseline and 4 to 16 years of age, wherein a reconstituted unit dose/tetravalent dengue virus composition as described herein or placebo is e.g. administered at least twice within less than 6 months, such as within 3 months, from 30 days post last administration until 12 to 18 months (e.g. at 12 months or at 18 months) after the last administration. In certain such embodiments, the difference between the vaccine efficacy provided by the seropositive subjects at baseline and the subjects seronegative against all serotypes at baseline is no more than 10%-points or no more than 5%-points.

In certain embodiments, the invention is directed to said methods having a relative risk, preferably a combined relative risk against all four serotypes, with a 2-sided 95% confidence interval, wherein the upper bound is less than 0.75, when measured against placebo in a subject population of at least 5,000 healthy subjects (or at least 10,000, or at least 15,000 healthy subjects) irrespective of serostatus at baseline, wherein a reconstituted unit dose/tetravalent dengue virus composition as described herein or placebo is administered at least twice within less than 6 months, such as within 3 months, 30 days after the second administration until at least 12 months after the second administration. In certain such embodiments, the upper bound is less than 0.70, less than 0.65, less than 0.60, less than 0.55, less than 0.50, less than 0.45, less than 0.40, less than 0.35, less than 0.30 or less than 0.28. Preferably said reconstituted unit dose or placebo is administered subcutaneously within about 3 month, such as on days 0 and 90.

In certain embodiments, the invention is directed to said methods having a relative risk, preferably a combined relative risk against all four serotypes, of less than 0.70, when measured against placebo in a subject population of at least 5,000 healthy subjects (or at least 10,000, or at least 15,000 healthy subjects) irrespective of serostatus at baseline, wherein a reconstituted unit dose/tetravalent dengue virus composition as described herein or placebo is administered at least twice within less than 6 months, such as within 3 months, 30 days after the second administration until at least 12 months after the second administration. In certain such embodiments, the relative risk is less than 0.65, less than 0.60, less than 0.55, less than 0.50, less than 0.45, less than 0.40, less than 0.35, less than 0.30, less than 0.25 or less than 0.23. Preferably said reconstituted unit dose or placebo is administered subcutaneously within about 3 month, such as on days 0 and 90.

In certain embodiments, the invention is directed to said methods, wherein virologically confirmable dengue disease occurs in less than 2.5% of the subjects, when measured against placebo in a subject population of at least 5,000 healthy subjects (or at least 10,000, or at least 15,000 healthy subjects) irrespective of serostatus at baseline, wherein a reconstituted unit dose/tetravalent dengue virus composition as described herein or placebo is administered at least twice within less than 6 months, such as within 3 months, 30 days after the second administration until at least 12 months or at least 18 months after the second administration. In certain such embodiments, virologically confirmable dengue disease occurs in less than 2.0% of the subjects, less than 1.5% of the subjects, less than 1.0% of the subjects, less than 0.8% of the subjects, or less than 0.6% of the subjects. Preferably said reconstituted unit dose or placebo is administered subcutaneously within about 3 month, such as on days 0 and 90.

In certain embodiments, the invention is directed to said methods having a combined vaccine efficacy against all four serotypes with a 2-sided 95% confidence interval, wherein the lower bound is more than 61.0%, or more than 65.0 or more than 70.0% or more than 72.0% when measured against placebo in a subject population of at least 5,000 healthy subjects (or at least 10,000, or at least 15,000 healthy subjects) from endemic irrespective of serostatus at baseline and being selected from the group consisting of 4 to 16 year old subjects at the time of randomization, wherein said unit dose/tetravalent dengue virus composition or said placebo is administered at least twice within 6 months or less, about 30 days after the last administration of the administration schedule until at least 12 or 13 months after the last administration of the administration schedule.

In certain embodiments, the invention is directed to said methods having a combined vaccine efficacy against all four serotypes of more than 66%, or of more than 70%, or of more than 75%, or of more than 77%, or of more than 80.0%, when measured against placebo in a subject population of at least 5,000 healthy subjects (or at least 10,000, or at least 15,000 healthy subjects) from endemic areas irrespective of serostatus at baseline and being selected from the group consisting of 4 to 16 year old subjects at the time of randomization, wherein said unit dose/tetravalent dengue virus composition or said placebo is administered at least twice within 6 months or less, about 30 days after the last administration of the administration schedule until at least 12 months or 13 month after the last administration of the administration schedule.

In certain embodiments, the invention is directed to said methods, wherein the combined vaccine efficacy against all four serotypes is measured about 30 days after the last administration of the administration schedule until 12 or 13 months after the last administration of the administration schedule.

In certain embodiments, the invention is directed to said methods, wherein said unit dose or said placebo is administered at least twice within three months, in particular at about day 1 and about day 90, and wherein the combined vaccine efficacy against all four serotypes is measured 30 days after the second administration until 12 or 13 months after the second administration of the administration schedule.

In certain embodiments, the invention is directed to said methods, wherein said methods are effective and safe. In some of these embodiments, the subject or subject population is under 9 years of age, under 4 years of age, or under 2 years of age or from 2 to 9 years of age, or from 2 to 5 years of age, or from 4 to 9 years of age or from 6 to 9 years of age. Optionally the subject is seronegative with respect to all dengue serotypes.

In certain embodiments, the invention is directed to said methods, wherein said methods having a relative risk for virologically confirmed dengue with hospitalization of 1 or less, or 0.8 or less, or 0.6 or less, when measured against placebo in a subject population of at least 5,000 healthy subjects (or at least 10,000, or at least 15,000 healthy subjects). In some of these embodiments, the subject or subject population is under 9 years of age, under 4 years of age, or under 2 years of age or from 2 to 9 years of age, or from 2 to 5 years of age, or from 4 to 9 years of age or from 6 to 9 years of age. Optionally the subject is seronegative with respect to all dengue serotypes.

In certain embodiments, the invention is directed to said methods, wherein the healthy subjects of the subject population are 4 to 16 years of age. In some of such embodiments, the healthy subjects of the subject population are 4 to 5 years of age, 6 to 11 years of age, or 12 to 16 years of age.

In certain embodiments, the invention is directed to said methods, wherein the healthy subjects of the subject population are defined as being healthy in view of the exclusion criteria specified in Example 6.

In certain embodiments, the invention is directed to said methods, wherein the healthy subjects of the subject population are from Asia Pacific or Latin America.

In certain embodiments, the invention is directed to said methods, wherein the healthy subjects of the subject population are seropositive with respect to at least one serotype. In other embodiments, the healthy subjects of the subject population are seronegative with respect to all serotypes.

In certain embodiments, the invention is directed to said methods, wherein the healthy subjects of the subject population are 4-5 years of age and from Asia Pacific, 6-11 years of age and from Asia Pacific, or 12-16 years of age and from Asia Pacific. In other embodiments, the healthy subjects of the subject population are 4-5 years of age and from Latin America, 6-11 years of age and from Latin America, or 12-16 years of age and from Latin America.

In certain embodiments, the invention is directed to said methods, wherein the healthy subjects of the subject population are 4-5 years of age and seropositive for at least 1 dengue serotype, 6-11 years of age and seropositive for at least 1 dengue serotype, or 12-16 years of age and seropositive for at least 1 dengue serotype. In other embodiments, the healthy subjects of the subject population are 4-5 years of age and seronegative for all dengue serotypes, 6-11 years of age and seronegative for all dengue serotypes, or 12-16 years of age and seronegative for all dengue serotypes.

In certain embodiments, the invention is directed to said methods, wherein the healthy subjects of the subject population are from Asia Pacific or Latin America and seropositive for at least one dengue serotype at baseline. In other embodiments, the healthy subjects of the subject population are from Asia Pacific or Latin America and seronegative for at all dengue serotype at baseline.

In certain embodiments, the invention is directed to said methods, wherein the healthy subjects of the subject population are from Asia Pacific, seropositive for at least one dengue serotype at baseline and 4-5 years of age, 6-11 years of age, or 12-16 years of age. In other embodiments, the healthy subjects of the subject population are from Asia Pacific, seronegative for all dengue serotypes at baseline and 4-5 years of age, 6-11 years of age, or 12-16 years of age. In yet other embodiments, the healthy subjects of the subject population are from Latin America, seropositive for at least one dengue serotype at baseline and 4-5 years of age, 6-11 years of age, or 12-16 years of age. In other embodiments, the healthy subjects of the subject population are from America, seronegative for all dengue serotypes at baseline and 4-5 years of age, 6-11 years of age, or 12-16 years of age.

In certain embodiments, the invention is directed to said methods, wherein the healthy subjects of the subject population had prior vaccination against Yellow Fever. In other embodiments, the healthy subjects of the subject population had no prior vaccination against Yellow Fever. Prior vaccination indicates a vaccination prior to the first vaccination with the reconstituted unit dose as described herein. For example for vaccine efficacy (VE) as determined in Example 6 from 30 days post-second vaccination, a prior vaccination of Yellow Fever is defined as a Yellow Fever vaccination occurring before 30 days post-second vaccination.

In certain embodiments, the invention is directed to said methods, wherein the healthy subjects of the subject population had prior vaccination against Japanese Encephalitis. In other embodiments, the healthy subjects of the subject population had no prior vaccination against Japanese Encephalitis.

In certain embodiments, the invention is directed to said methods, wherein the healthy subjects of the subject population received Dengvaxia® within the administration regimen as described herein or within 4.5 years after administration of the first dose. In certain embodiments, the invention is directed to said methods, wherein the occurrence of vaccine related serious adverse events is less than 0.1%.

In certain embodiments, the invention is directed to said methods, wherein the occurrence of vaccine related unsolicited adverse events occurring within 4 weeks of administration is less than 2%.

In certain embodiments, the invention is directed to said methods, wherein the occurrence of vaccine related solicited adverse events occurring within 2 weeks of administration is less than 35%.

In certain embodiments, the invention is directed to said methods, wherein the occurrence of vaccine related solicited local reactions occurring within 1 weeks of administration is less than 40%.

In certain embodiments, the invention is directed to said methods, wherein the method does not increase the risk of virologically-confirmed dengue with hospitalization in the individual, such as in a seronegative individual.

The above methods are also to be considered in the context of a unit dose for use in such methods or in the context of a use of such a unit dose for use in the manufacture of a medicament for such methods.

In certain embodiments, a tetravalent dengue vaccine such as Dengvaxia® is used for inoculating against dengue disease. Dengvaxia® is a tetravalent dengue vaccine based on a yellow fever backbone, CYD-TDV (Dengvaxia®, Sanofi Pasteur, Lyon, France), and has been licensed in several countries based on the clinical demonstration of an overall vaccine efficacy (VE) against virologically-confirmed dengue (VCD) of 56-61% in children in Asia and Latin America (Capeding M R et al. Clinical efficacy and safety of a novel tetravalent dengue vaccine in healthy children in Asia: a phase 3, randomised, observer-masked, placebo-controlled trial. Lancet 2014, 384:1358-65; Villar L A et al. Safety and immunogenicity of a recombinant tetravalent dengue vaccine in 9-16 year olds: a randomized, controlled, phase II trial in Latin America. Pediatr Infect Dis J 2013, 32:1102-9). The preparation of these particular strains CYD1, CYD2, CYD3 and CYD4 has been described in detail in international patent applications WO 98/37911, WO 03/101397, WO07/021672, WO 08/007021, WO 08/047023 and WO 08/065315, to which reference may be made for a precise description of the processes for their preparation. The corresponding nucleotide sequences of the prM-E regions of CYD1, CYD2, CYD3 and CYD4 are provided in WO2016034629 and SEQID NOs are set out in Table 16 of this reference.

In certain embodiments, the invention is directed to said methods, wherein the unit dose disclosed herein, which in particular comprises a chimeric dengue serotype 2/1 strain, a live attenuated dengue serotype 2 strain, a chimeric dengue serotype 2/3 strain and a chimeric dengue serotype 2/4 strain, and Dengvaxia® disclosed herein are administered simultaneously on the same day to the subject or to the subject population.

In certain embodiments, the invention is directed to said methods, wherein the unit dose disclosed herein, which in particular comprises a chimeric dengue serotype 2/1 strain, a live attenuated dengue serotype 2 strain, a chimeric dengue serotype 2/3 strain and a chimeric dengue serotype 2/4 strain, is administered to the subject or to the subject population on day 0/1 as a first administration and Dengvaxia® disclosed herein is subsequently administered to the subject or to the subject population within three months from the first administration, such as on day 90 from the first administration, as a second administration. Alternatively, Dengvaxia® disclosed herein is administered to the subject or to the subject population on day 0/1 as a first administration and the unit dose disclosed herein, which in particular comprises a chimeric dengue serotype 2/1 strain, a live attenuated dengue serotype 2 strain, a chimeric dengue serotype 2/3 strain and a chimeric dengue serotype 2/4 strain, is administered subsequently to the subject or the subject population within three months from the first administration, such as on day 90 from the first administration, as a second administration.

In certain embodiments, the invention is directed to said method, wherein the dengue vaccine composition comprises other dengue vaccines such as TV003 or TV005. TV003, developed by the U.S. National Institute of Allergy and Infectious Diseases, comprises vaccine components rDEN1A30, rDEN2/4430, rDEN3A30/31 and rDEN4A30, wherein each of these components is present at a concentration of 3 log₁₀ PFU. TV005 is similar to TV003 with the difference that the concentration of rDEN2/4A30 in TV005 is 4 log₁₀ PFU. The vaccines TV003 and TV005 and their vaccine components as well as their production are described in more detail in WO 2008/022196 A2 and S. S. Whitehead, Expert Rev Vaccines, 2016, 15 (4): 509 to 517. Using recombinant DNA technology, two attenuation strategies were utilized for the vaccine components of TV003 or TV005: deletions in the 3′ untranslated region and structural gene chimerization. For example, the component rDEN4430 contains all the structural and non-structural proteins of a wild type DENV-4, but is attenuated by a 30-nucleotide deletion in the 3′ untranslated region (denoted “A30”). The other vaccine components are also attenuated due to the 30-nucleotide deletion in the 3′ untranslated region. In addition, rDEN3A30/31 includes a 31 nucleotide deletion in the 3′ untranslated region (shown in detail in FIG. 1c and FIG. 13 of WO 2008/022196 A2). The rDEN2/4430 component was created by substituting the prM and E genes of DENV-2 into the rDEN4A30 genome. The complete genomic sequences of dengue strains which can be used to produce TV003 or TV005 are available under the Genbank accession numbers in Table A of WO 2008/022196 A1.

In certain embodiments, the invention is directed to said methods, wherein the unit dose disclosed herein, which in particular comprises a chimeric dengue serotype 2/1 strain, a live attenuated dengue serotype 2 strain, a chimeric dengue serotype 2/3 strain and a chimeric dengue serotype 2/4 strain, and TV003 or TV005 disclosed herein are administered simultaneously on the same day to the subject or to the subject population.

In certain embodiments, the invention is directed to said methods, wherein the unit dose disclosed herein, which in particular comprises a chimeric dengue serotype 2/1 strain, a live attenuated dengue serotype 2 strain, a chimeric dengue serotype 2/3 strain and a chimeric dengue serotype 2/4 strain, is administered to the subject or the subject population on day 0/1 as a first administration and wherein TV003 or TV005 disclosed herein is subsequently administered to the subject or to the subject population within three months from the first administration, such as on day 90 from the first administration, as a second administration. Alternatively, TV003 or TV005 disclosed herein is administered to the subject or to subject population on day 0/1 as a first administration and the unit dose disclosed herein, which in particular comprises a chimeric dengue serotype 2/1 strain, a live attenuated dengue serotype 2 strain, a chimeric dengue serotype 2/3 strain and a chimeric dengue serotype 2/4 strain, is administered subsequently to the subject or to the subject population within three months from the first administration, such as on day 90 from the first administration, as a second administration.

### Examples

The following Examples are included to demonstrate certain aspects and embodiments of the invention as described in the claims. It should be appreciated by those of skill in the art, however, that the following description is illustrative only and should not be taken in any way as a restriction of the invention.

### Example 1: Preparation of the Dengue Virus Strains

The methods used to generate the chimeric dengue strains TDV-1,-3 and -4 were standard molecular cloning and DNA engineering methods and are describe et al. (2003) J. Virology 77 (21): 11436-11447. The following well-known methods were used to construct and introduce the prM-E genes of dengue serotypes 1, 3 and 4 into the TDV-2 backbone: Reverse-transcriptase PCR (RT-PCR), PCR, restriction enzyme digestion, DNA fragment ligation, bacterial transformations by electroporation, plasmid DNA preparations, in vitro transcription by T7 RNA polymerase, and transfection of Vero cells by electroporation.

After growing and purifying the different dengue serotypes separately as described in Huang et al. (2013) PLOS Neglected Dis, 7 (5): e2243, they are mixed in certain concentrations provided in Example 4. The mixture of dengue serotypes is present in a dengue vaccine composition and combined with a composition of pharmaceutically acceptable excipients resulting in a dengue vaccine composition comprising 15% w/v α,α trehalose dihydrate, 1% w/v poloxamer 407, 0.1% w/v human serum albumin and 100 mM sodium chloride. The dengue vaccine composition is lyophilized and represents a lyophilized unit dose of TDV. The lyophilized unit dose is reconstituted with 37 mM aqueous sodium chloride solution and the reconstituted unit dose comprises 15% w/v α,α trehalose dihydrate, 1% w/v poloxamer 407, 0.1% w/v human serum albumin and 137 mM sodium chloride.

### Example 2: Microneutralization Test

Immunogenicity was measured by a microneutralization assay to each one of the four dengue serotypes with titers defined as the dilution resulting in a 50% reduction in plaque values (MNT50). Briefly, on day 1 Vero cells were seeded on 96-well assay plates in DMEM and 10% FBS at a density of 2.5×10⁵ cells/ml and incubated at 37° C. for 24 hours. On day 2 serial dilutions of the heat-inactivated antibody-containing test and control sera samples (dilutions range 1:10 to 1:20480) were prepared and mixed with a constant concentration of dengue viruses, in particular DENV-1 strain 16007, DENV-2 strain 16681, DENV-3 strain 16562 and DENV-4 strain 1036, (target 60-80 pfu/well) in a 96 well microtiter plate and incubated overnight at 2-8° C. to enable the neutralization of the virus by the antibodies present in the sera. After the incubation the mixture of virus and antibodies was transferred onto the 96 well plates with Vero cells and the plates were incubated at 37° C. for 90-120 minutes to infect the Vero cells. A 1% methylcellulose overlay in DMEM was applied to the plate to restrict spread of progeny virus and the plate was incubated for 46-70 hours at 34° C. depending on the Dengue serotype:
DENV1-66±2 hours
DENV2-70±2 hours
DENV3-66±2 hours

DENV4-46±2 hours

After the incubation the cells were washed twice with PBS and fixed by adding cold methanol and incubating for 60 minutes at a temperature of ≤−20° C. After fixing the plates were dried and washed three times with washing buffer (1×PBS, pH 7.4 with 0.5% Tween), before 50 μl of serotype-specific anti-dengue monoclonal antibodies in blocking solution (2.5% nonfat dry milk in PBST) per well were added and incubated with the cells for 18+4 hours at 2-8° C.

The monoclonal antibodies were made as described in Gentry et al. (1982) Am. J. Trop. Med. Hyg. 31, 548-555; Henchal et al. (1985) Am. J. Trop. Med. Hyg. 34, 162-169; and Henchal et al. (1982) Am. J. Trop. Med. Hyg. 31 (4): 830-6). Briefly, the anti-DENV-1 HBD was made against dengue 1 strain Hawaii, Envelope, the anti-DENV-2 was made against dengue 2 strain New Guinea C, Envelope, isotype 1, the anti-DENV-3 HBD was made against dengue 3 strain H87, Envelope, isotype 2A, and the anti-DENV-4 HBD was made against dengue 4 strain H241, Envelope, isotype 2A.

After incubation, the plates were washed three times with washing buffer and 50 μl of a secondary peroxidase labelled goat anti-mouse IgG (H+L) (KPL Cat #074-1806) in blocking solution was added and incubated for 90 to 120 minutes at 37° C. Then the plates were washed three times with washing buffer and 50 μl of precipitant substrate (2-amino-9-ethyl carbazole (AEC) tablet in 2.5 ml DMSO, 47.5 ml 50 mM acetate buffer and 250 μl hydrogen peroxide) were added and the mixture was incubated for 20 minutes at room temperature. Finally, the substrate was removed, the plates were rinsed with dH₂O and dried.

Sample titers are calculated using the linear regression method and reported as MNT50 titers for each sample. Clinical data are reported as a geometric mean titer for all the individual MNT50 titers in each treatment group. Briefly, the number of infectious foci in each well was counted and the titer of neutralizing antibodies was determined by comparing the percent reduction of infectious foci centers in wells containing antibody (test samples) in comparison to wells containing virus alone. The MNT50 was calculated using the following linear regression equation:

MNT50=10\hat{ }[(50-c)/m]) where c=γintercept of regression line and m=slope of regression line

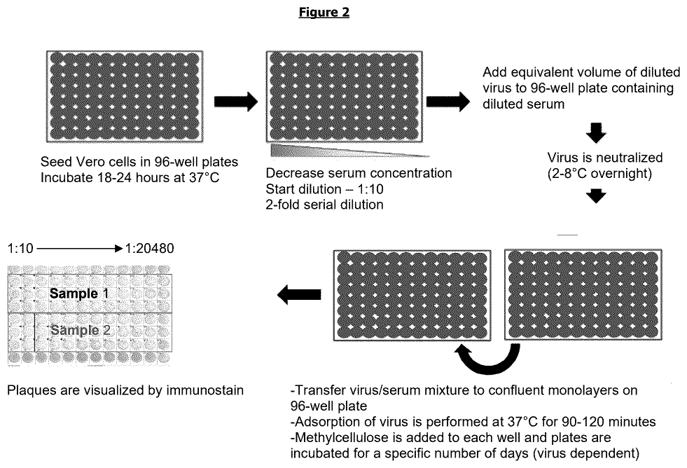

Each test sample was tested in triplicates and the titer was calculated from the average of the triplicates. A schematic drawing of the steps performed in this test is provided in FIG. 2.

### Example 3: Phase III Clinical Trial in Children

A Phase III, double-blind, randomized, and placebo-controlled trial in 20100 subjects aged 4 to 16 years living in Thailand, Sri Lanka, Philippines, Panama, Nicaragua, Dominican Republic, Colombia or Brazil was performed evaluating the efficacy, safety and immunogenicity of a tetravalent dengue vaccine referred to hereinafter as TDV (TDV-1, TDV-2, TDV-3 and TDV-4 as described herein). The trial includes 3 parts. Part 1 evaluates vaccine efficacy (VE) and lasts until both of the following 2 criteria are fulfilled: (i) 120 cases of dengue fever are confirmed and (ii) minimum duration of subject follow-up of 12 months post-second vaccination. Part 2 is for an additional 6 months to evaluate VE and for secondary efficacy analyses. Part 3 will evaluate long-term safety by following participants for side effects and will last an additional 3 years.

Part 1: Active surveillance for the primary assessment of efficacy in all subjects. During this time subjects were contacted at least weekly to ensure identification of febrile illness that could potentially be due to dengue. This part commenced on the day of vaccination and finished once both of the following 2 criteria were fulfilled: (i) 120 cases of dengue fever are confirmed and (ii) minimum duration of subject follow-up of 12 months post-second vaccination. The end of Part 1 was defined for each subject so that the duration of follow up after the second vaccination was approximately the same for all subjects. Virologically-confirmed cases in Part 1 count towards the primary efficacy objective if occurring at least 30 days post-second vaccination. Part 1 was finished 12 months post-second vaccination

Part 2: Active surveillance for an additional 6 months for each subject following the completion of Part 1, I, i.e. 18 month post second vaccination. During this time subjects were contacted at least weekly to ensure identification of febrile illness that could potentially be due to dengue. Virologically-confirmed cases in Parts 1 and 2 contribute towards the secondary efficacy objectives.

Part 3: Modified active surveillance for the assessment of safety in all subjects following the completion of Part 2 and lasting 3 years for each subject. The modified surveillance during Part 3 will maintain at least weekly contacts through Part 3 of the trial, but the intensity of investigation will be modified based on the need for hospitalization. Surveillance will identify febrile illness of any severity that could potentially be due to dengue.

### Criteria for Inclusion Include:

The subject was aged 4 to 16 years inclusive, at the time of randomization.
Individuals who were in good health at the time of entry into the trial as determined by medical history, physical examination (including vital signs) and clinical judgment of the Investigator.
The subject and/or the subject's parent/guardian signed and dated an assent/written informed consent form where applicable, and any required privacy authorization prior to the initiation of any trial procedures, after the nature of the trial has been explained according to local regulatory requirements.
Individuals who can comply with trial procedures and are available for the duration of follow-up.

### Exclusion Criteria Include:

1. Febrile illness (temperature≥38° C.) or moderate or severe acute illness or infection at the time of randomization.
2. History or any illness that, in the opinion of the Investigator, might interfere with the results of the trial or pose an additional risk to the subject due to participation in the trial, including but not limited to:
   a. Known hypersensitivity or allergy to any of the vaccine components.
   b. Female subjects (post-menarche) who are pregnant or breastfeeding.
   c. Individuals with any serious chronic or progressive disease according to judgment of the Investigator (e.g., neoplasm, insulin-dependent diabetes, cardiac, renal or hepatic disease, neurologic or seizure disorder or Guillain-Barré syndrome).
   d. Known or suspected impairment/alteration of immune function, including:
      i. Chronic use of oral steroids (equivalent to 20 mg/day prednisone≥12 weeks/≥2 mg/kg body weight/day prednisone>2 weeks) within 60 days prior to Day 1 (Month 0) (use of inhaled, intranasal, or topical corticosteroids is allowed).
      ii. Receipt of parenteral steroids (equivalent to 20 mg/day prednisone≥12 weeks/>2 mg/kg body weight/day prednisone>2 weeks) within 60 days prior to Day 1 (Month 0).
      iii. Administration of immunoglobulins and/or any blood products within the 3 months prior to Day 1 (Month 0) or planned administration during the trial.
      iv. Receipt of immunostimulants within 60 days prior to Day 1 (Month 0).
      v. Immunosuppressive therapy such as anti-cancer chemotherapy or radiation therapy within 6 months prior to Day 1 (Month 0).
      vi. Human Immunodeficiency Virus (HIV) infection or HIV-related disease.
      vii. Genetic immunodeficiency.
3. Receipt of any other vaccine within 14 days (for inactivated vaccines) or 28 days (for live vaccines) prior to Day 1 (Month 0) or planning to receive any vaccine within 28 days after Day 1 (Month 0).
4. Participation in any clinical trial with another investigational product 30 days prior to Day 1 (Month 0) or intent to participate in another clinical trial at any time during the conduct of this trial.
5. Previous participation in any clinical trial of a dengue candidate vaccine, or previous receipt of a dengue vaccine.
6. First degree relatives of individuals involved in trial conduct.
7. Females of childbearing potential who are sexually active, and who have not used any of the acceptable contraceptive method for at least 2 months prior to Day 1 (Month 0).
8. Females of childbearing potential who are sexually active, and who refuse to use an acceptable contraceptive method up to 6 weeks post-second vaccination.
9. Deprived of freedom by administrative or court order, or in an emergency setting, or hospitalized involuntarily.
10. Current alcohol abuse or drug addiction that may interfere with the subject's ability to comply with trial procedures.
11. Identified as an employee of the Investigator or trial center, with direct involvement in the proposed trial or other trials under the direction of that Investigator or trial center.

Eligible subjects were randomized (2:1) into two treatment groups: groups 1 received one subcutaneous (SC) dose of TDV in the upper arm on Day 1 and on Day 90, and group 2 received one subcutaneous dose of placebo in the upper arm on Day 1 and on Day 90. Randomization was stratified by region (Asia Pacific and Latin America) and age range (children aged 4-5 years, 6-11 years, and 12-16 years) to ensure each age range has the appropriate ratio of TDV to placebo in each region. After randomization dropouts were not replaced. Study Day 1 is defined to be the date of the first dose administration of TDV or placebo. The TDV was prepared as described in Example 1. Each subcutaneous dose of TDV was 0.5 mL and the concentration of the four dengue serotypes in the TDV vaccine in each dose was 3.6 log₁₀ PFU/dose, 4.0 log 10 PFU/dose, 4.6 log₁₀ PFU/dose and 5.1 log₁₀ PFU/dose of TDV-1, TDV-2, TDV-3 and TDV-4, respectively.

The “total concentration in pfu/0.5 ml” which serves as a base value for the calculation of the percentage concentration for each individual component of a tetravalent dengue vaccine is shown for one exemplary tetravalent vaccine composition comprising dengue serotype 1 in a concentration of 3.60 log₁₀ pfu/0.5 ml, a dengue serotype 2 concentration of 4.00 log₁₀ pfu/0.5 ml, a dengue serotype 3 concentration of 4.60 log 10 pfu/0.5 ml and a dengue serotype 4 concentration of 5.11 log₁₀ pfu/0.5 ml. Primarily, the logarithmic values of the concentrations are converted into numerical values. The results of this conversion are 4×10³ pfu/0.5 ml for serotype 1, 1×10⁴ pfu/0.5 ml for serotype 2, 4×10⁴ pfu/0.5 ml for serotype 3 and 1.3×10⁵ pfu/0.5 ml for serotype 4. The total concentration in pfu/0.5 ml is the sum of the preceding numerical values resulting in 1.84×10⁵ pfu/0.5 ml.

The “percentage concentration” for each of the serotypes 1, 2, 3 and 4 is obtained by dividing the numerical concentration value (expressed as pfu/0.5 ml) of an individual serotype by the total concentration (expressed in pfu/0.5 ml) and multiplying the result by 100 i.e.:

$$Percentage concentration of serotype 1 = ( 4 \times {10}^{3} pfu / 0.5 ml \div 1.84 \times {10}^{5} pfu / 0.5 ml ) \times 100 = 2 \%$$

$$Percentage concentration of serotype 2 = ( 1 \times {10}^{4} pfu / 0.5 ml \div 1.84 \times {10}^{5} pfu / 0.5 ml ) \times 100 = 5 \%$$

$$Percentage concentration of serotype 3 = ( 4 \times {10}^{4} pfu / 0.5 ml \div 1.84 \times {10}^{5} pfu / 0.5 ml ) \times 100 = 22 \%$$

$$Percentage concentration of serotype 4 = ( 1.3 \times {10}^{5} pfu / 0.5 ml \div 1.84 \times {10}^{5} pfu / 0.5 ml ) \times 100 = 71 \% .$$

The percentage concentrations are rounded to whole numbers.

Primary Outcome Measures included the vaccine efficacy (VE) of two doses of TDV in preventing virologically-confirmed dengue (VCD) fever induced by any dengue serotype [time frame: 30 days post-second vaccination (Day 120) until the end of Part 1]. VE is defined as 1-(λv/λc), wherein Av and Ac denote the hazard rates for the TDV and placebo groups, respectively. A virologically-confirmed dengue case is defined as febrile illness (defined as temperature≥38° C. on any 2 of 3 consecutive days) or illness clinically suspected to be dengue by the Investigator with a positive serotype-specific reverse transcriptase polymerase chain reaction (RT-PCR). A febrile illness will require an interval of at least 14 days from a previous febrile illness to avoid overlap of acute and convalescent visits from one episode with those from a second episode.

### Secondary Outcome Measures Include:

1) VE of two doses of TDV in preventing virologically-confirmed dengue fever induced by each dengue serotype [time frame: from 30 days post-second vaccination (Day 120) until the end of Part 2].
2) VE of two doses of TDV in preventing virologically-confirmed dengue fever induced by any dengue serotype in participants dengue seronegative at baseline [time frame: from 30 days post-second vaccination (Day 120) until the end of Part 2 (up to 21 months)].
3) VE of two doses of TDV in preventing virologically-confirmed dengue fever induced by any dengue serotype in participants dengue seropositive at baseline [time frame: from 30 days post-second vaccination (Day 120) until the end of Part 2].
4) VE of two doses of TDV in preventing hospitalization due to virologically-confirmed dengue fever induced by any dengue serotype [time frame: from 30 days post-second vaccination (Day 120) until the end of Part 2].
5) VE of two doses of TDV in preventing virologically-confirmed severe dengue fever induced by any dengue serotype [time frame: from 30 days post-second vaccination (Day 120) until the end of Part 2].
6) Percentage of participants with solicited local injection site adverse events (AEs) in the safety subset [time frame: Days 1 through 7 after each vaccination] and severity of solicited local injection AEs. Solicited local AEs at injection site are defined as pain, erythema and swelling that occurred within 7 days after each vaccination.
7) Percentage of participants with solicited systemic adverse events (AEs) in the safety subset [time frame: Days 1 through 14 after each vaccination] and severity of solicited systemic AEs. Solicited systemic AEs in children (<6 years) are defined as fever, irritability/fussiness, drowsiness and loss of appetite that occurred within 14 days after each vaccination. Solicited systemic AEs in children (≥6 years) are defined as fever, headache, asthenia, malaise and myalgia that occurred within 14 days after each vaccination.
8) Percentage of participants with any unsolicited adverse events (AEs) in the safety subset [time frame: Days 1 through 28 after each vaccination]. Unsolicited AEs are any AEs that are not solicited local or systemic AEs, as defined above.
9) Percentage of participants with serious adverse events (SAEs) during Parts 1 and 2 [time frame: from Day 1 until the end of Parts 1 and 2]. A serious adverse event (SAE) is any untoward medical occurrence or effect that at any dose results in death, is life-threatening, requires inpatient hospitalization or prolongation of existing hospitalization, results in persistent or significant disability/incapacity, is a congenital anomaly/birth defect or is medically important due to other reasons than the above mentioned criteria.
10) Percentage of participants with fatal SAEs and SAEs related to study drug during the first and second half of Part 3 [time frame: for 3 years (18 month halves) beginning at the end of Part 2 (approximately 21 months after the first vaccination)].
11) Percentage of participants with a seropositive response for each of the four dengue serotypes in the immunogenicity subset [time frame: Day 1 and months 1, 3, 4, 9, 15 and then annually (up to 3 years)]. Seropositive response is defined as a reciprocal neutralizing titer≥10. The four DENV serotypes are DEN-1, DEN-2, DEN-3 and DEN-4.
12) Percentage of participants with a seropositive response for multiple dengue serotypes in the immunogenicity subset [time frame: Day 1 and months 1, 3, 4, 9, 15 and then annually (up to 3 years)].
13) Geometric Mean Titers (GMTs) of neutralizing antibodies for each of the four dengue serotypes in the immunogenicity subset [time frame: Day 1 and months 1, 3, 4, 9, 15 and then annually (up to 3 years)]. GMTs of neutralizing antibodies will be measured via microneutralization test (MNT) as described in Example 2.

### A) Study Population

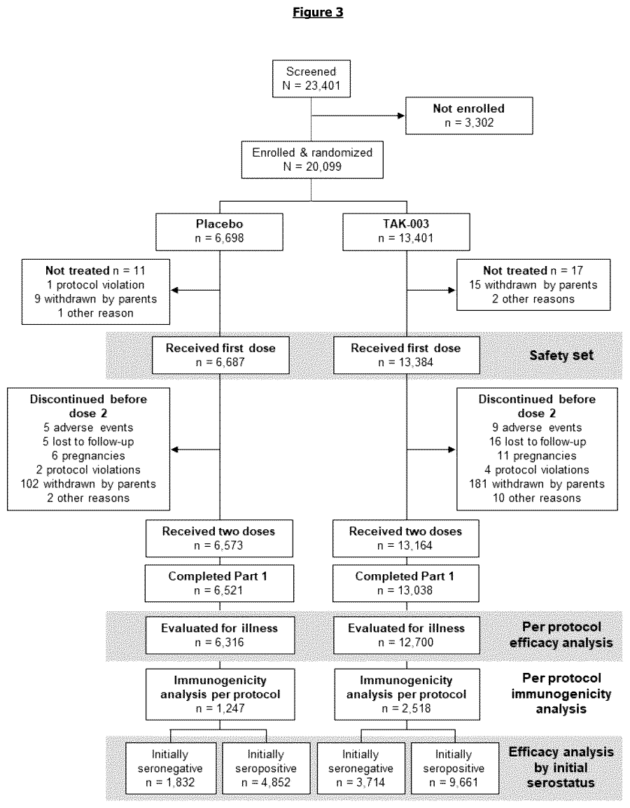

After screening, 20,099 participants were randomized, and 20,071 received at least one injection. In total, 97.4% of placebo participants (n/N: 6,521/6,698) and 97.3% of vaccinees (n/N: 13,038/13,401) completed Part 1 of the study (FIG. 3). Reasons for study withdrawals included AEs, participants lost to follow-up, pregnancy, protocol violations, and withdrawal by participants (or parents/guardians). Baseline characteristics were similar across both treatment groups (Table 5). Mean age of study participants was 9.6 years, with baseline seronegativity of 27.7%, and enrollment was broadly balanced across regions (46.5% in Asia, 53.5% in Latin America). The highest seronegative rate was in Panama (62.2%), followed by Sri Lanka (38.5%), Thailand (34.4%), Brazil (28.8%), Nicaragua (22.3%), Colombia (15.4%), the Philippines (12.4%), and the Dominican Republic (2.8%).

TABLE 5
Baseline characteristics of study population (number, %)

| | | |
|---|---|---|
| TDV | Placebo | Total |

Per Protocol Set

| | | | |
|---|---|---|---|
| Number of Participants | 12,704 | 6,317 | 19,021 |
| Mean Age (Years, SD) | 9.6 (3.35) | 9.6 (3.34) | 9.6 (3.35) |
| Baseline Seronegative^{a} | 3,533 (27.8) | 1,726 (27.3) | 5,259 (27.7) |
| Female | 6,314 (49.7) | 3,098 (49.0) | 9,412 (49.5) |
| Male | 6,390 (50.3) | 3,219 (51.0) | 9,609 (50.5) |
| Asia Pacific | 5,896 (46.4) | 2,942 (46.6) | 8,838 (46.5) |
| Baseline Seronegative^{a} | 1,503 (25.5) | 773 (26.3) | 2,276 (25.8) |
| Latin America | 6,808 (53.6) | 3,375 (53.4) | 10,183 (53.5) |
| Baseline Seronegative^{a} | 2,030 (29.8) | 953 (28.2) | 2,983 (29.3) |

Safety Set^{b}

| | | | |
|---|---|---|---|
| Number of Participants | 13,380 | 6,687 | 20,071 |
| Mean Age (Years, SD) | 9.6 (3.36) | 9.6 (3.34) | 9.6 (3.35) |
| Baseline Seronegative^{a} | 3,714 (27.8) | 1,832 (27.4) | 5,547 (27.6) |
| Female | 6,651 (49.7) | 3,276 (49.0) | 9,929 (49.5) |
| Male | 6,729 (50.3) | 3,411 (51.0) | 10,142 (50.5) |

Safety Set of Subset^{b}

| | | | |
|---|---|---|---|
| Number of Participants | 2,663 | 1,329 | 3,993 |
| Baseline Seronegative^{a} | 740 (27.8) | 369 (27.8) | 1,109 (27.8) |

^{a}Seronegative for all serotypes; seropositive defined as reciprocal neutralizing antibody titer ≥10; SD, standard deviation.
^{b}numbers of participants in TVD plus placebo groups are not equal to total numbers shown because misallocated participants (i.e. those who received both TVD and placebo due to an administrative error) are not included in the TDV and placebo group data.

### B) Febrile Illnesses and VCD

During Part 1, 5,754 and 4,663 episodes of febrile illness were reported in Asian and Latin American sites, respectively. Acute samples were obtained in 99.5% and 96.6% of these cases, with 98.3% and 85.1% of samples taken within five days, in Asia and Latin America, respectively. There were 278 VCD cases (76 hospitalized) in the safety set during the entire Part 1 period, of which 210 (58 hospitalized) were 30 days post-second vaccination in the PPS (Table 6; Table 8) and were included in primary endpoint analysis.

### C) Distribution of VCD Included in Primary Endpoint Analysis

DENV-1 was reported in all countries with VCD and included all the 21 cases in Panama. In Sri Lanka, 54 of 60 VCD were DENV-2, and 87 of 109 VCD in the Philippines were DENV-3. All seven DENV-4 VCD were reported in the Philippines. No VCD were reported in Nicaragua or the Dominican Republic. Of the associated 58 hospitalized VCD, 43 were reported in Sri Lanka. A total of two severe dengue (both DENV-3) and five dengue hemorrhagic fever (DHF; three DENV-2; two DENV-3) cases were reported (Table 7). These seven were also the only such cases in the entire part 1 safety set.

### D) Vaccine Efficacy

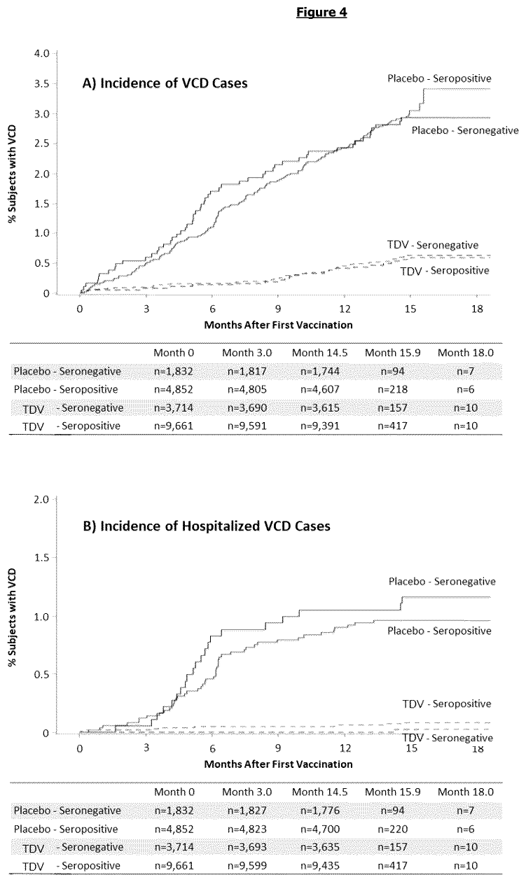

VE against VCD of any serotype was 80.2% (95% CI: 73.3-85.3; P<0.001). A similar efficacy of 81% (95% CI: 64.1-90.0) between the doses and from first dose onwards in the safety set (Table 6) suggests that the vaccine was efficacious after the first dose. Exploratory analysis of the secondary efficacy endpoints showed a trend of differential efficacy by serotype, with the highest efficacy against DENV-2 (97.7%), followed by DENV-1 (73.7%), DENV-4 (63.2% with CI containing zero), and DENV-3 (62.6%; Table 7). Overall, efficacy was similar in baseline seronegatives (74.9%) and seropositives (82.2%; FIG. 4 above); however, this varied by serotype. Efficacy against DENV-2 was not impacted by serostatus; efficacy against DENV-1 was slightly higher in baseline seropositives (79.8%; 95% CI: 51.3-91.6) than baseline seronegatives (67.2%; 95% CI: 23.2-86.0). No efficacy was observed against DENV-3 in baseline seronegatives (−38.7%; 95% CI:-335.7-55.8) compared to baseline seropositives (71.3%; 95% CI: 54.2-82.0). Efficacy by serostatus could not be calculated for DENV-4 because no cases were observed in baseline seronegatives. In the primary endpoint timeframe of the PPS, only five VCD requiring hospitalization were reported in the vaccine group compared with 53 cases in the placebo group, with a VE of 95.4% (95% CI: 88.4-98.2; 97.2% for baseline seronegatives and 94.4% for baseline seropositives; Table 7; FIG. 4 below), consistent with a VE of 93.3% (95% CI: 86.7-96.7) in the safety set from first dose onwards.

The primary vaccine efficacy (VE) of two doses of TDV in preventing virologically-confirmed dengue (VCD) fever induced by any dengue serotype is shown in Table 6.

TABLE 6
Vaccine efficacy of TDV in preventing virologically-confirmed dengue
(VCD) fever against any serotype from 30 days post-second vaccination
until end of part 1 Per Protocol Set (PPS), i.e. 12 months post-second
vaccination. Safety set analysis from first dose to end of Part 1
study period, i.e. 12 months post-second vaccination

| | Placebo | TDV (PPS) |
|---|---|---|
| | n = 6317 | n = 12,704 |
| number of subject evaluated | 6,316 | 12,700 |
| number of subjects with | 1,712 | 3,195 |
| febrile illness | | |
| number of febrile illness | 2,591 | 4,692 |
| cases | | |
| virologically confirmed | 149 [2.4] | 61 [0.5] |
| dengue fever (n [%]) | | |
| Person-years at risk | 5,670.1 | 11,578.7 |
| incident density | 2.6 | 0.5 |
| relative risk | 0.20 | |
| 95% CI of relative risk | (0.15, 0.27) | |
| vaccine efficacy (%) | 80.2 | |
| 95% CI of vaccine efficacy | (73.3, 85.3) | |
| p-value for vaccine efficacy | <0.001 | |
| | Placebo | TDV (Safety Set)* |
| number of subject evaluated | 6,687 | 13,380 |
| virologically confirmed | 199 [3.0] | 78 [0.6] |
| dengue fever (n [%]) | | |
| Person-years at risk | 8,072.0 | 16,351.5 |
| incident density | 2.5 | 0.5 |

| | |
|---|---|
| vaccine efficacy (%) | 80.9 |
| 95% CI of vaccine efficacy | (75.2, 85.3) |

Note 1:
Percentage of virologically confirmed dengue (VCD) fever are based on number of subjects evaluated.
Note 2:
Person-years at risks is defined as cumulative time in years until start of VCD fever or until end of Part 1 study period or discontinuation date, whichever comes first. Incident density is defined as the number of cases per 100 person-years at risk. Percentages are based on total number (denominator) of analysis set participants evaluated and may not be equal to the total number of participants in the per protocol analysis set.
*One participant had two instances of VCD during Part 1, only the first VCD was included in efficacy calculation
Note 3:
Vaccine efficacy (VE) and 2-sided 95% CIs are estimated from a Cox proportional hazard model with TDV as a factor, adjusted for age and stratified by region.
Note 4:
Statistical significance will be concluded if the lower bound of the 95% CI for VE is above 25%. Since the hypotheses will be tested in a confirmatory manner at a 2-sided significance level of 5%, the calculated p-value should be compared with 0.025.
Note 5:
Relative risk is calculated as the number of events divided by the number of subjects evaluated in the TDV group, over the number of events divided by the number of subjects evaluated in the placebo group.

For the efficacy evaluation shown in Table 6, a case of VCD was defined as febrile illness (defined as fever≥38° C. on any 2 of 3 consecutive days) with a positive serotype-specific RT-PCR (i.e., positive dengue detection RT-PCR) and occurring at any time starting from 30 days post-second vaccination (Day 120 [Month 4]) through the end of Part 1. The analysis was performed on the Per-Protocol Set (PPS) and

### Safety Set.

As used herein, the “Per-Protocol Set (PPS)” consist of all subjects in the Full Analysis Set (FAS) consisting of all randomized subjects who received at least one dose of TDV or placebo who had no major protocol violations. Major protocol violations are not receiving both doses of TDV or placebo administration, not receiving both doses in the correct interval, not having the correct administration of TDV or placebo, use of prohibited medications/vaccines by the subject, the subject meets any of the exclusion criteria of 2d, 3, 4 or 5 defined above or product preparation error.

The p-value is obtained by solving the critical value Z in the following equation:

Upper bound of 1-sided(1-p%)CI of HR=0.75, wherein HR is the hazard ratio and defined as HR=λV/λC.

e\hat{ }[B\hat{ }+Z*SE]=0.75, wherein β\hat{ }defines the treatment and SE the related standard error. The 1-sided p-value is 1-(area to the left of the critical value Z from a standard normal distribution). Since the hypotheses will be tested in a confirmatory manner 2-sided at a significance level of 5%, the calculated 1-sided p-value should be compared with 0.025.

In summary in Part 1 of this study, a high vaccine efficacy of 80.2% against virologically-confirmed dengue of any serotype in children 4-16 years of age was found. It included an efficacy of 74.9% in baseline seronegatives and a robust 95.4% reduction in hospitalizations. Onset of protection could be seen after the first dose with 81% efficacy between doses. Overall, these results suggest a potential benefit for each vaccine recipient regardless of prior dengue exposure or age. This finding is significant because vaccine development against dengue has been challenging, especially for dengue naïve individuals, and dengue remains one of the WHO's top ten threats to global health in 2019.19 Furthermore, the onset of protection after the first dose has potential utility in the context of outbreak control or travel vaccination, offering a reduction in the risk of dengue after only one dose.

Severe forms of dengue were assessed as follows: Dengue Hemorrhagic Fever (DHF) as defined by the 1997 WHO definition. Severe Dengue through the Dengue Case Adjudication Committee. The Dengue Case Adjudication Committee (DCAC) consisted of four members: a voting chairperson, two voting members, and an independent non-voting statistician. The three DCAC voting members are all physicians and clinical dengue experts. DCAC members are not study investigators and do not have any conflict of interest that would bias their review of the trial data. All non-hospitalized cases were considered non-severe. The DCAC severe dengue case criteria applied in a blinded manner to virologically-confirmed hospitalized dengue cases are as follows: 1) bleeding abnormality, for a case to be considered severe there needs to be a significant intervention required in response to the bleeding episode such as blood transfusion, nasal packing, hormonal therapy, or, bleeding occurred into critical organs such as the brain; 2) plasma leakage, for a case to be considered severe there needs to be evidence of both plasma leakage and functional impairment (plasma leakage includes clinical evidence, radiological evidence, or hematocrit elevated>20% above normal levels or baseline; functional impairment defined as shock or respiratory distress); 3) liver, for a case to be considered severe there needs to be evidence of both hepatitis and functional impairment (hepatitis defined as an aspartate aminotransferase [AST] or alanine aminotransferase [ALT]>10 upper limit of normal range [ULN]; functional impairment defined as prothrombin [PT]>1.5 ULN or hypoalbuminemia); 4) renal, serum creatinine>2.5 times ULN or requiring dialysis; 5) cardiac, abnormalities intrinsic to the heart (i.e. not resulting from intravascular volume depletion) and with evidence of functional impairment (examples of intrinsic abnormality: myocarditis, pericarditis, and myopericarditis; example of functional impairment: new conduction abnormality resulting in irregular heart rhythm [i.e. not transient first-degree heart block]); 6) central nervous system, any abnormality with the exception of a simple febrile convulsion or a brief delirium; 7) shock, all shock cases considered severe. At least 1 functional impairment (of criterion 3,4,5,6), needs to be present but the totality of data were considered by the members in their assessment.

Further results of part 1 and part 2 are presented in Tables 7a to c.

TABLE 7a
Distribution of cases contributing to primary endpoint by per protocol set subgroup (30 days
after second vaccination until end of Part 1, i.e. 12 months after second vaccination)

| | | | | |
|---|---|---|---|---|
| TDV | TDV | Placebo | Placebo | Vaccine Efficacy |
| Dengue Cases | Incidence Density | Dengue Cases | Incidence Density | (95% CI) |

VCD cases

| | | | | | |
|---|---|---|---|---|---|
| Baseline Seropositive^{a} | 41/9,165 (0.4%) | 0.5 | 110/4,587 (2.4%) | 2.7 | 82.2% (74.5%-87.6%) |
| Baseline Seronegative^{a} | 20/3,531 (0.6%) | 0.6 | 39/1,726 (2.3%) | 2.5 | 74.9% (57.0%-85.4%) |
| DENV-1 | 16/12,700 (0.1%) | 0.1 | 30/6,316 (0.5%) | 0.5 | 73.7% (51.7%-85.7%) |
| DENV-2 | 3/12,700 (<0.1%) | <0.1 | 64/6,316 (1.0%) | 1.1 | 97.7% (92.7%-99.3%) |
| DENV-3 | 39/12,700 (0.3%) | 0.3 | 51/6,316 (0.8%) | 0.9 | 62.6% (43.3%-75.4%) |
| DENV-4^{d} | 3/12,700 (<0.1%) | <0.1 | 4/6,316 (<0.1%) | <0.1 | 63.2% (−64.6%-91.8%) |
| 4-5 Years Old | 13/1,619 (0.8%) | 0.9 | 23/801 (2.9%) | 3.2 | 72.8% (46.2%-86.2%) |
| 6-11 Years Old | 34/7,009 (0.5%) | 0.5 | 85/3,491 (2.4%) | 2.7 | 80.7% (71.3%-87.0%) |
| 12-16 Years Old | 14/4,072 (0.3%) | 0.4 | 41/2,024 (2.0%) | 2.2 | 83.3% (69.3%-90.9%) |
| Asia | 54/5,894 (0.9%) | 1.0 | 127/2,942 (4.3%) | 4.9 | 79.5% (71.8%-85.1%) |
| Latin America | 7/6,806 (0.1%) | 0.1 | 22/3,374 (0.7%) | 0.7 | 84.3% (63.1%-93.3%) |

Hospitalized VCD cases

| | | | | | |
|---|---|---|---|---|---|
| Baseline Seropositive^{a} | 4/9,165 (<0.1%) | <0.1 | 35/4,587 (0.8%) | 0.8 | 94.4% (84.3%-98.0%) |
| Baseline Seronegative^{a} | 1/3,531 (<0.1%) | <0.1 | 18/1,726 (1.0%) | 1.2 | 97.2% (79.1%-99.6%) |

Cases of DHF^{b}

| | | | | | |
|---|---|---|---|---|---|
| All participants | 1/12,700 (<0.1%) | <0.1 | 4/6,316 (<0.1%) | <0.1 | 87.3% (−13.5%-98.6%) |

Severe VCD Cases^{c}

| | | | | | |
|---|---|---|---|---|---|
| All participants | 1/12,700 (<0.1%) | <0.1 | 1/6,316 (<0.1%) | <0.1 | 50.8% (−686.9%-96.9%) |

VCD, virologically-confirmed dengue;
DHF, dengue hemorrhagic fever
^{a}Seronegative for all serotypes; baseline seropositive defined as reciprocal neutralizing antibody titer ≥10 to one or more serotypes.
^{b}VCD cases meeting WHO 1997 DHF criteria; incidence density defined as the number of cases per 100 person-years at risk; percentages are based on total number (denominator) of per protocol set participants evaluated.
^{c}two severe VCD were not classified as DHF.
^{d}The number of cases identified was sufficient to provide reasonably precise estimates of vaccine efficacy against all individual serotypes, except DENV-4.

TABLE 7b
Distribution of cases contributing to secondary endpoint by per protocol set subgroup (30
days after second vaccination until end of Part 2, i.e. 18 months after second vaccination)

| | | | | |
|---|---|---|---|---|
| TDV | TDV | Placebo | Placebo | Vaccine Efficacy |
| Dengue Cases | Incidence Density | Dengue Cases | Incidence Density | (95% CI) |

VCD cases

| | | | | | |
|---|---|---|---|---|---|
| Overall | | | | | 73.3% (66.5%-78.8%) |
| Baseline Seropositive^{a} | 75 | 0.6 | 150 | 2.4 | 76.1% (68.5%-81.9%) |
| Baseline Seronegative^{a} | 39 | 0.8 | 56 | 2.4 | 66.2% (49.1%-77.5%) |
| DENV-1 | 38 | 0.2 | 62 | 0.7 | 69.8% (54.8%-79.9%) |
| Baseline Seropositive^{a} | 21 | 0.2 | 37 | 0.6 | 72.0 (52.2%-83.6%) |
| Baseline Seronegative^{a} | 17 | 0.3 | 25 | 1 | 67.8 (40.3%-82.6%) |
| DENV-2 | 8 | <0.1 | 80 | 0.9 | 95.1% (89.9%-97.6%) |
| Baseline Seropositive^{a} | 7 | <0.1 | 54 | 0.9 | 93.7 (86.1%-97.1%) |
| Baseline Seronegative^{a} | 1 | <0.1 | 26 | 1.1 | 98.1 (85.8%-99.7%) |

Hospitalized VCD cases

| | | | | | |
|---|---|---|---|---|---|
| Overall | 13 | <0.1 | 66 | 0.8 | 90.4% (82.6%-94.7%) |
| Baseline Seropositive^{a} | 8 | <0.1 | 45 | 0.7 | 91.4% (81.7%-95.9%) |
| Baseline Seronegative^{a} | 5 | 0.1 | 21 | 0.9 | 88.1% (68.5%-95.5%) |

VCD, virologically-confirmed dengue;
^{a}Seronegative for all serotypes; baseline seropositive defined as reciprocal neutralizing antibody titer ≥10 to one or more serotypes.

TABLE 7c
Distribution of cases contributing to secondary endpoint by safety set (first
vaccination until end of Part 2, i.e. 21 months after first vaccination)

| | | | | |
|---|---|---|---|---|
| TDV | TDV | Placebo | Placebo | Vaccine Efficacy |
| Dengue Cases | Incidence Density | Dengue Cases | Incidence Density | (95% CI) |

VCD cases

| | | | | | |
|---|---|---|---|---|---|
| Overall | | | | | 75.3% (69.5%-80.0%) |
| Overall in between^{a} | | | | | 81.0% (64.1%-90.0%) |
| Baseline Seropositive^{b} | 89 | 0.5 | 187 | 2.3 | 77.2% (70.6%-82.3%) |
| Baseline Seronegative^{b} | 42 | 0.7 | 70 | 2.3 | 70.6% (56.9%-79.9%) |
| DENV-1 | 41 | 0.2 | 78 | 0.7 | 73.9% (61.9%-82.1%) |
| DENV-2 | 14 | <0.1 | 109 | 1.0 | 93.7% (89.0%-96.4%) |

Hospitalized VCD cases

| | | | | | |
|---|---|---|---|---|---|
| Overall | 17 | <0.1 | 81 | 0.7 | 89.7% (82.6%-93.9%) |

VCD, virologically-confirmed dengue;
aIn between: VCD after first vaccination and before second vaccination.
^{b}Seronegative for all serotypes; baseline seropositive defined as reciprocal neutralizing antibody titer ≥10 to one or more serotypes.

TABLE 7d
Dengvaxia ® VCD (first vaccination until 25
months after first vaccination (i.e. 13 month after
third vaccination), ITT from CYD15, 9 to 16 years of age)^{a}

| | Vaccine Efficacy (95% CI) |
|---|---|
| Overall VCD | 64.7% (58.7%-69.8%) |
| Baseline Seropositive^{b} | 83.7% (62.2%-93.7%) |
| Baseline Seronegative^{b} | 43.2% (−61.5%-80.0%) |
| DENV-1 | 58.8% (40.2%-65.9%) |
| DENV-2 | 50.2% (31.8%-63.6%) |
| Overall Hospitalized VCD | 80.3% (64.7%-89.5%) |

^{a}Luis Villar et al. Efficacy of a tetravalent dengue vaccine in Children in Latin America: N Engl J of Med 2015 Vol. 372 No2, 113-123

Clinical signs and symptoms of virologically-confirmed dengue cases during Part 1 study period in safety set data are shown in Table 8.

TABLE 8
Clinical signs and symptoms of virologically-confirmed dengue
cases during Part 1 study period (safety set data)

| | TDV | Placebo | Relative |
|---|---|---|---|
| | (N = 13,380) | (N = 6,687) | Risk |
| Number of VCD Cases | 78 | 200 | — |
| Median Duration of Febrile Illness (days; 95% CI)^{a} | 6.0 (5.7-7.4) | 6.0 (5.9-6.8) | — |
| Median Duration of Fever (days; 95% CI) | 4.0 (3.9-4.6) | 5.0 (4.5-5.0) | — |
| Number of Hospitalized VCD Cases | 9 | 67 | — |
| Median Duration of Hospitalization (days; 95% CI) | 5.0 (2.8-5.4) | 5.0 (4.6-5.4) | — |
| Evidence of Bleeding (%, n/N) | 3.8% (3/78) | 3.5% (7/200) | 1.10 |
| Plasma Leakage (%, n/N) | 2.6% (2/78) | 6.5% (13/200) | 0.39 |
| Plasma Leakage-Pleural Effusion (%, n/N) | 1.3% (1/78) | 1.5% (3/200) | — |
| Plasma Leakage-Ascites (%, n/N) | 1.3% (1/78) | 3.0% (6/200) | — |
| Plasma Leakage-Radiological Signs (%, n/N) | 40.0% (2/5) | 19.6% (10/51) | — |
| Plasma Leakage-Hematocrit Increase ≥20% (%, n/N)^{b} | 3.8% (2/53) | 9.5% (13/137) | — |
| Platelet Count ≤100 × 10⁹ (%, n/N)^{c} | 6.4% (5/78) | 22.0% (44/200) | 0.29 |
| Platelet Count ≤50 × 10⁹ (%, n/N)^{c} | 3.8% (3/78) | 11.0% (22/200) | 0.35 |
| ALT or AST ≥1000 U/L (%, n/N)^{c} | 0% (0/78) | 0% (0/200) | — |

VCD, virologically-confirmed dengue;
ALT, alanine aminotransferase;
AST, aspartate aminotransferase
^{a}Duration of febrile illness defined as start date of earliest symptom to end date of latest symptom plus one day (symptoms considered include fever and any general symptoms).
^{b}Hematocrit increase defined as maximum hematocrit between Day 3 and Day 7 inclusive, from onset of fever ≥20% increase over minimum hematocrit before Day 3 or after Day 7 from onset of fever.
^{c}For platelet, ALT, and AST data, assessments within 14 days of onset of febrile illness have been considered.
N refers to number of VCD cases with available data for the specific parameter

### e) Immunogenicity

The highest geometric mean titers (GMTs) were observed against DENV-2 regardless of baseline serostatus (Table 10). A very high tetravalent seropositivity rate (99.5%) in baseline seronegatives one month after the second dose (Tables 9 and 10) was observed.

Seropositivity rate (% of seropositive subjects) for each of the four dengue serotypes is determined at prevaccination on Day 1 (Month 0), post-first vaccination on Day 30 (Month 1), prevaccination on Day 90 (Month 3), post-second vaccination on Day 120 (Month 4), Day 270 (Month 9), Day 450 (Month 15), and then annually. Seropositivity rates (% participants, 95% CI) by dengue serotype per protocol set for immunogenicity data for Day 0, Day 30, Day 90, Day 120, and Day 270 are shown in Table 9.

Seropositivity rates (% participants, 95% CI) by dengue serotype against three or more serotypes (trivalent) and against all four serotypes (tetravalent) per protocol set for immunogenicity data for Day 0, Day 30, Day 90, Day 120, and Day 270 are shown in Table 9. The tetravalent seropositivity rates were high (>91%) in baseline seronegatives six months after second dose.

TABLE 9
Seropositivity rates (% participants, 95% CI) by dengue
serotype (per protocol set for immunogenicity data)

| BASELINE SEROPOSITIVE | | BASELINE SERONEGATIVE | |
|---|---|---|---|
| TDV | Placebo | TDV | Placebo |
| N = 1,816 | N = 902 | N = 702 | N = 345 |

DENV-1

| | | | |
|---|---|---|---|
| 89.1 (87.6-90.5) | 90.6 (88.5-92.4) | 0 (0-0.5) | 0 (0-1.1) |
| 99.5 (99.1-99.8) | 88.6 (86.3-90.7) | 94.1 (92.0-95.8) | 4.9 (2.8-7.8) |
| 99.3 (98.8-99.6) | 90.2 (88.1-92.1) | 91.6 (89.3-93.5) | 6.1 (3.8-9.2) |
| >99.9 (99.7-100) | 90.3 (88.1-92.3) | 99.5 (98.6-99.9) | 8.3 (5.5-11.9) |
| 99.6 (99.1-99.8) | 89.8 (87.5-91.8) | 95.1 (93.0-96.6) | 9.0 (6.0-12.8) |

DENV-2

| | | | |
|---|---|---|---|
| 96.5 (95.6-97.3) | 97.2 (95.9-98.2) | 0 (0-0.5) | 0 (0-1.1) |
| 99.9 (99.6-100) | 93.3 (91.4-94.9) | 98.6 (97.4-99.4) | 10.7 (7.5-14.5) |
| >99.9 (99.7-100) | 94.0 (92.2-95.5) | 99.0 (98.0-99.6) | 12.2 (8.9-16.1) |
| 99.9 (99.6-100) | 93.6 (91.7-95.2) | 100 (99.4-100) | 14.7 (11.0-19.1) |
| 100 (99.8-100) | 94.6 (92.8-96.1) | 100 (99.4-100) | 18.3 (14.1-23.2) |

DENV-3

| | | | |
|---|---|---|---|
| 88.1 (86.5-89.6) | 88.0 (85.7-90.1) | 0 (0-0.5) | 0 (0-1.1) |
| 99.8 (99.4-99.9) | 87.6 (85.1-89.7) | 96.1 (94.3-97.4) | 4.0 (2.1-6.7) |
| 99.5 (99.1-99.8) | 87.3 (84.9-89.4) | 94.4 (92.5-96.0) | 2.0 (0.8-4.1) |
| 99.8 (99.5-100) | 87.9 (85.5-90.1) | 100 (99.4-100) | 5.1 (2.9-8.2) |
| 99.7 (99.4-99.9) | 87.1 (84.6-89.4) | 96.4 (94.6-97.7) | 7.7 (4.9-11.3) |

DENV-4

| | | | |
|---|---|---|---|
| 88.1 (86.5-89.6) | 87.4 (85.0-89.5) | 0 (0-0.5) | 0 (0-1.1) |
| 99.6 (99.2-99.9) | 86.6 (84.1-88.8) | 90.5 (88.0-92.6) | 1.8 (0.7-3.9) |
| 99.3 (98.8-99.7) | 86.9 (84.5-89.0) | 92.0 (89.8-93.9) | 2.9 (1.4-5.3) |
| >99.9 (99.7-100) | 88.3 (85.9-90.4) | 99.8 (99.1-100) | 4.8 (2.7-7.8) |
| 99.7 (99.3-99.9) | 87.6 (85.1-89.9) | 97.0 (95.4-98.2) | 6.3 (3.9-9.7) |

Three or More Serotypes

| | | | |
|---|---|---|---|
| 87.5 (85.9-89.0) | 87.3 (84.9-89.4) | 0 (0-0.5) | 0 (0-1.1) |
| 99.8 (99.5-100) | 87.2 (84.7-89.4) | 96.5 (94.8-97.8) | 1.2 (0.3-3.1) |
| 99.7 (99.3-99.9) | 87.7 (85.3-89.7) | 94.9 (93.0-96.4) | 1.7 (0.6-3.7) |
| 99.9 (99.6-100) | 88.4 (86.0-90.5) | 99.8 (99.1-100) | 4.2 (2.2-7.0) |
| 99.7 (99.4-99.9) | 87.3 (84.7-89.5) | 97.5 (96.0-98.6) | 5.7 (3.3-8.9) |

All Four Serotypes

| | | | |
|---|---|---|---|
| 83.5 (81.7-85.2) | 83.5 (80.9-85.8) | 0 (0-0.5) | 0 (0-1.1) |
| 99.1 (98.5-99.5) | 82.9 (80.2-85.4) | 85.3 (82.4-87.9) | 0.9 (0.2-2.6) |
| 98.6 (97.9-99.1) | 83.6 (81.0-86.0) | 84.3 (81.4-86.9) | 1.4 (0.5-3.3) |
| 99.8 (99.5-100) | 85.2 (82.6-87.6) | 99.5 (98.6-99.9) | 3.5 (1.8-6.2) |
| 99.2 (98.7-99.6) | 84.6 (81.9-87.0) | 91.3 (88.7-93.4) | 5.3 (3.1-8.5) |

Seropositivity rates (% participants, 95% CI) by dengue serotype (per protocol set for immunogenicity data; seropositive defined as a reciprocal neutralizing antibody titer ≥10; baseline seronegative defined as seronegative to all serotype; baseline seropositive defined as seropositive to one or more serotypes;
N refers to number of participants in the analysis set; number of participants evaluated at each time point may vary)

Geometric mean titers (GMTs) of neutralizing antibodies (microneutralization test [MNT]) for each dengue serotype are determined at pre-vaccination on Day 1 (Month 0), post-first vaccination on Day 30 (Month 1), pre-vaccination on Day 90 (Month 3), post-second vaccination on Day 120 (Month 4), Day 270 (Month 9), Day 450 (Month 15), and then annually. Geometric mean titers (95% CI) by dengue serotype per protocol set for immunogenicity data for Day 0, Day 30, Day 90, Day 120, and Day 270 are shown in Table 10.

TABLE 10
Geometric mean titers (95% CI) by dengue serotype
(per protocol set for immunogenicity data)

| BASELINE SEROPOSITIVE | | BASELINE SERONEGATIVE | |
|---|---|---|---|
| TDV | Placebo | TDV | Placebo |
| N = 1,816 | N-902 | N = 702 | N = 345 |

DENV-1

| | | | | |
|---|---|---|---|---|
| Day 1 | 410 (365-461) | 445 (377-524) | 5.0 (5.0-5.0) | 5.0 (5.0-5.0) |
| Day 30 | 2,404 (2,204-2,622) | 430 (361-512) | 118 (106-131) | 5.8 (5.3-6.3) |
| Day 90 | 1,945 (1,791-2,112) | 410 (349-481) | 91 (82-102) | 5.9 (5.4-6.3) |
| Day 120 | 2,115 (1,957-2,286) | 451 (381-534) | 184 (169-201) | 6.3 (5.7-7.0) |
| Day 270 | 1,447 (1,329-1,574) | 415 (350-492) | 87 (79-97) | 6.3 (5.7-6.9) |

DENV-2

| | | | | |
|---|---|---|---|---|
| Day 1 | 745 (674-825) | 802 (697-924) | 5.0 (5.0-5.0) | 5.0 (5.0-5.0) |
| Day 30 | 6,697 (6,301-7,117) | 744 (635-870) | 6,277 (5,648-6,977) | 6.6 (6.0-7.3) |
| Day 90 | 4,826 (4,571-5,096) | 729 (629-845) | 1,682 (1,544-1,834) | 7.0 (6.3-7.9) |
| Day 120 | 4,897 (4,646-5,163) | 766 (655-896) | 1,730 (1,614-1,855) | 7.7 (6.7-8.8) |
| Day 270 | 3,692 (3,496-3,898) | 776 (665-906) | 929 (856-1,010) | 8.7 (7.4-10.2) |

DENV-3

| | | | | |
|---|---|---|---|---|
| Day 1 | 357 (321-398) | 356 (305-415) | 5.0 (5.0-5.0) | 5.0 (5.0-5.0) |
| Day 30 | 2,255 (2,094-2,428) | 349 (298-409) | 194 (173-218) | 5.5 (5.2-5.9) |
| Day 90 | 1,563 (1,453-1,682) | 321 (277-374) | 94 (85-104) | 5.5 (5.1-5.9) |
| Day 120 | 1,761 (1,646-1,885) | 353 (301-414) | 228 (212-246) | 6.0 (5.4-6.6) |
| Day 270 | 1,089 (1,009-1,175) | 307 (261-360) | 72 (66-78) | 6.3 (5.7-7.0) |

DENV-4

| | | | | |
|---|---|---|---|---|
| Day 1 | 218 (198-241) | 234 (203-270) | 5.0 (5.0-5.0) | 5.0 (5.0-5.0) |
| Day 30 | 1,303 (1,221-1,391) | 222 (191-258) | 111 (98-125) | 5.4 (5.0-5.7) |
| Day 90 | 1,002 (940-1,069) | 215 (187-248) | 63 (57-70) | 5.5 (5.1-5.9) |
| Day 120 | 1,129 (1,066-1,196) | 241 (208-280) | 144 (134-155) | 5.8 (5.3-6.4) |
| Day 270 | 778 (730-830) | 229 (197-266) | 64 (59-70) | 6.2 (5.6-6.9) |

Vaccine viremia is assessed by three PCRs: dengue detection RT-PCR, vaccine screening PCR and TDV sequencing in subjects with febrile illness within 30 days after each vaccination.

### f) Safety

Rates of serious adverse events (SAEs) were similar in the vaccine and placebo groups (3.1% and 3.8% of participants, respectively; Table 11). One vaccinee and four placebo recipients experienced SAEs considered to be related to receiving blinded investigational product by the investigator (two experienced hypersensitivity, two were diagnosed with dengue, and one with DHF). There were five deaths during Part 1, and all were considered unrelated to the investigational product or study procedures. Total rates of unsolicited AEs were similar between the vaccine and placebo groups. The most commonly (≥1% of vaccine-recipients) reported unsolicited AEs within four weeks of any dose by preferred term were pyrexia (vaccine group 1.5%; placebo 1.4%), nasopharyngitis (vaccine 2.7%; placebo 3.0%), upper respiratory tract infection (vaccine 2.6%; placebo 2.9%), and viral infection (vaccine 1.1%; placebo 0.9%). Solicited local reactions were reported more frequently in the vaccine group.

TABLE 11a
Overview of safety data. Subjects with at least one adverse event after any vaccine
dose. Data presented as number of events (percentage of subjects; number [n]
of subjects/total [N] subjects) unless otherwise stated (safety set data)

| | TDV | Placebo |
|---|---|---|
| Safety Set | N = 13,380 | N = 6,687 |
| SAEs | 3.1% (409/13,380) | 3.8% (255/6,687) |
| Non-IP-Related^{a} SAEs | 3.0% (408/13,380) | 3.8% (251/6,687) |
| IP-Related^{a} SAEs | <0.1% (1/13,380) | <0.1% (4/6,687) |
| SAEs Leading to IP Withdrawal and/or Trial Discontinuation | 0.1% (18/13,380) | 0.1% (8/6,687) |
| Deaths | <0.1% (4/13,380) | <0.1% (1/6,687) |
| IP-Related Deaths | 0% (0/13,380) | 0% (0/6,687) |
| Safety Subset | N = 2,663 | N = 1,329 |
| Unsolicited AEs Occurring Within 4 Weeks of Any Dose | 18.4% (490/2,663) | 18.8% (250/1,329) |
| IP-Related^{a} Unsolicited AEs Occurring Within 4 Weeks of Any | 1.0% (27/2,663) | 1.6% (21/1,329) |
| Dose | | |
| Solicited Systemic AEs Occurring Within 2 Weeks of Any Dose^{b} | 42.0% (1,107/2,635) | 38.0% (501/1,317) |
| IP-Related^{a} Solicited Systemic AEs Occurring Within 2 Weeks of | 31.2% (821/2,635) | 28.2% (371/1,317) |
| Any Dose | | |
| Solicited Local Reactions Occurring Within 1 Week of Any Dose^{c} | 36.7% (967/2,633) | 25.7% (338/1,317) |

AE, adverse event;
SAE, serious adverse event;
IP, investigational product/TDV
^{a}IP-related, defined as related to the investigational product as assessed by investigator
^{b}only participants with diary data available were evaluated
^{c}all injection site (solicited local) reactions considered to be IP-related

TABLE 11b
Number of participants (%) with serious adverse events after any vaccination during
Part 1 by MedDRA (Medical Dictionary for Regulatory Activities)System Organ Class
in the order of decreasing frequency (safety set data presented by TDV and placebo
group for events that occurred in >3 participants due to risk of unblinding).

| | TDV | Placebo | Total* |
|---|---|---|---|
| MedDRA System Organ Class | N = 13,380 | N = 6,687 | N = 20,071 |
| Any Serious Adverse Events | 409 (3.1) | 255 (3.8) | 664 (3.3) |
| Infections and infestations | 235 (1.8) | 179 (2.7) | 414 (2.1) |
| Injury, poisoning and procedural complications | 87 (0.7) | 37 (0.6) | 124 (0.6) |
| Gastrointestinal disorders | 23 (0.2) | 9 (0.1) | 32 (0.2) |
| Nervous system disorders | 14 (0.1) | 6 (<0.1) | 20 (<0.1) |
| Respiratory, thoracic and mediastinal disorders | 14 (0.1) | 6 (<0.1) | 20 (<0.1) |
| Renal and urinary disorders | 15 (0.1) | 3 (<0.1) | 18 (<0.1) |
| Blood and lymphatic system disorders | 8 (<0.1) | 2 (<0.1) | 10 (<0.1) |
| Pregnancy, puerperium and perinatal conditions | 8 (<0.1) | 2 (<0.1) | 10 (<0.1) |
| Skin and subcutaneous tissue disorders | 7 (<0.1) | 3 (<0.1) | 10 (<0.1) |
| Psychiatric disorders | 7 (<0.1) | 2 (<0.1) | 9 (<0.1) |
| General disorders and administration site conditions | 5 (<0.1) | 3 (<0.1) | 8 (<0.1) |
| Immune system disorders | 3 (<0.1) | 4 (<0.1) | 7 (<0.1) |
| Metabolism and nutrition disorders | 6 (<0.1) | 1 (<0.1) | 7 (<0.1) |
| Musculoskeletal and connective tissue | 1 (<0.1) | 5 (<0.1) | 6 (<0.1) |
| Social circumstances | 2 (<0.1) | 4 (<0.1) | 6 (<0.1) |
| Congenital, familial and genetic disorders | 3 (<0.1) | 2 (<0.1) | 5 (<0.1) |
| Neoplasms benign, malignant and unspecified | 3 (<0.1) | 1 (<0.1) | 4 (<0.1) |
| (including cysts and polyps) | | | |
| Endocrine disorders | — | — | 3 (<0.1) |
| Hepatobiliary disorders | — | — | 3 (<0.1) |
| Reproductive system and breast disorders | — | — | 3 (<0.1) |
| Vascular disorders | — | — | 3 (<0.1) |
| Cardiac disorders | — | — | 2 (<0.1) |
| Eye disorders | — | — | 2 (<0.1) |
| Investigations | — | — | 1 (<0.1) |
| Product issues | — | — | 1 (<0.1) |
| Surgical and medical procedures | — | — | 1 (<0.1) |

*Total column includes participants who received both TAK-003 and placebo due to administration error and are excluded from the TAK-003 and placebo groups.
N in column header refers to number of participants in the safety set

TABLE 11c
Number of participants (%) with unsolicited adverse events
of any severity up to 28-days after any vaccination by MedDRA
System Organ Class in the order of decreasing frequency
(Subset of safety set data presented by TDV and placebo group for
events that occurred in >6 participants due to risk of unblinding).

| | TDV | Placebo | Total |
|---|---|---|---|
| MedDRA System Organ Class | N = 2,663 | N = 1,329 | N = 3,993 |
| Any Unsolicited Adverse Events | 487 (18.3) | 249 (18.7) | 736 (18.4) |
| Infections and infestations | 368 (13.8) | 190 (14.3) | 558 (14.0) |
| Injury, poisoning and procedural | 21 (0.8) | 22 (1.7) | 43 (1.1) |
| complications | | | |
| Gastrointestinal disorders | 33 (1.2) | 9 (0.7) | 42 (1.1) |
| General disorders and administration | 30 (1.1) | 11 (0.8) | 41 (1.0) |
| site conditions | | | |
| Skin and subcutaneous tissue disorders | 27 (1.0) | 7 (0.5) | 34 (0.9) |
| Nervous system disorders | 18 (0.7) | 13 (1.0) | 31 (0.8) |
| Respiratory, thoracic and mediastinal | 18 (0.7) | 10 (0.8) | 28 (0.7) |
| disorders | | | |
| Blood and lymphatic system disorders | 6 (0.2) | 5 (0.4) | 11 (0.3) |
| Musculoskeletal and connective tissue | 6 (0.2) | 5 (0.4) | 11 (0.3) |
| disorders | | | |
| Immune system disorders | — | — | 6 (0.2) |
| Psychiatric disorders | — | — | 3 (<0.1) |
| Reproductive system and breast | — | — | 3 (<0.1) |
| disorders | | | |
| Ear and labyrinth disorders | — | — | 2 (<0.1) |
| Cardiac disorders | — | — | 1 (<0.1) |
| Congenital, familial and genetic | — | — | 1 (<0.1) |
| disorders | | | |
| Eye disorders | — | — | 1 (<0.1) |
| Renal and urinary disorders | — | — | 1 (<0.1) |
| Social circumstances | — | — | 1 (<0.1) |

*Total column includes participants who received both TAK-003 and placebo due to administration error and are excluded from the TAK-003 and placebo groups. N in column header refers to number of participants in the subset of safety set.

TABLE 11d
Summary of diary reported injection site reactions up to 7 days
and systemic adverse events up to 14 days after any vaccination
(Subset of safety set data). Data presented as number of participants
with events/number of evaluated participants in the analysis
set (% of evaluated participants with events).

| | | |
|---|---|---|
| Solicited Events | TDV | Placebo |

Injection site reactions (Age < 6 years)

| | | |
|---|---|---|
| Any | 106/331 (32.0) | 43/169 (25.4) |
| Pain | 104/331 (31.4) | 43/169 (25.4) |
| Erythema | 5/331 (1.5) | 1/169 (0.6) |
| Swelling | 11/331 (3.3) | 2/169 (1.2) |

Injection site reactions (Age ≥ 6 years)

| | | |
|---|---|---|
| Any | 861/2302 (37.4) | 295/1148 (25.7) |
| Pain | 853/2302 (37.1) | 293/1148 (25.5) |
| Erythema | 33/2301 (1.4) | 1/1147 (<0.1) |
| Swelling | 33/2300 (1.4) | 6/1147 (0.5) |

Systemic adverse events (Age < 6 years)

| | | |
|---|---|---|
| Any | 88/331 (26.6) | 35/169 (20.7) |
| Irritability/Fussiness | 41/331 (12.4) | 16/169 (9.5) |
| Drowsiness | 45/331 (13.6) | 21/169 (12.4) |
| Loss of Appetite | 57/331 (17.2) | 22/169 (13.0) |
| Fever (Body temperature >= | 45/327 (13.8) | 23/169 (13.6) |
| 38° C. or 100.4° F.) | | |

Systemic adverse events (Age ≥ 6 years)

| | | |
|---|---|---|
| Any | 941/2302 (40.9) | 422/1147 (36.8) |
| Headache | 715/2302 (31.1) | 326/1147 (28.4) |
| Asthenia | 404/2302 (17.5) | 187/1147 (16.3) |
| Malaise | 510/2301 (22.2) | 226/1147 (19.7) |
| Myalgia | 554/2302 (24.1) | 216/1147 (18.8) |
| Fever (Body temperature >= | 221/2279 (9.7) | 124/1134 (10.9) |
| 38° C. or 100.4° F.) | | |

Additionally, a study to assess the efficacy of a booster dose as a follow-on study of the above-described phase III study, such that booster will be given at 4 to 4.5 years post the second dose in a large enough subset of the above-described phase III study, wherein said subset e.g. includes at least 20 subjects or at least 200 subjects, is possible.

### Example 4: Concomitant Administration of a Hepatitis a Vaccine and a Dengue Vaccine

### 4.1 Introduction, Purpose and Objectives of the Study

A randomized, observer blind, phase 3 trial was conducted in 900 healthy adult subjects aged 18 to 60 years (distributed across the entire age range) in non-endemic countries for dengue disease and hepatitis A virus (HAV) to investigate the immunogenicity and safety of two doses of tetravalent dengue vaccine TDV (subcutaneous (SC) injection), and of the simultaneous on the same day administration of a single dose of HAV vaccine (containing an inactivated HAV; intramuscular (IM) injection) and TDV (SC injection).

A purpose of the study was to assess whether HAV vaccine can be safely administered simultaneously on the same day with TDV as travel vaccines before an international travel of a subject to HAV and dengue (DENV)-endemic countries.

The primary objective of this study was demonstrate non-inferiority (NI) of the immune response to one dose of HAV vaccine simultaneously administered on the same day with one dose TDV on the same day, compared to one dose HAV vaccine simultaneously on the same day administered with placebo on the same day, in DENV/HAV-naïve subjects one month after vaccination.

The secondary objectives of this study were to describe TDV-induced immunogenicity after a single dose of TDV in DENV/HAV-naïve subjects; to describe TDV-induced immunogenicity after two doses of TDV administered 90 days apart in DENV/HAV-naïve subjects; to describe HAV vaccine-induced immunogenicity in DENV/HAV-naïve subjects; and to assess the safety profile after each vaccine injection in all trial groups.

### 4.2 Eligibility Criteria

### Criteria for Inclusion Include:

1. The participant is aged 18 to 60 years, inclusive.
2. Participants who are in good health at the time of entry into the trial as determined by medical history, physical examination (including vital signs) and the clinical judgment of the Investigator.
3. The participant signs and dates a written informed consent form and any required privacy authorization prior to the initiation of any trial procedures, after the nature of the trial has been explained according to local regulatory requirements.
4. Participants who can comply with trial procedures and are available for the duration of follow-up.

### Exclusion Criteria Include:

1. Participants with an elevated oral temperature (>38° C. or 100.4° F.) within 3 days of the intended date of vaccination.
2. Known hypersensitivity or allergy to any of the vaccine components (including excipients of the investigational vaccines or placebo).
3. Participants with behavioral or cognitive impairment or psychiatric disease that, in the opinion of the Investigator, may interfere with the participant's ability to participate in the trial.
4. Participants with any history of progressive or severe neurologic disorder, seizure disorder or neuro-inflammatory disease (e.g., Guillain-Barre syndrome).
5. Participants with history or any illness that, in the opinion of the Investigator, might interfere with the results of the trial or pose additional risk to the participant due to participation in the trial.
6. Known or suspected impairment/alteration of immune function, including:
   1. Chronic use of oral steroids (equivalent to 20 mg/day prednisone≥12 weeks/≥2 mg/kg body weight/day prednisone>2 weeks) within 60 days prior to Day 1 (M0) (use of inhaled, intranasal, or topical corticosteroids is allowed).
   2. Receipt of parenteral steroids (equivalent to 20 mg/day prednisone≥12 weeks/≥2 mg/kg body weight/day prednisone>2 weeks) within 60 days prior to Day 1 (M0).
   3. Administration of immunoglobulins and/or any blood products within the 3 months prior to Day 1 (M0) or planned administration during the trial.
   4. Receipt of immunostimulants within 60 days prior to Day 1 (M0).
   5. Immunosuppressive therapy such as anti-cancer chemotherapy or radiation therapy within
   6 months prior to Day 1 (M0). 6. Human immunodeficiency virus (HIV) infection or HIV-related disease.
   7. Hepatitis A virus (HAV) infection.
   8. Hepatitis C virus infection.
   9. Genetic immunodeficiency.
7. Abnormalities of splenic or thymic function.
8. Participants with a known bleeding diathesis, or any condition that may be associated with a prolonged bleeding time.
9. Participants with any serious chronic or progressive disease according to judgment of the Investigator (e.g., neoplasm, insulin dependent diabetes, cardiac, renal or hepatic disease).
10. Participants with body mass index (BMI) greater than or equal to 35 kg/m\hat{ }2 (=weight in kg/[height in meters2]).
11. Participants participating in any clinical trial with another investigational product 30 days prior to Day 1 (M0) or intent to participate in another clinical trial at any time during the conduct of this trial.
12. Participants who received any other vaccine within 14 days (for inactivated vaccines) or 28 days (for live vaccines) prior to enrollment in this trial or who are planning to receive any vaccine within 28 days of trial vaccine administration.
13. Previous HAV vaccination (in a clinical trial or with an approved product).
14. Participants involved in the trial conduct or their first degree relatives.
15. Participants with history of substance or alcohol abuse within the past 2 years.
16. Female participants who are pregnant or breastfeeding.
17. Females of childbearing potential who are sexually active, and who have not used any of the acceptable contraceptive methods for at least 2 months prior to Day 1 (M0).
   1. Of childbearing potential is defined as status post onset of menarche and not meeting any of the following conditions: bilateral tubal ligation (at least 1 year previously), bilateral oophorectomy (at least 1 year previously) or hysterectomy
   2. Acceptable birth control methods are defined as one or more of the following:
      i. Hormonal contraceptive (such as oral, injection, transdermal patch, implant, cervical ring).
      ii. Barrier method (condom with spermicide or diaphragm with spermicide) each and every time during intercourse.
      iii. Intrauterine device (IUD). iv. Monogamous relationship with vasectomized partner (partner must have been vasectomized for at least 6 months prior to Day 1 [M0]).
      Other contraceptive methods may be considered in agreement with the Sponsor and implemented only after approval of a substantial amendment by the regulatory authorities and by the appropriate ethics committee.
18. Females of childbearing potential who are sexually active, and who refuse to use an acceptable contraceptive method up to 6 weeks after the last dose of trial vaccine (Day 90 [M3]). In addition, they must be advised not to donate ova during this period.
19. Any positive or indeterminate pregnancy test.
20. Previous and planned vaccination (during the trial conduct) against any flaviviruses including dengue, yellow fever (YF), Japanese Encephalitis (JE) viruses or tick-borne encephalitis.
21. Previous participation in any clinical trial of a dengue or other flavivirus (e.g., West Nile [WN] virus) candidate vaccine, except for participants who received placebo in those trials.
22. Participants with a current or previous infection with a flavivirus such as dengue, Zika, YF, JE, WN fever, tick-borne encephalitis or Murray Valley encephalitis and participants with a history of prolonged (≥1 year) habitation in a dengue endemic area.
23. Participants with contraindications, warnings and/or precautions to vaccination with the HAV vaccine as specified within the product information.

### 4.3 Study Design & Vaccinations

Eligible subjects were randomized equally (1:1:1 ratio) to one of the following 3 trial groups (300 subjects per group):
Group 1: HAV vaccine (IM) and placebo (SC), simultaneously on the same day administered on day 1 (month 0); placebo (SC) administered at day 90 (month 3).
Group 2: TDV (SC) and placebo (IM), simultaneously on the same day administered on day 1 (month 0); TDV (SC) administered at day 90 (month 3).
Group 3: TDV (SC) and HAV vaccine (IM), simultaneously on the same day administered on day 1 (month 0); TDV (SC) administered at day 90 (month 3).

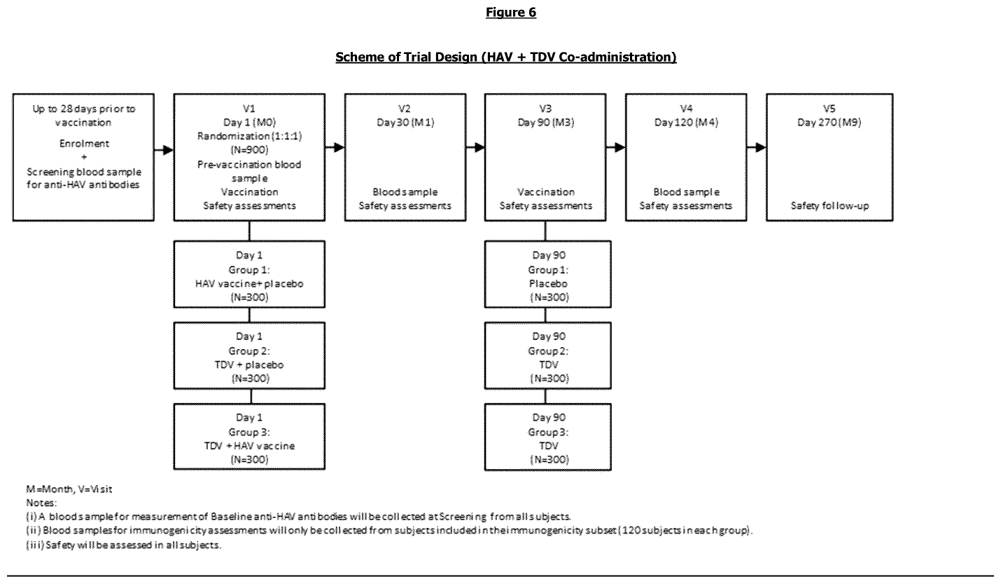

A more detailed scheme of the study design is shown in FIG. 6. Up to 28 days prior to the first vaccination, enrolment was carried out and blood samples were taken for screening anti-HAV antibodies. On day 1, pre-vaccination blood samples were taken. On day 30 (after the first vaccination on day 1) post-vaccination blood samples were taken. On day 120 (after the first vaccination on day 1) another blood sample was taken. Safety follow-up took place on day 270 (after the first vaccination on day 1).

The TDV was prepared as described in Example 1. Each subcutaneous dose of the TDV had a volume of 0.5 ml and the concentration of the four dengue serotypes in the TDV in each dose was 5.1 log₁₀ pfu/0.5 ml, 4.5 log₁₀ pfu/0.5 ml, 5.4 log₁₀ pfu/0.5 ml and 5.9 log₁₀ pfu/0.5 ml of TDV-1, TDV-2, TDV-3 and TDV-4, respectively. Each subcutaneous dose comprises the TDV dispersed in 0.5 ml of an aqueous solution containing Pluronic F127 (10.6 mg/ml), trehalose dihydrate (170 mg/ml) and human serum albumin (1.08 mg/ml).

The HAV vaccine includes an inactivated hepatitis A virus, derived from a hepatitis A virus strain HM-175 (see definitions above), and is commercially available under the tradename HAVRIX® as described above. The intramuscular dose of the HAV vaccine administered to groups 1) and 3) was 1 ml and each 1 ml dose has a viral antigen activity of about 1440 EL.U., wherein the viral antigen is adsorbed on 0.5 mg of aluminum in the form of aluminum hydroxide. The hepatitis A vaccine contains excipients in the form of an amino acid supplement (about 0.3% w/v) and in the form of polysorbate (about 0.05 mg/ml) dissolved in a phosphate-buffered saline solution.

Simultaneously on the same day administered trial vaccines were injected to opposite arms. Normal saline solution for injection (0.9% NaCl) was used as placebo. A blood sample for an anti-HAV antibody test were collected at screening from all subjects to exclude subjects who are positive for anti-HAV antibodies up to 28 days prior to vaccination (see FIG. 6). All subjects were followed-up for 6 months after the second vaccination at day 90 (month 3), so the trial duration was 270 days or 9 months for each subject (not including the screening period). Outside the context of this trial, subjects in Groups 1 and 3 will be offered a HAV vaccine booster dose after the completion of trial procedures at day 270 (month 9).

Dengue neutralizing antibodies (microneutralization test (MNT50)) were measured using blood samples collected at pre-first trial vaccination (day 1 (month 0)), 1 month post first trial vaccination (day 30 (month 1)), and 1 month post second trial vaccination (day 120 (month 4)). Blood samples for the measurement of anti-HAV antibodies (enzyme-linked immunosorbent assay (ELISA)) were collected at pre-first trial vaccination (day 1 (month 0)) and 1 month post first trial vaccination (day 30 (month 1)). 4.4 Primary Endpoint

The primary endpoint included the proportion of HAV/DENV-naive subjects at baseline who are seroprotected against HAV at day 30 (month 1) as measured by enzyme-linked immunosorbent assay (ELISA)). In other words, the primary endpoint includes the seroprotection rates (SPRs). Seroprotection is defined as serum anti-HAV antibody levels≥10 mIU/mL. Immunological naivety to HAV/DENV is defined as anti-HAV antibody levels<10 mIU/mL and reciprocal neutralizing titers for all 4 dengue serotypes<10.

### 4.5 Secondary Endpoints

### A) Secondary Immunogenicity Endpoints

The secondary endpoints included the geometric mean titers of neutralizing antibodies (GMTs) (microneutralization test (MNT50)) for each of the 4 dengue serotypes at day 30 (month 1) and day 120 (month 4) which were determined in HAV/DENV-naive subjects at baseline; the proportion of HAV/DENV-naive subjects at baseline who are seropositive for each of the 4 dengue serotypes at day 30 (month 1) and day 120 (month 4) (seroprotection rate); and geometric mean concentrations (GMC) of anti-HAV antibodies at day 30 (month 1) in subjects HAV/DENV-naive at baseline.

Seropositivity for dengue virus is defined as a reciprocal neutralizing titer>10 for any of the four dengue serotypes within the secondary immunogenicity endpoints.

### B) Secondary Safety Endpoints

Secondary safety endpoints included the frequency and severity of solicited local adverse events (AE) for 7 days after each trial vaccination; the frequency and severity of solicited systemic AEs for 14 days after each trial vaccination; the percentage of subjects with any unsolicited AEs for 28 days after each trial vaccination; the percentage of subjects with serious adverse events (SAE) throughout the trial; and the percentage of subjects with medically attended adverse events (MAAE) throughout the trial.

### 4.6 Analysis Sets of the Study

Table 12 below displays each analysis set of the present study. In total, 1199 subjects belonging to the group “all screened” included all subjects who signed the informed consent, regardless of whether the subjects were screen failures. After initial screening, 900 subjects were included into the “randomized set” which includes all randomized subjects, regardless of whether any dose of the IPs was received. The safety set, consisting of 897 subjects, includes all randomized subjects who received≥1 dose of the IPs. The immunogenicity subjects included a total of 359 subjects and is subdivided into the following four subsets. The HAV-full analysis set (HAV-FAS) includes all randomized subjects in the immunogenicity subset who received>1 dose of the trial vaccine with available day 1 and day 30 anti-HAV antibody measurements. The HAV-per-protocol set (HAV-PPS) includes all HAV- and DENV-naïve subjects from the HAV-FAS who have no major protocol violations. The TDV-full analysis set (TDV-FAS) includes all randomized subjects in the immunogenicity subset who received>1 dose of trial vaccine and with available day 1 and ≥1 post-dose measurements. The TDV-per-protocol set (TDV-PPS), consisting of 197 subjects, includes all HAV- and DENV-naïve subjects from the TDV-FAS who have no major protocol violations.

TABLE 12
Analysis sets of the study.

| | HAV/Pbo | TDV/Pbo | HAV/TDV | Total |
|---|---|---|---|---|
| All Screened¹ | NA | NA | NA | 1199 |
| Randomized Set² | 300 | 300 | 300 | 900 |
| Safety Set (SS)³ | 299 | 300 | 298 | 897 |
| Immunogenicity Subset | 119 | 120 | 120 | 359 |
| HAV Full Analysis Set | 115 | 117 | 114 | 346 |
| (HAV-FAS)⁴ | | | | |
| HAV Per-Protocol Set | 75 | 71 | 81 | 227 |
| (HAV-PPS)⁵ | | | | |
| TDV-FAS⁶ | 116 | 117 | 115 | 348 |
| TDV-PPS⁷ | 67 | 63 | 67 | 197 |
| Subjects analyzed for | 69/115 | NA | 79/114 | 148/227 |
| primary non-inferiority | | | | |
| objective⁸ | | | | |

¹All Screened: All subjects who signed the informed consent, regardless of whether subjects were screen failures
²Randomized Set: All randomized subjects, regardless of whether any dose of the trial vaccines was received.
³Safety Set: All randomized subjects who received ≥1 dose of trial vaccines.
⁴HAV-FAS: All randomized subjects in the immunogenicity subset who received ≥1 dose of trial vaccine, with available Day 1 and Day 30 HAV measurements.
⁵HAV-PPS: All HAV & DENV-naïve subjects from the HAV-FAS who have no major protocol violations.
⁶TDV-FAS: All randomized subjects in the immunogenicity subset who received ≥1 dose of trial vaccine and available Day 1 and ≥1 post-dose measurement.
⁷TDV-PPS: All HAV & DENV-naïve subjects from the TDV-FAS who have no major protocol violations.
⁸Subject excluded from TDV-PPS but included into analysis for primary non-inferiority objective had their Day 30 measurement outside the protocol defined visit window.

From a total number of 359 subjects in the immunogenicity subset (including all subjects which received≥1 vaccination), 13 subjects have been excluded from HAV-FAS because of not providing a valid baseline and post-dosing measurement (on day 30) for HAV. Furthermore, HAV-PPS includes all HAV- and DENV-naïve subjects of HAV-FAS who had no major protocol violations. The subjects analyzed for primary non-inferiority objective are based on the HAV-PPS subjects of the HAV/Pbo and HAV/TDV group, wherein based on HAV-PPS of these two groups (HAV/Pbo and HAV//TDV), some subjects were not included in the 10 30 days analysis (6 subjects of the HAV/Pbo group and 2 Subjects of the HAV/TDV group), since these subjects had their day 30 measurement outside the visit window defined in the protocol. Therefore, a total of 69 subjects was included into the TDV/Pbo group and a total of 79 subjects was included in the HAV/TDV group for analyzing the primary non-inferiority objective.

From a total number of 359 subjects in the immunogenicity subset (including all subjects which received>1 vaccination), a total of 11 subjects have been excluded from TDV-FAS, because they did not provide a valid baseline and at least one post-dosing measurement (day 30 and/or day 120) for TDV. Furthermore, a total number of 151 subjects had been excluded from the TDV-PPS for not being HAV & DENV-naïve at baseline or for not receiving both vaccinations 1 and 2 or if vaccination 2 (usually on day 90) is outside the window-15/±25 days or if major protocol violations occur.

### 4.7 HAV Baseline Serostatus and Demographic & Baseline Characteristics

The safety set evaluated for baseline HAV antibody levels included a total of 362 subjects of which 27.3% were HAV seropositive at baseline (see Table 13).

HAS-FAS included a total of 346 subjects evaluated for baseline HAV antibody levels (see Table 13). HAV-naivety was defined as anti-HAV antibody (ab) level of <10 mIU/ml. However, the ELISA used for serological analysis could not be validated below levels of 12.5 mIU/ml. The qualitative screening test had a specification that effectively amounted to a lower limit of quantification of 70 mIU/ml. In view of these criteria, 72.5% of the subjects of said HAS-FAS evaluated for baseline HAV antibody levels were HAV naive at baseline (see Table 13).

TABLE 13
HAV baseline serostatus in the safety set and in the HAV-FAS

| | HAV/Pbo | | TDV/Pbo | | HAV/TDV | | Total | |
|---|---|---|---|---|---|---|---|---|
| Safety set evaluated for | 119 | | 121 | | 122 | | 362 | |
| baseline HAV antibody levels | | | | | | | | |
| HAV seropositive | 31 | (26.1%) | 38 | (31.4%) | 30 | (24.6%) | 99 | (27.3%) |
| at baseline | | | | | | | | |
| HAV-FAS Evaluated For | 115 | (100%) | 117 | (100%) | 114 | (100%) | 346 | (100%) |
| Baseline HAV antibody levels | | | | | | | | |
| HAV seronegative at | 86 | (74.8%) | 79 | (67.5%) | 86 | (74.4%) | 251 | (72.5%) |
| baseline <12.5 mIU/ml (a) | | | | | | | | |
| HAV seropositive at baseline (b) | 29 | (25.2%) | 38 | (32.5%) | 28 | (24.6%) | 95 | (27.5%) |

| | | | | |
|---|---|---|---|---|
| baseline 12.5-70 mIU/ml | 18 | 23 | 13 | 54 |
| baseline 70-1000 mIU/ml | 11 | 14 | 14 | 39 |
| baseline >1000 | 0 | 1 | 1 | 2 |

(a) HAV-naivety was defined as anti-HAV ab level of <10 mIU/ml; The ELISA used for serological analysis could not be validated below levels of 12.5 mIU/ml.
(b) The qualitative screening test had a specification that effectively amounted to a lower limit of quantification of 70 mIU/ml

The HAV-PPS includes a total number of 227 subjects (see Table 14). The mean age of the total number of subjects of the HAV-PPS, which are DENV- and HAV-naïve was 34.8. 31.3% of the total number of subjects of the HAV-PPS were female (see Table 14). In total, 97.8% of the total subjects of the HAV-PPS were of an ethnicity which is NOT Hispanic or Latino, and, in particular, 89.9% of the HAV-PPS participants were of race “white European”, especially in order to reflect the situation of travelers of HAV- and dengue non-endemic countries (see Table 14).

The safety set includes a total number of 897 subjects (see Table 14). The mean age of the total number of subjects of the safety set was 35.4 years of which 31.3% were female. In total, 97.7% of the total subjects of the HAV-PPS were of an ethnicity which is NOT Hispanic or Latino, and, in particular, 87.1% of the HAV-PPS participants were of race “white European”, especially in order to reflect the situation of travelers of HAV- and dengue non-endemic countries (see Table 14).

TABLE 14
Demographic and baseline characteristics (HAV-PPS and Safety Set)

| | | HAV/Pbo | TDV/Pbo | HAV/TDV | Total |
|---|---|---|---|---|---|
| HAV-PPS (DENV/HAV-naïve) | N | 75 | 71 | 81 | 227 |
| Age Years | Mean (SD) | 34.3 (11.68) | 35.5 (11.24) | 34.8 (11.70) | 34.8 (11.51) |
| Gender | Female n (%) | 24 (32.0%) | 30 (42.3%) | 17 (21.0%) | 71 (31.3%) |
| Ethnicity NOT Hispanic or | n (%) | 72 (96.0%) | 71 (100.0%) | 79 (97.5%) | 222 (97.8%) |
| Latino | | | | | |
| Race White European | n (%) | 64 (85.3%) | 64 (90.1%) | 76 (93.8%) | 204 (89.9%) |
| Safety set | N | 299 | 300 | 298 | 897 |
| Age Years | Mean (SD) | 34.7 (12.04) | 36.0 (11.88) | 35.5 (11.96%) | 35.4 (11.96) |
| Gender | Female n (%) | 107 (35.8%) | 120 (40.0%) | 90 (30.2%) | 317 (35.3%) |
| Ethnicity NOT Hispanic or | n (%) | 289 (96.7%) | 293 (97.7%) | 294 (98.7%) | 876 (97.7%) |
| Latino | | | | | |
| Race White European | n (%) | 255 (85.3%) | 265 (88.3%) | 261 (87.6%) | 781 (87.1%) |

4.8 Study Results
a) Primary endpoint and sensitivity analyses

The present study was successful in meeting the primary objective of non-inferiority for the simultaneous on the same day administration of HAV and TDV. Table 15 displays the seroprotection rates (SPRs) of groups 1 (received HAV/Pbo) and 3 (received HAV/TDV) on day 30 after the first vaccination (on day 1), the SPR differences between the HAV/Pbo group and the HAV/TDV group on day 30, and the confidence intervals (CIs) of these SPR differences for HAV and DENV-baseline naïve subjects. These values (SPRs, SPR differences; CIs) were used for the primary endpoint evaluation of the study. Table 15 further shows these values for the results of three sensitivity analyses (also used for non-inferiority assessments), wherein the subjects had different, i.e. mixed, HAV/TDV serostatuses at baseline. Non-inferiority between the hepatitis A vaccine and the tetravalent dengue vaccine, when simultaneously on the same day administered, in the present study is concluded, if the seroprotection rate (SPR) difference between group 1 (received HAV and placebo on the same day 1) and group 3 (received HAV and TDV on the same day 1) has an upper bound of a two-sided 95% confidence interval, calculated using the Newcombe score method, which is lower than the 10% non-inferiority margin. This criterion is fulfilled for each of the groups in Table 15. In the primary endpoint group, the upper bound of the 95% CI of the SPR difference is 4.31% which is less than the non-inferiority margin of 10% (see second line from above in Table 15).

As mentioned above, sensitivity analyses 1 to 3 were used to evaluate populations that included subjects who were seropositive for dengue and/or for hepatitis A at baseline, in particular reflecting “real life” travel clinic settings in non-endemic countries in which subjects, i.e. travelers who plan to go to dengue and HAV endemic countries, are not always aware of their HAV and/or dengue serostatus before requesting pre-travel vaccinations.

The object of non-inferiority of the simultaneous on the same day administration was also met in sensitivity analyses 1 to 3 (upper bounds of the 95% CI of the SPR differences: 3.21%; 2.93%; and 2.55% which are each less than the non-inferiority margin of 10%). Furthermore, the SPRs of the HAV/TDV groups (98.8%; 99.0%; 99.1%, respectively, see Table 15) were respectively higher than the SPRs of the HAV/Pbo group (96.2%; 96.9%; 97.2%, respectively, see Table 15).

Therefore, due to the non-inferiority of the simultaneous on the same day administration of the HAV vaccine and TDV to subjects with mixed baseline serostatus (and baseline naivety) with respect to HAV and all dengue serotypes, there is no need for determining or knowing the subject's baseline serostatus with respect to each of the two diseases, prior to simultaneously on the same day administering the HAV vaccine and TDV.

TABLE 15
Primary endpoint: Non-inferiority (NI) assessments & sensitivity analysis

| | | HAV/Pbo | HAV/TDV | | 95% CI¹ |
|---|---|---|---|---|---|
| | | (Group 1) | (Group3) | | (of SPR |
| Analysis | Analysis Set | SPR % (n/n) | SPR % (n/n) | SPR difference | difference) |
| Primary | HAV PPS-includes | 97.1% | 98.7% | −1.63 | (−8.78, 4.31) |
| endpoint | baseline HAV− and DENV− | (67/69) | (78/79) | | |
| | subjects | | | | |
| Sensitivity | HAV PPS-includes | 96.2% | 98.8% | −2.61 | (−9.46, 3.21) |
| Analysis 1 | baseline HAV−, DENV−, | (76/79) | (83/84) | | |
| | and DENV+ subjects | | | | |
| Sensitivity | HAV PPS-includes | 96.9% | 99.0% | −2.09 | (−7.82, 2.93) |
| Analysis 2 | baseline HAV−; HAV+ | (93/96) | (96/97) | | |
| | (12.5-70 mIU/ml); DENV−; | | | | |
| | DENV+ | | | | |
| Sensitivity | HAV PPS-includes | 97.2% | 99.1% | −1.92 | (−7.14, 2.55) |
| Analysis 3 | baseline HAV−; HAV+; | (103/106) | (109/110) | | |
| | DENV−; DENV+ | | | | |

¹CI = Confidence Interval
Non-inferiority Assessment: Seroprotection Rates (SPRs) for HAV Group 1 (HAV + placebo simultaneous on the same day administration) vs Group 3 (HAV + TDV simultaneous on the same day administration)
Rates difference for primary comparison (Group 1-Group 3) are presented together with 95% CI calculated using Newcombe score method
NI of simultaneous on the same day administration of HAV and TDV to HAV alone will be concluded if the upper bound of the 95% CI is less than NI margin of 10%.

### B) Secondary Immunogenicity Endpoints

Table 16 shows GMTs (with respect to each of the four dengue virus serotypes DENV-1 to DENV-4) when measured on day 1 pre-first vaccination, on day 30 after the first vaccination (on day 1), and on day 120 after the first vaccination of the subjects (on day 1) of the DENV-PPS including the groups receiving HAV/TDV, TDV and placebo, as well as HAV and placebo (Pbo), respectively. Table 16 shows positive trends in favor of the simultaneous on the same day administration group (received HAV/TDV) with respect to all dengue GMTs.

Said positive trend in favor of the simultaneous on the same day administration and the day 30 synergism of the simultaneously on the same day administered vaccines is also confirmed in Table 17.

TABLE 16
Immunogenicity of DENV-PPS: GMTs of DENV MNT50. In particular, DENV GMTs against each serotype with respect to
mean titers of neutralizing antibodies measured by MNT50 for each dengue serotype by trial visit are shown.

| | | DENV-1 | | | DENV-2 | | | DENV-3 | | | DENV-4 | | |
|---|---|---|---|---|---|---|---|---|---|---|---|---|---|
| | | HAV/ | TDV/ | HAV/ | HAV/ | TDV/ | HAV/ | HAV/ | TDV/ | HAV/ | HAV/ | TDV/ | HAV/ |
| | | p | p | TDV | p | p | TDV | p | p | TDV | p | p | TDV |
| Day | n | 67 | 63 | 67 | 67 | 63 | 67 | 67 | 63 | 67 | 67 | 63 | 67 |
| 1 | GMT | 5.0 | 5.0 | 5.0 | 5.0 | 5.0 | 5.0 | 5.0 | 5.0 | 5.0 | 5.0 | 5.0 | 5.0 |
| | (SD¹) | (1.00) | (1.00) | (1.00) | (1.00) | (1.00) | (1.00) | (1.00) | (1.00) | (1.00) | (1.00) | (1.00) | (1.00) |
| Day | n | 62 | 60 | 65 | 62 | 60 | 65 | 62 | 60 | 65 | 62 | 60 | 65 |
| 30 | GMT | 5.0 | 108.2 | 152.5 | 6.0 | 2897.9 | 3960.0 | 5.3 | 95.4 | 140.5 | 5.0 | 74.3 | 142.1 |
| | (SD) | (1.00) | (5.64) | (4.62) | (1.87) | (13.86) | (8.79) | (1.49) | (6.24) | (4.50) | (1.00) | (5.03) | (6.19) |
| Day | n | 50 | 55 | 62 | 50 | 55 | 62 | 50 | 55 | 62 | 50 | 55 | 62 |
| 120 | GMT | 5.0 | 171.3 | 173.7 | 5.7 | 2064.1 | 1764.3 | 5.0 | 83.8 | 92.6 | 5.0 | 56.1 | 81.4 |
| | (SD) | (1.00) | (6.23) | (4.28) | (1.72) | (3.60) | (4.03) | (1.00) | (3.66) | (2.80) | (1.00) | (3.19) | (3.49) |

¹SD = standard deviation.

TABLE 17
Secondary endpoint: HAV Geometric
Mean Concentrations (GMCs)

| | HAV/Pbo | TDV/Pbo | HAV/TDV | Total |
|---|---|---|---|---|
| Baseline | | | | |
| n | 75 | 71 | 81 | 227 |
| Geometric | 6.3 (1.00) | 6.3 (1.00) | 6.3 (1.00) | 6.3 (1.00) |
| Mean (SD¹) | | | | |
| 95% CI² | (NE, NE) | (NE, NE) | (NE, NE) | (NE, NE) |
| Median | 6.3 | 6.3 | 6.3 | 6.3 |
| Min-max | 6, 6 | 6, 6 | 6, 6 | 6, 6 |
| Day 30 | | | | |
| n | 69 | 66 | 79 | 214 |
| Geometric | 80.5 (3.01) | 6.7 (1.27) | 93.0 (2.44) | 39.5 (4.27) |
| Mean (SD) | | | | |
| 95% CI | (61.8, 105.0) | (6.4, 7.2) | (76.1, 113.6) | (32.5, 48.0) |
| Median | 81.5 | 6.3 | 94.5 | 52.4 |
| Min-max | 6, 1044 | 6, 16 | 6, 1859 | 6, 1859 |

¹SD = standard deviation.
²CI = confidence interval.

### C) Secondary Safety Endpoints

The safety set was investigated for solicited adverse events, solicited systemic adverse events, unsolicited adverse events and serious adverse events throughout the study. Tables 18a to 21c show the study results of the safety set with respect to each secondary safety endpoint.

TABLE 18a
Frequency of Solicited Local AEs and Solicited Systemic
AEs after first vaccination-Safety Set

| | HAV/Pbo | TDV/Pbo | HAV/TDV |
|---|---|---|---|
| | (N = 270) | (N = 271) | (N = 257) |
| Solicited Local | 141/289 (48.8) | 152/292 (52.1) | 196/285 (68.8) |
| (within 7 days) | | | |
| Solicited Systemic | 139/289 (48.1) | 132/292 (45.2) | 141/285 (49.5) |
| (within 14 days) | | | |
| Related to IP | 98/289 (33.9) | 106/292 (36.3) | 117/285 (41.1) |
| Not related to IP | 41/289 (14.2) | 26/292 (8.9) | 24/285 (8.4) |

Note:
For solicited AEs, excluding prolonged solicited AEs, percentages are calculated based on number of subjects with non-missing data (n) evaluated in each trial group. Subjects with 1 or more AEs for a particular category of AEs are counted only once using the most related event.

TABLE 18b
Frequency of Solicited Local AEs and Solicited Systemic
AEs after second vaccination-Saftey Set

| | Pbo | TDV | TDV |
|---|---|---|---|
| | (N = 270) | (N = 271) | (N = 257) |
| Solicited Local | 28/255 (11.0) | 100/264 (37.9) | 103/251 (41.0) |
| (within 7 days) | | | |
| Solicited Systemic | 73/254 (28.7) | 82/263 (31.2) | 85/251 (33.9) |
| (within 14 days) | | | |
| Related to IP | 49/254 (19.3) | 60/263 (22.8) | 61/251 (24.3) |
| Not related to IP | 24/254 (9.4) | 22/263 (8.4) | 24/251 (9.6) |

Note:
For solicited AEs, excluding prolonged solicited AEs, percentages are calculated based on number of subjects with non-missing data (n) evaluated in each trial group. Subjects with 1 or more AEs for a particular category of AEs are counted only once using the most related event.

TABLE 18c
Frequency of Solicited Local AEs and Solicited
Systemic AEs after any vaccination-Safety Set

| | HAV/Pbo | TDV/Pbo | HAV/TDV |
|---|---|---|---|
| | (n = 299) | (n = 300) | (n = 298) |
| Solicited Local | 151/289 (52.2) | 175/292 (59.9) | 216/285 (75.8) |
| (within 7 days) | | | |
| Solicited Systemic | 161/289 (55.7) | 167/292 (57.2) | 167/285 (58.6) |
| (within 14 days) | | | |
| Related to IP | 121/289 (41.9) | 133/292 (45.5) | 141/285 (49.5) |
| Not related to IP | 40/289 (13.8) | 34/292 (11.6) | 26/285 (9.1) |

Note:
For solicited AEs, excluding prolonged solicited AEs, percentages are calculated based on number of subjects with non-missing data (n) evaluated in each trial group. Subjects with 1 or more AEs for a particular category of AEs are counted only once using the most related event.

TABLE 19a
Overview of Unsolicited AE up to 28 Days Post-vaccination
(after first vaccination)-Safety Set

| | HAV/Pbo | TDV/Pbo | HAV/TDV |
|---|---|---|---|
| | N = 299 | N = 300 | N = 298 |
| [Any AE], n (%) | 43 (14.4%) | 51 (17.0%) | 56 (18.8%) |
| Mild | 32 (10.7%) | 32 (10.7%) | 43 (14.4%) |
| Moderate | 11 (3.7%) | 18 (6.0%) | 12 (4.0%) |
| Severe | 0 | 1 (0.3%) | 1 (0.3%) |

Notes:
This summary includes all unsolicited AEs with a date of onset within 28 days after each trial vaccination.
N is the number of subjects who received the specific vaccination. Percentages are calculated based on N for corresponding column.
Table shows the number of subjects that reported unsolicited AE
Subjects with 1 or more AEs for a particular category of adverse event are counted only once using the most related/most severe/most serious event.

TABLE 19b
Overview of Unsolicited AE up to 28 Days Post-vaccination
(after second vaccination)-Safety Set

| | Pbo | TDV | TDV |
|---|---|---|---|
| | N = 270 | N = 271 | N = 257 |
| Any AE, n (%) | 39 (14.4%) | 27 (10.0%) | 30 (11.7%) |
| Mild | 17 (6.3%) | 14 (5.2%) | 15 (5.8%) |
| Moderate | 22 (8.1%) | 13 (4.8%) | 13 (5.1%) |
| Severe | 0 | 0 | 2 (0.8%) |

Notes:
This summary includes all unsolicited AEs with a date of onset within 28 days after each trial vaccination.
N is the number of subjects who received the specific vaccination. Percentages are calculated based on N for corresponding column.
Table shows the totals of AE that were experienced by the number of subjects that reported unsolicited AE
Subjects with 1 or more AEs for a particular category of adverse event are counted only once using the most related/most severe/most serious event.

TABLE 20
Most Common Unsolicited AEs Up to 28 Days Post-Vaccination
(after Any Vaccination)*-Safety Set

| | HAV/Pbo | TDV/Pbo | HAV/TDV |
|---|---|---|---|
| AE, n (%) | N = 299 | N = 300 | N = 298 |
| Nasopharyngitis | 9 (3.0%) | 8 (2.7%) | 11 (3.7%) |

*Reported by ≥2.0% of subjects

TABLE 21a
Safety: Overview of Serious
Adverse Events (SAEs) After any dose-Safety Set

| Number of | | | | | | | | |
|---|---|---|---|---|---|---|---|---|
| events, | HAV/Pbo | | TDV/Pbo | | HAV/TDV | | Total | |
| number (%) | (n = 299) | | (n = 300) | | (n = 298) | | (N = 597) | |
| subjects with | Events | | Events | | Events | | Events | |
| SAE | Subjects | | Subjects | | Subjects | | Subjects | |
| SAEs-any | 3 | 2 | 10 | 8 | 10 | 7 | 23 | 17 |
| | | (0.7%) | | (2.7%) | | (2.3%) | | (2.8%) |
| After 1^{st} dose | 0 | 0 | 3 | 3 | 3 | 2 | 6 | 5 |
| up to 2^{nd} dose | | | | (1.0%) | | (0.7%) | | (0.8%) |
| After 2^{nd} dose | 3 | 2 | 7 | 5 | 7 | 5 | 17 | 12 |
| up to trial end | | (0.7%) | | (1.8%) | | (1.9%) | | (2.0%) |
| SAEs-related | 0 | 0 | 0 | 0 | 0 | 0 | 0 | 0 |
| SAE-premature | 0 | 0 | 1 | 1 | 0 | 0 | 1 | 1 |
| vaccine and/or | | | | (0.3%) | | | | (0.2%) |
| trial | | | | | | | | |
| discontinuation | | | | | | | | |
| Deaths | 0 | 0 | 0 | 0 | 0 | 0 | 0 | 0 |

p = Placebo

TABLE 21b
Safety: Serious Adverse Events by System Organ Class and Preferred
Term after first and after second dose-Safety Set

| Number (%) subjects with SAE | | HAV/Pbo | TDV/Pbo | HAV/TDV |
|---|---|---|---|---|
| SOC | PT | (N = 299) | (N = 300) | (N = 298) |
| After 1^{st} dose up to | | 0 | 3 (1.0) | 2 (0.7) |
| 2^{nd} dose-any SAE | | | | |
| Gastrointestinal disorders | Crohn's disease* | 0 | 1 (0.3) | 0 |
| Injury, poisoning and procedural | Intentional | 0 | 0 | 1 (0.3) |
| complications | overdose | | | |
| Neoplasms benign, malignant and | Bladder cancer | 0 | 1 (0.3) | 0 |
| unspecified | stage II | | | |
| Nervous system disorders | Loss of | 0 | 1 (0.3) | 0 |
| | consciousness** | | | |
| Psychiatric disorders | Intentional self-injury | 0 | 0 | 1 (0.3) |
| | | Pbo | TDV | TDV |
| | | (N = 270) | (N = 271) | (N = 257) |
| After 2^{nd} dose up to | | 2 (0.7) | 5 (1.8) | 5 (7.9) |
| end of trial-any SAE | | | | |
| Cardiac disorders | Supraventricular | 0 | 0 | 1 (0.4) |
| | tachycardia | | | |
| Gastrointestinal disorders | Abdominal pain | 0 | 1 (0.4) | 0 |
| | Abdominal | 1 (0.4) | 0 | 0 |
| | strangulated | | | |
| | hernia | | | |
| | Intestinal | 0 | 0 | 1 (0.4) |
| | ischaemia | | | |
| | Mesenteric vein | 0 | 0 | 1 (0.4) |
| | thrombosis | | | |
| | Oesophagitis | 0 | 1 (0.4) | 0 |

*Subject had a history of irritable bowel syndrome
**Occurred >2 months after vaccination

TABLE 21c
Safety: Serious Adverse Events by System Organ Class and Preferred
TermAfter first and after second dose-Safety Set-Continued

| Number (%) | | | | |
|---|---|---|---|---|
| subjects with SAE | | Pbo | TDV | TDV |
| SOC | PT | (N = 270) | (N = 271) | (N = 257) |
| Infections and | Appendicitis | 0 | 1 (0.4) | 0 |
| infestations | | | | |
| | Wound infection | 1 (0.4) | 0 | 0 |
| Injury, poisoning | Abdominal injury | 0 | 1 (0.4) | 0 |
| and procedural | | | | |
| complications | | | | |
| | Cervical vertebral | 0 | 0 | 1 (0.4) |
| | fracture | | | |
| | Fractured coccyx | 1 (0.4) | 0 | 0 |
| | Joint dislocation | 0 | 1 (0.4) | 0 |
| | Lower limb fracture | 0 | 0 | 1 (0.4) |
| | Thermal burn | 0 | 1 (0.4) | 0 |
| Neoplasms | Invasive ductal breast | 0 | 1 (0.4) | 0 |
| benign, malignant | carcinoma | | | |
| and unspecified | | | | |
| | Prostate cancer | 0 | 0 | 1 (0.4) |
| Respiratory, | Acute respiratory | 0 | 0 | 1 (0.4) |
| thoracic and | distress syndrome | | | |
| mediastinal | | | | |
| disorders | | | | |

1-40. (canceled)

41. A method of effective vaccination against dengue disease in a subject or subject population, the method comprising administering a unit dose of a dengue vaccine composition concomitantly, simultaneously or sequentially with a yellow fever vaccine to the subject or subject population, wherein said unit dose comprises a tetravalent dengue virus composition including four live, attenuated dengue virus strains representing serotype 1, serotype 2, serotype 3 and serotype 4.

42. The method according to claim 41,
wherein each of the four live, attenuated dengue virus strains is selected from the following:
(i) a non-chimeric dengue virus comprising all components from the same dengue serotype, and
(ii) a chimeric dengue virus having parts from two dengue serotypes, or
wherein the dengue vaccine composition includes at least one chimeric dengue virus and at least one non-chimeric dengue virus, or
wherein the dengue vaccine composition includes a chimeric dengue serotype 2/1 strain, a non-chimeric serotype 2 strain, a chimeric dengue serotype 2/3 strain, and a chimeric dengue serotype 2/4 strain, or
wherein each of the four live attenuated dengue virus serotypes comprises dengue structural proteins capsid protein (C), pre-membrane protein (prM), and envelope protein (E), and dengue non-structural proteins NS1, NS2A, NS2B, NS3, NS4A, NS4B, and NS5.

43. The method according to claim 42, wherein the dengue serotype 2 strain is a non-chimeric dengue strain derived from the wild type virus strain DEN-2 16681 and is characterized by differing in at least a) to c) from the wild type as follows:
a) 5′-noncoding region (NCR)-57
b) NS1-53 Gly-to-Asp
c) NS3-250 Glu-to-Val; and
wherein the dengue serotype 1, dengue serotype 3, and dengue serotype 4 strains are chimeric dengue strains that are derived from the serotype 2 strain by replacing the structural proteins prM and E from serotype 2 strain with the corresponding structural proteins from the other dengue serotypes, resulting in the following chimeric dengue strains:
a DENV-2/1 chimera,
a DENV-2/3 chimera and
a DENV-2/4 chimera.

44. The method according to claim 42, wherein
the dengue serotype 1 is a chimeric dengue serotype 2/1 strain, the dengue serotype 2 is a non-chimeric serotype 2 strain, the dengue serotype 3 is as a chimeric dengue serotype 2/3 strain, and the dengue serotype 4 is a chimeric dengue serotype 2/4 strain,
wherein the three chimeric dengue strains are derived from the serotype 2 strain by replacing the structural proteins prM and E from the serotype 2 strain with the corresponding structural proteins from the other dengue serotypes,
wherein in the DENV-2/1 chimeric strain the structural proteins prM and E from the serotype 2 strain are replaced with the corresponding structural proteins derived from DENV-1 16007, and
wherein in the DENV-2/3 chimeric strain the structural proteins prM and E from the serotype 2 strain are replaced with the corresponding structural proteins derived from a DENV-3 16562, and
wherein in the DENV-2/4 chimeric strain the structural proteins prM and E from the serotype 2 strain are replaced with the corresponding structural proteins derived from a DENV-4 1036.

45. The method according to claim 41, wherein
the dengue serotype 1 strain is characterized by an amino acid sequence of SEQ ID NO. 2, and/or
the dengue serotype 2 strain is characterized by an amino acid sequence of SEQ ID NO. 4, and/or
the dengue serotype 3 strain is characterized by an amino acid sequence of SEQ ID NO. 6, and/or,
the dengue serotype 4 strain is characterized by an amino acid sequence of SEQ ID NO. 8.

46. The method according to claim 41,
wherein the subject or subject population is under 9 years of age, or 4 to 5 years of age, or 6 to 11 years of age, or 12 to 16 years of age, or 6 to 16 years of age, or 4 to 16 years of age, or 2 to 17 years of age, or 9 years of age, or over 9 years of age, or 9 to 19 years of age, or 18 to 60 years of age, or 18 to 45 years of age, or 46 to 60 years of age, or
wherein the subject belongs to the elderly population of more than 60 years of age, or 61 years to 100 years of age, or 61 years to 90 years of age, or 61 years to 80 years of age, or 61 years to 75 years of age, or 61 years to 70 years of age.

47. The method according to claim 41, wherein the administration results in a reduction of risk of dengue disease with hospitalization in the subject or subject population, or in a reduction of risk of severe dengue in the subject or subject population, or in a reduction of risk of dengue haemorrhagic fever (DHF) and/or dengue shock syndrome (DSS) in the subject or subject population.

48. The method according to claim 41, wherein one dose of the dengue vaccine composition is administered to the subject or subject population.

49. The method according to claim 48, wherein the administration of the one dose results in a reduction of risk of dengue disease within three months, within two months, within one month, or within two weeks after said one dose.

50. The method according to claim 41, wherein
the yellow fever vaccine is administered on day 0,
the first dose or first amount of the dengue vaccine composition is administered after said administration of the yellow fever vaccine, such as 3 months later and preferably on day 90, and
a second dose or second amount of the dengue vaccine composition is administered after said administration of the first dose or first amount of the dengue vaccine composition, such as 3 months later and preferably on day 180,
or
the first dose or first amount of the dengue vaccine composition is administered on day 0,
a second dose or second amount of the dengue vaccine composition is administered after said administration of the first dose or first amount of the dengue vaccine composition, such as 3 months later and preferably on day 90, and
the yellow fever vaccine is administered after said administration of the second dose or second amount of dengue vaccine composition, such as 3 months later and preferably on day 180,
or
the first dose or the first amount of the dengue vaccine composition and the yellow fever vaccine are simultaneously administered on day 0,
a second dose or a second amount of the dengue vaccine composition is administered after said simultaneous administration of the first dose or first amount of the dengue vaccine composition and the yellow fever vaccine, such as 3 months later and preferably on day 90.

51. The method according to claim 41, further comprising administering a booster dose of the dengue vaccine composition to the subject or subject population after the primary vaccination, optionally wherein the booster dose is the third dose of the dengue vaccine composition that is administered to the subject or subject population, optionally
wherein the booster dose is administered at least 12 months or at least 5 years after the administration of said unit dose, optionally wherein the booster dose is administered 12 months after the administration of said unit dose.

52. The method according to claim 41,
wherein the dengue vaccine composition is administered without the need to determine the occurrence of a previous dengue infection in a subject, or
wherein the dengue vaccine composition is administered without determination whether there was a previous dengue infection in a subject, or
not including a step of determination whether there was a previous dengue infection in the subject or subject population before administration of the dengue vaccine composition, wherein the determination of the previous dengue infection is characterized by a laboratory confirmed history of dengue or a validated serological test, or
wherein the serostatus of the subject or subject population is unknown before the administration of the dengue vaccine composition, or
not including a step of determination whether there was a previous dengue infection in the subject or subject population at any time before, during or after the administration of the dengue vaccine composition, wherein the determination of the previous dengue infection is characterized by a laboratory confirmed history of dengue or a validated serological test, or
wherein the serostatus of the subject or subject population is unknown at any time before, during or after the administration of the dengue vaccine composition.

53. The method according to claim 41, wherein the subject or subject population is seronegative at baseline, or
wherein the subject or subject population is seropositive with respect to at least one dengue serotype at baseline.

54. The method according to claim 41, wherein the yellow fever vaccine is used to prevent yellow fever.

55. The method according to claim 41, wherein the yellow fever vaccine is YF-17D, optionally wherein the yellow fever vaccine is prepared by culturing the YF-17D strain of yellow fever virus in living avian leukosis virus-free (ALV-free) chicken embryos.

56. The method according to claim 41,
wherein the yellow fever vaccine contains sorbitol and gelatin as a stabilizer, is lyophilized and contains no preservative, or
wherein the yellow fever vaccine contains not less than 4.74 log₁₀ pfu per 0.5 mL throughout the life of the product.

57. The method according to claim 41, comprising administering a second dengue vaccine composition 3 months after the administration of said first dengue vaccine composition.

58. The method according to claim 41, wherein the dengue vaccine composition and the yellow fever vaccine are administered simultaneously, wherein
a first yellow fever vaccine and a first dengue vaccine composition are administered on day 0, and
the second dengue vaccine composition is administered after said first dengue vaccine composition administration,
or
the first dengue vaccine composition is administered on day 0,
the second dengue vaccine composition and a first yellow fever vaccine are administered after said administration of the first dengue vaccine composition.

59. The method according to claim 41,
wherein the dengue vaccine composition and the yellow fever vaccine are administered sequentially, preferably wherein the yellow fever vaccine is administered before or after the dengue vaccine composition, such as within about 6 weeks, or such as within about 4 weeks, or such as within about 2 weeks, or such as about within 1 week, or
wherein two doses of the dengue vaccine composition are administered, preferably within 12 months or more, or within 6 months, or within three months, such as at day 0/1 and day 90.

60. The method according to claim 41, wherein the dengue vaccine composition and the yellow fever vaccine are administered to a subject or subject population from a dengue endemic region or from a dengue non-endemic region, such as in the context of traveling to a dengue endemic region and yellow fever endemic region.

61. The method according to claim 41, wherein the dengue disease is due to dengue serotype 1 and/or dengue serotype 2, or wherein the subject or subject population is exposed to a dengue virus outbreak, optionally wherein the dengue virus outbreak is due to a dengue serotype 2 and/or due to a dengue serotype 1.

62. The method according to claim 41, wherein the subject or subject population is from a dengue endemic region, optionally wherein the dengue endemic region is characterized by a seroprevalence rate with respect to dengue serotype 1 and/or dengue serotype 2 of at least about 80% or at least about 90%.

63. The method according to claim 41, wherein the tetravalent dengue virus composition is TAK-003.


SEQUENCE LISTING (16 sequences)

SEQ ID NO: 1 - DNA, 10723 bp, synthetic construct
  misc_feature 1..10723: note = Chimeric Dengue Serotype 2/1 (MVS)
  source 1..10723: mol_type = other DNA
agttgttagtctacgtggaccgacaaagacagattctttgagggagctaagctcaatgtagttctaacagttttttaattagagagcagatctctgatgaataaccaacggaaaaaggcgaaaaacacgcctttcaatatgctgaaacgcgagagaaaccgcgtgtcgactgtgcaacagctgacaaagagattctcacttggaatgctgcagggacgaggaccattaaaactgttcatggccctggtggcgttccttcgtttcctaacaatcccaccaacagcagggatattgaagagatggggaacaattaaaaaatcaaaagctattaatgttttgagagggttcaggaaagagattggaaggatgctgaacatcttgaataggagacgcagatctgcaggcatgatcattatgctgattccaacagtgatggcgttccatttaaccacgcgtgggggagagccgcatatgatagttagcaagcaggaaagaggaaagtcacttttgttcaagacctctgcaggtgtcaacatgtgcaccctcattgcgatggatttgggagagttgtgtgaggacacgatgacctacaaatgcccccggatcactgaggcggaaccagatgacgttgactgttggtgcaatgccacggacacatgggtgacctatggaacgtgctctcaaactggcgaacaccgacgagacaaacgttccgtcgcattggccccacacgtggggcttggcctagaaacaagagccgaaacgtggatgtcctctgaaggtgcttggaaacagatacaaaaagtagagacttgggctctgagacatccaggattcacggtgatagccctttttctagcacatgccataggaacatccatcacccagaaagggatcattttcattttgctgatgctggtaacaccatctatggccatgcgatgcgtgggaataggcaacagagacttcgtggaaggactgtcaggagcaacatgggtggatgtggtactggagcatggaagttgcgtcaccaccatggcaaaaaacaaaccaacactggacattgaactcttgaagacggaggtcacaaaccctgcagttctgcgtaaattgtgcattgaagctaaaatatcaaacaccaccaccgattcgagatgtccaacacaaggagaagccacactggtggaagaacaagacgcgaactttgtgtgccgacgaacgttcgtggacagaggctggggcaatggctgtgggctattcggaaaaggtagtctaataacgtgtgccaagtttaagtgtgtgacaaaactagaaggaaagatagttcaatatgaaaacctaaaatattcagtgatagtcaccgtccacactggagatcagcaccaggtgggaaatgagactacagaacatggaacaactgcaaccataacacctcaagctcctacgtcggaaatacagctgaccgactacggaacccttacattagattgttcacctaggacagggctagattttaacgagatggtgttgctgacaatgaaagaaagatcatggcttgtccacaaacaatggttcctagacttaccactgccttggacctctggggcttcaacatcccaagagacttggaacagacaagatttactggtcacatttaagacagctcatgcaaagaagcaggaagtagtcgtactaggatcacaagaaggagcaatgcacactgcgctgactggagcgacagaaatccaaacgtcaggaacgacaacaattttcgcaggacacctaaaatgcagactaaaaatggacaaactaactttaaaagggatgtcatatgtgatgtgcacaggctcattcaagttagagaaagaagtggctgagacccagcatggaactgttctggtgcaggttaaatatgaaggaacagacgcaccatgcaagattcccttttcgacccaagatgagaaaggagcaacccagaatgggagattaataacagccaaccccatagtcactgacaaagaaaaaccagtcaatattgaggcagaaccaccctttggtgagagctacatcgtggtaggagcaggtgaaaaagctttgaaactaagctggttcaagaaaggaagcagcatagggaaaatgtttgaagcaactgcccgaggagcacgaaggatggccattctgggagacaccgcatgggacttcggttctataggaggagtgttcacgtctatgggaaaactggtacaccaggtttttggaactgcatatggagttttgtttagcggagtttcttggaccatgaaaataggaatagggattctgctgacatggctaggattaaattcaaggaacacgtccctttcgatgatgtgcatcgcagccggcattgtgacactgtatttgggagtcatggtgcaggccgatagtggttgcgttgtgagctggaaaaacaaagaactgaaatgtggcagtgggattttcatcacagacaacgtgcacacatggacagaacaatacaagttccaaccagaatccccttcaaaactagcttcagctatccagaaagcccatgaagaggacatttgtggaatccgctcagtaacaagactggagaatctgatgtggaaacaaataacaccagaattgaatcacattctatcagaaaatgaggtgaagttaactattatgacaggagacatcaaaggaatcatgcaggcaggaaaacgatctctgcggcctcagcccactgagctgaagtattcatggaaaacatggggcaaagcaaaaatgctctctacagagtctcataaccagacctttctcattgatggccccgaaacagcagaatgccccaacacaaatagagcttggaattcgttggaagttgaagactatggctttggagtattcaccaccaatatatggctaaaattgaaagaaaaacaggatgtattctgcgactcaaaactcatgtcagcggccataaaagacaacagagccgtccatgccgatatgggttattggatagaaagtgcactcaatgacacatggaagatagagaaagcctctttcattgaagttaaaaactgccactggccaaaatcacacaccctctggagcaatggagtgctagaaagtgagatgataattccaaagaatctcgctggaccagtgtctcaacacaactatagaccaggctaccatacacaaataacaggaccatggcatctaggtaagcttgagatggactttgatttctgtgatggaacaacagtggtagtgactgaggactgcggaaatagaggaccctctttgagaacaaccactgcctctggaaaactcataacagaatggtgctgccgatcttgcacattaccaccgctaagatacagaggtgaggatgggtgctggtacgggatggaaatcagaccattgaaggagaaagaagagaatttggtcaactccttggtcacagctggacatgggcaggtcgacaacttttcactaggagtcttgggaatggcattgttcctggaggaaatgcttaggacccgagtaggaacgaaacatgcaatactactagttgcagtttcttttgtgacattgatcacagggaacatgtcctttagagacctgggaagagtgatggttatggtaggcgccactatgacggatgacataggtatgggcgtgacttatcttgccctactagcagccttcaaagtcagaccaacttttgcagctggactactcttgagaaagctgacctccaaggaattgatgatgactactataggaattgtactcctctcccagagcaccctaccagagaccattcttgagttgactgatgcgttagccttaggcatgatggtcctcaaaatggtgagaaatatggaaaagtatcaattggcagtgactatcatggctatcttgtgcgtcccaaacgcagtgatattacaaaacgcatggaaagtgagttgcacaatattggcagtggtgtccgtttccccactgttcttaacatcctcacagcaaaaaacagattggataccattagcattgacgatcaaaggtctcaatccaacagctatttttctaacaaccctctcaagaaccagcaagaaaaggagctggccattaaatgaggctatcatggcagtcgggatggtgagcattttagccagttctctcctaaaaaatgatattcccatgacaggaccattagtggctggagggctcctcactgtgtgctacgtgctcactggacgatcggccgatttggaactggagagagcagccgatgtcaaatgggaagaccaggcagagatatcaggaagcagtccaatcctgtcaataacaatatcagaagatggtagcatgtcgataaaaaatgaagaggaagatcaaacactgaccatactcattagaacaggattgctggtgatctcaggactttttcctgtatcaataccaatcacggcagcagcatggtacctgtgggaagtgaagaaacaacgggccggagtattgtgggatgttccttcacccccacccatgggaaaggctgaactggaagatggagcctatagaattaagcaaaaagggattcttggatattcccagatcggagccggagtttacaaagaaggaacattccatacaatgtggcatgtcacacgtggcgctgttctaatgcataaaggaaagaggattgaaccatcatgggcggacgtcaagaaagacctaatatcatatggaggaggctggaagttagaaggagaatggaaggaaggagaagaagtccaggtattggcactggagcctggaaaaaatccaagagccgtccaaacgaaacctggtcttttcaaaaccaacgccggaacaataggtgctgtatctctggacttttctcctggaacgtcaggatctccaattatcgacaaaaaaggaaaagttgtgggtctttatggtaatggtgttgttacaaggagtggagcatatgtgagtgctatagcccagactgaaaaaagcattgaagacaacccagagatcgaagatgacattttccgaaagagaagactgaccatcatggacctccacccaggagcgggaaagacgaagagataccttccggccatagtcagagaagctataaaacggggtttgagaacattaatcttggcccccactagagttgtggcagctgaaatggaggaagcccttagaggacttccaataagataccagaccccagccatcagagctgtgcacaccgggcgggagattgtggacctaatgtgtcatgccacatttaccatgaggctgctatcaccagttagagtgccaaactacaacctgattatcatggacgaagcccatttcacagacccagcaagtatagcagctagaggatacatctcaactcgagtggagatgggtgaggcagctgggatttttatgacagccactcccccgggaagcagagacccatttcctcagagcaatgcaccaatcatagatgaagaaagagaaatccctgaacgctcgtggaattccggacatgaatgggtcacggattttaaagggaagactgtttggttcgttccaagtataaaagcaggaaatgatatagcagcttgcctgaggaaaaatggaaagaaagtgatacaactcagtaggaagacctttgattctgagtatgtcaagactagaaccaatgattgggacttcgtggttacaactgacatttcagaaatgggtgccaatttcaaggctgagagggttatagaccccagacgctgcatgaaaccagtcatactaacagatggtgaagagcgggtgattctggcaggacctatgccagtgacccactctagtgcagcacaaagaagagggagaataggaagaaatccaaaaaatgagaatgaccagtacatatacatgggggaacctctggaaaatgatgaagactgtgcacactggaaagaagctaaaatgctcctagataacatcaacacgccagaaggaatcattcctagcatgttcgaaccagagcgtgaaaaggtggatgccattgatggcgaataccgcttgagaggagaagcaaggaaaacctttgtagacttaatgagaagaggagacctaccagtctggttggcctacagagtggcagctgaaggcatcaactacgcagacagaaggtggtgttttgatggagtcaagaacaaccaaatcctagaagaaaacgtggaagttgaaatctggacaaaagaaggggaaaggaagaaattgaaacccagatggttggatgctaggatctattctgacccactggcgctaaaagaatttaaggaatttgcagccggaagaaagtctctgaccctgaacctaatcacagaaatgggtaggctcccaaccttcatgactcagaaggcaagagacgcactggacaacttagcagtgctgcacacggctgaggcaggtggaagggcgtacaaccatgctctcagtgaactgccggagaccctggagacattgcttttactgacacttctggctacagtcacgggagggatctttttattcttgatgagcgcaaggggcatagggaagatgaccctgggaatgtgctgcataatcacggctagcatcctcctatggtacgcacaaatacagccacactggatagcagcttcaataatactggagttttttctcatagttttgcttattccagaacctgaaaaacagagaacaccccaagacaaccaactgacctacgttgtcatagccatcctcacagtggtggccgcaaccatggcaaacgagatgggtttcctagaaaaaacgaagaaagatctcggattgggaagcattgcaacccagcaacccgagagcaacatcctggacatagatctacgtcctgcatcagcatggacgctgtatgccgtggccacaacatttgttacaccaatgttgagacatagcattgaaaattcctcagtgaatgtgtccctaacagctatagccaaccaagccacagtgttaatgggtctcgggaaaggatggccattgtcaaagatggacatcggagttccccttctcgccattggatgctactcacaagtcaaccccataactctcacagcagctcttttcttattggtagcacattatgccatcatagggccaggactccaagcaaaagcaaccagagaagctcagaaaagagcagcggcgggcatcatgaaaaacccaactgtcgatggaataacagtgattgacctagatccaataccttatgatccgaagtttgaaaagcagttgggacaagtaatgctcctagtcctctgcgtgactcaagtattgatgatgaggactacatgggctctgtgtgaggctttaaccttagctaccgggcccatctccacattgtgggaaggaaatccagggaggttttggaacactaccattgcggtgtcaatggctaacatttttagagggagttacttggccggagctggacttctcttttctattatgaagaacacaaccaacacaagaaggggaactggcaacataggagagacgcttggagagaaatggaaaagccgattgaacgcattgggaaaaagtgaattccagatctacaagaaaagtggaatccaggaagtggatagaaccttagcaaaagaaggcattaaaagaggagaaacggaccatcacgctgtgtcgcgaggctcagcaaaactgagatggttcgttgagagaaacatggtcacaccagaagggaaagtagtggacctcggttgtggcagaggaggctggtcatactattgtggaggactaaagaatgtaagagaagtcaaaggcctaacaaaaggaggaccaggacacgaagaacccatccccatgtcaacatatgggtggaatctagtgcgtcttcaaagtggagttgacgttttcttcatcccgccagaaaagtgtgacacattattgtgtgacataggggagtcatcaccaaatcccacagtggaagcaggacgaacactcagagtccttaacttagtagaaaattggttgaacaacaacactcaattttgcataaaggttctcaacccatatatgccctcagtcatagaaaaaatggaagcactacaaaggaaatatggaggagccttagtgaggaatccactctcacgaaactccacacatgagatgtactgggtatccaatgcttccgggaacatagtgtcatcagtgaacatgatttcaaggatgttgatcaacagatttacaatgagatacaagaaagccacttacgagccggatgttgacctcggaagcggaacccgtaacatcgggattgaaagtgagataccaaacctagatataattgggaaaagaatagaaaaaataaagcaagagcatgaaacatcatggcactatgaccaagaccacccatacaaaacgtgggcataccatggtagctatgaaacaaaacagactggatcagcatcatccatggtcaacggagtggtcaggctgctgacaaaaccttgggacgtcgtccccatggtgacacagatggcaatgacagacacgactccatttggacaacagcgcgtttttaaagagaaagtggacacgagaacccaagaaccgaaagaaggcacgaagaaactaatgaaaataacagcagagtggctttggaaagaattagggaagaaaaagacacccaggatgtgcaccagagaagaattcacaagaaaggtgagaagcaatgcagccttgggggccatattcactgatgagaacaagtggaagtcggcacgtgaggctgttgaagatagtaggttttgggagctggttgacaaggaaaggaatctccatcttgaaggaaagtgtgaaacatgtgtgtacaacatgatgggaaaaagagagaagaagctaggggaattcggcaaggcaaaaggcagcagagccatatggtacatgtggcttggagcacgcttcttagagtttgaagccctaggattcttaaatgaagatcactggttctccagagagaactccctgagtggagtggaaggagaagggctgcacaagctaggttacattctaagagacgtgagcaagaaagagggaggagcaatgtatgccgatgacaccgcaggatgggatacaagaatcacactagaagacctaaaaaatgaagaaatggtaacaaaccacatggaaggagaacacaagaaactagccgaggccattttcaaactaacgtaccaaaacaaggtggtgcgtgtgcaaagaccaacaccaagaggcacagtaatggacatcatatcgagaagagaccaaagaggtagtggacaagttggcacctatggactcaatactttcaccaatatggaagcccaactaatcagacagatggagggagaaggagtctttaaaagcattcagcacctaacaatcacagaagaaatcgctgtgcaaaactggttagcaagagtggggcgcgaaaggttatcaagaatggccatcagtggagatgattgtgttgtgaaacctttagatgacaggttcgcaagcgctttaacagctctaaatgacatgggaaagattaggaaagacatacaacaatgggaaccttcaagaggatggaatgattggacacaagtgcccttctgttcacaccatttccatgagttaatcatgaaagacggtcgcgtactcgttgttccatgtagaaaccaagatgaactgattggcagagcccgaatctcccaaggagcagggtggtctttgcgggagacggcctgtttggggaagtcttacgcccaaatgtggagcttgatgtacttccacagacgcgacctcaggctggcggcaaatgctatttgctcggcagtaccatcacattgggttccaacaagtcgaacaacctggtccatacatgctaaacatgaatggatgacaacggaagacatgctgacagtctggaacagggtgtggattcaagaaaacccatggatggaagacaaaactccagtggaatcatgggaggaaatcccatacttggggaaaagagaagaccaatggtgcggctcattgattgggttaacaagcagggccacctgggcaaagaacatccaagcagcaataaatcaagttagatcccttataggcaatgaagaatacacagattacatgccatccatgaaaagattcagaagagaagaggaagaagcaggagttctgtggtagaaagcaaaactaacatgaaacaaggctagaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 2 - protein, 3391 aa, synthetic construct
  REGION 1..3391: note = Chimeric Dengue Serotype 2/1 (MVS)
MNNQRKKAKNTPFNMLKRERNRVSTVQQLTKRFSLGMLQGRGPLKLFMALVAFLRFLTIPPTAGILKRWGTIKKSKAINVLRGFRKEIGRMLNILNRRRRSAGMIIMLIPTVMAFHLTTRGGEPHMIVSKQERGKSLLFKTSAGVNMCTLIAMDLGELCEDTMTYKCPRITEAEPDDVDCWCNATDTWVTYGTCSQTGEHRRDKRSVALAPHVGLGLETRAETWMSSEGAWKQIQKVETWALRHPGFTVIALFLAHAIGTSITQKGIIFILLMLVTPSMAMRCVGIGNRDFVEGLSGATWVDVVLEHGSCVTTMAKNKPTLDIELLKTEVTNPAVLRKLCIEAKISNTTTDSRCPTQGEATLVEEQDANFVCRRTFVDRGWGNGCGLFGKGSLITCAKFKCVTKLEGKIVQYENLKYSVIVTVHTGDQHQVGNETTEHGTTATITPQAPTSEIQLTDYGTLTLDCSPRTGLDFNEMVLLTMKERSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSTQDEKGATQNGRLITANPIVTDKEKPVNIEAEPPFGESYIVVGAGEKALKLSWFKKGSSIGKMFEATARGARRMAILGDTAWDFGSIGGVFTSMGKLVHQVFGTAYGVLFSGVSWTMKIGIGILLTWLGLNSRNTSLSMMCIAAGIVTLYLGVMVQADSGCVVSWKNKELKCGSGIFITDNVHTWTEQYKFQPESPSKLASAIQKAHEEDICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTLPETILELTDALALGMMVLKMVRNMEKYQLAVTIMAILCVPNAVILQNAWKVSCTILAVVSVSPLFLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEDQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAVHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKARDALDNLAVLHTAEAGGRAYNHALSELPETLETLLLLTLLATVTGGIFLFLMSARGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW

SEQ ID NO: 3 - DNA, 10723 bp, Dengue virus
  source 1..10723: mol_type = other DNA
agttgttagtctacgtggaccgacaaagacagattctttgagggagctaagctcaatgtagttctaacagttttttaattagagagcagatctctgatgaataaccaacggaaaaaggcgaaaaacacgcctttcaatatgctgaaacgcgagagaaaccgcgtgtcgactgtgcaacagctgacaaagagattctcacttggaatgctgcagggacgaggaccattaaaactgttcatggccctggtggcgttccttcgtttcctaacaatcccaccaacagcagggatattgaagagatggggaacaattaaaaaatcaaaagctattaatgttttgagagggttcaggaaagagattggaaggatgctgaacatcttgaataggagacgcagatctgcaggcatgatcattatgctgattccaacagtgatggcgttccatttaaccacacgtaacggagaaccacacatgatcgtcagcagacaagagaaagggaaaagtcttctgtttaaaacagaggttggcgtgaacatgtgtaccctcatggccatggaccttggtgaattgtgtgaagacacaatcacgtacgagtgtccccttctcaggcagaatgagccagaagacatagactgttggtgcaactctacgtccacgtgggtaacttatgggacgtgtaccaccatgggagaacatagaagagaaaaaagatcagtggcactcgttccacatgtgggaatgggactggagacacgaactgaaacatggatgtcatcagaaggggcctggaaacatgtccagagaattgaaacttggatcttgagacatccaggcttcaccatgatggcagcaatcctggcatacaccataggaacgacacatttccaaagagccctgatcttcatcttactgacagctgtcactccttcaatgacaatgcgttgcataggaatgtcaaatagagactttgtggaaggggtttcaggaggaagctgggttgacatagtcttagaacatggaagctgtgtgacgacgatggcaaaaaacaaaccaacattggattttgaactgataaaaacagaagccaaacagcctgccaccctaaggaagtactgtatagaggcaaagctaaccaacacaacaacagaatctcgctgcccaacacaaggggaacccagcctaaatgaagagcaggacaaaaggttcgtctgcaaacactccatggtagacagaggatggggaaatggatgtggactatttggaaagggaggcattgtgacctgtgctatgttcagatgcaaaaagaacatggaaggaaaagttgtgcaaccagaaaacttggaatacaccattgtgataacacctcactcaggggaagagcatgcagtcggaaatgacacaggaaaacatggcaaggaaatcaaaataacaccacagagttccatcacagaagcagaattgacaggttatggcactgtcacaatggagtgctctccaagaacgggcctcgacttcaatgagatggtgttgctgcagatggaaaataaagcttggctggtgcacaggcaatggttcctagacctgccgttaccatggttgcccggagcggacacacaagggtcaaattggatacagaaagagacattggtcactttcaaaaatccccatgcgaagaaacaggatgttgttgttttaggatcccaagaaggggccatgcacacagcacttacaggggccacagaaatccaaatgtcatcaggaaacttactcttcacaggacatctcaagtgcaggctgagaatggacaagctacagctcaaaggaatgtcatactctatgtgcacaggaaagtttaaagttgtgaaggaaatagcagaaacacaacatggaacaatagttatcagagtgcaatatgaaggggacggctctccatgcaagatcccttttgagataatggatttggaaaaaagacatgtcttaggtcgcctgattacagtcaacccaattgtgacagaaaaagatagcccagtcaacatagaagcagaacctccatttggagacagctacatcatcataggagtagagccgggacaactgaagctcaactggtttaagaaaggaagttctatcggccaaatgtttgagacaacaatgaggggggcgaagagaatggccattttaggtgacacagcctgggattttggatccttgggaggagtgtttacatctataggaaaggctctccaccaagtctttggagcaatctatggagctgccttcagtggggtttcatggactatgaaaatcctcataggagtcattatcacatggataggaatgaattcacgcagcacctcactgtctgtgacactagtattggtgggaattgtgacactgtatttgggagtcatggtgcaggccgatagtggttgcgttgtgagctggaaaaacaaagaactgaaatgtggcagtgggattttcatcacagacaacgtgcacacatggacagaacaatacaagttccaaccagaatccccttcaaaactagcttcagctatccagaaagcccatgaagaggacatttgtggaatccgctcagtaacaagactggagaatctgatgtggaaacaaataacaccagaattgaatcacattctatcagaaaatgaggtgaagttaactattatgacaggagacatcaaaggaatcatgcaggcaggaaaacgatctctgcggcctcagcccactgagctgaagtattcatggaaaacatggggcaaagcaaaaatgctctctacagagtctcataaccagacctttctcattgatggccccgaaacagcagaatgccccaacacaaatagagcttggaattcgttggaagttgaagactatggctttggagtattcaccaccaatatatggctaaaattgaaagaaaaacaggatgtattctgcgactcaaaactcatgtcagcggccataaaagacaacagagccgtccatgccgatatgggttattggatagaaagtgcactcaatgacacatggaagatagagaaagcctctttcattgaagttaaaaactgccactggccaaaatcacacaccctctggagcaatggagtgctagaaagtgagatgataattccaaagaatctcgctggaccagtgtctcaacacaactatagaccaggctaccatacacaaataacaggaccatggcatctaggtaagcttgagatggactttgatttctgtgatggaacaacagtggtagtgactgaggactgcggaaatagaggaccctctttgagaacaaccactgcctctggaaaactcataacagaatggtgctgccgatcttgcacattaccaccgctaagatacagaggtgaggatgggtgctggtacgggatggaaatcagaccattgaaggagaaagaagagaatttggtcaactccttggtcacagctggacatgggcaggtcgacaacttttcactaggagtcttgggaatggcattgttcctggaggaaatgcttaggacccgagtaggaacgaaacatgcaatactactagttgcagtttcttttgtgacattgatcacagggaacatgtcctttagagacctgggaagagtgatggttatggtaggcgccactatgacggatgacataggtatgggcgtgacttatcttgccctactagcagccttcaaagtcagaccaacttttgcagctggactactcttgagaaagctgacctccaaggaattgatgatgactactataggaattgtactcctctcccagagcaccataccagagaccattcttgagttgactgatgcgttagccttaggcatgatggtcctcaaaatggtgagaaatatggaaaagtatcaattggcagtgactatcatggctatcttgtgcgtcccaaacgcagtgatattacaaaacgcatggaaagtgagttgcacaatattggcagtggtgtccgtttccccactgttcttaacatcctcacagcaaaaaacagattggataccattagcattgacgatcaaaggtctcaatccaacagctatttttctaacaaccctctcaagaaccagcaagaaaaggagctggccattaaatgaggctatcatggcagtcgggatggtgagcattttagccagttctctcctaaaaaatgatattcccatgacaggaccattagtggctggagggctcctcactgtgtgctacgtgctcactggacgatcggccgatttggaactggagagagcagccgatgtcaaatgggaagaccaggcagagatatcaggaagcagtccaatcctgtcaataacaatatcagaagatggtagcatgtcgataaaaaatgaagaggaagaacaaacactgaccatactcattagaacaggattgctggtgatctcaggactttttcctgtatcaataccaatcacggcagcagcatggtacctgtgggaagtgaagaaacaacgggccggagtattgtgggatgttccttcacccccacccatgggaaaggctgaactggaagatggagcctatagaattaagcaaaaagggattcttggatattcccagatcggagccggagtttacaaagaaggaacattccatacaatgtggcatgtcacacgtggcgctgttctaatgcataaaggaaagaggattgaaccatcatgggcggacgtcaagaaagacctaatatcatatggaggaggctggaagttagaaggagaatggaaggaaggagaagaagtccaggtattggcactggagcctggaaaaaatccaagagccgtccaaacgaaacctggtcttttcaaaaccaacgccggaacaataggtgctgtatctctggacttttctcctggaacgtcaggatctccaattatcgacaaaaaaggaaaagttgtgggtctttatggtaatggtgttgttacaaggagtggagcatatgtgagtgctatagcccagactgaaaaaagcattgaagacaacccagagatcgaagatgacattttccgaaagagaagactgaccatcatggacctccacccaggagcgggaaagacgaagagataccttccggccatagtcagagaagctataaaacggggtttgagaacattaatcttggcccccactagagttgtggcagctgaaatggaggaagcccttagaggacttccaataagataccagaccccagccatcagagctgtgcacaccgggcgggagattgtggacctaatgtgtcatgccacatttaccatgaggctgctatcaccagttagagtgccaaactacaacctgattatcatggacgaagcccatttcacagacccagcaagtatagcagctagaggatacatctcaactcgagtggagatgggtgaggcagctgggatttttatgacagccactcccccgggaagcagagacccatttcctcagagcaatgcaccaatcatagatgaagaaagagaaatccctgaacgctcgtggaattccggacatgaatgggtcacggattttaaagggaagactgtttggttcgttccaagtataaaagcaggaaatgatatagcagcttgcctgaggaaaaatggaaagaaagtgatacaactcagtaggaagacctttgattctgagtatgtcaagactagaaccaatgattgggacttcgtggttacaactgacatttcagaaatgggtgccaatttcaaggctgagagggttatagaccccagacgctgcatgaaaccagtcatactaacagatggtgaagagcgggtgattctggcaggacctatgccagtgacccactctagtgcagcacaaagaagagggagaataggaagaaatccaaaaaatgagaatgaccagtacatatacatgggggaacctctggaaaatgatgaagactgtgcacactggaaagaagctaaaatgctcctagataacatcaacacgccagaaggaatcattcctagcatgttcgaaccagagcgtgaaaaggtggatgccattgatggcgaataccgcttgagaggagaagcaaggaaaacctttgtagacttaatgagaagaggagacctaccagtctggttggcctacagagtggcagctgaaggcatcaactacgcagacagaaggtggtgttttgatggagtcaagaacaaccaaatcctagaagaaaacgtggaagttgaaatctggacaaaagaaggggaaaggaagaaattgaaacccagatggttggatgctaggatctattctgacccactggcgctaaaagaatttaaggaatttgcagccggaagaaagtctctgaccctgaacctaatcacagaaatgggtaggctcccaaccttcatgactcagaaggcaagagacgcactggacaacttagcagtgctgcacacggctgaggcaggtggaagggcgtacaaccatgctctcagtgaactgccggagaccctggagacattgcttttactgacacttctggctacagtcacgggagggatctttttattcttgatgagcgcaaggggcatagggaagatgaccctgggaatgtgctgcataatcacggctagcatcctcctatggtacgcacaaatacagccacactggatagcagcttcaataatactggagttttttctcatagttttgcttattccagaacctgaaaaacagagaacaccccaagacaaccaactgacctacgttgtcatagccatcctcacagtggtggccgcaaccatggcaaacgagatgggtttcctagaaaaaacgaagaaagatctcggattgggaagcattgcaacccagcaacccgagagcaacatcctggacatagatctacgtcctgcatcagcatggacgctgtatgccgtggccacaacatttgttacaccaatgttgagacatagcattgaaaattcctcagtgaatgtgtccctaacagctatagccaaccaagccacagtgttaatgggtctcgggaaaggatggccattgtcaaagatggacatcggagttccccttctcgccattggatgctactcacaagtcaaccccataactctcacagcagctcttttcttattggtagcacattatgccatcatagggccaggactccaagcaaaagcaaccagagaagctcagaaaagagcagcggcgggcatcatgaaaaacccaactgtcgatggaataacagtgattgacctagatccaataccttatgatccaaagtttgaaaagcagttgggacaagtaatgctcctagtcctctgcgtgactcaagtattgatgatgaggactacatgggctctgtgtgaggctttaaccttagctaccgggcccatctccacattgtgggaaggaaatccagggaggttttggaacactaccattgcggtgtcaatggctaacatttttagagggagttacttggccggagctggacttctcttttctattatgaagaacacaaccaacacaagaaggggaactggcaacataggagagacgcttggagagaaatggaaaagccgattgaacgcattgggaaaaagtgaattccagatctacaagaaaagtggaatccaggaagtggatagaaccttagcaaaagaaggcattaaaagaggagaaacggaccatcacgctgtgtcgcgaggctcagcaaaactgagatggttcgttgagagaaacatggtcacaccagaagggaaagtagtggacctcggttgtggcagaggaggctggtcatactattgtggaggactaaagaatgtaagagaagtcaaaggcctaacaaaaggaggaccaggacacgaagaacccatccccatgtcaacatatgggtggaatctagtgcgtcttcaaagtggagttgacgttttcttcatcccgccagaaaagtgtgacacattattgtgtgacataggggagtcatcaccaaatcccacagtggaagcaggacgaacactcagagtccttaacttagtagaaaattggttgaacaacaacactcaattttgcataaaggttctcaacccatatatgccctcagtcatagaaaaaatggaagcactacaaaggaaatatggaggagccttagtgaggaatccactctcacgaaactccacacatgagatgtactgggtatccaatgcttccgggaacatagtgtcatcagtgaacatgatttcaaggatgttgatcaacagatttacaatgagatacaagaaagccacttacgagccggatgttgacctcggaagcggaacccgtaacatcgggattgaaagtgagataccaaacctagatataattgggaaaagaatagaaaaaataaagcaagagcatgaaacatcatggcactatgaccaagaccacccatacaaaacgtgggcataccatggtagctatgaaacaaaacagactggatcagcatcatccatggtcaacggagtggtcaggctgctgacaaaaccttgggacgtcgtccccatggtgacacagatggcaatgacagacacgactccatttggacaacagcgcgtttttaaagagaaagtggacacgagaacccaagaaccgaaagaaggcacgaagaaactaatgaaaataacagcagagtggctttggaaagaattagggaagaaaaagacacccaggatgtgcaccagagaagaattcacaagaaaggtgagaagcaatgcagccttgggggccgtattcactgatgagaacaagtggaagtcggcacgtgaggctgttgaagatagtaggttttgggagctggttgacaaggaaaggaatctccatcttgaaggaaagtgtgaaacatgtgtgtacaacatgatgggaaaaagagagaagaagctaggggaattcggcaaggcaaaaggcagcagagccatatggtacatgtggcttggagcacgcttcttagagtttgaagccctaggattcttaaatgaagatcactggttctccagagagaactccctgagtggagtggaaggagaagggctgcacaagctaggttacattctaagagacgtgagcaagaaagagggaggagcaatgtatgccgatgacaccgcaggatgggatacaagaatcacactagaagacctaaaaaatgaagaaatggtaacaaaccacatggaaggagaacacaagaaactagccgaggccattttcaaactaacgtaccaaaacaaggtggtgcgtgtgcaaagaccaacaccaagaggcacagtaatggacatcatatcgagaagagaccaaagaggtagtggacaagttggcacctatggactcaatactttcaccaatatggaagcccaactaatcagacagatggagggagaaggagtctttaaaagcattcagcacctaacaatcacagaagaaatcgctgtgcaaaactggttagcaagagtggggcgcgaaaggttatcaagaatggccatcagtggagatgattgtgttgtgaaacctttagatgacaggttcgcaagcgctttaacagctctaaatgacatgggaaagattaggaaagacatacaacaatgggaaccttcaagaggatggaatgattggacacaagtgcccttctgttcacaccatttccatgagttaatcatgaaagacggtcgcgtactcgttgttccatgtagaaaccaagatgaactgattggcagagcccgaatctcccaaggagcagggtggtctttgcgggagacggcctgtttggggaagtcttacgcccaaatgtggagcttgatgtacttccacagacgcgacctcaggctggcggcaaatgctatttgctcggcagtaccatcacattgggttccaacaagtcgaacaacctggtccatacatgctaaacatgaatggatgacaacggaagacatgctgacagtctggaacagggtgtggattcaagaaaacccatggatggaagacaaaactccagtggaatcatgggaggaaatcccatacttggggaaaagagaagaccaatggtgcggctcattgattgggttaacaagcagggccacctgggcaaagaacatccaagcagcaataaatcaagttagatcccttataggcaatgaagaatacacagattacatgccatccatgaaaagattcagaagagaagaggaagaagcaggagttctgtggtagaaagcaaaactaacatgaaacaaggctagaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 4 - protein, 3391 aa, Dengue virus
MNNQRKKAKNTPFNMLKRERNRVSTVQQLTKRFSLGMLQGRGPLKLFMALVAFLRFLTIPPTAGILKRWGTIKKSKAINVLRGFRKEIGRMLNILNRRRRSAGMIIMLIPTVMAFHLTTRNGEPHMIVSRQEKGKSLLFKTEVGVNMCTLMAMDLGELCEDTITYECPLLRQNEPEDIDCWCNSTSTWVTYGTCTTMGEHRREKRSVALVPHVGMGLETRTETWMSSEGAWKHVQRIETWILRHPGFTMMAAILAYTIGTTHFQRALIFILLTAVTPSMTMRCIGMSNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFRCKKNMEGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVTLVLVGIVTLYLGVMVQADSGCVVSWKNKELKCGSGIFITDNVHTWTEQYKFQPESPSKLASAIQKAHEEDICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTIPETILELTDALALGMMVLKMVRNMEKYQLAVTIMAILCVPNAVILQNAWKVSCTILAVVSVSPLFLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEEQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAVHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKARDALDNLAVLHTAEAGGRAYNHALSELPETLETLLLLTLLATVTGGIFLFLMSARGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAVFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW

SEQ ID NO: 5 - DNA, 10717 bp, synthetic construct
  misc_feature 1..10717: note = Chimeric Dengue Serotype 2/3 (MVS)
  source 1..10717: mol_type = other DNA
agttgttagtctacgtggaccgacaaagacagattctttgagggagctaagctcaatgtagttctaacagttttttaattagagagcagatctctgatgaataaccaacggaaaaaggcgaaaaacacgcctttcaatatgctgaaacgcgagagaaaccgcgtgtcgactgtgcaacagctgacaaagagattctcacttggaatgctgcagggacgaggaccattaaaactgttcatggccctggtggcgttccttcgtttcctaacaatcccaccaacagcagggatattgaagagatggggaacaattaaaaaatcaaaagctattaatgttttgagagggttcaggaaagagattggaaggatgctgaacatcttgaataggagacgcagatctgcaggcatgatcattatgctgattccaacagtgatggcgttccatttaaccacgcgtgatggagagccgcgcatgattgtggggaagaatgaaagaggaaaatccctacttttcaagacagcctctggaatcaacatgtgcacactcatagctatggatctgggagagatgtgtgatgacacggtcacttacaaatgcccccacattaccgaagtggagcctgaagacattgactgctggtgcaaccttacatcgacatgggtgacttatggaacatgcaatcaagctggagagcatagacgcgataagagatcagtggcgttagctccccatgttggcatgggactggacacacgcactcaaacctggatgtcggctgaaggagcttggagacaagtcgagaaggtagagacatgggcccttaggcacccagggtttaccatactagccctatttcttgcccattacataggcacttccttgacccagaaagtggttatttttatactattaatgctggttaccccatccatgacaatgagatgtgtaggagtaggaaacagagattttgtggaaggcctatcgggagctacgtgggttgacgtggtgctcgagcacggtgggtgtgtgactaccatggctaagaacaagcccacgctggacatagagcttcagaagaccgaggccacccaactggcgaccctaaggaagctatgcattgagggaaaaattaccaacataacaaccgactcaagatgtcccacccaaggggaagcgattttacctgaggagcaggaccagaactacgtgtgtaagcatacatacgtggacagaggctggggaaacggttgtggtttgtttggcaagggaagcttggtgacatgcgcgaaatttcaatgtttagaatcaatagagggaaaagtggtgcaacatgagaacctcaaatacaccgtcatcatcacagtgcacacaggagaccaacaccaggtgggaaatgaaacgcagggagtcacggctgagataacaccccaggcatcaaccgctgaagccattttacctgaatatggaaccctcgggctagaatgctcaccacggacaggtttggatttcaatgaaatgatctcattgacaatgaagaacaaagcatggatggtacatagacaatggttctttgacttacccctaccatggacatcaggagcttcagcagaaacaccaacttggaacaggaaagagcttcttgtgacatttaaaaatgcacatgcaaaaaagcaagaagtagttgttcttggatcacaagagggagcaatgcatacagcactgacaggagctacagagatccaaacctcaggaggcacaagtatctttgcggggcacttaaaatgtagactcaagatggacaaattggaactcaaggggatgagctatgcaatgtgcttgagtagctttgtgttgaagaaagaagtctccgaaacgcagcatgggacaatactcattaaggttgagtacaaaggggaagatgcaccctgcaagattcctttctccacggaggatggacaaggaaaagctctcaatggcagactgatcacagccaatccagtggtgaccaagaaggaggagcctgtcaacattgaggctgaacctccttttggagaaagtaacatagtaattggaattggagacaaagccctgaaaatcaactggtacaagaagggaagctcgattgggaagatgttcgaggccactgccagaggtgcaaggcgcatggccatcttgggagacacagcctgggactttggatcagtgggtggtgttttgaattcattagggaaaatggtccaccaaatatttgggagtgcttacacagccctatttggtggagtctcctggatgatgaaaattggaataggtgtcctcttaacctggatagggttgaactcaaaaaatacttctatgtcattttcatgcatcgcggccggcattgtgacactgtatttgggagtcatggtgcaggccgatagtggttgcgttgtgagctggaaaaacaaagaactgaaatgtggcagtgggattttcatcacagacaacgtgcacacatggacagaacaatacaagttccaaccagaatccccttcaaaactagcttcagctatccagaaagcccatgaagaggacatttgtggaatccgctcagtaacaagactggagaatctgatgtggaaacaaataacaccagaattgaatcacattctatcagaaaatgaggtgaagttaactattatgacaggagacatcaaaggaatcatgcaggcaggaaaacgatctctgcggcctcagcccactgagctgaagtattcatggaaaacatggggcaaagcaaaaatgctctctacagagtctcataaccagacctttctcattgatggccccgaaacagcagaatgccccaacacaaatagagcttggaattcgttggaagttgaagactatggctttggagtattcaccaccaatatatggctaaaattgaaagaaaaacaggatgtattctgcgactcaaaactcatgtcagcggccataaaagacaacagagccgtccatgccgatatgggttattggatagaaagtgcactcaatgacacatggaagatagagaaagcctctttcattgaagttaaaaactgccactggccaaaatcacacaccctctggagcaatggagtgctagaaagtgagatgataattccaaagaatctcgctggaccagtgtctcaacacaactatagaccaggctaccatacacaaataacaggaccatggcatctaggtaagcttgagatggactttgatttctgtgatggaacaacagtggtagtgactgaggactgcggaaatagaggaccctctttgagaacaaccactgcctctggaaaactcataacagaatggtgctgccgatcttgcacattaccaccgctaagatacagaggtgaggatgggtgctggtacgggatggaaatcagaccattgaaggagaaagaagagaatttggtcaactccttggtcacagctggacatgggcaggtcgacaacttttcactaggagtcttgggaatggcattgttcctggaggaaatgcttaggacccgagtaggaacgaaacatgcaatactactagttgcagtttcttttgtgacattgatcacagggaacatgtcctttagagacctgggaagagtgatggttatggtaggcgccactatgacggatgacataggtatgggcgtgacttatcttgccctactagcagccttcaaagtcagaccaacttttgcagctggactactcttgagaaagctgacctccaaggaattgatgatgactactataggaattgtactcctctcccagagcaccataccagagaccattcttgagttgactgatgcgttagccttaggcatgatggtcctcaaaatggtgagaaatatggaaaagtatcaattggcagtgactatcatggctatcttgtgcgtcccaaacgcagtgatattacaaaacgcatggaaagtgagttgcacaatattggcagtggtgtccgtttccccactgttcttaacatcctcacagcaaaaaacagattggataccattagcattgacgatcaaaggtctcaatccaacagctatttttctaacaaccctctcaagaaccagcaagaaaaggagctggccattaaatgaggctatcatggcagtcgggatggtgagcattttagccagttctctcctaaaaaatgatattcccatgacaggaccattagtggctggagggctcctcactgtgtgctacgtgctcactggacgatcggccgatttggaactggagagagcagccgatgtcaaatgggaagaccaggcagagatatcaggaagcagtccaatcctgtcaataacaatatcagaagatggtagcatgtcgataaaaaatgaagaggaagaacaaacactgaccatactcattagaacaggattgctggtgatctcaggactttttcctgtatcaataccaatcacggcagcagcatggtacctgtgggaagtgaagaaacaacgggccggagtattgtgggatgttccttcacccccacccatgggaaaggctgaactggaagatggagcctatagaattaagcaaaaagggattcttggatattcccagatcggagccggagtttacaaagaaggaacattccatacaatgtggcatgtcacacgtggcgctgttctaatgcataaaggaaagaggattgaaccatcatgggcggacgtcaagaaagacctaatatcatatggaggaggctggaagttagaaggagaatggaaggaaggagaagaagtccaggtattggcactggagcctggaaaaaatccaagagccgtccaaacgaaacctggtcttttcaaaaccaacgccggaacaataggtgctgtatctctggacttttctcctggaacgtcaggatctccaattatcgacaaaaaaggaaaagttgtgggtctttatggtaatggtgttgttacaaggagtggagcatatgtgagtgctatagcccagactgaaaaaagcattgaagacaacccagagatcgaagatgacattttccgaaagagaagactgaccatcatggacctccacccaggagcgggaaagacgaagagataccttccggccatagtcagagaagctataaaacggggtttgagaacattaatcttggcccccactagagttgtggcagctgaaatggaggaagcccttagaggacttccaataagataccagaccccagccatcagagctgtgcacaccgggcgggagattgtggacctaatgtgtcatgccacatttaccatgaggctgctatcaccagttagagtgccaaactacaacctgattatcatggacgaagcccatttcacagacccagcaagtatagcagctagaggatacatctcaactcgagtggagatgggtgaggcagctgggatttttatgacagccactcccccgggaagcagagacccatttcctcagagcaatgcaccaatcatagatgaagaaagagaaatccctgaacgctcgtggaattccggacatgaatgggtcacggattttaaagggaagactgtttggttcgttccaagtataaaagcaggaaatgatatagcagcttgcctgaggaaaaatggaaagaaagtgatacaactcagtaggaagacctttgattctgagtatgtcaagactagaaccaatgattgggacttcgtggttacaactgacatttcagaaatgggtgccaatttcaaggctgagagggttatagaccccagacgctgcatgaaaccagtcatactaacagatggtgaagagcgggtgattctggcaggacctatgccagtgacccactctagtgcagcacaaagaagagggagaataggaagaaatccaaaaaatgagaatgaccagtacatatacatgggggaacctctggaaaatgatgaagactgtgcacactggaaagaagctaaaatgctcctagataacatcaacacgccagaaggaatcattcctagcatgttcgaaccagagcgtgaaaaggtggatgccattgatggcgaataccgcttgagaggagaagcaaggaaaacctttgtagacttaatgagaagaggagacctaccagtctggttggcctacagagtggcagctgaaggcatcaactacgcagacagaaggtggtgttttgatggagtcaagaacaaccaaatcctagaagaaaacgtggaagttgaaatctggacaaaagaaggggaaaggaagaaattgaaacccagatggttggatgctaggatctattctgacccactggcgctaaaagaatttaaggaatttgcagccggaagaaagtctctgaccctgaacctaatcacagaaatgggtaggctcccaaccttcatgactcagaaggcaagagacgcactggacaacttagcagtgctgcacacggctgaggcaggtggaagggcgtacaaccatgctctcagtgaactgccggagaccctggagacattgcttttactgacacttctggctacagtcacgggagggatctttttattcttgatgagcgcaaggggcatagggaagatgaccctgggaatgtgctgcataatcacggctagcatcctcctatggtacgcacaaatacagccacactggatagcagcttcaataatactggagttttttctcatagttttgcttattccagaacctgaaaaacagagaacaccccaagacaaccaactgacctacgttgtcatagccatcctcacagtggtggccgcaaccatggcaaacgagatgggtttcctagaaaaaacgaagaaagatctcggattgggaagcattgcaacccagcaacccgagagcaacatcctggacatagatctacgtcctgcatcagcatggacgctgtatgccgtggccacaacatttgttacaccaatgttgagacatagcattgaaaattcctcagtgaatgtgtccctaacagctatagccaaccaagccacagtgttaatgggtctcgggaaaggatggccattgtcaaagatggacatcggagttccccttctcgccattggatgctactcacaagtcaaccccataactctcacagcagctcttttcttattggtagcacattatgccatcatagggccaggactccaagcaaaagcaaccagagaagctcagaaaagagcagcggcgggcatcatgaaaaacccaactgtcgatggaataacagtgattgacctagatccaataccttatgatccaaagtttgaaaagcagttgggacaagtaatgctcctagtcctctgcgtgactcaagtattgatgatgaggactacatgggctctgtgtgaggctttaaccttagctaccgggcccatctccacattgtgggaaggaaatccagggaggttttggaacactaccattgcggtgtcaatggctaacatttttagagggagttacttggccggagctggacttctcttttctattatgaagaacacaaccaacacaagaaggggaactggcaacataggagagacgcttggagagaaatggaaaagccgattgaacgcgttgggaaaaagtgaattccagatctacaagaaaagtggaatccaggaagtggatagaaccttagcaaaagaaggcattaaaagaggagaaacggaccatcacgctgtgtcgcgaggctcagcaaaactgagatggttcgttgagagaaacatggtcacaccagaagggaaagtagtggacctcggttgtggcagaggaggctggtcatactattgtggaggactaaagaatgtaagagaagtcaaaggcctaacaaaaggaggaccaggacacgaagaacccatccccatgtcaacatatgggtggaatctagtgcgtcttcaaagtggagttgacgttttcttcatcccgccagaaaagtgtgacacattattgtgtgacataggggagtcatcaccaaatcccacagtggaagcaggacgaacactcagagtccttaacttagtagaaaattggttgaacaacaacactcaattttgcataaaggttctcaacccatatatgccctcagtcatagaaaaaatggaagcactacaaaggaaatatggaggagccttagtgaggaatccactctcacgaaactccacacatgagatgtactgggtatccaatgcttccgggaacatagtgtcatcagtgaacatgatttcaaggatgttgatcaacagatttacaatgagatacaagaaagccacttacgagccggatgttgacctcggaagcggaacccgtaacatcgggattgaaagtgagataccaaacctagatataattgggaaaagaatagaaaaaataaagcaagagcatgaaacatcatggcactatgaccaagaccacccatacaaaacgtgggcataccatggtagctatgaaacaaaacagactggatcagcatcatccatggtcaacggagtggtcaggctgctgacaaaaccttgggacgtcgtccccatggtgacacagatggcaatgacagacacgactccatttggacaacagcgcgtttttaaagagaaagtggacacgagaacccaagaaccgaaagaaggcacgaagaaactaatgaaaataacagcagagtggctttggaaagaattagggaagaaaaagacacccaggatgtgcaccagagaagaattcacaagaaaggtgagaagcaatgcagccttgggggccatattcactgatgagaacaagtggaagtcggcacgtgaggctgttgaagatagtaggttttgggagctggttgacaaggaaaggaatctccatcttgaaggaaagtgtgaaacatgtgtgtacaacatgatgggaaaaagagagaagaagctaggggaattcggcaaggcaaaaggcagcagagccatatggtacatgtggcttggagcacgcttcttagagtttgaagccctaggattcttaaatgaagatcactggttctccagagagaactccctgagtggagtggaaggagaagggctgcacaagctaggttacattctaagagacgtgagcaagaaagagggaggagcaatgtatgccgatgacaccgcaggatgggatacaagaatcacactagaagacctaaaaaatgaagaaatggtaacaaaccacatggaaggagaacacaagaaactagccgaggccattttcaaactaacgtaccaaaacaaggtggtgcgtgtgcaaagaccaacaccaagaggcacagtaatggacatcatatcgagaagagaccaaagaggtagtggacaagttggcacctatggactcaatactttcaccaatatggaagcccaactaatcagacagatggagggagaaggagtctttaaaagcattcagcacctaacaatcacagaagaaatcgctgtgcaaaactggttagcaagagtggggcgcgaaaggttatcaagaatggccatcagtggagatgattgtgttgtgaaacctttagatgacaggttcgcaagcgctttaacagctctaaatgacatgggaaagattaggaaagacatacaacaatgggaaccttcaagaggatggaatgattggacacaagtgcccttctgttcacaccatttccatgagttaatcatgaaagacggtcgcgtactcgttgttccatgtagaaaccaagatgaactgattggcagagcccgaatctcccaaggagcagggtggtctttgcgggagacggcctgtttggggaagtcttacgcccaaatgtggagcttgatgtacttccacagacgcgacctcaggctggcggcaaatgctatttgctcggcagtaccatcacattgggttccaacaagtcgaacaacctggtccatacatgctaaacatgaatggatgacaacggaagacatgctgacagtctggaacagggtgtggattcaagaaaacccatggatggaagacaaaactccagtggaatcatgggaggaaatcccatacttggggaaaagagaagaccaatggtgcggctcattgattgggttaacaagcagggccacctgggcaaagaacatccaagcagcaataaatcaagttagatcccttataggcaatgaagaatacacagattacatgccatccatgaaaagattcagaagagaagaggaagaagcaggagttctgtggtagaaagcaaaactaacatgaaacaaggctagaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 6 - protein, 3389 aa, synthetic construct
  REGION 1..3389: note = Chimeric Dengue Serotype 2/3 (MVS)
MNNQRKKAKNTPFNMLKRERNRVSTVQQLTKRFSLGMLQGRGPLKLFMALVAFLRFLTIPPTAGILKRWGTIKKSKAINVLRGFRKEIGRMLNILNRRRRSAGMIIMLIPTVMAFHLTTRDGEPRMIVGKNERGKSLLFKTASGINMCTLIAMDLGEMCDDTVTYKCPHITEVEPEDIDCWCNLTSTWVTYGTCNQAGEHRRDKRSVALAPHVGMGLDTRTQTWMSAEGAWRQVEKVETWALRHPGFTILALFLAHYIGTSLTQKVVIFILLMLVTPSMTMRCVGVGNRDFVEGLSGATWVDVVLEHGGCVTTMAKNKPTLDIELQKTEATQLATLRKLCIEGKITNITTDSRCPTQGEAILPEEQDQNYVCKHTYVDRGWGNGCGLFGKGSLVTCAKFQCLESIEGKVVQHENLKYTVIITVHTGDQHQVGNETQGVTAEITPQASTAEAILPEYGTLGLECSPRTGLDFNEMISLTMKNKAWMVHRQWFFDLPLPWTSGASAETPTWNRKELLVTFKNAHAKKQEVVVLGSQEGAMHTALTGATEIQTSGGTSIFAGHLKCRLKMDKLELKGMSYAMCLSSFVLKKEVSETQHGTILIKVEYKGEDAPCKIPFSTEDGQGKALNGRLITANPVVTKKEEPVNIEAEPPFGESNIVIGIGDKALKINWYKKGSSIGKMFEATARGARRMAILGDTAWDFGSVGGVLNSLGKMVHQIFGSAYTALFGGVSWMMKIGIGVLLTWIGLNSKNTSMSFSCIAAGIVTLYLGVMVQADSGCVVSWKNKELKCGSGIFITDNVHTWTEQYKFQPESPSKLASAIQKAHEEDICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTIPETILELTDALALGMMVLKMVRNMEKYQLAVTIMAILCVPNAVILQNAWKVSCTILAVVSVSPLFLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEEQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAVHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKARDALDNLAVLHTAEAGGRAYNHALSELPETLETLLLLTLLATVTGGIFLFLMSARGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW

SEQ ID NO: 7 - DNA, 10723 bp, synthetic construct
  misc_feature 1..10723: note = Chimeric Dengue Serotype 2/4 (MVS)
  variation 3773
  source 1..10723: mol_type = other DNA
agttgttagtctacgtggaccgacaaagacagattctttgagggagctaagctcaatgtagttctaacagttttttaattagagagcagatctctgatgaataaccaacggaaaaaggcgaaaaacacgcctttcaatatgctgaaacgcgagagaaaccgcgtgtcgactgtgcaacagctgacaaagagattctcacttggaatgctgcagggacgaggacctttaaaactgttcatggccctggtggcgttccttcgtttcctaacaatcccaccaacagcagggatattgaagagatggggaacaattaaaaaatcaaaagctattaatgttttgagagggttcaggaaagagattggaaggatgctgaacatcttgaataggagacgcagctctgcaggcatgatcattatgctgattccaacagtgatggcgttccatttaaccacgcgtgatggcgaacccctcatgatagtggcaaaacatgaaagggggagacctctcttgtttaagacaacagaggggatcaacaaatgcactctcattgccatggacttgggtgaaatgtgtgaggacactgtcacgtataaatgccccttactggtcaataccgaacctgaagacattgattgctggtgcaatctcacgtctacctgggtcatgtatgggacatgcacccagagcggagaacggagacgagagaagcgctcagtagctttaacaccacattcaggaatgggattggaaacaagagctgagacatggatgtcatcggaaggggcttggaagcatgctcagagagtagagagctggatactcagaaacccaggattcgcgctcttggcaggatttatggcttatatgattgggcaaacaggaatccagcgaactgtcttctttgtcctaatgatgctggtcgccccatcctacggaatgcgatgcgtaggagtaggaaacagagactttgtggaaggagtctcaggtggagcatgggtcgatctggtgctagaacatggaggatgcgtcacaaccatggcccagggaaaaccaaccttggattttgaactgactaagacaacagccaaggaagtggctctgttaagaacctattgcattgaagcctcaatatcaaacataaccacggcaacaagatgtccaacgcaaggagagccttatctaaaagaggaacaagaccaacagtacatttgccggagagatgtggtagacagagggtggggcaatggctgtggcttgtttggaaaaggaggagttgtgacatgtgcgaagttttcatgttcggggaagataacaggcaatttggtccaaattgagaaccttgaatacacagtggttgtaacagtccacaatggagacacccatgcagtaggaaatgacacgtccaatcatggagttacagccacgataactcccaggtcaccatcggtggaagtcaaattgccggactatggagaactaacactcgattgtgaacccaggtctggaattgactttaatgagatgattctgatgaaaatgaaaaagaaaacatggcttgtgcataagcaatggtttttggatctacctctaccatggacagcaggagcagacacatcagaggttcactggaattacaaagagagaatggtgacatttaaggttcctcatgccaagagacaggatgtgacagtgctgggatctcaggaaggagccatgcattctgccctcgctggagccacagaagtggactccggtgatggaaatcacatgtttgcaggacatctcaagtgcaaagtccgtatggagaaattgagaatcaagggaatgtcatacacgatgtgttcaggaaagttctcaattgacaaagagatggcagaaacacagcatgggacaacagtggtgaaagtcaagtatgaaggtgctggagctccgtgtaaagtccccatagagataagagatgtgaacaaggaaaaagtggttgggcgtatcatctcatccacccctttggctgagaataccaacagtgtaaccaacatagagttagaacccccctttggggacagctacatagtgataggtgttggaaacagtgcattaacactccattggttcaggaaagggagttccattggcaagatgtttgagtccacatacagaggtgcaaaacgaatggccattctaggtgaaacagcttgggattttggttccgttggtggactgttcacatcattgggaaaggctgtgcaccaggtttttggaagtgtgtatacaaccctgtttggaggagtctcatggatgattagaatcctaattgggttcctagtgttgtggattggcacgaactcaaggaacacttcaatggctatgacgtgcatagctgccggcattgtgacactgtatttgggggtcatggtgcaggccgatagtggttgcgttgtgagctggaaaaacaaagaactgaaatgtggcagtgggattttcatcacagacaacgtgcacacatggacagaacaatacaagttccaaccagaatccccttcaaaactagcttcagctatccagaaagcccatgaagaggacatttgtggaatccgctcagtaacaagactggagaatctgatgtggaaacaaataacaccagaattgaatcacattctatcagaaaatgaggtgaagttaactattatgacaggagacatcaaaggaatcatgcaggcaggaaaacgatctctgcggcctcagcccactgagctgaagtattcatggaaaacatggggcaaagcaaaaatgctctctacagagtctcataaccagacctttctcattgatggccccgaaacagcagaatgccccaacacaaatagagcttggaattcgttggaagttgaagactatggctttggagtattcaccaccaatatatggctaaaattgaaagaaaaacaggatgtattctgcgactcaaaactcatgtcagcggccataaaagacaacagagccgtccatgccgatatgggttattggatagaaagtgcactcaatgacacatggaagatagagaaagcctctttcattgaagttaaaaactgccactggccaaaatcacacaccctctggagcaatggagtgctagaaagtgagatgataattccaaagaatctcgctggaccagtgtctcaacacaactatagaccaggctaccatacacaaataacaggaccatggcatctaggtaagcttgagatggactttgatttctgtgatggaacaacagtggtagtgactgaggactgcggaaatagaggaccctctttgagaacaaccactgcctctggaaaactcataacagaatggtgctgccgatcttgcacattaccaccgctaagatacagaggtgaggatgggtgctggtacgggatggaaatcagaccattgaaggagaaagaagagaatttggtcaactccttggtcacagctggacatgggcaggtcgacaacttttcactaggagtcttgggaatggcattgttcctggaggaaatgcttaggacccgagtaggaacgaaacatgcaatactactagttgcagtttcttttgtgacattgatcacagggaacatgtcctttagagacctgggaagagtgatggttatggtaggcgccactatgacgggtgacataggtatgggcgtgacttatcttgccctactagcagccttcaaagtcagaccaacttttgcagctggactactcttgagaaagctgacctccarggaattgatgatgactactataggaattgtactcctctcccagagcaccataccagagaccattcttgagttgactgatgcgttagccttaggcatgatggtcctcaaaatggtgagaaatatggaaaagtatcaattggcagtgactatcatggctatcttgtgcgtcccaaacgcagtgatattacaaaacgcatggaaagtgagttgcacaatattggcagtggtgtccgtttccccactgttcttaacatcctcacagcaaaaaacagattggataccattagcattgacgatcaaaggtctcaatccaacagctatttttctaacaaccctctcaagaaccagcaagaaaaggagctggccattaaatgaggctatcatggcagtcgggatggtgagcattttagccagttctctcctaaaaaatgatattcccatgacaggaccattagtggctggagggctcctcactgtgtgctacgtgctcactggacgatcggccgatttggaactggagagagcagccgatgtcaaatgggaagaccaggcagagatatcaggaagcagtccaatcctgtcaataacaatatcagaagatggtagcatgtcgataaaaaatgaagaggaagaacaaacactgaccatactcattagaacaggattgctggtgatctcaggactttttcctgtatcaataccaatcacggcagcagcatggtacctgtgggaagtgaagaaacaacgggccggagtattgtgggatgttccttcacccccacccatgggaaaggctgaactggaagatggagcctatagaattaagcaaaaagggattcttggatattcccagatcggagccggagtttacaaagaaggaacattccatacaatgtggcatgtcacacgtggcgctgttctaatgcataaaggaaagaggattgaaccatcatgggcggacgtcaagaaagacctaatatcatatggaggaggctggaagttagaaggagaatggaaggaaggagaagaagtccaggtattggcactggagcctggaaaaaatccaagagccgtccaaacgaaacctggtcttttcaaaaccaacgccggaacaataggtgctgtatctctggacttttctcctggaacgtcaggatctccaattatcgacaaaaaaggaaaagttgtgggtctttatggtaatggtgttgttacaaggagtggagcatatgtgagtgctatagcccagactgaaaaaagcattgaagacaacccagagatcgaagatgacattttccgaaagagaagactgaccatcatggacctccacccaggagcgggaaagacgaagagataccttccggccatagtcagagaagctataaaacggggtttgagaacattaatcttggcccccactagagttgtggcagctgaaatggaggaagcccttagaggacttccaataagataccagaccccagccatcagagctgtgcacaccgggcgggagattgtggacctaatgtgtcatgccacatttaccatgaggctgctatcaccagttagagtgccaaactacaacctgattatcatggacgaagcccatttcacagatccagcaagtatagcagctagaggatacatctcaactcgagtggagatgggtgaggcagctgggatttttatgacagccactcccccgggaagcagagacccatttcctcagagcaatgcaccaatcatagatgaagaaagagaaatccctgaacgctcgtggaattccggacatgaatgggtcacggattttaaagggaagactgtttggttcgttccaagtataaaagcaggaaatgatatagcagcttgcctgaggaaaaatggaaagaaagtgatacaactcagtaggaagacctttgattctgagtatgtcaagactagaaccaatgattgggacttcgtggttacaactgacatttcagaaatgggtgccaatttcaaggctgagagggttatagaccccagacgctgcatgaaaccagtcatactaacagatggtgaagagcgggtgattctggcaggacctatgccagtgacccactctagtgcagcacaaagaagagggagaataggaagaaatccaaaaaatgagaatgaccagtacatatacatgggggaacctctggaaaatgatgaagactgtgcacactggaaagaagctaaaatgctcctagataacatcaacacgccagaaggaatcattcctagcatgttcgaaccagagcgtgaaaaggtggatgccattgatggcgaataccgcttgagaggagaagcaaggaaaacctttgtagacttaatgagaagaggagacctaccagtctggttggcctacagagtggcagctgaaggcatcaactacgcagacagaaggtggtgttttgatggagtcaagaacaaccaaatcctagaagaaaacgtggaagttgaaatctggacaaaagaaggggaaaggaagaaattgaaacccagatggttggatgctaggatctattctgacccactggcgctaaaagaatttaaggaatttgcagccggaagaaagtctctgaccctgaacctaatcacagaaatgggtaggctcccaaccttcatgactcagaaggtaagagacgcactggacaacttagcagtgctgcacacggctgaggcaggtggaagggcgtacaaccatgctctcagtgaactgccggagaccctggagacattgcttttactgacacttctggctacagtcacgggagggatctttttattcttgatgagcgcaaggggcatagggaagatgaccctgggaatgtgctgcataatcacggctagcatcctcctatggtacgcacaaatacagccacactggatagcagcttcaataatactggagttttttctcatagttttgcttattccagaacctgaaaaacagagaacaccccaagacaaccaactgacctacgttgtcatagccatcctcacagtggtggccgcaaccatggcaaacgagatgggtttcctagaaaaaacgaagaaagatctcggattgggaagcattgcaacccagcaacccgagagcaacatcctggacatagatctacgtcctgcatcagcatggacgctgtatgccgtggccacaacatttgttacaccaatgttgagacatagcattgaaaattcctcagtgaatgtgtccctaacagccatagccaaccaagccacagtgttaatgggtctcgggaaaggatggccattgtcaaagatggacatcggagttccccttctcgccattggatgctactcacaagtcaaccccataactctcacagcagctcttttcttattggtagcacattatgccatcatagggccaggactccaagcaaaagcaaccagagaagctcagaaaagagcagcggcgggcatcatgaaaaacccaactgtcgatggaataacagtgattgacctagatccaataccttatgatccaaagtttgaaaagcagttgggacaagtaatgctcctagtcctctgcgtgactcaagtattgatgatgaggactacatgggctctgtgtgaggctttaaccttagctaccgggcccatctccacattgtgggaaggaaatccagggaggttttggaacactaccattgcggtgtcaatggctaacatttttagagggagttacttggccggagctggacttctcttttctattatgaagaacacaaccaacacaagaaggggaactggcaacataggagagacgcttggagagaaatggaaaagccgattgaacgcattgggaaaaagtgaattccagatctacaagaaaagtggaatccaggaagtggatagaaccttagcaaaagaaggcattaaaagaggagaaacggaccatcacgctgtgtcgcgaggctcagcaaaactgagatggttcgttgagagaaacatggtcacaccagaagggaaagtagtggacctcggttgtggcagaggaggctggtcatactattgtggaggactaaagaatgtaagagaagtcaaaggcctaacaaaaggaggaccaggacacgaagaacccatccccatgtcaacatatgggtggaatctagtgcgtcttcaaagtggagttgacgttttcttcatcccgccagaaaagtgtgacacattattgtgtgacataggggagtcatcaccaaatcccacagtggaagcaggacgaacactcagagtccttaacttagtagaaaattggttgaacaacaacactcaattttgcataaaggttctcaacccatatatgccctcagtcatagaaaaaatggaagcactacaaaggaaatatggaggagccttagtgaggaatccactctcacgaaactccacacatgagatgtactgggtatccaatgcttccgggaacatagtgtcatcagtgaacatgatttcaaggatgttgatcaacagatttacaatgagatacaagaaagccacttacgagccggatgttgacctcggaagcggaacccgtaacatcgggattgaaagtgagataccaaacctagatataattgggaaaagaatagaaaaaataaagcaagagcatgaaacatcatggcactatgaccaagaccacccatacaaaacgtgggcataccatggtagctatgaaacaaaacagactggatcagcatcatccatggtcaacggagtggtcaggctgctgacaaaaccttgggacgtcgtccccatggtgacacagatggcaatgacagacacgactccatttggacaacagcgcgtttttaaagagaaagtggacacgagaacccaagaaccgaaagaaggcacgaagaaactaatgaaaataacagcagagtggctttggaaagaattagggaagaaaaagacacccaggatgtgcaccagagaagaattcacaagaaaggtgagaagcaatgcagccttgggggccatattcactgatgagaacaagtggaagtcggcacgtgaggctgttgaagatagtaggttttgggagctggttgacaaggaaaggaatctccatcttgaaggaaagtgtgaaacatgtgtgtacaacatgatgggaaaaagagagaagaagctaggggaattcggcaaggcaaaaggcagcagagccatatggtacatgtggcttggagcacgcttcttagagtttgaagccctaggattcttaaatgaagatcactggttctccagagagaactccctgagtggagtggaaggagaagggctgcacaagctaggttacattctaagagacgtgagcaagaaagagggaggagcaatgtatgccgatgacaccgcaggatgggatacaagaatcacactagaagacctaaaaaatgaagaaatggtaacaaaccacatggaaggagaacacaagaaactagccgaggccattttcaaactaacgtaccaaaacaaggtggtgcgtgtgcaaagaccaacaccaagaggcacagtaatggacatcatatcgagaagagaccaaagaggtagtggacaagttggcacctatggactcaatactttcaccaatatggaagcccaactaatcagacagatggagggagaaggagtctttaaaagcattcagcacctaacaatcacagaagaaatcgctgtgcaaaactggttagcaagagtggggcgcgaaaggttatcaagaatggccatcagtggagatgattgtgttgtgaaacctttagatgacaggttcgcaagcgctttaacagctctaaatgacatgggaaagattaggaaagacatacaacaatgggaaccttcaagaggatggaatgattggacacaagtgcccttctgttcacaccatttccatgagttaatcatgaaagacggtcgcgtactcgttgttccctgtagaaaccaagatgaactgattggcagagcccgaatctcccaaggagcagggtggtctttgcgggagacggcctgtttggggaagtcttacgcccaaatgtggagcttgatgtacttccacagacgcgacctcaggctggcggcaaatgctatttgctcggcagtaccatcacattgggttccaacaagtcgaacaacctggtccatacatgctaaacatgaatggatgacaacggaagacatgctgacagtctggaacagggtgtggattcaagaaaacccatggatggaagacaaaactccagtggaatcatgggaggaaatcccatacttggggaaaagagaagaccaatggtgcggctcattgattgggttaacaagcagggccacctgggcaaagaacatccaagcagcaataaatcaagttagatcccttataggcaatgaagaatacacagattacatgccatccatgaaaagattcagaagagaagaggaagaagcaggagttctgtggtagaaagcaaaactaacatgaaacaaggctagaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 8 - protein, 3391 aa, synthetic construct
  REGION 1..3391: note = Chimeric Dengue Serotype 2/4 (MVS)
  SITE 1226: note = Xaa - Arg or Lys
MNNQRKKAKNTPFNMLKRERNRVSTVQQLTKRFSLGMLQGRGPLKLFMALVAFLRFLTIPPTAGILKRWGTIKKSKAINVLRGFRKEIGRMLNILNRRRSSAGMIIMLIPTVMAFHLTTRDGEPLMIVAKHERGRPLLFKTTEGINKCTLIAMDLGEMCEDTVTYKCPLLVNTEPEDIDCWCNLTSTWVMYGTCTQSGERRREKRSVALTPHSGMGLETRAETWMSSEGAWKHAQRVESWILRNPGFALLAGFMAYMIGQTGIQRTVFFVLMMLVAPSYGMRCVGVGNRDFVEGVSGGAWVDLVLEHGGCVTTMAQGKPTLDFELTKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDTSNHGVTATITPRSPSVEVKLPDYGELTLDCEPRSGIDFNEMILMKMKKKTWLVHKQWFLDLPLPWTAGADTSEVHWNYKERMVTFKVPHAKRQDVTVLGSQEGAMHSALAGATEVDSGDGNHMFAGHLKCKVRMEKLRIKGMSYTMCSGKFSIDKEMAETQHGTTVVKVKYEGAGAPCKVPIEIRDVNKEKVVGRIISSTPLAENTNSVTNIELEPPFGDSYIVIGVGNSALTLHWFRKGSSIGKMFESTYRGAKRMAILGETAWDFGSVGGLFTSLGKAVHQVFGSVYTTLFGGVSWMIRILIGFLVLWIGTNSRNTSMAMTCIAAGIVTLYLGVMVQADSGCVVSWKNKELKCGSGIFITDNVHTWTEQYKFQPESPSKLASAIQKAHEEDICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVMVMVGATMTGDIGMGVTYLALLAAFKVRPTFAAGLLLRKLTSXELMMTTIGIVLLSQSTIPETILELTDALALGMMVLKMVRNMEKYQLAVTIMAILCVPNAVILQNAWKVSCTILAVVSVSPLFLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEEQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAVHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKVRDALDNLAVLHTAEAGGRAYNHALSELPETLETLLLLTLLATVTGGIFLFLMSARGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW

SEQ ID NO: 9 - DNA, 10735 bp, Dengue virus
  source 1..10735: mol_type = genomic DNA
agttgttagtctacgtggaccgacaagaacagtttcgaatcggaagcttgcttaacgtagttctaacagttttttattagagagcagatctctgatgatcaaccaacgaaaaaagacgggtcgaccgtctttcaatatgctgaaacgcgcgagaaaccgcgtgtcaactgtttcacagttggcgaagagattctcaaaaggattgctctcaggccaaggacccatgaaattggtgatggctttcatagcattcttaagatttctagccatacccccaacagcaggaattttggctagatggggctcattcaagaagaatggagcgattaaagtgttacggggtttcaagagagaaatctcaaacatgctaaacataatgaacaggaggaaaagatccgtgaccatgctccttatgctgctgcccacagccctggcgttccatctgacgacacgagggggagagccgcatatgatagttagcaagcaggaaagaggaaagtcacttttgttcaagacctctgcaggtgtcaacatgtgcaccctcattgcgatggatttgggagagttgtgtgaggacacgatgacctacaaatgcccccggatcactgaggcggaaccagatgacgttgactgttggtgcaatgccacggacacatgggtgacctatggaacgtgctctcaaactggcgaacaccgacgagacaaacgttccgtcgcattggccccacacgtggggcttggcctagaaacaagagccgaaacgtggatgtcctctgaaggtgcttggaaacagatacaaaaagtagagacttgggctctgagacatccaggattcacggtgatagccctttttctagcacatgccataggaacatccatcacccagaaagggatcattttcattttgctgatgctggtaacaccatctatggccatgcgatgcgtgggaataggcaacagagacttcgtggaaggactgtcaggagcaacatgggtggatgtggtactggagcatggaagttgcgtcaccaccatggcaaaaaacaaaccaacactggacattgaactcttgaagacggaggtcacaaaccctgcagttctgcgtaaattgtgcattgaagctaaaatatcaaacaccaccaccgattcgagatgtccaacacaaggagaagccacactggtggaagaacaagacgcgaactttgtgtgccgacgaacgttcgtggacagaggctggggcaatggctgtgggctattcggaaaaggtagtctaataacgtgtgccaagtttaagtgtgtgacaaaactagaaggaaagatagttcaatatgaaaacctaaaatattcagtgatagtcaccgtccacactggagatcagcaccaggtgggaaatgagactacagaacatggaacaactgcaaccataacacctcaagctcctacgtcggaaatacagctgaccgactacggaacccttacattagattgttcacctaggacagggctagattttaacgagatggtgttgctgacaatgaaagaaagatcatggcttgtccacaaacaatggtttctagacttaccactgccttggacctctggggcttcaacatcccaagagacttggaacagacaagatttactggtcacatttaagacagctcatgcaaagaagcaggaagtagtcgtactaggatcacaagaaggagcaatgcacactgcgctgactggagcgacagaaatccaaacgtcaggaacgacaacaattttcgcaggacacctaaaatgcagactaaaaatggacaaactaactttaaaagggatgtcatatgtgatgtgcacaggctcattcaagttagagaaagaagtggctgagacccagcatggaactgttctggtgcaggttaaatatgaaggaacagacgcaccatgcaagattcccttttcgacccaagatgagaaaggagcaacccagaatgggagattaataacagccaaccccatagtcactgacaaagaaaaaccagtcaatattgaggcagaaccaccctttggtgagagctacatcgtggtaggagcaggtgaaaaagctttgaaactaagctggttcaagaaaggaagcagcatagggaaaatgtttgaagcaactgcccgaggagcacgaaggatggccattctgggagacaccgcatgggacttcggttctataggaggagtgttcacgtctatgggaaaactggtacaccaggtttttggaactgcatatggagttttgtttagcggagtttcttggaccatgaaaataggaatagggattctgctgacatggctaggattaaattcaaggaacacgtccctttcgatgatgtgcatcgcagttggcatggtcacactgtacctaggagtcatggttcaggcagattcgggatgtgtaatcaactggaaaggcagagaacttaaatgtggaagcggcatttttgtcactaatgaagttcacacttggacagagcaatacaaattccaggctgactcccccaagagactatcagcagccattgggaaggcatgggaggagggtgtgtgtggaatccgatcagccactcgtctcgagaacatcatgtggaaacaaatatcaaatgaattgaaccacatcctacttgaaaatgacatgaaatttacagtggtcgtgggagatgttagtggaatcttggcccaagggaaaaaaatgattaggccacaacccatggaacacaaatactcgtggaaaagctggggaaaagccaaaatcataggagcggatgtacagaacaccaccttcatcatcgacggcccaaacaccccagaatgccctgacaatcaaagagcatggaatatttgggaagtagaggactatggatttgggattttcacgacaaacatatggttgaaattgcgtgactcctacacccaagtatgtgaccaccggctgatgtcagctgccattaaggacagcaaggcagtccatgctgacatggggtactggatagaaagtgaaaagaacgagacatggaagttggcgagagcctcctttatagaagttaagacatgcatctggccaaaatcccacactctatggagcaatggagttctggaaagtgaaatgataattccaaagatatatggaggaccaatatctcagcacaactacagaccaggatatttcacacaaacagcagggccgtggcacctaggcaagttggaactagatttcgatttttgtgaaggtaccacagttgttgtggatgaacattgtggaaatcgaggaccatctctcagaaccacaacagtcacaggaaagataatccatgaatggtgctgcagatcttgtacgctaccccccctacgtttcaaaggggaagacgggtgttggtacggcatggaaatcagaccagtgaaggacaaggaagagaacctggtcaagtcaatggtctctgcagggtcaggagaagtggacagcttttcactaggactgctatgcatatcaataatgattgaagaagtgatgagatccagatggagcaaaaaaatgctgatgactggaacactggctgtgttcctccttcttataatgggacaattgacatggagtgatctgatcaggttatgtattatggttggagccaacgcttcagacaagatggggatgggaacaacgtacctagctttaatggccactttcaaaatgagaccaatgttcgccgtcgggctattatttcgcagactaacatctagagaagttcttcttcttacaattggcttgagcctggtggcatccgtggagctaccaagttccctagaggagctgggggatggacttgcaataggcatcatgatgttgaaattattgactgattttcagtcacaccagctatgggctactctgctatccttgacatttattaaaacaactttttcattgcactatgcatggaagacaatggctatggtactgtcaattgtatctctcttccctttatgcctgtccacgacctctcaaaaaacaacatggcttccggtgctgttgggatctcttggatgcaaaccactacccatgtttcttataacagaaaacaaaatctggggaaggaagagttggcccctcaatgaaggaattatggctgttggaatagttagtattctactaagttcacttttaaaaaatgatgtgccgctagccggcccattaatagctggaggcatgctaatagcatgttatgtcatatccggaagctcagctgatttatcactggagaaagcggctgaggtctcctgggaggaagaagcagaacactcaggcgcctcacacaacatactagtagaggttcaagatgatggaaccatgaagataaaagatgaagagagagatgacacgctcaccattctccttaaagcaactctgctggcagtctcaggggtgtacccaatgtcaataccagcgaccctttttgtgtggtatttttggcagaaaaagaaacagagatcaggagtgctatgggacacacccagccccccagaagtggaaagagcagttcttgatgatggcatctatagaattttgcaaagaggactgttgggcaggtcccaagtaggagtaggagttttccaagaaggcgtgttccacacaatgtggcacgtcactaggggagctgtcctcatgtatcaaggaaaaaggctggaaccaagctgggccagtgtcaaaaaagacttgatctcatatggaggaggttggaggtttcaaggatcctggaacacgggagaagaagtacaggtgattgctgttgaaccgggaaaaaaccccaaaaatgtacaaacaacgccgggtaccttcaagacccctgaaggcgaagttggagccatagccttagactttaaacctggcacatctggatctcccatcgtaaacagagagggaaaaatagtaggtctttatggaaatggagtggtgacaacaagcggaacttacgttagtgccatagctcaagctaaggcatcacaagaagggcctctaccagagattgaggacgaggtgtttaggaaaagaaacttaacaataatggacctacatccaggatcgggaaaaacaagaagataccttccagccatagtccgtgaggccataaaaaggaagctgcgcacgctaatcctagctcccacaagagttgtcgcttctgaaatggcagaggcactcaagggagtgccaataaggtatcagacaacagcagtgaagagtgaacacacaggaaaggagatagttgaccttatgtgccacgccactttcaccatgcgcctcctgtctcccgtgagagttcccaattataacatgattatcatggatgaagcacacttcaccgatccagccagcatagcagccagagggtacatctcaacccgagtgggtatgggtgaagcagctgcgatctttatgacagccactcccccaggatcggtggaggcctttccacagagcaatgcaattatccaagatgaggaaagagacattcctgagagatcatggaactcaggctatgactggatcactgattttccaggtaaaacagtctggtttgttccaagcatcaaatcaggaaatgacattgccaactgtttaagaaaaaacgggaaacgggtgatccaattgagcagaaaaacctttgacactgagtaccagaaaacaaaaaacaacgactgggactatgtcgtcacaacagacatttccgaaatgggagcaaatttccgggccgacagggtaatagacccaaggcggtgtctgaaaccggtaatactaaaagatggtccagagcgcgtcattctagccggaccgatgccagtgactgtggccagtgccgcccagaggagaggaagaattggaaggaaccaaaacaaggaaggtgatcagtatatttacatgggacagcctttaaacaatgatgaggaccacgctcattggacagaagcaaagatgctccttgacaatataaacacaccagaagggattatcccagccctctatgagccggagagagaaaagagtgcagctatagacggggaatacagactgcggggtgaagcaaggaaaacgttcgtggagctcatgagaagaggggatctaccagtctggctatcctacaaagttgcctcagaaggcttccagtactccgacagaaggtggtgcttcgatggggaaaggaacaaccaggtgttggaggagaacatggacgtggagatctggacaaaagaaggagaaagaaagaaactacgacctcgctggttggacgccagaacatactctgacccactggctctgcgcgagtttaaagagtttgcagcaggaagaagaagcgtctcaggtgacctaatattagaaatagggaaacttccacaacatttgacgcaaagggcccagaatgctttggacaacttggtcatgttgcacaattccgaacaaggaggaaaagcctatagacatgctatggaagaactgccagacacaatagaaacgttgatgctcctagccttgatagctgtgttgactggtggagtgacgctgttcttcctatcaggaagaggtctaggaaaaacatctatcggcttactctgcgtgatggcctcaagcgcactgttatggatggccagtgtggagccccattggatagcggcctccatcatactggagttctttctgatggtactgcttattccagagccagacagacagcgcactccacaggacaaccagctagcatatgtggtgataggtctgttattcatgatattgacagtggcagccaatgagatgggattattggaaaccacaaagaaagacctggggattggccatgtagctgctgaaaaccaccaccatgctacaatgctggacgtagacctacatccagcttcagcctggaccctctatgcagtggccacaacaatcatcactcctatgatgagacacacaattgaaaacacaacggcaaatatttccctgacagccatcgcaaaccaagcagctatattgatgggacttgacaagggatggccaatatcgaagatggacataggagttccacttctcgccttggggtgctattcccaagtgaatccgctgacactgatagcggcagtattgatgctagtagctcattacgccataattggacctggactgcaagcaaaagctactagagaagctcaaaaaagaacagcggctggaataatgaaaaatccaactgtcgacgggattgttgcaatagacttagatcccgtggtttacgatgcaaaatttgaaaaacaactaggccaaataatgttgttgatactttgcacatcacagattcttttgatgcggactacatgggccttgtgtgaatccatcacattggctactggacctctgaccactctttgggagggatctccaggaaaattctggaacaccacaatagcggtatccatggcaaacattttcagggggagttatctagcaggagcaggtctggccttctcattaatgaaatctctaggaggaggtaggagaggcacgggagcccaaggggaaacactgggagaaaaatggaaaagacaactaaaccaactgagcaagtcagaattcaatacttacaagaggagtgggattatggaggtggatagatccgaagccaaagagggactgaaaagaggagaaacaaccaaacacgcagtatcgagaggaacggccaaactgaggtggttcgtggagaggaaccttgtgaaaccagaagggaaagtcatagacctcggttgtggaagaggtggctggtcatattattgcgctgggctgaagaaagtcacagaagtgaaaggatacacaaaaggaggacctggacatgaggaaccaatcccaatggcgacctatggatggaacctagtaaagctgcactccggaaaagatgtattttttataccacctgagaaatgtgacacccttttgtgtgatattggtgagtcctctccgaacccaactatagaggaaggaagaacgttacgtgttctgaaaatggtggaaccatggctcagaggaaaccaattttgcataaaaattctaaatccctatatgccgagcgtggtagaaactctggaacaaatgcaaagaaaacatggaggaatgctagtgcgaaacccactctcaagaaattccacccatgaaatgtactgggtttcatgtggaacaggaaacattgtgtcagcagtaaacatgacatctagaatgttgctaaatcggttcacaatggctcacaggaagccaacatatgaaagagacgtggacttaggcgctggaacaagacatgtggcagtagaaccagaggtagccaacctagatatcattggccagaggatagagaatataaaaaatgaacataagtcaacatggcattatgatgaggacaatccatacaaaacatgggcctatcatggatcatatgaggttaagccatcaggatcggcctcatccatggtcaatggcgtggtgagattgctcaccaaaccatgggatgttatccccatggtcacacaaatagccatgactgataccacaccctttggacaacagagggtgtttaaagagaaagttgacacgcgcacaccaaaagcaaaacgtggcacagcacaaattatggaagtgacagccaggtggttatggggtttcctttctagaaacaaaaaacccagaatttgcacaagagaggagtttacaagaaaagttaggtcaaacgcagctattggagcagtgttcgttgatgaaaatcaatggaactcggcaaaagaagcagtggaagacgaacggttctgggaacttgtccacagagagagggagcttcataaacaggggaaatgtgccacgtgtgtctacaatatgatggggaagagagagaaaaaattaggagagttcggaaaggcaaaaggaagtcgtgcaatatggtacatgtggttgggagcacgcttcctagagtttgaagcccttggtttcatgaatgaagatcactggttcagtagagagaattcactcagtggagtggaaggagaaggactccacaaacttggatacatactcagagacatatcaaggattccaggggggaacatgtatgcagatgacacagccggatgggacacaagaataacagaggatgatctccagaatgaggctaaaatcactgacatcatggagcccgaacatgccctgctggctacgtcaatctttaagctgacctaccaaaataaggtggtaagggtgcagagaccagcaaaaaatggaaccgtgatggatgttatatccagacgtgaccagagaggcagtggacaggttggaacttatggcttaaacactttcaccaacatggaggcccaactgataagacaaatggagtccgagggaatctttttacccagcgaattggaaaccccaaatctagccggaagagttctcgactggttggaaaaatatggtgtcgaaaggctgaaaagaatggcaatcagcggagatgactgtgtggtgaaaccaattgatgacaggttcgcaacagccttaacagctttgaatgacatgggaaaagtaagaaaagacataccacaatgggaaccttcaaaaggatggaatgattggcaacaagtgcctttctgttcacaccacttccaccagctaattatgaaggatgggagggagatagtggtgccatgccgcaaccaagatgaacttgtggggagggccagagtatcacaaggcgccggatggagcctgagagaaaccgcatgcctaggcaagtcatatgcacaaatgtggcagctgatgtatttccacaggagagacctgagactggcggctaacgctatttgttcagccgttccagttgattgggtcccaaccagccgcaccacctggtcgatccatgcccatcaccaatggatgacaacagaagacatgttatcagtatggaatagggtctggatagaggaaaacccatggatggaggataagactcatgtgtccagttgggaagaagttccatacctaggaaagagggaagatcagtggtgtggatccctgataggcttaacagcaagggccacctgggccactaatatacaagtggccataaaccaagtgagaaggctcattgggaatgagaattatctagattacatgacatcaatgaagagattcaagaatgagagtgatcccgaaggggcactctggtaagtcaacacattcacaaaataaaggaaaataaaaaatcaaatgaggcaagaagtcaggccagattaagccatagtacggtaagagctatgctgcctgtgagccccgtccaaggacgtaaaatgaagtcaggccgaaagccacggtttgagcaagccgtgctgcctgtggctccatcgtggggatgtaaaaacccgggaggctgcaacccatggaagctgtacgcatggggtagcagactagtggttagaggagacccctcccaagacacaacgcagcagcggggcccaacaccaggggaagctgtaccctggtggtaaggactagaggttagaggagaccccccgcgtaacaataaacagcatattgacgctgggagagaccagagatcctgctgtctctacagcatcattccaggcacagaacgccagaaaatggaatggtgctgttgaatcaacaggttct

SEQ ID NO: 10 - protein, 3392 aa, Dengue virus
MINQRKKTGRPSFNMLKRARNRVSTVSQLAKRFSKGLLSGQGPMKLVMAFIAFLRFLAIPPTAGILARWGSFKKNGAIKVLRGFKREISNMLNIMNRRKRSVTMLLMLLPTALAFHLTTRGGEPHMIVSKQERGKSLLFKTSAGVNMCTLIAMDLGELCEDTMTYKCPRITEAEPDDVDCWCNATDTWVTYGTCSQTGEHRRDKRSVALAPHVGLGLETRAETWMSSEGAWKQIQKVETWALRHPGFTVIALFLAHAIGTSITQKGIIFILLMLVTPSMAMRCVGIGNRDFVEGLSGATWVDVVLEHGSCVTTMAKNKPTLDIELLKTEVTNPAVLRKLCIEAKISNTTTDSRCPTQGEATLVEEQDANFVCRRTFVDRGWGNGCGLFGKGSLITCAKFKCVTKLEGKIVQYENLKYSVIVTVHTGDQHQVGNETTEHGTTATITPQAPTSEIQLTDYGTLTLDCSPRTGLDFNEMVLLTMKERSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSTQDEKGATQNGRLITANPIVTDKEKPVNIEAEPPFGESYIVVGAGEKALKLSWFKKGSSIGKMFEATARGARRMAILGDTAWDFGSIGGVFTSMGKLVHQVFGTAYGVLFSGVSWTMKIGIGILLTWLGLNSRNTSLSMMCIAVGMVTLYLGVMVQADSGCVINWKGRELKCGSGIFVTNEVHTWTEQYKFQADSPKRLSAAIGKAWEEGVCGIRSATRLENIMWKQISNELNHILLENDMKFTVVVGDVSGILAQGKKMIRPQPMEHKYSWKSWGKAKIIGADVQNTTFIIDGPNTPECPDNQRAWNIWEVEDYGFGIFTTNIWLKLRDSYTQVCDHRLMSAAIKDSKAVHADMGYWIESEKNETWKLARASFIEVKTCIWPKSHTLWSNGVLESEMIIPKIYGGPISQHNYRPGYFTQTAGPWHLGKLELDFDFCEGTTVVVDEHCGNRGPSLRTTTVTGKIIHEWCCRSCTLPPLRFKGEDGCWYGMEIRPVKDKEENLVKSMVSAGSGEVDSFSLGLLCISIMIEEVMRSRWSKKMLMTGTLAVFLLLIMGQLTWSDLIRLCIMVGANASDKMGMGTTYLALMATFKMRPMFAVGLLFRRLTSREVLLLTIGLSLVASVELPSSLEELGDGLAIGIMMLKLLTDFQSHQLWATLLSLTFIKTTFSLHYAWKTMAMVLSIVSLFPLCLSTTSQKTTWLPVLLGSLGCKPLPMFLITENKIWGRKSWPLNEGIMAVGIVSILLSSLLKNDVPLAGPLIAGGMLIACYVISGSSADLSLEKAAEVSWEEEAEHSGASHNILVEVQDDGTMKIKDEERDDTLTILLKATLLAVSGVYPMSIPATLFVWYFWQKKKQRSGVLWDTPSPPEVERAVLDDGIYRILQRGLLGRSQVGVGVFQEGVFHTMWHVTRGAVLMYQGKRLEPSWASVKKDLISYGGGWRFQGSWNTGEEVQVIAVEPGKNPKNVQTTPGTFKTPEGEVGAIALDFKPGTSGSPIVNREGKIVGLYGNGVVTTSGTYVSAIAQAKASQEGPLPEIEDEVFRKRNLTIMDLHPGSGKTRRYLPAIVREAIKRKLRTLILAPTRVVASEMAEALKGVPIRYQTTAVKSEHTGKEIVDLMCHATFTMRLLSPVRVPNYNMIIMDEAHFTDPASIAARGYISTRVGMGEAAAIFMTATPPGSVEAFPQSNAIIQDEERDIPERSWNSGYDWITDFPGKTVWFVPSIKSGNDIANCLRKNGKRVIQLSRKTFDTEYQKTKNNDWDYVVTTDISEMGANFRADRVIDPRRCLKPVILKDGPERVILAGPMPVTVASAAQRRGRIGRNQNKEGDQYIYMGQPLNNDEDHAHWTEAKMLLDNINTPEGIIPALYEPEREKSAAIDGEYRLRGEARKTFVELMRRGDLPVWLSYKVASEGFQYSDRRWCFDGERNNQVLEENMDVEIWTKEGERKKLRPRWLDARTYSDPLALREFKEFAAGRRSVSGDLILEIGKLPQHLTQRAQNALDNLVMLHNSEQGGKAYRHAMEELPDTIETLMLLALIAVLTGGVTLFFLSGRGLGKTSIGLLCVMASSALLWMASVEPHWIAASIILEFFLMVLLIPEPDRQRTPQDNQLAYVVIGLLFMILTVAANEMGLLETTKKDLGIGHVAAENHHHATMLDVDLHPASAWTLYAVATTIITPMMRHTIENTTANISLTAIANQAAILMGLDKGWPISKMDIGVPLLALGCYSQVNPLTLIAAVLMLVAHYAIIGPGLQAKATREAQKRTAAGIMKNPTVDGIVAIDLDPVVYDAKFEKQLGQIMLLILCTSQILLMRTTWALCESITLATGPLTTLWEGSPGKFWNTTIAVSMANIFRGSYLAGAGLAFSLMKSLGGGRRGTGAQGETLGEKWKRQLNQLSKSEFNTYKRSGIMEVDRSEAKEGLKRGETTKHAVSRGTAKLRWFVERNLVKPEGKVIDLGCGRGGWSYYCAGLKKVTEVKGYTKGGPGHEEPIPMATYGWNLVKLHSGKDVFFIPPEKCDTLLCDIGESSPNPTIEEGRTLRVLKMVEPWLRGNQFCIKILNPYMPSVVETLEQMQRKHGGMLVRNPLSRNSTHEMYWVSCGTGNIVSAVNMTSRMLLNRFTMAHRKPTYERDVDLGAGTRHVAVEPEVANLDIIGQRIENIKNEHKSTWHYDEDNPYKTWAYHGSYEVKPSGSASSMVNGVVRLLTKPWDVIPMVTQIAMTDTTPFGQQRVFKEKVDTRTPKAKRGTAQIMEVTARWLWGFLSRNKKPRICTREEFTRKVRSNAAIGAVFVDENQWNSAKEAVEDERFWELVHRERELHKQGKCATCVYNMMGKREKKLGEFGKAKGSRAIWYMWLGARFLEFEALGFMNEDHWFSRENSLSGVEGEGLHKLGYILRDISRIPGGNMYADDTAGWDTRITEDDLQNEAKITDIMEPEHALLATSIFKLTYQNKVVRVQRPAKNGTVMDVISRRDQRGSGQVGTYGLNTFTNMEAQLIRQMESEGIFLPSELETPNLAGRVLDWLEKYGVERLKRMAISGDDCVVKPIDDRFATALTALNDMGKVRKDIPQWEPSKGWNDWQQVPFCSHHFHQLIMKDGREIVVPCRNQDELVGRARVSQGAGWSLRETACLGKSYAQMWQLMYFHRRDLRLAANAICSAVPVDWVPTSRTTWSIHAHHQWMTTEDMLSVWNRVWIEENPWMEDKTHVSSWEEVPYLGKREDQWCGSLIGLTARATWATNIQVAINQVRRLIGNENYLDYMTSMKRFKNESDPEGALW

SEQ ID NO: 11 - DNA, 10723 bp, Dengue virus
  source 1..10723: mol_type = genomic DNA
agttgttagtctacgtggaccgacaaagacagattctttgagggagctaagctcaacgtagttctaacagttttttaattagagagcagatctctgatgaataaccaacggaaaaaggcgaaaaacacgcctttcaatatgctgaaacgcgagagaaaccgcgtgtcgactgtgcaacagctgacaaagagattctcacttggaatgctgcagggacgaggaccattaaaactgttcatggccctggtggcgttccttcgtttcctaacaatcccaccaacagcagggatattgaagagatggggaacaattaaaaaatcaaaagctattaatgttttgagagggttcaggaaagagattggaaggatgctgaacatcttgaataggagacgcagatctgcaggcatgatcattatgctgattccaacagtgatggcgttccatttaaccacacgtaacggagaaccacacatgatcgtcagcagacaagagaaagggaaaagtcttctgtttaaaacagaggatggcgtgaacatgtgtaccctcatggccatggaccttggtgaattgtgtgaagacacaatcacgtacaagtgtccccttctcaggcagaatgagccagaagacatagactgttggtgcaactctacgtccacgtgggtaacttatgggacgtgtaccaccatgggagaacatagaagagaaaaaagatcagtggcactcgttccacatgtgggaatgggactggagacacgaactgaaacatggatgtcatcagaaggggcctggaaacatgtccagagaattgaaacttggatcttgagacatccaggcttcaccatgatggcagcaatcctggcatacaccataggaacgacacatttccaaagagccctgattttcatcttactgacagctgtcactccttcaatgacaatgcgttgcataggaatgtcaaatagagactttgtggaaggggtttcaggaggaagctgggttgacatagtcttagaacatggaagctgtgtgacgacgatggcaaaaaacaaaccaacattggattttgaactgataaaaacagaagccaaacagcctgccaccctaaggaagtactgtatagaggcaaagctaaccaacacaacaacagaatctcgctgcccaacacaaggggaacccagcctaaatgaagagcaggacaaaaggttcgtctgcaaacactccatggtagacagaggatggggaaatggatgtggactatttggaaagggaggcattgtgacctgtgctatgttcagatgcaaaaagaacatggaaggaaaagttgtgcaaccagaaaacttggaatacaccattgtgataacacctcactcaggggaagagcatgcagtcggaaatgacacaggaaaacatggcaaggaaatcaaaataacaccacagagttccatcacagaagcagaattgacaggttatggcactgtcacaatggagtgctctccaagaacgggcctcgacttcaatgagatggtgttgctgcagatggaaaataaagcttggctggtgcacaggcaatggttcctagacctgccgttaccatggttgcccggagcggacacacaagggtcaaattggatacagaaagagacattggtcactttcaaaaatccccatgcgaagaaacaggatgttgttgttttaggatcccaagaaggggccatgcacacagcacttacaggggccacagaaatccaaatgtcatcaggaaacttactcttcacaggacatctcaagtgcaggctgagaatggacaagctacagctcaaaggaatgtcatactctatgtgcacaggaaagtttaaagttgtgaaggaaatagcagaaacacaacatggaacaatagttatcagagtgcaatatgaaggggacggctctccatgcaagatcccttttgagataatggatttggaaaaaagacatgtcttaggtcgcctgattacagtcaacccaattgtgacagaaaaagatagcccagtcaacatagaagcagaacctccattcggagacagctacatcatcataggagtagagccgggacaactgaagctcaactggtttaagaaaggaagttctatcggccaaatgtttgagacaacaatgaggggggcgaagagaatggccattttaggtgacacagcctgggattttggatccttgggaggagtgtttacatctataggaaaggctctccaccaagtctttggagcaatctatggagctgccttcagtggggtttcatggactatgaaaatcctcataggagtcattatcacatggataggaatgaattcacgcagcacctcactgtctgtgacactagtattggtgggaattgtgacactgtatttgggagtcatggtgcaggccgatagtggttgcgttgtgagctggaaaaacaaagaactgaaatgtggcagtgggattttcatcacagacaacgtgcacacatggacagaacaatacaagttccaaccagaatccccttcaaaactagcttcagctatccagaaagcccatgaagagggcatttgtggaatccgctcagtaacaagactggagaatctgatgtggaaacaaataacaccagaattgaatcacattctatcagaaaatgaggtgaagttaactattatgacaggagacatcaaaggaatcatgcaggcaggaaaacgatctctgcggcctcagcccactgagctgaagtattcatggaaaacatggggcaaagcaaaaatgctctctacagagtctcataaccagacctttctcattgatggccccgaaacagcagaatgccccaacacaaatagagcttggaattcgttggaagttgaagactatggctttggagtattcaccaccaatatatggctaaaattgaaagaaaaacaggatgtattctgcgactcaaaactcatgtcagcggccataaaagacaacagagccgtccatgccgatatgggttattggatagaaagtgcactcaatgacacatggaagatagagaaagcctctttcattgaagttaaaaactgccactggccaaaatcacacaccctctggagcaatggagtgctagaaagtgagatgataattccaaagaatctcgctggaccagtgtctcaacacaactatagaccaggctaccatacacaaataacaggaccatggcatctaggtaagcttgagatggactttgatttctgtgatggaacaacagtggtagtgactgaggactgcggaaatagaggaccctctttgagaacaaccactgcctctggaaaactcataacagaatggtgctgccgatcttgcacattaccaccgctaagatacagaggtgaggatgggtgctggtacgggatggaaatcagaccattgaaggagaaagaagagaatttggtcaactccttggtcacagctggacatgggcaggtcgacaacttttcactaggagtcttgggaatggcattgttcctggaggaaatgcttaggacccgagtaggaacgaaacatgcaatactactagttgcagtttcttttgtgacattgatcacagggaacatgtcctttagagacctgggaagagtgatggttatggtaggcgccactatgacggatgacataggtatgggcgtgacttatcttgccctactagcagccttcaaagtcagaccaacttttgcagctggactactcttgagaaagctgacctccaaggaattgatgatgactactataggaattgtactcctctcccagagcaccataccagagaccattcttgagttgactgatgcgttagccttaggcatgatggtcctcaaaatggtgagaaatatggaaaagtatcaattggcagtgactatcatggctatcttgtgcgtcccaaacgcagtgatattacaaaacgcatggaaagtgagttgcacaatattggcagtggtgtccgtttccccactgctcttaacatcctcacagcaaaaaacagattggataccattagcattgacgatcaaaggtctcaatccaacagctatttttctaacaaccctctcaagaaccagcaagaaaaggagctggccattaaatgaggctatcatggcagtcgggatggtgagcattttagccagttctctcctaaaaaatgatattcccatgacaggaccattagtggctggagggctcctcactgtgtgctacgtgctcactggacgatcggccgatttggaactggagagagcagccgatgtcaaatgggaagaccaggcagagatatcaggaagcagtccaatcctgtcaataacaatatcagaagatggtagcatgtcgataaaaaatgaagaggaagaacaaacactgaccatactcattagaacaggattgctggtgatctcaggactttttcctgtatcaataccaatcacggcagcagcatggtacctgtgggaagtgaagaaacaacgggccggagtattgtgggatgttccttcacccccacccatgggaaaggctgaactggaagatggagcctatagaattaagcaaaaagggattcttggatattcccagatcggagccggagtttacaaagaaggaacattccatacaatgtggcatgtcacacgtggcgctgttctaatgcataaaggaaagaggattgaaccatcatgggcggacgtcaagaaagacctaatatcatatggaggaggctggaagttagaaggagaatggaaggaaggagaagaagtccaggtattggcactggagcctggaaaaaatccaagagccgtccaaacgaaacctggtcttttcaaaaccaacgccggaacaataggtgctgtatctctggacttttctcctggaacgtcaggatctccaattatcgacaaaaaaggaaaagttgtgggtctttatggtaatggtgttgttacaaggagtggagcatatgtgagtgctatagcccagactgaaaaaagcattgaagacaacccagagatcgaagatgacattttccgaaagagaagactgaccatcatggacctccacccaggagcgggaaagacgaagagataccttccggccatagtcagagaagctataaaacggggtttgagaacattaatcttggcccccactagagttgtggcagctgaaatggaggaagcccttagaggacttccaataagataccagaccccagccatcagagctgagcacaccgggcgggagattgtggacctaatgtgtcatgccacatttaccatgaggctgctatcaccagttagagtgccaaactacaacctgattatcatggacgaagcccatttcacagacccagcaagtatagcagctagaggatacatctcaactcgagtggagatgggtgaggcagctgggatttttatgacagccactcccccgggaagcagagacccatttcctcagagcaatgcaccaatcatagatgaagaaagagaaatccctgaacgttcgtggaattccggacatgaatgggtcacggattttaaagggaagactgtttggttcgttccaagtataaaagcaggaaatgatatagcagcttgcctgaggaaaaatggaaagaaagtgatacaactcagtaggaagacctttgattctgagtatgtcaagactagaaccaatgattgggacttcgtggttacaactgacatttcagaaatgggtgccaatttcaaggctgagagggttatagaccccagacgctgcatgaaaccagtcatactaacagatggtgaagagcgggtgattctggcaggacctatgccagtgacccactctagtgcagcacaaagaagagggagaataggaagaaatccaaaaaatgagaatgaccagtacatatacatgggggaacctctggaaaatgatgaagactgtgcacactggaaagaagctaaaatgctcctagataacatcaacacgccagaaggaatcattcctagcatgttcgaaccagagcgtgaaaaggtggatgccattgatggcgaataccgcttgagaggagaagcaaggaaaacctttgtagacttaatgagaagaggagacctaccagtctggttggcctacagagtggcagctgaaggcatcaactacgcagacagaaggtggtgttttgatggagtcaagaacaaccaaatcctagaagaaaacgtggaagttgaaatctggacaaaagaaggggaaaggaagaaattgaaacccagatggttggatgctaggatctattctgacccactggcgctaaaagaatttaaggaatttgcagccggaagaaagtctctgaccctgaacctaatcacagaaatgggtaggctcccaaccttcatgactcagaaggcaagagacgcactggacaacttagcagtgctgcacacggctgaggcaggtggaagggcgtacaaccatgctctcagtgaactgccggagaccctggagacattgcttttactgacacttctggctacagtcacgggagggatctttttattcttgatgagcggaaggggcatagggaagatgaccctgggaatgtgctgcataatcacggctagcatcctcctatggtacgcacaaatacagccacactggatagcagcttcaataatactggagttttttctcatagttttgcttattccagaacctgaaaaacagagaacaccccaagacaaccaactgacctacgttgtcatagccatcctcacagtggtggccgcaaccatggcaaacgagatgggtttcctagaaaaaacgaagaaagatctcggattgggaagcattgcaacccagcaacccgagagcaacatcctggacatagatctacgtcctgcatcagcatggacgctgtatgccgtggccacaacatttgttacaccaatgttgagacatagcattgaaaattcctcagtgaatgtgtccctaacagctatagccaaccaagccacagtgttaatgggtctcgggaaaggatggccattgtcaaagatggacatcggagttccccttctcgccattggatgctactcacaagtcaaccccataactctcacagcagctcttttcttattggtagcacattatgccatcatagggccaggactccaagcaaaagcaaccagagaagctcagaaaagagcagcggcgggcatcatgaaaaacccaactgtcgatggaataacagtgattgacctagatccaataccttatgatccaaagtttgaaaagcagttgggacaagtaatgctcctagtcctctgcgtgactcaagtattgatgatgaggactacatgggctctgtgtgaggctttaaccttagctaccgggcccatctccacattgtgggaaggaaatccagggaggttttggaacactaccattgcggtgtcaatggctaacatttttagagggagttacttggccggagctggacttctcttttctattatgaagaacacaaccaacacaagaaggggaactggcaacataggagagacgcttggagagaaatggaaaagccgattgaacgcattgggaaaaagtgaattccagatctacaagaaaagtggaatccaggaagtggatagaaccttagcaaaagaaggcattaaaagaggagaaacggaccatcacgctgtgtcgcgaggctcagcaaaactgagatggttcgttgagagaaacatggtcacaccagaagggaaagtagtggacctcggttgtggcagaggaggctggtcatactattgtggaggactaaagaatgtaagagaagtcaaaggcctaacaaaaggaggaccaggacacgaagaacccatccccatgtcaacatatgggtggaatctagtgcgtcttcaaagtggagttgacgttttcttcatcccgccagaaaagtgtgacacattattgtgtgacataggggagtcatcaccaaatcccacagtggaagcaggacgaacactcagagtccttaacttagtagaaaattggttgaacaacaacactcaattttgcataaaggttctcaacccatatatgccctcagtcatagaaaaaatggaagcactacaaaggaaatatggaggagccttagtgaggaatccactctcacgaaactccacacatgagatgtactgggtatccaatgcttccgggaacatagtgtcatcagtgaacatgatttcaaggatgttgatcaacagatttacaatgagatacaagaaagccacttacgagccggatgttgacctcggaagcggaacccgtaacatcgggattgaaagtgagataccaaacctagatataattgggaaaagaatagaaaaaataaagcaagagcatgaaacatcatggcactatgaccaagaccacccatacaaaacgtgggcataccatggtagctatgaaacaaaacagactggatcagcatcatccatggtcaacggagtggtcaggctgctgacaaaaccttgggacgtcgtccccatggtgacacagatggcaatgacagacacgactccatttggacaacagcgcgtttttaaagagaaagtggacacgagaacccaagaaccgaaagaaggcacgaagaaactaatgaaaataacagcagagtggctttggaaagaattagggaagaaaaagacacccaggatgtgcaccagagaagaattcacaagaaaggtgagaagcaatgcagccttgggggccatattcactgatgagaacaagtggaagtcggcacgtgaggctgttgaagatagtaggttttgggagctggttgacaaggaaaggaatctccatcttgaaggaaagtgtgaaacatgtgtgtacaacatgatgggaaaaagagagaagaagctaggggaattcggcaaggcaaaaggcagcagagccatatggtacatgtggcttggagcacgcttcttagagtttgaagccctaggattcttaaatgaagatcactggttctccagagagaactccctgagtggagtggaaggagaagggctgcacaagctaggttacattctaagagacgtgagcaagaaagagggaggagcaatgtatgccgatgacaccgcaggatgggatacaagaatcacactagaagacctaaaaaatgaagaaatggtaacaaaccacatggaaggagaacacaagaaactagccgaggccattttcaaactaacgtaccaaaacaaggtggtgcgtgtgcaaagaccaacaccaagaggcacagtaatggacatcatatcgagaagagaccaaagaggtagtggacaagttggcacctatggactcaatactttcaccaatatggaagcccaactaatcagacagatggagggagaaggagtctttaaaagcattcagcacctaacaatcacagaagaaatcgctgtgcaaaactggttagcaagagtggggcgcgaaaggttatcaagaatggccatcagtggagatgattgtgttgtgaaacctttagatgacaggttcgcaagcgctttaacagctctaaatgacatgggaaagattaggaaagacatacaacaatgggaaccttcaagaggatggaatgattggacacaagtgcccttctgttcacaccatttccatgagttaatcatgaaagacggtcgcgtactcgttgttccatgtagaaaccaagatgaactgattggcagagcccgaatctcccaaggagcagggtggtctttgcgggagacggcctgtttggggaagtcttacgcccaaatgtggagcttgatgtacttccacagacgcgacctcaggctggcggcaaatgctatttgctcggcagtaccatcacattgggttccaacaagtcgaacaacctggtccatacatgctaaacatgaatggatgacaacggaagacatgctgacagtctggaacagggtgtggattcaagaaaacccatggatggaagacaaaactccagtggaatcatgggaggaaatcccatacttggggaaaagagaagaccaatggtgcggctcattgattgggttaacaagcagggccacctgggcaaagaacatccaagcagcaataaatcaagttagatcccttataggcaatgaagaatacacagattacatgccatccatgaaaagattcagaagagaagaggaagaagcaggagttctgtggtagaaagcaaaactaacatgaaacaaggctagaagtcaggtcggattaagccatagtacggaaaaaactatgctacctgtgagccccgtccaaggacgttaaaagaagtcaggccatcataaatgccatagctggagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 12 - protein, 3391 aa, Dengue virus
MNNQRKKAKNTPFNMLKRERNRVSTVQQLTKRFSLGMLQGRGPLKLFMALVAFLRFLTIPPTAGILKRWGTIKKSKAINVLRGFRKEIGRMLNILNRRRRSAGMIIMLIPTVMAFHLTTRNGEPHMIVSRQEKGKSLLFKTEDGVNMCTLMAMDLGELCEDTITYKCPLLRQNEPEDIDCWCNSTSTWVTYGTCTTMGEHRREKRSVALVPHVGMGLETRTETWMSSEGAWKHVQRIETWILRHPGFTMMAAILAYTIGTTHFQRALIFILLTAVTPSMTMRCIGMSNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFRCKKNMEGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVTLVLVGIVTLYLGVMVQADSGCVVSWKNKELKCGSGIFITDNVHTWTEQYKFQPESPSKLASAIQKAHEEGICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTIPETILELTDALALGMMVLKMVRNMEKYQLAVTIMAILCVPNAVILQNAWKVSCTILAVVSVSPLLLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEEQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAEHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKARDALDNLAVLHTAEAGGRAYNHALSELPETLETLLLLTLLATVTGGIFLFLMSGRGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW

SEQ ID NO: 13 - DNA, 10696 bp, Dengue virus
  source 1..10696: mol_type = genomic DNA
agttgttagtctacgtggaccgacaagaacagtttcgactcggaagcttgcttaacgtagtgctgacagttttttattagagagcagatctctgatgaacaaccaacggaaaaagacgggaaaaccgtctatcaatatgctgaaacgcgtgagaaaccgtgtgtcaactggatcacagttggcgaagagattctcaagaggattgctgaacggccaaggaccaatgaaattggttatggcatttatagctttcctcagatttctagccattccaccgacagcaggagtcttggctagatggggtacctttaagaagtcgggggctattaaggtcttaaaaggcttcaagaaggagatctcaaacatgctgagcattatcaacaaacggaaaaagacatcgctctgtctcatgatgatgttaccagcaacacttgctttccacttaacttcacgagatggagagccgcgcatgattgtggggaagaatgaaagaggaaaatccctacttttcaagacagcctctggaatcaacatgtgcacactcatagccatggatctgggagagatgtgtgatgacacggtcacttacaaatgcccccacattaccgaagtggagcctgaagacattgactgctggtgcaaccttacatcgacatgggtgacttatggaacatgcaatcaagctggagagcatagacgcgataagagatcagtggcgttagctccccatgttggcatgggactggacacacgcactcaaacctggatgtcggctgaaggagcttggagacaagtcgagaaggtagagacatgggcccttaggcacccagggtttaccatactagccctatttcttgcccattacataggcacttccttgacccagaaagtggttatttttatactattaatgctggttaccccatccatgacaatgagatgtgtaggagtaggaaacagagattttgtggaaggcctatcgggagctacgtgggttgacgtggtgctcgagcacggtgggtgtgtgactaccatggctaagaacaagcccacgctggacatagagcttcagaagaccgaggccacccaactggcgaccctaaggaagctatgcattgagggaaaaattaccaacataacaaccgactcaagatgtcccacccaaggggaagcgattttacctgaggagcaggaccagaactacgtgtgtaagcatacatacgtggacagaggctggggaaacggttgtggtttgtttggcaagggaagcttggtgacatgcgcgaaatttcaatgtttagaatcaatagagggaaaagtggtgcaacatgagaacctcaaatacaccgtcatcatcacagtgcacacaggagaccaacaccaggtgggaaatgaaacgcagggagtcacggctgagataacaccccaggcatcaaccgctgaagtcattttacctgaatatggaaccctcgggctagaatgctcaccacggacaggtttggatttcaatgaaatgatctcattgacaatgaagaacaaagcatggatggtacatagacaatggttctttgacttacccctaccatggacatcaggagctacagcagaaacaccaacttggaacaggaaagagcttcttgtgacatttaaaaatgcacatgcaaaaaagcaagaagtagttgttcttggatcacaagagggagcaatgcatacagcactgacaggagctacagagatccaaacctcaggaggcacaagtatctttgcggggcacttaaaatgtagactcaagatggacaaattggaactcaaggggatgagctatgcaatgtgcttgagtagctttgtgttgaagaaagaagtctccgaaacgcagcatgggacaatactcattaaggttgagtacaaaggggaagatgcaccctgcaagattcctttctccacggaggatggacaaggaaaagctcacaatggcagactgatcacagccaatccagtggtgaccaagaaggaggagcctgtcaacattgaggctgaacctccttttggagaaagtaacatagtaattggaattggagacaaagccctgaaaatcaactggtacaagaagggaagctcgattgggaagatgttcgaggccactgccagaggtgcaaggcgcatggccatcttgggagacacagcctgggactttggatcagtgggtggtgttttgaattcattagggaaaatggtccaccaaatatttgggagtgcttacacagccctatttggtggagtctcctggatgatgaaaattggaataggtgtcctcttaacctggatagggttgaactcaaaaaatacttctatgtcattttcatgcatcgcgataggaatcattacactctatctgggagccgtggtgcaagctgacatggggtgtgtcataaactggaaaggcaaagaactcaaatgtggaagtggaattttcgtcactaatgaggtccacacctggacagagcaatacaaatttcaagcagactcccccaagagactggcaacagccattgcaggcgcttgggaaaatggagtgtgcggaattaggtcaacaaccagaatggagaacctcttgtggaagcaaatagccaatgaactgaattacatattatgggaaaacaacattaaattaacggtagttgtaggcgacataactggggtcttagagcaagggaaaagaacactaacaccacaacccatggagctaaaatattcttggaaaacatggggaaaggcaaaaatagtgacagctgaaacacaaaattcctctttcataatagatgggccaagcacaccggagtgtccaagtgcctcaagagcatggaatgtgtgggaggtggaggattacgggttcggagttttcacaaccaacatatggctgaaactccgagaggtgtacacccaactatgtgaccataggctaatgtcggcagccgtcaaggatgagagggctgtacatgccgacatgggctattggatagaaagccaaaagaatgggagttggaagctagaaaaagcatccttcatagaggtgaaaacctgcacatggccaaaatcacacactctctggagcaatggtgtgctagagagtgacatgattatcccaaagagtctagctggtcccatttcgcaacacaaccacaggcccgggtaccacacccaaacggcaggaccctggcacttaggaaaattggagctggacttcaactattgtgaaggaacaacagttgtcatctcagaaaactgtgggacaagaggcccatcattgagaacaacaacggtgtcagggaagttgatacacgaatggtgctgccgctcgtgcacacttcctcccctacgatacatgggagaagacggctgctggtatggcatggaaatcagacccattaatgagaaagaagagaatatggtaaagtctctagcctcagcagggagtggaaaggtggacaacttcacaatgggtgtcttgtgtttggcaatcctctttgaagaggtgatgagaggaaaatttgggaaaaaacacatgattgcaggggttctcttcacgtttgtgctcctcctctcagggcaaataacatggagagacatggcgcacacactcataatgattgggtccaacgcctctgacagaatggggatgggcgtcacttacctagctctaattgcaacatttaaaattcagccattcctggctttgggattcttcctgaggaaactgacatctagagaaaatttattgctgggagttgggttggccatggcagcaacgttacgactgccagaggacattgaacagatggcgaatggaattgctttggggctcatggctcttaaactgataacacaatttgaaacataccaactatggacggcattagtttccctaacgtgttcaaatacaattttcacgttgactgttgcctggagaacagccactctgattttagccggaatttcgcttttgccagtgtgccagtcttcgagcatgaggaaaacagattggctcccaatgactgtggcagctatgggagctcaacccctaccactttttattttcagtctgaaagatacactcaaaaggagaagctggccactgaatgagggggtgatggcagttggacttgtgagcattctagctagttctctccttaggaatgatgtgcctatggctggaccattagtggctgggggcttgctgatagcgtgctacgtcataactggcacgtcagcagacctcactgtagaaaaagcagcagatgtaacatgggaggaagaggccgagcaaacaggagtgtcccacaatttaatggtcacagttgatgatgatggaacaatgagaataaaagatgacgagactgagaacatcttaacagtgcttttaaaaacagcactactaatagtatcaggcatctttccatactccatacccgcaacactgttggtctggcatacttggcaaaagcaaacccaaagatccggcgtcctatgggacgtacccagccccccagagacacagaaagcggaactggaagaaggggtctataggatcaaacagcaaggaatttttgggaaaacccaagtgggggttggagtacagaaagaaggagttttccacaccatgtggcatgtcacaagaggggcagtgttgacacacaatgggaaaagactggaaccaaactgggctagcgtgaaaaaagatctgatttcatacggaggaggatggagattgagtgcacaatggcaaaagggggaggaggtgcaggttattgccgtagagcctgggaagaacccaaagaactttcaaaccatgccaggcatttttcagacaacaacaggggaaataggagcaattgcactggatttcaagcctggaacttcaggatctcccatcataaacagagagggaaaggtagtgggactgtatggcaatggagtggttacaaagaatggaggctatgtcagtggaatagcgcaaacaaatgcagaaccagatggaccgacaccagagttggaagaagagatgttcaaaaagcgaaatctaaccataatggatctccatcctgggtcaggaaagacgcggaaatatcttccagctattgttagagaggcaatcaagagacgcttaaggactctaattttggcaccaacaagggtagttgcagctgagatggaagaagcattgaaagggctcccaataaggtatcaaacaactgcaacaaaatctgaacacacaggaagagagattgttgatctaatgtgtcacgcaacgttcacaatgcgcttgctgtcaccagtcagggttccaaactacaacttgataataatggatgaggcccatttcacagacccagccagtatagcggctagagggtacatatcaactcgtgtaggaatgggagaggcagccgcaattttcatgacagcaacaccccctggaacagctgatgcctttcctcagagcaacgctccaattcaagatgaagagagagacataccggaacgctcatggaattcaggcaatgaatggattactgactttgttgggaagacagtgtggtttgtccctagcatcaaagccggaaatgacatagcaaactgcttgcggaaaaatggaaaaaaggttattcaactcagcaggaagacctttgacacagaatatcaaaagaccaaactgaatgattgggactttgtggtgacaacagacatttcagaaatgggagccaatttcaaagcagatagagtgatcgacccaagaagatgtctcaagccggtgattttgacagatggacccgagcgggtgatcctggctggaccaatgccagtcaccgtagcgagcgctgcgcaaaggagagggagagttggcaggaacccacaaaaagaaaatgaccagtacatattcatgggccagcctctcaacaatgatgaagaccatgctcactggacagaagcaaaaatgctgctggacaacatcaacacaccagaagggattataccagctctctttgaaccagaaagggagaagtcagccgccatagacggcgaataccgcctgaagggtgagtccaggaagactttcgtggaactcatgaggaggggtgacctcccagtttggctagcccataaagtagcatcagaagggatcaaatatacagatagaaaatggtgctttgatggagaacgtaataatcaaattttagaggagaatatggatgtggaaatctggacaaaggaaggagaaaagaaaaaactgagacctaggtggcttgatgcccgcacttattcagatcctttagcactcaaagaattcaaggattttgcagctggcagaaagtcaatcgcccttgatcttgtgacagaaataggaagagtgccttcacacttagcccacagaacgagaaacgccctggataatttggtgatgctgcacacgtcagaacatggcggtagggcctacaggcatgcagtggaggaactaccagaaacgatggaaacactcttactcctgggactgatgatcttgttaacaggtggagcaatgctcttcttgatatcaggtaaagggattggaaagacttcaataggactcatttgtgtaattgcttccagcggcatgttatggatggctgatgtcccactccaatggatcgcatcggctatagtcctggagttttttatgatggtgttgctcataccagaaccagaaaagcagagaactccccaagacaaccaactcgcatatgtcgtgataggcatacttacattggctgcaatagtagcggccaatgaaatgggactgttggaaactacaaagagagatttaggaatgtctaaagaaccaggtgttgtttctccaaccagctatttggatgtggacttgcacccagcatcagcctggacattgtacgccgtggccacaacagtaataacaccaatgttgagacacaccatagagaattccacagcaaatgtgtctctggcagccatagctaaccaggcagtggtcctgatgggtttagacaaaggatggccgatatcgaaaatggacttgggcgtaccactattggcactgggttgctattcacaagtgaacccactaactcttgcagcggcagtacttttgctagtcacacattatgcaattataggtccaggattgcaggcaaaagccacccgtgaagctcagaaaaggacagctgctggaataatgaagaatccaacggtggatggaataatgacaatagacctagatcctgtaatatatgattcaaaatttgaaaagcaactaggacaggtcatgctcctggttctgtgtgcagtccaacttttattgatgagaacatcatgggccttgtgtgaagttctaaccctagccacaggaccaataacaacactctgggaaggatcacctgggaagttctggaacaccacgatagctgtttccatggcgaacatctttagagggagctatttagcaggagctgggcttgctttttctatcatgaaatcagttggaacaggaaagagaggaacagggtcacaaggtgaaaccttaggagaaaagtggaaaaagaaattaaatcagttatcccggaaagagtttgacctttacaagaaatccggaatcaccgaagtggatagaacagaagccaaagaagggttaaaaagaggagaaataacacaccatgccgtgtccagaggcagcgcaaaacttcaatggttcgtggagagaaacatggtcattcctgaaggaagagtcatagacctaggctgtggaagaggaggctggtcatattactgtgcaggactgaaaaaagttacagaagtgcgaggatacacaaaaggcggcccaggacacgaagaaccagtacctatgtctacatacggatggaacatagtcaagttaatgagtggaaaggatgttttttatctgccacctgaaaagtgtgataccctattgtgtgacattggagaatcttcaccaagcccaacagtggaagaaagcagaaccataagagttttgaagatggttgaaccatggctaaagaacaaccagttttgcattaaagtattgaacccatacatgccaactgtgattgagcacttagaaagactacaaaggaaacatggaggaatgcttgtgagaaatccactctcacgaaactccacgcacgaaatgtattggatatccaatggtacaggcaatatcgtctcttcagtcaacatggtatccagattgctactgaacagattcacaatgacacacaggagacccaccatagagaaagatgtggatctaggagcaggaacccgacatgtcaatgcggaaccagaaacacccaacatggatgtcattggggaaagaataaaaaggatcaaagaggagcatagttcaacatggcactatgatgatgaaaatccttacaaaacgtgggcttaccatggatcctatgaagtaaaagccacaggctcagcctcctccatgataaatggagtcgtgaaactcctcacaaaaccatgggatgtggtgcccatggtgacacagatggcaatgacagatacaactccattcggccagcaaagagtttttaaagagaaagtggacaccaggacacctaggcccatgccaggaacaagaaaggttatggagatcacagcggagtggctttggaggaccctgggaaggaacaaaagacccagattatgcacaagggaggaattcacaaagaaggtcagaaccaacgcagctatgggcgctgtcttcacagaagagaaccaatgggacagtgcgagagctgctgttgaggacgaagaattttggaaacttgtggacagagaacgtgaactccacaaactgggcaagtgtggaagctgcgtttacaacatgatgggcaagagagagaaaaaacttggagagtttggtaaagcaaaaggcagtagggctatatggtacatgtggttgggagccaggtaccttgagttcgaggcgctcggattcctcaatgaagaccactggttctcgcgtgaaaactcttacagtggagtagaaggagaaggactgcacaagctgggatacatcttgagagatatttccaagatacccggaggagccatgtatgctgatgacacagccggttgggacacaagaataacagaagatgacctgcacaatgaggaaaaaatcacacagcagatggaccctgaacacaggcagctagcgaacgctatattcaagctcacataccaaaacaaagtggtcaaagtccaacgaccaactccaaagggcacggtaatggacatcatatctaggaaagaccaaagaggcagtggacaggtgggaacttatggtctgaacacattcaccaacatggaagcccagctaatcagacaaatggaaggagaaggcgtgttgtcaaaggcagacctcgagaacccccatccgctagagaagaaaattacacaatggttggaaactaaaggagtggaaaggttaaaaagaatggccatcagcggggatgattgcgttgtgaaaccaatcgacgacagattcgccaatgccctgcttgccctgaacgatatgggaaaggttagaaaggacatacctcaatggcagccatcaaagggatggcatgattggcaacaggtccccttctgctcccaccactttcatgaattgatcatgaaagatggaagaaagttggtagttccctgcagaccccaggacgaactaataggaagagcgagaatctcccaaggagcaggatggagccttagagaaactgcatgtctagggaaagcctacgctcaaatgtgggctctcatgtattttcacagaagagatcttagactagcatccaacgccatatgttcagcagtaccagtccactgggtccccacgagcagaacgacatggtctattcatgctcaccatcagtggatgactacagaagacatgcttactgtctggaacagggtgtggatagaggacaatccatggatggaagacaaaactccagtcacaacgtgggaagatgttccatatctagggaagagagaagaccaatggtgcggatcactcataggtctcacttccagagcaacctgggcccagaacatactcacagcaatccaacaggtgagaagcctcataggcaatgaagagtttctggactacatgccttcgatgaagagattcaggaaggaggaggagtcagagggagccatttggtaaaagcaggaggtaaactgtcaggccacattaagccacagtacggaagaagctgtgcagcctgtgagccccgtccaaggacgttaaaagaagaagtcaggcccaaaagccacggtttgagcaaaccgtgctgcctgtagctccgtcgtggggacgtaaagcctgggaggctgcaaaccgtggaagctgtacgcacggtgtagcagactagtggttagaggagacccctcccatgacacaacgcagcagcggggcccgagcactgagggaagctgtacctccttgcaaaggactagaggttagaggagaccccccgcaaacaaaaacagcatattgacgctgggagagaccagagatcctgctgtctcctcagcatcattccaggcacagaacgccagaaaatggaatggtgctgttgaatcaacaggttct

SEQ ID NO: 14 - protein, 3390 aa, Dengue virus
MNNQRKKTGKPSINMLKRVRNRVSTGSQLAKRFSRGLLNGQGPMKLVMAFIAFLRFLAIPPTAGVLARWGTFKKSGAIKVLKGFKKEISNMLSIINKRKKTSLCLMMMLPATLAFHLTSRDGEPRMIVGKNERGKSLLFKTASGINMCTLIAMDLGEMCDDTVTYKCPHITEVEPEDIDCWCNLTSTWVTYGTCNQAGEHRRDKRSVALAPHVGMGLDTRTQTWMSAEGAWRQVEKVETWALRHPGFTILALFLAHYIGTSLTQKVVIFILLMLVTPSMTMRCVGVGNRDFVEGLSGATWVDVVLEHGGCVTTMAKNKPTLDIELQKTEATQLATLRKLCIEGKITNITTDSRCPTQGEAILPEEQDQNYVCKHTYVDRGWGNGCGLFGKGSLVTCAKFQCLESIEGKVVQHENLKYTVIITVHTGDQHQVGNETQGVTAEITPQASTAEVILPEYGTLGLECSPRTGLDFNEMISLTMKNKAWMVHRQWFFDLPLPWTSGATAETPTWNRKELLVTFKNAHAKKQEVVVLGSQEGAMHTALTGATEIQTSGGTSIFAGHLKCRLKMDKLELKGMSYAMCLSSFVLKKEVSETQHGTILIKVEYKGEDAPCKIPFSTEDGQGKAHNGRLITANPVVTKKEEPVNIEAEPPFGESNIVIGIGDKALKINWYKKGSSIGKMFEATARGARRMAILGDTAWDFGSVGGVLNSLGKMVHQIFGSAYTALFGGVSWMMKIGIGVLLTWIGLNSKNTSMSFSCIAIGIITLYLGAVVQADMGCVINWKGKELKCGSGIFVTNEVHTWTEQYKFQADSPKRLATAIAGAWENGVCGIRSTTRMENLLWKQIANELNYILWENNIKLTVVVGDITGVLEQGKRTLTPQPMELKYSWKTWGKAKIVTAETQNSSFIIDGPSTPECPSASRAWNVWEVEDYGFGVFTTNIWLKLREVYTQLCDHRLMSAAVKDERAVHADMGYWIESQKNGSWKLEKASFIEVKTCTWPKSHTLWSNGVLESDMIIPKSLAGPISQHNHRPGYHTQTAGPWHLGKLELDFNYCEGTTVVISENCGTRGPSLRTTTVSGKLIHEWCCRSCTLPPLRYMGEDGCWYGMEIRPINEKEENMVKSLASAGSGKVDNFTMGVLCLAILFEEVMRGKFGKKHMIAGVLFTFVLLLSGQITWRDMAHTLIMIGSNASDRMGMGVTYLALIATFKIQPFLALGFFLRKLTSRENLLLGVGLAMAATLRLPEDIEQMANGIALGLMALKLITQFETYQLWTALVSLTCSNTIFTLTVAWRTATLILAGISLLPVCQSSSMRKTDWLPMTVAAMGAQPLPLFIFSLKDTLKRRSWPLNEGVMAVGLVSILASSLLRNDVPMAGPLVAGGLLIACYVITGTSADLTVEKAADVTWEEEAEQTGVSHNLMVTVDDDGTMRIKDDETENILTVLLKTALLIVSGIFPYSIPATLLVWHTWQKQTQRSGVLWDVPSPPETQKAELEEGVYRIKQQGIFGKTQVGVGVQKEGVFHTMWHVTRGAVLTHNGKRLEPNWASVKKDLISYGGGWRLSAQWQKGEEVQVIAVEPGKNPKNFQTMPGIFQTTTGEIGAIALDFKPGTSGSPIINREGKVVGLYGNGVVTKNGGYVSGIAQTNAEPDGPTPELEEEMFKKRNLTIMDLHPGSGKTRKYLPAIVREAIKRRLRTLILAPTRVVAAEMEEALKGLPIRYQTTATKSEHTGREIVDLMCHATFTMRLLSPVRVPNYNLIIMDEAHFTDPASIAARGYISTRVGMGEAAAIFMTATPPGTADAFPQSNAPIQDEERDIPERSWNSGNEWITDFVGKTVWFVPSIKAGNDIANCLRKNGKKVIQLSRKTFDTEYQKTKLNDWDFVVTTDISEMGANFKADRVIDPRRCLKPVILTDGPERVILAGPMPVTVASAAQRRGRVGRNPQKENDQYIFMGQPLNNDEDHAHWTEAKMLLDNINTPEGIIPALFEPEREKSAAIDGEYRLKGESRKTFVELMRRGDLPVWLAHKVASEGIKYTDRKWCFDGERNNQILEENMDVEIWTKEGEKKKLRPRWLDARTYSDPLALKEFKDFAAGRKSIALDLVTEIGRVPSHLAHRTRNALDNLVMLHTSEHGGRAYRHAVEELPETMETLLLLGLMILLTGGAMLFLISGKGIGKTSIGLICVIASSGMLWMADVPLQWIASAIVLEFFMMVLLIPEPEKQRTPQDNQLAYVVIGILTLAAIVAANEMGLLETTKRDLGMSKEPGVVSPTSYLDVDLHPASAWTLYAVATTVITPMLRHTIENSTANVSLAAIANQAVVLMGLDKGWPISKMDLGVPLLALGCYSQVNPLTLAAAVLLLVTHYAIIGPGLQAKATREAQKRTAAGIMKNPTVDGIMTIDLDPVIYDSKFEKQLGQVMLLVLCAVQLLLMRTSWALCEVLTLATGPITTLWEGSPGKFWNTTIAVSMANIFRGSYLAGAGLAFSIMKSVGTGKRGTGSQGETLGEKWKKKLNQLSRKEFDLYKKSGITEVDRTEAKEGLKRGEITHHAVSRGSAKLQWFVERNMVIPEGRVIDLGCGRGGWSYYCAGLKKVTEVRGYTKGGPGHEEPVPMSTYGWNIVKLMSGKDVFYLPPEKCDTLLCDIGESSPSPTVEESRTIRVLKMVEPWLKNNQFCIKVLNPYMPTVIEHLERLQRKHGGMLVRNPLSRNSTHEMYWISNGTGNIVSSVNMVSRLLLNRFTMTHRRPTIEKDVDLGAGTRHVNAEPETPNMDVIGERIKRIKEEHSSTWHYDDENPYKTWAYHGSYEVKATGSASSMINGVVKLLTKPWDVVPMVTQMAMTDTTPFGQQRVFKEKVDTRTPRPMPGTRKVMEITAEWLWRTLGRNKRPRLCTREEFTKKVRTNAAMGAVFTEENQWDSARAAVEDEEFWKLVDRERELHKLGKCGSCVYNMMGKREKKLGEFGKAKGSRAIWYMWLGARYLEFEALGFLNEDHWFSRENSYSGVEGEGLHKLGYILRDISKIPGGAMYADDTAGWDTRITEDDLHNEEKITQQMDPEHRQLANAIFKLTYQNKVVKVQRPTPKGTVMDIISRKDQRGSGQVGTYGLNTFTNMEAQLIRQMEGEGVLSKADLENPHPLEKKITQWLETKGVERLKRMAISGDDCVVKPIDDRFANALLALNDMGKVRKDIPQWQPSKGWHDWQQVPFCSHHFHELIMKDGRKLVVPCRPQDELIGRARISQGAGWSLRETACLGKAYAQMWALMYFHRRDLRLASNAICSAVPVHWVPTSRTTWSIHAHHQWMTTEDMLTVWNRVWIEDNPWMEDKTPVTTWEDVPYLGKREDQWCGSLIGLTSRATWAQNILTAIQQVRSLIGNEEFLDYMPSMKRFRKEEESEGAIW

SEQ ID NO: 15 - DNA, 10648 bp, Dengue virus
  source 1..10648: mol_type = genomic DNA
agttgttagtctgtgtggaccgacaaggacagttccaaatcggaagcttgcttaacacagttctaacagtttgtttgaatagagagcagatctctggaaaaatgaaccaacgaaaaaaggtggttagaccacctttcaatatgctgaaacgcgagagaaaccgcgtatcaacccctcaagggttggtgaagagattctcaaccggacttttttctgggaaaggacccttacggatggtgctagcattcatcacgtttttgcgagtcctttccatcccaccaacagcagggattctgaaaagatggggacagttgaagaaaaataaggccatcaggatactgattggattcaggaaggagataggccgcatgctgaacatcttgaacgggagaaaaaggtcaacgataacattgctgtgcttgattcccaccgtaatggcgtttcacttgtcaacaagagatggcgaacccctcatgatagtggcaaaacatgaaagggggagacctctcttgtttaagacaacagaggggatcaacaaatgcactctcattgccatggacttgggtgaaatgtgtgaggacactgtcacgtataaatgccccttactggtcaataccgaacctgaagacattgattgctggtgcaatctcacgtctacctgggtcatgtatgggacatgcacccagagcggagaacggagacgagagaagcgctcagtagctttaacaccacattcaggaatgggattggaaacaagagctgagacatggatgtcatcggaaggggcttggaagcatgctcagagagtagagagctggatactcagaaacccaggattcgcgctcttggcaggatttatggcttatatgattgggcaaacaggaatccagcgaactgtcttctttgtcctaatgatgctggtcgccccatcctacggaatgcgatgcgtaggagtaggaaacagagactttgtggaaggagtctcaggtggagcatgggtcgatctggtgctagaacatggaggatgcgtcacaaccatggcccagggaaaaccaaccttggattttgaactgactaagacaacagccaaggaagtggctctgttaagaacctattgcattgaagcctcaatatcaaacataaccacggcaacaagatgtccaacgcaaggagagccttatctaaaagaggaacaagaccaacagtacatttgccggagagatgtggtagacagagggtggggcaatggctgtggcttgtttggaaaaggaggagttgtgacatgtgcgaagttttcatgttcggggaagataacaggcaatttggtccaaattgagaaccttgaatacacagtggttgtaacagtccacaatggagacacccatgcagtaggaaatgacacatccaatcatggagttacagccacgataactcccaggtcaccatcggtggaagtcaaattgccggactatggagaactaacactcgattgtgaacccaggtctggaattgactttaatgagatgattctgatgaaaatgaaaaagaaaacatggcttgtgcataagcaatggtttttggatctacctctaccatggacagcaggagcagacacatcagaggttcactggaattacaaagagagaatggtgacatttaaggttcctcatgccaagagacaggatgtgacagtgctgggatctcaggaaggagccatgcattctgccctcgctggagccacagaagtggactccggtgatggaaatcacatgtttgcaggacatctcaagtgcaaagtccgtatggagaaattgagaatcaagggaatgtcatacacgatgtgttcaggaaagttctcaattgacaaagagatggcagaaacacagcatgggacaacagtggtgaaagtcaagtatgaaggtgctggagctccgtgtaaagtccccatagagataagagatgtgaacaaggaaaaagtggttgggcgtatcatctcatccacccctttggctgagaataccaacagtgcaaccaacatagagttagaacccccctttggggacagctacatagtgataggtgttggaaacagtgcattaacactccattggttcaggaaagggagttccattggcaagatgtttgagtccacatacagaggtgcaaaacgaatggccattctaggtgaaacagcttgggattttggttccgttggtggactgttcacatcattgggaaaggctgtgcaccaggtttttggaagtgtgtatacaaccatgtttggaggagtctcatggatgattagaatcctaattgggttcctagtgttgtggattggcacgaactcaaggaacacttcaatggctatgacgtgcatagctgttggaggaatcactctgtttctgggcttcacagttcaagcagacatgggttgtgtggtgtcatggagtgggaaagaattgaagtgtggaagcggaatttttgtggttgacaacgtgcacacttggacagaacagtacaaatttcaaccggagtccccagcgagactagcgtctgcaatattgaatgcccacaaagatggggtctgtggaattagatcaaccacgaggctggaaaatgtcatgtggaagcaaataaccaacgagctaaattatgttctctgggaaggaggacatgacctcactgtagtggctggggatgtgaagggggtgttgaccaaaggcaagagagcactcacacccccagtgaatgatctgaaatattcatggaagacatggggaaaagcaaaaatcttcaccccagaagcaagaaatagcacatttttaatagacggaccagacacctccgaatgccccaatgaacgaagagcatggaactttcttgaggtggaagactatggatttggcatgttcacgaccaacatatggatgaaattccgagaaggaagttcagaagtgtgtgaccacaggttaatgtcagcggcaattaaagatcagaaagctgtgcatgctgacatgggttattggatagagagctcaaaaaaccagacctggcagatagagaaagcatctcttattgaagtgaaaacatgtctgtggcccaagacccacacattgtggagcaatggagtgctggaaagccagatgctcattccaaaatcatatgcgggccctttttcacagcacaattaccgccagggctatgccacgcaaaccgtgggcccatggcacttaggcaaattagagatagactttggagaatgccccggaacaacagtcgcaattcaggaggattgtgaccatagaggcccatctttgaggaccaccactgcatctggaaaactagtcacgcaatggtgctgccgctcctgcacgatgcctcccttaaggttcttgggagaagatgggtgctggtatgggatggagattaggcccttgagtgaaaaagaagagaacatggtcaaatcacaggtaacggccggacagggcacatcagaaactttttctatgggtctgttgtgcctgaccttgtttgtggaagaatgcttgaggagaagagtcactaggaaacacatgatattggttgtggtgatcactctttgtgccatcatcctaggaggcctcacatggatggacttactacgagccctcatcatgttgggggacactatgtctggtagaataggaggacagatccacctagccatcatggcagtgttcaagatgtcaccaggatacgtgctgggtgtgtttttaaggaaactcacttcaagagagacagcactaatggtaataggaatggccatgacaacggtgctttcaattccacatgaccttatggaactcattgatggaatatcactggggctaattttgctaaaaatagtgacacattttgacaacacccaagtgggaaccttagccctttccttgaccttcataagatcaacaatgccattggtcatggcttggaggaccattatggctgtgttgtttgtggtcacactcattcctttgtgcaggacaagctgtcttcaaaaacagtctcattgggtagaaataacagcactcatcctaggagcccaagctctgccagtgtacctaatgactcttatgaaaggagcctcaagaagatcttggcctcttaacgagggcataatggctgtgggtttggttagtctcttaggaagcgctcttttaaagaatgatgtccctttagctggcccaatggtggcaggaggcttacttctggcggcttacgtgatgagtggtagctcagcagatctgtcactagagaaggccgccaatgtgcagtgggatgaaatggcagacataacaggctcaagcccaatcatagaagtgaagcaggatgaagatggctctttctccatacgggacgtcgaggaaaccaatatgataacccttttggtgaaactggcactgataacagtgtcaggtctctaccccttggcaattccagtcacaatgaccttatggtacatgtggcaagtgaaaacacaaagatcaggagccctgtgggacgtcccctcacccgctgccactcaaaaagccgcactgtctgaaggagtgtacaggatcatgcaaagagggttatttgggaaaactcaggttggagtagggatacacatggaaggtgtatttcacacaatgtggcatgtaacaagaggatcagtgatctgccatgagactgggagattggagccatcttgggctgacgtcaggaatgacatgatatcatacggtgggggatggagacttggagacaaatgggacaaagaagaagatgttcaggtcctcgccatagaaccaggaaaaaatcctaaacatgtccaaacgaaacccggccttttcaagaccctaactggagaaattggagcagtaacattagatttcaaacccggaacgtctggttctcccatcatcaacaggaaaggaaaagtcatcggactctatggaaatggagtagttaccaaatcaggtgattacgtcagtgccataacgcaagccgaaagaattggagagccagattatgaagtggatgaggacatttttcgaaagaaaagattaactataatggacttacaccccggagctggaaagacaaaaagaattcttccatcaatagtgagagaagccttaaaaaggaggctgcgaaccttgattttggctcccacgagagtggtggcggccgagatggaagaggccctacgtggactgccaatccgttatcagaccccagctgtgaaatcagaacacacaggaagagagattgtagacctcatgtgtcatgcaaccttcacaacaagacttttgtcatcaaccagagttccaaattacaacctcatagtgatggatgaagcacatttcaccgatccttctagtgtcgcggctagaggatacatctcgaccagggtggaaatgggagaggcagcagccatcttcatgaccgcaacccctcccggagcgacagatccctttccccagagcaacagcccaatagaagacatcgagagggaaattccggaaaggtcatggaacacagggttcgactggataacagactaccaagggaaaactgtgtggtttgttcccagcataaaagctggaaatgacattgcaaattgtttgagaaagtcgggaaagaaagttatccagttgagtaggaaaacctttgatacagagtatccaaaaacgaaactcacggactgggattttgtggtcactacagacatatctgaaatgggggccaattttagagctgggagagtgatagaccctaggagatgcctcaagccagttatcctaacagatgggccagagagagtcattttagcaggtcctattccagtgactccagcaagcgctgctcagagaagagggcgaataggaaggaacccagcacaagaagacgaccaatacgttttctccggagacccactaaaaaatgatgaagatcatgcccactggacagaagcaaagatgctgcttgacaatatctacaccccagaagggatcattccaacattgtttggtccggaaagggaaaaaacccaagccattgatggagagtttcgcctcagaggggaacaaaggaagacttttgtggaattaatgaggagaggagaccttccggtgtggctgagctataaggtagcttctgctggcatttcttacaaagatcgggaatggtgcttcacaggggaaaggaataaccaaattttagaagaaaacatggaggttgaaatttggactagagagggagaaaagaaaaagctaaggccaagatggttagatgcacgtgtatacgctgaccccatggctttgaaggattttaaggagtttgctagtggaaggaagagcataactctcgacatcctaacagagattgccagtttgccaacttacctttcctctagggccaagctcgcccttgataacatagtcatgctccacacaacagaaagaggagggagggcctaccaacacgccctgaacgaactcccggagtcactggaaacacttatgcttgtagctttactaggtgctatgacagcaggtatcttcctgtttttcatgcaagggaaaggaatagggaaattgtcaatgggtttgataaccattgcggtggctagtggcttgctctgggtagcagaaattcaaccccagtggatagcggcctcaatcatactagagttttttctcatggtactgttgataccggaaccagaaaaacaaaggaccccacaagacaatcaattgatctacgtcatattgaccattctcaccattattggtctcatagcagccaacgagatggggctgattgaaaaaacaaaaacggattttgggttttaccaggtaaaaacagaaaccaccatcctcgatgtggacttgagaccagcttcagcatggacgctctatgcagtagccaccacaattctgactcccatgctgagacacaccatagaaaacacgtcggccaacctatctctagcagccattgccaaccaggcggccgtcctaatggggcttggaaaaggatggccgctccacagaatggacctcggtgtgccgctgttagcaatgggatgctattctcaagtgaacccaacaactttgacagcatccttagtcatgcttttagtccattatgcaataataggtccaggattgcaggcaaaagccacaagagaggcccagaaaaggacagctgctgggatcatgaaaaaccccacggtggacgggataacagtaatagatctagaaccaatatcctatgacccaaaatttgaaaagcaattagggcaggtcatgctactcgtcttgtgtgctggacaactactcttgatgagaacaacatgggctttctgtgaagtcttgactttggccacaggaccaatcttgaccttgtgggagggcaacccgggaaggttttggaacacgaccatagccgtatccaccgccaacattttcaggggaagttacctggcgggagctggactggctttttcactcataaagaatgtacaaacccctaggaggggaactgggaccacaggagagacactgggagagaagtggaagagacagctaaactcattagacagaaaagagtttgaagagtataaaagaagtggaatactagaagtggacaggactgaagccaagtctgccctgaaagatgggtctaaaatcaagcatgcagtatctagagggtccagtaagattagatggattgttgagagagggatggtaaagccaaaagggaaagttgtagatcttggctgtgggagaggaggatggtcttattacatggcgacgctcaagaacgtgactgaagtgaaagggtatacaaaaggaggtccaggacatgaagaaccgattcccatggctacttatggctggaatttggtcaaactccattcaggggttgacgtgttctacaaacccacagagcaagtggacaccctgctctgtgatattggggagtcatcttctaatccaacaatagaggaaggaagaacattaagagttttgaagatggtggagccatggctctcttcaaaacctgaattctgcatcaaagtccttaacccctacatgccaacagtcatagaagagctggagaaactgcagagaaaacatggtgggaaccttgtcagatgcccgctgtccaggaactccacccatgagatgtattgggtgtcaggagcgtcgggaaacattgtgagctctgtgaacacaacatcaaagatgttgttgaacaggttcacaacaaggcataggaaacccacttatgagaaggacgtagatcttggggcaggaacgagaagtgtctccactgaaacagaaaaaccagacatgacaattattgggagaaggcttcagcgattgcaagaggagcacaaagaaacctggcattatgatcaggaaaacccatacagaacctgggcgtatcatggaagctatgaagctccttcgacaggctctgcatcctccatggtgaacggggtagtaaaactgctaacaaaaccttgggatgtggttccaatggtgacccagttagccatgacagacacaaccccttttgggcaacaaagagtgttcaaagagaaggtggataccagaacaccacaaccaaaacccggtacacgaatggttatgaccacgacagccaattggctgtgggccctccttgggaagaagaaaaatcccagactgtgcacaagggaagagttcatctcaaaagttagatcaaacgcagccataggcgcagtctttcaggaagaacagggatggacatcagccagtgaagctgtgaatgacagccggttttgggaactggttgacaaagaaagggccctacaccaggaagggaaatgtgaatcgtgtgtctacaacatgatgggaaaacgtgagaaaaagttaggagagtttggcagagccaagggaagccgagcaatctggtacatgtggctgggagcgcggtttctggaatttgaagccctgggttttttgaatgaagatcactggtttggcagagaaaattcatggagtggagtggaaggggaaggtctgcacagattgggatatatcctggaggagatagacaagaaggatggagacctaatgtatgctgatgacacagcaggctgggacacaagaatcactgaggatgaccttcaaaatgaagaactgatcacggaacagatggccccccaccacaagatcctagccaaagccattttcaaactaacctatcaaaacaaagtggtgaaagtcctcagacccacaccgagaggagcggtgatggatatcatatccaggaaagaccaaagaggtagtggacaagttggaacatatggtttgaacacattcaccaacatggaagttcaactcatccgccaaatggaagctgaaggagtcatcacacaagatgacatgcagaacccaaaagggttgaaagaaagagttgagaaatggctgaaagagtgtggtgtcgacaggttaaagaggatggcaatcagtggagacgattgcgtggtgaagcccctggatgagaggtttggcacttccctcctcttcttgaacgacatgggaaaggtgaggaaagacattccgcagtgggaaccatctaagggatggaaaaactggcaagaggttcctttttgctcccaccactttcacaagatcttcatgaaggatggccgctcactagttgttccatgtagaaaccaggatgaactgatagggagagccagaatctcgcagggggctggatggagcttaagagaaacagcctgcctgggcaaagcttacgcccagatgtggtcgctcatgtacttccacagaagggatctgcgtttagcctccatggccatatgctcagcagttccaacggaatggtttccaacaagcagaacaacatggtcaatccacgctcatcatcagtggatgaccactgaagatatgctcaaagtgtggaacagagtgtggatagaagacaaccctaatatgactgacaagactccagtccattcgtgggaagatataccttacctagggaaaagagaggatttgtggtgtggatccctgattggactttcttccagagccacctgggcgaagaacattcacacggccataacccaggtcagaaacctgatcggaaaagaggaatacgtggattacatgccagtaatgaaaagatacagcgctccttcagagagtgaaggagttctgtaattaccaacaacaaacaccaaaggctattgaagtcaggccacttgtgccacggcttgagcaaaccgtgctgcctgtagctccgccaataatgggaggcgtgaaatccctagggaggccatgcgccacggaagctgtacgcgtggcatattggactagcggttagaggagacccctcccatcactgacaaaacgcagcaaaagggggcccgaagccaggaggaagctgtactcctggtggaaggactagaggttagaggagacccccccaacacaaaaacagcatattgacgctgggaaagaccagagatcctgctgtctctgcaacatcaatccaggcacagagcgccgcaagatggattggtgttgttgatccaacaggttct

SEQ ID NO: 16 - protein, 3387 aa, Dengue virus
MNQRKKVVRPPFNMLKRERNRVSTPQGLVKRFSTGLFSGKGPLRMVLAFITFLRVLSIPPTAGILKRWGQLKKNKAIRILIGFRKEIGRMLNILNGRKRSTITLLCLIPTVMAFHLSTRDGEPLMIVAKHERGRPLLFKTTEGINKCTLIAMDLGEMCEDTVTYKCPLLVNTEPEDIDCWCNLTSTWVMYGTCTQSGERRREKRSVALTPHSGMGLETRAETWMSSEGAWKHAQRVESWILRNPGFALLAGFMAYMIGQTGIQRTVFFVLMMLVAPSYGMRCVGVGNRDFVEGVSGGAWVDLVLEHGGCVTTMAQGKPTLDFELTKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDTSNHGVTATITPRSPSVEVKLPDYGELTLDCEPRSGIDFNEMILMKMKKKTWLVHKQWFLDLPLPWTAGADTSEVHWNYKERMVTFKVPHAKRQDVTVLGSQEGAMHSALAGATEVDSGDGNHMFAGHLKCKVRMEKLRIKGMSYTMCSGKFSIDKEMAETQHGTTVVKVKYEGAGAPCKVPIEIRDVNKEKVVGRIISSTPLAENTNSATNIELEPPFGDSYIVIGVGNSALTLHWFRKGSSIGKMFESTYRGAKRMAILGETAWDFGSVGGLFTSLGKAVHQVFGSVYTTMFGGVSWMIRILIGFLVLWIGTNSRNTSMAMTCIAVGGITLFLGFTVQADMGCVVSWSGKELKCGSGIFVVDNVHTWTEQYKFQPESPARLASAILNAHKDGVCGIRSTTRLENVMWKQITNELNYVLWEGGHDLTVVAGDVKGVLTKGKRALTPPVNDLKYSWKTWGKAKIFTPEARNSTFLIDGPDTSECPNERRAWNFLEVEDYGFGMFTTNIWMKFREGSSEVCDHRLMSAAIKDQKAVHADMGYWIESSKNQTWQIEKASLIEVKTCLWPKTHTLWSNGVLESQMLIPKSYAGPFSQHNYRQGYATQTVGPWHLGKLEIDFGECPGTTVAIQEDCDHRGPSLRTTTASGKLVTQWCCRSCTMPPLRFLGEDGCWYGMEIRPLSEKEENMVKSQVTAGQGTSETFSMGLLCLTLFVEECLRRRVTRKHMILVVVITLCAIILGGLTWMDLLRALIMLGDTMSGRIGGQIHLAIMAVFKMSPGYVLGVFLRKLTSRETALMVIGMAMTTVLSIPHDLMELIDGISLGLILLKIVTHFDNTQVGTLALSLTFIRSTMPLVMAWRTIMAVLFVVTLIPLCRTSCLQKQSHWVEITALILGAQALPVYLMTLMKGASRRSWPLNEGIMAVGLVSLLGSALLKNDVPLAGPMVAGGLLLAAYVMSGSSADLSLEKAANVQWDEMADITGSSPIIEVKQDEDGSFSIRDVEETNMITLLVKLALITVSGLYPLAIPVTMTLWYMWQVKTQRSGALWDVPSPAATQKAALSEGVYRIMQRGLFGKTQVGVGIHMEGVFHTMWHVTRGSVICHETGRLEPSWADVRNDMISYGGGWRLGDKWDKEEDVQVLAIEPGKNPKHVQTKPGLFKTLTGEIGAVTLDFKPGTSGSPIINRKGKVIGLYGNGVVTKSGDYVSAITQAERIGEPDYEVDEDIFRKKRLTIMDLHPGAGKTKRILPSIVREALKRRLRTLILAPTRVVAAEMEEALRGLPIRYQTPAVKSEHTGREIVDLMCHATFTTRLLSSTRVPNYNLIVMDEAHFTDPSSVAARGYISTRVEMGEAAAIFMTATPPGATDPFPQSNSPIEDIEREIPERSWNTGFDWITDYQGKTVWFVPSIKAGNDIANCLRKSGKKVIQLSRKTFDTEYPKTKLTDWDFVVTTDISEMGANFRAGRVIDPRRCLKPVILTDGPERVILAGPIPVTPASAAQRRGRIGRNPAQEDDQYVFSGDPLKNDEDHAHWTEAKMLLDNIYTPEGIIPTLFGPEREKTQAIDGEFRLRGEQRKTFVELMRRGDLPVWLSYKVASAGISYKDREWCFTGERNNQILEENMEVEIWTREGEKKKLRPRWLDARVYADPMALKDFKEFASGRKSITLDILTEIASLPTYLSSRAKLALDNIVMLHTTERGGRAYQHALNELPESLETLMLVALLGAMTAGIFLFFMQGKGIGKLSMGLITIAVASGLLWVAEIQPQWIAASIILEFFLMVLLIPEPEKQRTPQDNQLIYVILTILTIIGLIAANEMGLIEKTKTDFGFYQVKTETTILDVDLRPASAWTLYAVATTILTPMLRHTIENTSANLSLAAIANQAAVLMGLGKGWPLHRMDLGVPLLAMGCYSQVNPTTLTASLVMLLVHYAIIGPGLQAKATREAQKRTAAGIMKNPTVDGITVIDLEPISYDPKFEKQLGQVMLLVLCAGQLLLMRTTWAFCEVLTLATGPILTLWEGNPGRFWNTTIAVSTANIFRGSYLAGAGLAFSLIKNVQTPRRGTGTTGETLGEKWKRQLNSLDRKEFEEYKRSGILEVDRTEAKSALKDGSKIKHAVSRGSSKIRWIVERGMVKPKGKVVDLGCGRGGWSYYMATLKNVTEVKGYTKGGPGHEEPIPMATYGWNLVKLHSGVDVFYKPTEQVDTLLCDIGESSSNPTIEEGRTLRVLKMVEPWLSSKPEFCIKVLNPYMPTVIEELEKLQRKHGGNLVRCPLSRNSTHEMYWVSGASGNIVSSVNTTSKMLLNRFTTRHRKPTYEKDVDLGAGTRSVSTETEKPDMTIIGRRLQRLQEEHKETWHYDQENPYRTWAYHGSYEAPSTGSASSMVNGVVKLLTKPWDVVPMVTQLAMTDTTPFGQQRVFKEKVDTRTPQPKPGTRMVMTTTANWLWALLGKKKNPRLCTREEFISKVRSNAAIGAVFQEEQGWTSASEAVNDSRFWELVDKERALHQEGKCESCVYNMMGKREKKLGEFGRAKGSRAIWYMWLGARFLEFEALGFLNEDHWFGRENSWSGVEGEGLHRLGYILEEIDKKDGDLMYADDTAGWDTRITEDDLQNEELITEQMAPHHKILAKAIFKLTYQNKVVKVLRPTPRGAVMDIISRKDQRGSGQVGTYGLNTFTNMEVQLIRQMEAEGVITQDDMQNPKGLKERVEKWLKECGVDRLKRMAISGDDCVVKPLDERFGTSLLFLNDMGKVRKDIPQWEPSKGWKNWQEVPFCSHHFHKIFMKDGRSLVVPCRNQDELIGRARISQGAGWSLRETACLGKAYAQMWSLMYFHRRDLRLASMAICSAVPTEWFPTSRTTWSIHAHHQWMTTEDMLKVWNRVWIEDNPNMTDKTPVHSWEDIPYLGKREDLWCGSLIGLSSRATWAKNIHTAITQVRNLIGKEEYVDYMPVMKRYSAPSESEGVL